(19) United States
(12) Patent Application Publication
(10) Pub. No.: US 2025/0011764 A1
(43) Pub. Date: Jan. 9, 2025

(54) IN VIVO CRISPR SCREENING SYSTEM FOR DISCOVERING THERAPEUTIC TARGETS IN CD8 T CELLS

(71) Applicant: THE TRUSTEES OF THE UNIVERSITY OF PENNSYLVANIA, Philadelphia, PA (US)

(72) Inventors: E. John Wherry, Havertown, PA (US); Zeyu Chen, Boston, MA (US); Junwei Shi, Philadelphia, PA (US); Omar Khan, San Francisco, CA (US); Josephine R. Giles, Philadelphia, PA (US); Sasikanth Manne, Havertown, PA (US)

(21) Appl. No.: 18/547,445

(22) PCT Filed: Feb. 23, 2022
(86) PCT No.: PCT/US22/17564

§ 371 Date: Aug. 22, 2023

Related U.S. Application Data
(60) Provisional application No. 63/153,191, filed on Feb. 24, 2021.

(51) Int. Cl. C12N 15/11; A61K 39/00; A61P 35/00; C12N 5/0783; C12N 9/22

(52) U.S. Cl. CPC C12N 15/11; A61K 39/4611; A61K 39/464452; A61P 35/00; C12N 5/0636; C12N 9/22; C12N 2310/20; C12N 2320/12; C12N 2510/00

(57) ABSTRACT

The present disclosure provides modified immune cells or precursors thereof comprising disrupted Fli1. Compositions and methods of treatment are also provided. The disclosure also provides methods for screening T cells, including assessing T cell exhaustion.

### CROSS-REFERENCE TO RELATED APPLICATION

The present application is entitled to priority under 35 U.S.C. § 119(e) to U.S. Provisional Patent Application No. 63/153,191, filed Feb. 24, 2021, which is hereby incorporated by reference in its entirety herein.

### STATEMENT REGARDING FEDERALLY SPONSORED RESEARCH OR DEVELOPMENT

This invention was made with government support under AI105343, AI117950, AI1082630, AI112521, AI115712, AI1108545, CA210944, CA234842, CA009140, MH109905, and HG010480 awarded by the National Institutes of Health. The government has certain rights in the invention.

### BACKGROUND OF THE INVENTION

Understanding the mechanisms that regulate effector CD8 T cell (T_{EFF}) differentiation is crucial to improve therapeutic approaches for cancer and other diseases. Activation of naïve CD8 T cells (T_{N}) during acutely resolved infections or following vaccination results in differentiation into T_{EFF} cells accompanied by transcriptional and epigenetic remodeling. After antigen clearance, a terminally differentiated subset of T_{EFF} cells dies over the ensuing days to weeks, while a small proportion of memory precursors (T_{MP}) differentiates into long-term memory CD8 T cells (T_{MEM}). During chronic infections and cancers, however, CD8 T cell differentiation is diverted down a path of exhaustion. Under these conditions, T_{EFF} cells become over-stimulated and persist poorly, whereas a population of activated precursors differentiates into exhausted CD8 T cells (T_{EX}). T_{EX} cells have high expression of multiple inhibitory receptors including PD-1, decreased effector functions, altered homeostatic regulation compared to T_{MEM} cells, and a distinct transcriptional and epigenetic program. Blocking inhibitory receptors such as PD-1 can reinvigorate T_{EX} temporarily restoring proliferation and some effector-like properties, with clinical benefit demonstrated in multiple cancer types. Despite the success of checkpoint blockade, however, most patients do not achieve durable clinical benefit and there is a great need to augment T cell differentiation and effector-like activity following checkpoint blockade or during cellular therapies in cancer or other diseases.

There has been considerable interest in defining the populations of T cells responding to checkpoint blockade and interrogating the optimal differentiation states for cellular therapies. T_{EX} cells are prominent in human tumors and likely represent a major source of tumor reactive T cells. PD-1 pathway blockade mediates clinical benefit, at least in part, due to reinvigoration of T_{EX} cells allowing these cells to re-access parts of the T_{EFF} cell program. However, limited therapeutic efficacy is associated with suboptimal reinvigoration of T_{EX} cells. Therapeutic failures for CAR T cells are also associated with exhaustion and approaches that antagonize exhaustion are actively being investigated. However, a key to both response to checkpoint blockade and cellular therapies to control cancer is the ability to effectively engage a robust effector program, including numerical expansion and elicitation of effector activity. Understanding the underlying molecular mechanisms that control this effector activity is needed to effectively design therapeutic interventions for chronic infections and cancer.

The role of transcription factors (TFs) in regulating differentiation of T_{EFF} versus T_{MEM} or T_{EX} has received considerable attention. For example, the TFs Batf and Irf4 have an early role in T cell activation and also induce the second wave of transcriptional induction of effector genes. Runx3 induces T_{EFF} gene expression through T-bet and Eomes and is important for the tissue resident memory CD8 T cells (T_{RM}). Runx3 also antagonizes a follicular-like CD8 T cell fate by inhibiting TCF-1 expression. Runx1, in contrast, is antagonized by Runx3 during T_{EFF} differentiation. Most T_{EFF}-associated genes and their cognate cis-regulatory regions are inaccessible in the T_{N} state linking the role of effector-driving TFs to chromatin accessibility changes that occur during the T_{N} to T_{EFF} transition. Indeed, there is evidence that some of these early operating TFs, such as Batf, may contribute to T_{EFF} gene accessibility through chromatin remodeling, but other mechanisms of control remain to be defined.

In addition to TF that foster T_{EFF} formation, opposing mechanisms temper complete commitment to effector differentiation to preserve more durable T cell populations for future or ongoing responses. The two alternate cell fates, T_{MEM} and T_{EX}, cannot form from fully committed T_{EFF}, suggesting that parts of the T_{EFF} program must be antagonized to allow T_{MEM} and T_{EX} to differentiate. The high mobility group (HMG) TF, TCF-1, for example, is essential for development and maintenance of both T_{MEM} and T_{EX}. TCF-1 represses T_{EFF}-driving TF such as T-bet and Blimp-1 and may foster epigenetic changes. Moreover, a second HMG TF, Tox, is essential for the development of the T_{EX} cell fate represses T_{EFF} lineage differentiation. Despite this work, mechanisms that safeguard against commitment to T_{EFF} differentiation remain poorly understood. Such information could enable immunotherapies for cancer and chronic infections. However, whereas inactivating pathways like TCF-1 or Tox that would de-repress the entire program of T_{EFF} differentiation are of interest, such approaches result in terminal T_{EFF} and may have limited therapeutic benefit because such cells cannot sustain durable responses.

Thus, there is a need in the art for the discovery of mechanisms that selectively de-repress key aspects of T_{EFF} differentiation, particularly those involved in control of numerical expansion and/or protective immunity. The present invention addresses this need.

### SUMMARY OF THE INVENTION

In one aspect, provided herein is a modified immune cell or precursor thereof, comprising a modification in an endogenous gene locus encoding Fliu.

In another aspect, provided herein is a modified immune cell or precursor thereof, wherein the endogenous Fli1 gene or protein is disrupted.

In certain embodiments, the modification or disruption is made by a method selected from the group consisting of a CRISPR system, an antibody, an siRNA, a miRNA, an antagonist, a drug, a small molecule inhibitor, a PROTAC target, a TALEN, and a Zinc Finger Nuclease.

In certain embodiments, the CRISPR system comprises at least one sgRNA comprising any one of SEQ ID NOs: 152-156 or SEQ ID NOs: 676-713.

In certain embodiments, the cell is a human cell. In certain embodiments, the cell is a T cell. In certain embodiments, T cell is resistant to T cell exhaustion.

In another aspect, provided herein is a pharmaceutical composition comprising an inhibitor of Fli1. In certain embodiments, the inhibitor is selected from the group consisting of a CRISPR system, an antibody, an siRNA, a miRNA, an antagonist, a drug, a small molecule inhibitor, a PROTAC target, a TALEN, and a Zinc Finger Nuclease. In certain embodiments, the CRISPR system comprises at least one sgRNA comprising any one of SEQ ID NOs: 152-156 or SEQ ID NOs: 676-713.

In another aspect, the invention includes a method of treating a disease or disorder in a subject in need thereof. The method comprises administering to the subject any of the cells or any of the compositions contemplated herein.

In certain embodiments, the disease or disorder is an infection. In certain embodiments, the disease is cancer.

In another aspect, provided herein is a method of screening a T cell. The method comprises i) introducing into an activated T cell a Cas enzyme (or nucleic acid encoding Cas) and an sgRNA library, ii) administering the T cell to an infected mouse, iii) isolating the T cell from the infected mouse, and iv) analyzing the T cell.

In certain embodiments, the sgRNA library comprises a plurality of sgRNAs that target a plurality of transcription factors. In certain embodiments, the plurality of transcription factors comprise any of the transcription factors listed in Table 1. In certain embodiments, each sgRNA targets the DNA binding domain of each transcription factor. In certain embodiments, the sgRNA library comprises at least one sequence selected from the group consisting of SEQ ID NOs: 1-675. In certain embodiments, the sgRNA library consists of the nucleotide sequences set forth in SEQ ID NOs: 1-675.

In certain embodiments, the screening assesses T cell exhaustion. In certain embodiments, the method identifies novel transcription factors governing T_{EFF} and T_{EX} cell differentiation.

In certain embodiments, analyzing the cell comprises a method selected from the group consisting of sequencing, PCR, MACS, and FACS. In certain embodiments, sequencing reveals a target of interest. In certain embodiments, a drug is designed against the target of interest. In certain embodiments, when the drug is administered to the T cell, at least one T cell response is increased.

In certain embodiments, 1×10⁵ T cells are administered to the infected mouse.

### BRIEF DESCRIPTION OF THE DRAWINGS

The foregoing and other features and advantages of the present disclosure will be more fully understood from the following detailed description of illustrative embodiments taken in conjunction with the accompanying drawings.

FIGS. 1A-1F: Dissecting transcriptional programs of CD8 T cells using the OpTICS system. FIG. 1A: Experimental design for Optimized T cell In vivo CRISPR Screening (OpTICS). On Day 0(D0), CD8 T cells were isolated from CD45.2⁺ C9P14 mice and activated in vitro; CD45.1V WT recipient mice were infected with LCMV. On D1 p.i., activated C9P14 cells were transduced with the RV-sgRNA library for 6 hours. On D2 p.i., Cas9+sgRNA⁺P14 cells were purified, 5-10% of the sorted cells frozen for D2 baseline (TO time point), and the rest were adoptively transferred into LCMV-infected recipient mice. Cas9⁺sgRNA⁺P14 cells were isolated from recipient mice on the indicated days by MACS and FACS (T1 time point). Targeted PCR with sequencing adaptors for the sgRNA cassettes was performed and PCR products were sequenced. The CRISPR Score (CS) was calculated as shown. FIG. 1B: The CS comparing T1 time point to T0 time point (D2 baseline) for target genes from Cas9⁺sgRNA⁺cells from spleen on D8 or D15 p.i. of Arm, D9 or D14 p.i. of C113. X-axis shows targeted genes; y-axis shows the CS of each targeted gene (using 4-5 sgRNAs). FIG. 1C: Heatmap of CS for targeted genes. Heatmap ranks the geometric means of CS for each gene. FIG. 1D: Distribution of Ctrl, Pdcd1 and Fli1 sgRNAs. Axis represents log 2 fold change (FC). Histogram shows distribution of all sgRNAs. Black bars represent targeted sgRNAs, grey bars represent all other sgRNAs. FIG. 1E: Western blot for Fli1 protein from sorted Cas9+sgFli1V P14 cells and paired Cas9+sgFli1-P14 cells from spleen. Two Fli1-sgRNAs (sgFli1_290 and sgFli1_360) were used. Pooled mice were used (3-5 mice for Arm, 10-15 mice for C113). Bar graph represents the normalized band intensity of Fli1. Fli1 was first normalized to GAPDH, then a ratio between Cas9⁺sgFli1⁺ versus Cas9+sgFli1⁻ was calculated and is shown. FIG. 1F: Normalized Cas9⁺sgRNA (VEX)⁺ cell numbers from spleen for Ctrl-sgRNA(sgCtrl) and 2 Fli1-sgRNA (sgFli1_290 and sgFli1_360) groups on D8 p.i. of Arm, D15 p.i. of Arm, D9 p.i. of C113 and D14 p.i. of C113. Cell numbers normalized to the sgCtrl group based on D2 in vitro transduction efficiency (see FIGS. 10B and 10D). *P<0.05, **P<0.01, ***P<0.001, ****P<0.001 versus control (One-Way Anova analysis). Data are representative of 4 independent experiments (mean±s.e.m.) with at least 4 mice/group for FIG. 1F.

FIGS. 2A-2E: Fli1 restrains T_{EFF} cell proliferation and differentiation during acute infection. FIG. 2A: Flow cytometry plots and statistical analysis of KLRG1^{Hi} CD127^{Lo} Terminal Effectors (TEs) and KLRG1^{Lo}CD127^{Hi} Memory Precursors (MPs). Frequencies (left) and numbers (right) from spleen for sgCtrl and 2 sgFli1 groups on D8 and D15 p.i. of Arm. Gated on Cas9(GFP)⁺ sgRNA(VEX)⁺ P14 cells. FIG. 2B: Flow cytometry plots and statistical analysis of CX3CR1⁺CXCR3⁻ T_{EFF} cells and CX3CR1⁻CXCR3⁺ early T_{MEM} cell frequencies (left) and numbers (right) from spleen for sgCtrl and 2 sgFli1 groups on D8 and D15 p.i. of Arm. Gated on Cas9⁺sgRNA⁺P14 cells. FIGS. 2C-2E: Experimental design. On D0, CD45.1⁺P14 cells were activated and recipient mice were infected with Arm; On D1 p.i., activated P14 cells were transduced with Empty-RV or Fli1-over expressing (OE)-RV. On D2 p.i., VEX⁺P14 cells were purified for each group, and 5×10⁴ cells were adoptively transferred into infected recipient mice. FIG. 2C: Flow cytometry plots of CD45.2⁺VEX⁺ cell frequency and statistical analysis of CD45.2⁺VEX⁺ cell number for Empty-RV and Fli1-OE-RV conditions. FIG. 2D: Flow cytometry plots and statistical analysis of KLRG1^{Hi}CD127 ^{Lo} TE and KLRG1^{Lo} CD127^{Hi} MP frequencies from spleen for Empty-RV and Fli1-OE-RV groups on D8 and D15 p.i. of Arm. Gated on VEX⁺ P14 cells. FIG. 2E: Flow cytometry plots and statistical analysis of CX3CR1⁺CXCR3⁻T_{EFF} cell and CX3CR1-CXCR3⁺early T_{MEM} cell frequencies from spleen for Empty-RV and Fli1-OE-RV groups on D8 and D15 p.i. of Arm. Gated on VEX⁺P14 cells. *P<0.05, **P<0.01, ***P<0.001, ****P<0.001 versus control (two-tailed Student's t-test and One-Way Anova analysis). Data are representative of 2-4 independent experiments (mean±s.e.m.) with at least 3 mice/group.

FIGS. 3A-3G: Fli1 antagonizes T_{EFF}-like cell differentiation during chronic infection. FIG. 3A: Flow cytometry plots and statistical analysis of Ly108-CD39⁺ or TCF-1-Gzmb⁺ T_{EFF}-like cells and Ly108⁺CD39⁻ or TCF-1⁺Gzmb- T_{EX} precursor frequencies from spleen for sgCtrl and 2 sgFli1 groups on D8 and D15 p.i. of C113. Gated on the Cas9(GFP)*sgRNA(VEX)⁺ P14 cells. FIG. 3B: Statistical analysis of CX3CR1⁺ and Tim-3⁺frequencies, and KLRG1 and PD-1 MFI from spleen for sgCtrl and 2 sgFli1 groups on D8 and D15 p.i. of C113. Gated on the Cas9⁺sgRNA⁺P14 cells. FIG. 3C: Heatmap of differentially expressed genes between sgCtrl and 2 sgFli1 groups. FIG. 3D: Gene Ontology (GO) enrichment analysis for the sgFli1 groups. FIG. 3E: GO enrichment analysis for the sgCtrl groups. FIG. 3F: Gene Set Enrichment Analysis (GSEA) of T_{EX} precursor signature between sgCtrl and sgFli1 groups. FIG. 3G: GSEA of T_{EFF}-like signature between sgCtrl and sgFli1 groups. *P<0.05, **P<0.01, ***P<0.001, ****P<0.001 versus control (two-tailed Student's t-test and One-Way Anova analysis). Data are representative of 4 independent experiments (mean±s.e.m.) with at least 4 mice/group for A and B.

FIGS. 4A-4K: Fli1 reshapes the epigenetic profile of CD8 T cells and inhibits T_{EFF}—associated gene expression. FIG. 4A: PCA plot of ATAC-seq data for sgCtrl, sgFli1_290 and sgFli1_360 groups on D9 p.i. of C113. FIG. 4B: Overall open chromatin region (OCR) peak changes for sgFli1 groups compared to sgCtrl group. FIG. 4C: Categories of Cis-element OCR peaks that changed between sgCtrl and sgFli1 groups. Left plot represents all changes; right plot represents changes for increased or decreased accessibility. FIG. 4D: Heatmap shows differentially accessible peaks between sgCtrl group and 2 sgFli1 groups (adjusted p-value<0.05, Log₁₀ Fold Change>0.6). Selected genes assigned to the peaks are indicated. FIG. 4E: Overlapping Venn plot of the genes with differentially accessible (DA) peaks and differentially expressed genes from FIG. 3C. FIG. 4F: Pearson correlation of the peak accessibility of the nearest genes versus the differential expression of the genes. FIG. 4G: Transcription factor (TF) motif gain or loss associated with loss of Fli1. X-axis represents the logP-value of the motif enrichment. Y-axis represents the fold change of the motif enrichment. Targeted motifs in the changed OCR between the sgCtrl and sgFli1 groups were compared to the whole genome background to calculate p-value and fold change. FIG. 4H: IgG or Fli1 binding signals from CUT&RUN on P14 cells on D8 p.i. of C113 and OCR signals detected by ATAC-seq for sgCtrl-sgRNA, sgFli1_290 and sgFli1_360 groups at the Cd28, Cx3cr1, and Havcr2 loci. FIG. 4I: Histogram of CD28 staining and statistical analysis for sgCtrl, sgFli1_290 and sgFli1_360 groups on D8 C113 p.i. Grey shows CD28 staining of CD44-naïve T cell. FIG. 4J: Heatmap shown differentially accessible (DA) peaks overlapped with the Fli1 CUT&RUN binding peaks between sgCtrl group and 2 sgFli1 groups. Selected genes assigned to the peaks are indicated. FIG. 4K: Top 4 enriched TF motifs in the Fli1 CUT&RUN peaks are shown. *P<0.05, **P<0.01 versus control (One-Way Anova analysis). Data are representative of 2 independent experiments (mean±s.e.m.) with at least 5 mice/group for FIG. 4I.

FIGS. 5A-5G: Overexpression of Runx1 or Runx3 in the context of Fli1-deficiency in CD8 T cells. FIG. 5A: Experimental design. On D0, CD8 T cells were isolated from CD45.2⁺C9P14 donor mice and activated; CD45.1V WT recipient mice were infected with C113. On D1 p.i., activated C9P14 cells were transduced with sgRNA-RV or OE-RV and 1×10⁵ transduced cells adoptively transferred into infected recipient mice. FIG. 5B-5D: Flow cytometry plots (FIG. 5B) and statistical analysis (FIG. 5C-5D) of VEX⁺mCherry+C9P14 cells and Ly108CD39⁺/Ly108⁺CD39⁻ C9P14 cells from spleen for sgCtrl-VEX⁺Empty-mCherry, sgCtrl-VEX⁺Runx1-mCherry, sgFli1_290-VEX⁺Empty-mCherry and sgFli1_290-VEX⁺Runx1-mCherry on D8 p.i. of C113. Gated on Cas9(GFP)*CD45.2⁺P14 cells. FIGS. 5E-5G: Flow cytometry plots (FIG. 5E) and statistical analysis (FIG. 5F-5G) of VEX⁺mCherry⁺ C9P14 cells and Ly108-CD39⁺/Ly108⁺CD39⁻ C9P14 cells from spleen for sgCtrl-mCherry+Empty-VEX, sgCtrl-mCherry+Runx3-VEX, sgFli1_290-mCherry+Empty-VEX and sgFli1_290-mCherry+Runx3-VEX at D8 p.i. of C113. Gated on Cas9⁺CD45.2⁺P14 cells. *P<0.05, **P<0.01, ***P<0.001, ****P<0.001 versus control (One-Way Anova analysis). Data are representative of 2 independent experiments (mean±s.e.m.) with at least 5 mice/group.

FIGS. 6A-6F: Fli1-deficiency in CD8 T cells enhances protective immunity against infections. FIG. 6A: Experimental design. On D0, CD8 T cells were isolated from CD45.2⁺ C9P14 donor mice and activated; CD45.1⁺WT recipient mice were infected with LCMV C113, influenza virus PR8-GP33 or Listeria monocytogenes-GP33 (LM-GP33). On D1 p.i., activated C9P14 cells were transduced with sgCtrl or sgFli1 RV. On D2 p.i., Cas9⁺sgRNA(VEX)⁺ P14 cells were purified by flow cytometry for sgCtrl or sgFli1 groups, and adoptively transferred into the infected recipient mice. For C113, 1.5×10⁵ VEX⁺C9P14 cells were transferred per mouse; for PR8-GP33 and LM-GP33, 1.0×10⁵ VEX⁺C9P14 cells were transferred per mouse. FIG. 6B: LCMV viral load was measured by plaque assay on D15 p.i. with C113 in liver, kidney and serum of the indicated mice. Data pooled from 2 independent experiments. FIG. 6C: Weight curve for PR8-GP33 infected mice from NT, sgCtrl⁺ cell-transferred, or sgFli1⁺ cell-transferred groups. Dashed line represents the time of C9P14 adoptive transfer. FIG. 6D: PR8-GP33 viral RNA load in the lung of NT, sgCtrl⁺ or sgFli1⁺C9P14 recipient mice. Dashed line indicates the limit of detection. Lung samples from naïve mice and spleen samples from PR8-GP33 infected mice were used as negative controls. FIG. 6E: Adjusted survival curve of LM-GP33 infected mice for NT, sgCtrl⁺ or sgFli1⁺C9P14 recipient mice. Dashed line represents the time of C9P14 adoptive transfer. FIG. 6F: LM-GP33 bacteria load in spleen and liver of the surviving NT, sgCtrl⁺ or sgFli1⁺C9P14 recipient mice on D7 p.i. *P<0.05, **P<0.01, ***P<0.001, ****P<0.001 versus control (One-Way Anova analysis for 6B-6D, 6F and Mantel-Cox test for 6E). Data are representative of 2 independent experiments (mean±s.e.m.) with at least 3 mice/group.

FIGS. 7A-7G: Loss of Fli1 in CD8 T cells improved anti-tumor immunity. FIG. 7A: Experimental design. On D0, CD45.2⁺Rag2−/− mice were inoculated with 1.0×10⁵ B16-Dbgp33 cells. On D3 post tumor inoculation (p.t.), CD8 T cells were isolated from CD45.1⁺C9P14 mice and activated. On D4 p.t., activated C9P14 cells were transduced with sgCtrl or sgFli1 RVs. On D5 p.t., sgRNA (VEX)⁺Cas9 (GFP)*P14 cells were sorted from sgCtrl or sgFli1 groups, and 1×10⁶ purified VEX⁺C9P14 cells were adoptively transferred into tumor-bearing mice. FIG. 7B: Tumor volume curve for mice receiving NT, sgCtrl⁺ or sgFli1⁺ C9P14 cells. FIG. 7C: Tumor weight on D23 p.t. for mice receiving NT, sgCtrl⁺ or sgFli1l C9P14 cells. FIGS. 7D-7E: Flow cytometry plots (FIG. 7D) and statistical analysis (FIG. 7E) of CD45.1⁺sgRNA(VEX)⁺Cas9⁺P14 cells and Ly108-CD39⁺/Ly108⁺CD39⁻ C9P14 cells from tumor for sgCtrl or sgFli1 groups on D23 p.t. FIGS. 7F-7G: Flow cytometry plots (FIG. 7F) and statistical analysis (FIG. 7G) of CD45.1⁺sgRNA⁺Cas9⁺ P14 cells and Ly108-CD39⁺/Ly108⁺CD39⁻ C9P14 cells from spleen for sgCtrl or sgFli1 groups at D23 p.t. *P<0.05, **P<0.01, ***P<0.001, ****P<0.001 versus control (two-tailed Student's t-test and One-Way Anova analysis). Data are representative of 2 independent experiments (mean±s.e.m.) with at least 5 mice/group.

FIGS. 8A-8K: High-efficiency in vivo gene editing and screening using retroviral-transduced sgRNA in Cas9⁺ antigen specific CD8 T cells. FIG. 8A: Optimizing sgRNA backbone compared to the original sgRNA. FIG. 8B: Experimental design for in vivo gene editing test. At Day 0 (D0), CD8 T cells were isolated from CD45.1⁺LSL-Cas9⁺CD4^{CRE+}P14 (C9P14) donor mice and activated with anti-CD3, anti-CD28 and IL-2; CD45.2⁺recipient mice were infected with C113. On D1 p.i., activated C9P14 cells were transduced with either Ctrl-sgRNA(sgCtrl) or Pdcd1-sgRNA (sgPdcd1). Six hours after transduction, 5×10⁴ activated donor cells were adoptively transferred into infected recipient mice. C9P14 cells were then isolated, at the indicated times, from different organs of recipient mice for analysis. FIG. 8C: D2 in vitro transduction efficiency of activated C9P14 cells with sgRNA vector (mCherry). Gates are set based on the non-transduced control. FIG. 8D: Flow cytometry plots of the Cas9(GFP)⁺sgRNA(mCherry)⁺ population in the spleen at D9 p.i. for the sgCtrl group and sgPdcd1 groups. FIG. 8E: Histogram of PD-1 expression and statistical analysis of the PD-1⁺population from the Cas9⁺sgRNA+P14 cells in the blood (D7 p.i.), spleen (D9 p.i.) or liver (D9 p.i.). FIG. 8F: Sanger sequencing results for the Pdcd1 locus from FACS purified Cas9⁺sgRNA+P14 cells from the sgCtrl group (pooled 5 mice) or the sgPdcd1 group (pooled 2 mice). FIG. 8G: Histogram of KLRG1 or CXCR3 expression and statistical analysis of KLRG1⁺or CXCR3⁺populations from Cas9⁺sgRNA+P14 cells from the spleen (D8 p.i. of Arm) between targeted-sgRNA and sgCtrl groups. FIG. 8H: Log2 fold change (L2FC) of D8 p.i.(T1) to D2 baseline(TO) across different sgRNAs from spleen or liver under 3 conditions during Arm infection. The x-axis represents different sgRNAs, y-axis represents L2FC of D8 p.i.(T1) to D2 baseline(TO). Condition 1: No optimized sorting, average input coverage ˜100 cells/sgRNA, Cas9⁺/⁺P14 donor. Condition 2: Optimized sorting (in Materials and Methods), average input coverage ˜400 cells/sgRNA, Cas9⁺/⁺ P14 donor. Condition 3: Optimized sorting, average input coverage ˜400 cells/sgRNA, Cas9⁺/⁺ P14 donor. Example target genes are highlighted with the indicated color. FIG. 8I: Rank correlation of targeted genes between 2 independent screenings of Cas9⁺/⁺ or Cas9⁺/⁺ donor P14 groups. The mean values of sgRNA L2FC from each targeted gene were calculated and ranked from the independent screenings. Pearson correlation of the rankings was calculated. FIG. 8J: Fold change enrichment of sgPdcd1 at D14 p.i. of C113 in the spleen. Data from the screening performed in FIGS. 1A-1C. FIG. 8K: Pearson correlation of different samples from the screening performed in FIGS. 1A-1C. Sample collection time (days p.i.), LCMV infection and organ of sorted Cas9⁺sgRNA⁺cells are presented. *P<0.05, **P<0.01, ***P<0.001, ****P<0.001 versus control (two-tailed Student's t-test). Data are representative of 2 independent experiments (mean±s.e.m.) with at least 3 mice/group.

FIGS. 9A-9E: Genetic deletion of Fli1 leads to a greater T cell expansion. FIG. 9A: TIDE assay results showing genome disruption efficiency of the Fli1 locus for the Cas9⁺Fli1-sgRNA (sgFli1)⁺ cells. Genome disruption was detected by Sanger sequencing. FIG. 9B: Flow cytometry plots (gated on donor P14 cells) of the Cas9 (GFP)*sgRNA(VEX)⁺ population on D2 after in vitro transduction; D8 and D15 p.i. of Arm from splenocytes from sgCtrl or 2 Fli1-sgRNA(sgFli1_290 and sgFli1_360) groups. 5×104 activated donor cells were adoptively transferred into congenic infected recipient mice on D1 p.i. FIG. 9C: Normalized Cas9⁺sgRNA⁺cell numbers from blood, liver and lung from sgCtrl and the two sgFli1 group at D8 and D15 p.i. of Arm. Cell numbers normalized to the sgCtrl group based on D2 in vitro transduction efficiency. FIG. 9D: Flow cytometry plots (gated on donor P14 cells) of Cas9⁺sgRNA⁺P14 cells on D2 post in vitro transduction, D9 and D15 p.i. with C113 (speen) for sgCtrl and the two sgFli1 groups. 5×10⁴ activated donor P14 cells were adoptively transferred into the infected recipient mice on D1 p.i. FIG. 9E: Normalized Cas9⁺sgRNA⁺cell numbers from blood, liver and lung from sgCtrl and the two sgFli1 groups at D9 and D15 p.i. of C113. Cell number normalized to the sgCtrl group based on D2 in vitro transduction efficiency. *P<0.05, **P<0.01, ***P<0.001, ****P<0.001 versus control (One-Way Anova analysis). Data are representative of 2-4 independent experiments (mean±s.e.m.) with at least 3 mice/group.

FIGS. 10A-10J: Fli1 inhibits terminal T_{EFF} differentiation without impacting T_{MEM} cell formation. FIG. 10A: Flow cytometry plots and statistical analysis of KLRG1^{Lo}CD127^{Lo} cells. Frequencies (left) and numbers (right) from spleen for sgCtrl and 2 sgFli1 groups on D8 and D15 p.i. of Arm. Gated on Cas9(GFP)⁺ sgRNA (VEX)⁺ P14 cells. FIG. 10B: Flow cytometry plots and statistical analysis of GzmB+ and TCF-1⁺ C9P14 cells. Frequencies from spleen for sgCtrl and sgFli1 (2 sgRNA combined) groups on D8 p.i. of Arm. Gated on Cas9(GFP)⁺ sgRNA (VEX)⁺P14 cells. FIG. 10C: Histogram and statistical analysis of T-bet and Eomes expression in C9P14 cells from spleen for sgCtrl and sgFli1 (2 sgRNA combined) groups on D8 p.i. of Arm. Gated on Cas9(GFP)⁺ sgRNA (VEX)⁺P14 cells. Naïve T cell (CD44−) staining shown in grey. FIG. 10D: Flow cytometry plots and statistical analysis of cytokine producing C9P14 cells after 5 hours of stimulation. IFNγ⁺, IFNγ⁺TNF⁺ and MIP1α⁺CD107a⁺ frequencies (left) and numbers (right) from spleen for non-stim P14 cells, sgCtrl and sgFli1 (2 sgRNA combined) groups on D8 p.i. of Arm. Gated on Cas9(GFP)⁺ sgRNA (VEX)⁺ P14 cells. FIG. 10E: Statistical analysis of total, terminal effector (TE) or memory precursor (MP) C9P14 cell numbers in the blood of sgCtrl and sgFli1 (2 sgRNA combined) groups on D8, D20 and D29 p.i. with Arm. Data normalized to sgCtrl⁺ D2 p.i. transduction efficiency. Normalized sgRNA⁺C9P14 cell number is around 75 cells/1×10⁶ PBMCs on the transfer day (D1 p.i.). FIG. 10F: Statistical analysis of TE or MP C9P14 frequency and total, TE or MP C9P14 cell number in the spleen of sgCtrl and sgFli1 (2 sgRNA combined) groups on D29 p.i. of Arm. Data normalized to sgCtrl⁺ D2 p.i. transduction efficiency. FIG. 10G: Histograms of Bcl-2, Bcl-XL and Bim expression of total C9P14 cells from spleen of sgCtrl and sgFli1 (2 sgRNA combined) groups on D15 p.i. of Arm. Gated on Cas9(GFP)⁺ sgRNA (VEX)⁺ P14 cells. Naïve T cell (CD44⁻) staining shown in grey. FIGS. 10H-10J: Statistical analysis of Bcl-2, Bcl-XL, Bim expression and Bcl-2/Bim and Bcl-XL/Bim ratios for total (FIG. 10H), TE (FIG. 101) and MP(FIG. 10J) C9P14 cells from spleen for sgCtrl and sgFli1 (2 sgRNA combined) groups on D15 p.i. with Arm. Gated on Cas9(GFP)⁺ sgRNA (VEX)⁺ P14 cells. *P<0.05, **P<0.01, ***P<0.001, ****P<0.001 versus control (One-Way Anova analysis). Data are representative of 2 independent experiments (mean±s.e.m.) with at least 4 mice/group.

FIGS. 11A-11H: Fli1 restrains T_{EFF}-like differentiation during chronic infection. FIGS. 11A-11B: Statistical analysis of Ly108-CD39⁺ or TCF-1-GzmB+T_{EFF}-like cells and Ly108⁺CD39⁻ or TCF-1⁺GzmB TExprecursor cell numbers from spleen for sgCtrl and the two sgFli1 groups at D8 (FIG. 11A) and D15 (FIG. 11B) p.i. with C113. Gated on Cas9(GFP)⁺ sgRNA (VEX)⁺ P14 cells. FIG. 11C: Histogram and statistical analysis of Eomes, T-bet and Tox expression in C9P14 cells from spleen for sgCtrl and sgFli1 groups on D8 and D15 p.i. with C113. Gated on Cas9(GFP)⁺ sgRNA (VEX)⁺ P14 cells. Naïve T cell (CD44−) staining shown in grey. FIG. 11D: Flow cytometry plots and statistical analysis of cytokine producing C9P14 cells after 5 hours of stimulation. IFNγ⁺, IFNγ⁺TNF⁺ and MIP1α⁺CD107a⁺frequencies (left) and numbers (right) from spleen for non-stim P14 cells, sgCtrl and sgFli1 (2 sgRNA combined) groups on D8 p.i. with C113. Gated on Cas9(GFP)⁺ sgRNA (VEX)⁺ P14 cells. FIG. 11E: Flow cytometry plots and statistical analysis of cell number of CD45.2⁺VEX⁺P14 for Empty-RV and Fli1-OE-RV groups at D8 and D16 p.i. with C113. At D0, CD45.2⁺P14 cells were activated and CD45.1V recipient mice were infected with C113. On D1 p.i., activated P14 were transduced with either Empty-RV or Fli1-OE-RV for 6 hours. On D2 p.i., VEX⁺P14 cells were sorted from each RV transduced group and 1×10⁵ cells were adoptively transferred into the infected recipients.

FIGS. 11F-11G: Flow cytometry plots and statistical analysis of Ly108-CD39⁺ or TCF-1-Gzmb+T_{EFF}-like cells and Ly108⁺CD39⁻ or TCF-1⁺Gzmb- T_{EX} precursor frequencies for Empty-RV and Fli1-OE-RV groups on D8 and D16 p.i. of C113. Gated on VEX⁺P14 cells. FIG. 11H: Statistical analysis of CX3CR1⁺ and Tim-3⁺frequencies for Empty-RV and Fli1-OE-RV groups on D8 and D16 p.i. of C113. Gated on VEX⁺P14 cells. *P<0.05, **P<0.01, ***P<0.001 versus control (One-Way Anova analysis). Data are representative of 3 independent experiments (mean±s.e.m.) with at least 4 mice/group for FIGS. 11A-11D.

FIGS. 12A-12I: Transcriptional and epigenetic profiling addresses Fli1 inhibiting T_{EFF}-like differentiation by coordinating with Runx1 and antagonizing Runx3 function. FIG. 12A: PCA plot for RNA-seq data for sgCtrl, sgFli1_290 and sgFli1_360 groups on D8 p.i. with C113.

FIG. 12B: Overlap of all CUT&RUN Fli1 binding peaks with ATAC-seq detected peaks in sgCtrl and sgFli1 groups. FIG. 12C: Histogram of all CUT&RUN peaks co-localized with the ATAC-seq peaks. Peaks co-localized with the ATAC-seq peaks are grey; peaks not co-localized are black. FIG. 12D: CUT&RUN IgG or Fli1 binding signals for P14 cells on D9 p.i. and open chromatin region signals detected by ATAC-seq for sgCtrl, sgFli1_290 and sgFli1_360 groups in the Tcf7 and Id3 loci. FIG. 12E: Flow cytometry plots and statistical analysis of CD45.1⁺mCherry+ and Ly108-CD39⁺/Ly108⁺CD39⁻ P14 cell numbers for Empty-RV or Runx1-OE-RV on D2 in vitro and D7 p.i. On D0, CD45.1⁺P14 cells were activated and CD45.2⁺ recipient mice were infected with C113; On D1 p.i., activated P14 cells were transduced with Empty-RV or Runx1-OE-RV for 6 hours, and 1×10⁵ transduced P14 cells were adoptively transferred into infected recipient mice. Flow cytometry plots gated on the CD45.1⁺ P14 cells. FIG. 12F: D2 in vitro transduction efficiency of sgCtrl-VEX⁺Empty-mCherry, sgCtrl-VEX+Runx1-mCherry, sgFli1_290-VEX⁺Empty-mCherry, and sgFli1_290-VEX⁺Runx1-mCherry C9P14 cells. FIG. 12G: Statistical analysis of VEX⁺mCherry⁺ C9P14 cells and Ly108-CD39⁺/Ly108⁺CD39⁻ cell numbers from spleen for sgCtrl-VEX+Empty-mCherry, sgCtrl-VEX+Runx1-mCherry, sgFli1_290-VEX⁺Empty-mCherry, and sgFli1_290-VEX+Runx1-mCherry groups on D7 p.i. with C113. Gated on Cas9(GFP)*CD45.2⁺P14 cells. FIG. 12H: D2 in vitro transduction efficiency of sgCtrl-mCherry+Empty-VEX, sgCtrl-mCherry+Runx3-VEX, sgFli1_290-mCherry+Empty-VEX, and sgFli1_290-mCherry+Runx3-VEX C9P14 cells. FIG. 12I: Statistical analysis of VEX⁺mCherry⁺ C9P14 cells and Ly108⁻CD39*/Ly108*CD39⁻ cell numbers from spleen for sgCtrl−mCherry+Empty−VEX, sgCtrl−mCherry+Runx3−VEX, sgFli1_290-mCherry+Empty-VEX and sgFli1_290-mCherry+Runx3-VEX at D8 p.i. with C113. Gated on Cas9⁺CD45.2⁺P14 cells. *P<0.05, **P<0.01 versus control (two-tailed Student's t-test). Data are representative of 2 independent experiments (mean±s.e.m.) with at least 5 mice/group.

FIGS. 13A-13E: Fli1-deficiency results in CD8 T cell expansion during influenza virus or Listeria monocytogenes infection. FIG. 13A: Adjusted survival curve of LCMV C113 infected mice for sgCtrl⁺, sgFli1_290′ and sgFli1_360⁺C9P14 recipient mice (1.5×10⁵ cell/mouse, N=5 per group) Note, JAX recipient mice were used here instead of NCI recipients resulting in the difference in pathogenesis. Dashed line represents the time of C9P14 adoptive transfer. FIG. 13B: Flow cytometry plots showing sgRNA(VEX)⁺ C9P14 cells in the lungs of influenza virus (PR8-GP33) infected mice, comparing non-transfer(NT), sgCtrl and sgFli1 groups at D8 p.i. “Recovered” was defined by complete weight recovery on D8 p.i. FIG. 13C: Correlation of sgRNA⁺C9P14 cell numbers and weight ratio (D8 p.i./D2 p.i.) during the PR8-GP33 infection. In the “weight not recovered” group (weight ratio between 0.7 and 1.0), sgRNA⁺C9P14 cell numbers were further compared. FIG. 13D: Flow cytometry plots and statistical analysis of sgRNA⁺ C9P14 cells in the spleens of PR8-GP33-infected recipient mice for non-transfer (NT), sgCtrl and sgFli1 groups on D8 p.i. FIG. 13E: Flow cytometry plots and statistical analysis of sgRNA⁺C9P14 cells in the spleens of Listeria monocytogenes-infected recipients for non-transfer (NT), sgCtrl and sgFli1 groups on D7 p.i. *P<0.05, **P<0.01 versus control (two-tailed Student's t-test and One-Way Anova analysis). Data are representative of 2 independent experiments (mean±s.e.m.) with at least 6 mice/group.

FIGS. 14A-14G: Deleting Fli1 in CD8 T cells leads to better tumor protection in an immune competent mice. FIG. 14A: Experimental design. On D0, CD45.2⁺Cas9⁺P14- mice were inoculated with 2×10⁵ B16-Dbgp33 cells. On D3 post tumor inoculation (p.t.), CD8 T cells were isolated from CD45.2⁺ C9P14 donor mice and activated. The next day, activated C9P14 cells were transduced with sgCtrl or sgFli1 RV for 6 hours. On D5 p.t., sgRNA (VEX)⁺ P14 cells were sorted from sgCtrl or sgFli1 groups, and 3×10⁶ purified VEX⁺C9P14 cells were adoptively transferred into tumor-bearing mice. FIG. 14B: Tumor volume curve of tumor-bearing mice from NT, sgCtrl⁺ and sgFli1V C9P14 cell transferred groups. FIG. 14C: Tumor weight from NT, sgCtrl⁺ and sgFli1⁺ C9P14 transferred mice on D24 p.t. FIGS. 14D-14E: Flow cytometry plots (FIG. 14D) and statistical analysis (FIG. 14E) of sgRNA (VEX)⁺ C9P14 cells and Ly108-CD39⁺/Ly108⁺CD39⁻ populations from tumor for sgCtrl and sgFli1 groups on D24 p.t. FIGS. 14F-14G: Statistical analysis of sgRNA⁺C9P14 cells and Ly108-CD39⁺ or Ly108⁺CD39-populations from draining lymph node (dLN, FIG. 14F) and spleen (FIG. 14G) for sgCtrl and sgFli1 groups on D24 p.t. *P<0.05, **P<0.01, ***P<0.001, ****P<0.001 versus control (two-tailed Student's t-test and One-Way Anova analysis). Data are representative of 3 independent experiments (mean±s.e.m.) with at least 6 mice/group.

### DETAILED DESCRIPTION

Improving effector activity of antigen specific T cells is a major goal in cancer immunotherapy. Despite the identification of several effector T cell (T_{EFF})-driving transcription factors (TF), the transcriptional coordination of T_{EFF} biology remains poorly understood. Herein, an in vivo T cell CRISPR screening platform was developed. A novel mechanism was identified that restrains T_{EFF} biology through the ETS family TF, Fli1. Genetic deletion of Fli1 enhanced T_{EFF} responses without compromising memory or exhaustion precursors. Fli1 restrained T_{EFF} lineage differentiation by binding to cis-regulatory elements of effector-associated genes. Loss of Fli1 increased chromatin accessibility at ETS:RUNX motifs allowing more efficient Runx3-driven T_{EFF} biology. CD8 T cells lacking Fli1 provided substantially better protection against multiple infections and tumors. These data indicate that Fli1 safeguards the developing CD8 T cell transcriptional landscape from excessive ETS:RUNX-driven T_{EFF} cell differentiation. Moreover, genetic deletion of Fli1 improves T_{EFF} differentiation and protective immunity in infections and cancer.

It is to be understood that the methods described in this disclosure are not limited to particular methods and experimental conditions disclosed herein as such methods and conditions may vary. It is also to be understood that the terminology used herein is for the purpose of describing particular embodiments only, and is not intended to be limiting.

Furthermore, the experiments described herein, unless otherwise indicated, use conventional molecular and cellular biological and immunological techniques within the skill of the art. Such techniques are well known to the skilled worker, and are explained fully in the literature. See, e.g., Ausubel, et al., ed., Current Protocols in Molecular Biology, John Wiley & Sons, Inc., NY, N.Y. (1987-2008), including all supplements, Molecular Cloning: A Laboratory Manual (Fourth Edition) by MR Green and J. Sambrook and Harlow et al., Antibodies: A Laboratory Manual, Chapter 14, Cold Spring Harbor Laboratory, Cold Spring Harbor (2013, 2nd edition).

### A. Definitions

Unless otherwise defined, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

Generally, nomenclature used in connection with cell and tissue culture, molecular biology, immunology, microbiology, genetics and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the disclosure may be more readily understood, select terms are defined below.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“Activation,” as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

As used herein, to “alleviate” a disease means reducing the severity of one or more symptoms of the disease.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen.

Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “autologous” is meant to refer to any material derived from the same individual to which it is later to be re-introduced into the individual.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to T cell proliferation and/or upregulation or downregulation of key molecules.

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The term “downregulation” as used herein refers to the decrease or elimination of gene expression of one or more genes.

“Effective amount” or “therapeutically effective amount” are used interchangeably herein, and refer to an amount of a compound, formulation, material, or composition, as described herein effective to achieve a particular biological result or provides a therapeutic or prophylactic benefit. Such results may include, but are not limited to an amount that when administered to a mammal, causes a detectable level of immune suppression or tolerance compared to the immune response detected in the absence of the composition of the present disclosure. The immune response can be readily assessed by a plethora of art-recognized methods. The skilled artisan would understand that the amount of the composition administered herein varies and can be readily determined based on a number of factors such as the disease or condition being treated, the age and health and physical condition of the mammal being treated, the severity of the disease, the particular compound being administered, and the like.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

The term “epitope” as used herein is defined as a small chemical molecule on an antigen that can elicit an immune response, inducing B and/or T cell responses. An antigen can have one or more epitopes. Most antigens have many epitopes; i.e., they are multivalent. In general, an epitope is roughly about 10 amino acids and/or sugars in size. Preferably, the epitope is about 4-18 amino acids, more preferably about 5-16 amino acids, and even more most preferably 6-14 amino acids, more preferably about 7-12, and most preferably about 8-10 amino acids. One skilled in the art understands that generally the overall three-dimensional structure, rather than the specific linear sequence of the molecule, is the main criterion of antigenic specificity and therefore distinguishes one epitope from another. Based on the present disclosure, a peptide used in the present disclosure can be an epitope.

As used herein, the term “exogenous” refers to any material introduced from or produced outside an organism, cell, tissue or system.

The term “expand” as used herein refers to increasing in number, as in an increase in the number of T cells. In one embodiment, the T cells that are expanded ex vivo increase in number relative to the number originally present in the culture. In another embodiment, the T cells that are expanded ex vivo increase in number relative to other cell types in the culture. The term “ex vivo,” as used herein, refers to cells that have been removed from a living organism, (e.g., a human) and propagated outside the organism (e.g., in a culture dish, test tube, or bioreactor).

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Identity” as used herein refers to the subunit sequence identity between two polymeric molecules particularly between two amino acid molecules, such as, between two polypeptide molecules. When two amino acid sequences have the same residues at the same positions; e.g., if a position in each of two polypeptide molecules is occupied by an arginine, then they are identical at that position. The identity or extent to which two amino acid sequences have the same residues at the same positions in an alignment is often expressed as a percentage. The identity between two amino acid sequences is a direct function of the number of matching or identical positions; e.g., if half (e.g., five positions in a polymer ten amino acids in length) of the positions in two sequences are identical, the two sequences are 50% identical; if 90% of the positions (e.g., 9 of 10), are matched or identical, the two amino acids sequences are 90% identical.

The term “immune response” as used herein is defined as a cellular response to an antigen that occurs when lymphocytes identify antigenic molecules as foreign and induce the formation of antibodies and/or activate lymphocytes to remove the antigen.

The term “immunosuppressive” is used herein to refer to reducing overall immune response.

“Insertion/deletion”, commonly abbreviated “indel,” is a type of genetic polymorphism in which a specific nucleotide sequence is present (insertion) or absent (deletion) in a genome.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “knockdown” as used herein refers to a decrease in gene expression of one or more genes.

The term “knockin” as used herein refers to an exogenous nucleic acid sequence that has been inserted into a target sequence (e.g., endogenous gene locus. In some embodiments, where the target sequence is a gene, a knockin is generated resulting in the exogenous nucleic acid sequence being in operable linkage with any upstream and/or downstream regulatory elements controlling expression of the target gene. In some embodiments, the knockin is generated resulting in the exogenous nucleic acid sequence not being in operable linkage with any upstream and/or downstream regulatory elements controlling expression of the target gene.

The term “knockout” as used herein refers to the ablation of gene expression of one or more genes.

A “lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

By the term “modified” as used herein, is meant a changed state or structure of a molecule or cell of the present disclosure. Molecules may be modified in many ways, including chemically, structurally, and functionally. Cells may be modified through the introduction of nucleic acids.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

The term “oligonucleotide” typically refers to short polynucleotides. It will be understood that when a nucleotide sequence is represented by a DNA sequence (i.e., A, T, C, G), this also includes an RNA sequence (i.e., A, U, C, G) in which “U” replaces “T.”

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

By the term “stimulation,” is meant a primary response induced by binding of a stimulatory molecule (e.g., a TCR/CD3 complex) with its cognate ligand thereby mediating a signal transduction event, such as, but not limited to, signal transduction via the TCR/CD3 complex. Stimulation can mediate altered expression of certain molecules, such as downregulation of TGF-beta, and/or reorganization of cytoskeletal structures, and the like.

A “stimulatory molecule,” as the term is used herein, means a molecule on a T cell that specifically binds with a cognate stimulatory ligand present on an antigen presenting cell.

A “stimulatory ligand,” as used herein, means a ligand that when present on an antigen presenting cell (e.g., an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, inter alia, an MHC Class I molecule loaded with a peptide, an anti-CD3 antibody, a superagonist anti-CD28 antibody, and a superagonist anti-CD2 antibody.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

A “target site” or “target sequence” refers to a nucleic acid sequence that defines a portion of a nucleic acid to which a binding molecule may specifically bind under conditions sufficient for binding to occur. In some embodiments, a target sequence refers to a genomic nueleic acid sequence that defines a portion of a nucleic acid to which a binding molecule may specifically bind under conditions sufficient for binding to occur.

The term “therapeutic” as used herein means a treatment and/or prophylaxis. A therapeutic effect is obtained by suppression, remission, or eradication of a disease state.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver. A transplant can also refer to any material that is to be administered to a host. For example, a transplant can refer to a nucleic acid or a protein.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

To “treat” a disease as the term is used herein, means to reduce the frequency or severity of at least one sign or symptom of a disease or disorder experienced by a subject.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various aspects of the disclosure can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### B. Modified Immune Cells

Provided herein are modified immune cells or precursors thereof (e.g., T cells) wherein the endogenous Fli1 has been disrupted. Endogenous Fli1 can be disrupted at the gene or protein level by any means known to one of ordinary skill in the art. Such methods of disrupting Fli1 include but are not limited to a CRISPR system, an antibody, a siRNA, a miRNA, a drug, an antagonist, a small molecule inhibitor, and a PROTAC target.

In one aspect, the disclosure provides modified immune cells or precursors thereof (e.g., T cells) comprising a modification in an endogenous gene locus encoding Fli1. In certain embodiments, the cell comprises a nucleic acid capable of downregulating gene expression of endogenous Fli1.

In one aspect, the disclosure provides a modified immune cell or precursor thereof (e.g., T cell) comprising a CRISPR-mediated modification in an endogenous gene locus encoding Fli1 that is capable of downregulating gene expression of endogenous Fli1.

In certain embodiments, the modified cell is a human cell.

The present disclosure provides gene edited modified cells. In some embodiments, a modified of the present disclosure is genetically edited to disrupt the expression of an endogenous gene locus encoding Fli1. In some embodiments, the gene-edited immune cells (e.g., T cells) have a downregulation, reduction, deletion, elimination, knockout or disruption in expression of the endogenous Fli1.

Immunotherapies have shown various efficacies in the treatment of cancer patients. One of the major problems limiting their effects is that T cells are exhausted after persistent stimulation by tumor cells. Exhausted T cells have reduced effector functions such as production of cytokines and cytotoxicity against tumor cells, and they express higher levels of checkpoint inhibitory molecules, such as PD-1 and CTLA-4. PD-1 and CTLA-4 antibodies have been used clinically to treat multiple types of cancers.

In some embodiments, the modified cell of the present disclosure is genetically edited to disrupt the expression of an additional endogeneous gene. For example, the cell may be further edited to disrupt an endogenous PDCD1 gene product (e.g. Programmed Death 1 receptor; PD-1). Disrupting the expression of endogenous PD-1 may create “checkpoint” resistant modified cells, resulting in increased tumor control. Checkpoint resistant modified cells may also be created by disrupting the expression of, for example, without limitation, the Adenosine A2A receptor (A2AR), B7-H3 (CD276), B7-H4 (VTCN1), the B and T Lymphocyte Attenuator protein (BTLA/CD272), CD96, the Cytotoxic T-Lymphocyte Associated protein 4 (CTLA-4/CD152), Indoleamine 2,3-dioxygenase (IDO), the Killer-cell Immunoglobulin-like Receptor (KIR), the Lymphocyte Activation Gene-3 (LAG3), the T cell immunoreceptor with Ig and ITIM domains (TIGIT), T-cell Immunoglobulin domain and Mucin domain 3 (TIM-3), or the V-domain Ig suppressor of T cell activation (VISTA).

Various gene editing technologies are known to those skilled in the art. Gene editing technologies include, without limitation, homing endonucleases, zinc-finger nucleases (ZFNs), transcription activator-like effector (TALE) nucleases (TALENs), and clustered regularly interspaced short palindromic repeats (CRISPR)-associated protein 9 (Cas9). Homing endonucleases generally cleave their DNA substrates as dimers, and do not have distinct binding and cleavage domains. ZFNs recognize target sites that consist of two zinc-finger binding sites that flank a 5- to 7-base pair (bp) spacer sequence recognized by the FokI cleavage domain. TALENs recognize target sites that consist of two TALE DNA-binding sites that flank a 12- to 20-bp spacer sequence recognized by the FokI cleavage domain. The Cas9 nuclease is targeted to DNA sequences complementary to the targeting sequence within the single guide RNA (gRNA) located immediately upstream of a compatible protospacer adjacent motif (PAM). Accordingly, one of skill in the art would be able to select the appropriate gene editing technology for the present disclosure.

In some aspects, the disruption is carried out by gene editing using an RNA-guided nuclease such as a CRISPR-Cas system, such as CRISPR-Cas9 system, specific for the gene (e.g., Fli1) being disrupted. In some embodiments, an agent containing a Cas9 and a guide RNA (gRNA) containing a targeting domain, which targets a region of the genetic locus, is introduced into the cell. In some embodiments, the agent is or comprises a ribonucleoprotein (RNP) complex of a Cas9 polypeptide and a gRNA (Cas9/gRNA RNP). In some embodiments, the introduction includes contacting the agent or portion thereof with the cells in vitro, which can include cultivating or incubating the cell and agent for up to 24, 36 or 48 hours or 3, 4, 5, 6, 7, or 8 days. In some embodiments, the introduction further can include effecting delivery of the agent into the cells. In various embodiments, the methods, compositions and cells according to the present disclosure utilize direct delivery of ribonucleoprotein (RNP) complexes of Cas9 and gRNA to cells, for example by electroporation. In some embodiments, the RNP complexes include a gRNA that has been modified to include a 3′ poly- A tail and a 5′ Anti-Reverse Cap Analog (ARCA) cap.

The CRISPR/Cas9 system is a facile and efficient system for inducing targeted genetic alterations. Target recognition by the Cas9 protein requires a ‘seed’ sequence within the guide RNA (gRNA) and a conserved di-nucleotide containing protospacer adjacent motif (PAM) sequence upstream of the gRNA-binding region. The CRISPR/Cas9 system can thereby be engineered to cleave virtually any DNA sequence by redesigning the gRNA in cell lines (such as 293T cells), primary cells, and TCR T cells. The CRISPR/Cas9 system can simultaneously target multiple genomic loci by co-expressing a single Cas9 protein with two or more gRNAs, making this system suited for multiple gene editing or synergistic activation of target genes.

The Cas9 protein and guide RNA form a complex that identifies and cleaves target sequences. Cas9 is comprised of six domains: REC I, REC II, Bridge Helix, PAM interacting, HNH, and RuvC. The REC I domain binds the guide RNA, while the Bridge helix binds to target DNA. The HNH and RuvC domains are nuclease domains. Guide RNA is engineered to have a 5′ end that is complementary to the target DNA sequence. Upon binding of the guide RNA to the Cas9 protein, a conformational change occurs activating the protein. Once activated, Cas9 searches for target DNA by binding to sequences that match its protospacer adjacent motif (PAM) sequence. A PAM is a two or three nucleotide base sequence within one nucleotide downstream of the region complementary to the guide RNA. In one non-limiting example, the PAM sequence is 5′-NGG-3′. When the Cas9 protein finds its target sequence with the appropriate PAM, it melts the bases upstream of the PAM and pairs them with the complementary region on the guide RNA. Then the RuvC and HNH nuclease domains cut the target DNA after the third nucleotide base upstream of the PAM.

One non-limiting example of a CRISPR/Cas system used to inhibit gene expression, CRISPRi, is described in U.S. Patent Appl. Publ. No. US20140068797. CRISPRi induces permanent gene disruption that utilizes the RNA-guided Cas9 endonuclease to introduce DNA double stranded breaks which trigger error-prone repair pathways to result in frame shift mutations. A catalytically dead Cas9 lacks endonuclease activity. When coexpressed with a guide RNA, a DNA recognition complex is generated that specifically interferes with transcriptional elongation, RNA polymerase binding, or transcription factor binding. This CRISPRi system efficiently represses expression of targeted genes.

CRISPR/Cas gene disruption occurs when a guide nucleic acid sequence specific for a target gene and a Cas endonuclease are introduced into a cell and form a complex that enables the Cas endonuclease to introduce a double strand break at the target gene. In certain embodiments, the CRISPR/Cas system comprises an expression vector, such as, but not limited to, a pAd5F35-CRISPR vector. In other embodiments, the Cas expression vector induces expression of Cas9 endonuclease. Other endonucleases may also be used, including but not limited to, Cas12a (Cpf1), T7, Cas3, Cas8a, Cas8b, Cas10d, Csel, Csyl, Csn2, Cas4, Cas10, Csm2, Cmr5, Fok1, other nucleases known in the art, and any combinations thereof.

In certain embodiments, inducing the Cas expression vector comprises exposing the cell to an agent that activates an inducible promoter in the Cas expression vector. In such embodiments, the Cas expression vector includes an inducible promoter, such as one that is inducible by exposure to an antibiotic (e.g., by tetracycline or a derivative of tetracycline, for example doxycycline). Other inducible promoters known by those of skill in the art can also be used. The inducing agent can be a selective condition (e.g., exposure to an agent, for example an antibiotic) that results in induction of the inducible promoter. This results in expression of the Cas expression vector.

As used herein, the term “guide RNA” or “gRNA” refer to any nucleic acid that promotes the specific association (or “targeting”) of an RNA-guided nuclease such as a Cas9 to a target sequence (e.g., a genomic or episomal sequence) in a cell.

As used herein, a “modular” or “dual RNA” guide comprises more than one, and typically two, separate RNA molecules, such as a CRISPR RNA (crRNA) and a trans-activating crRNA (tracrRNA), which are usually associated with one another, for example by duplexing. gRNAs and their component parts are described throughout the literature (see, e.g., Briner et al. Mol. Cell, 56(2), 333-339 (2014), which is incorporated by reference).

As used herein, a “unimolecular gRNA,” “chimeric gRNA,” or “single guide RNA (sgRNA)” comprises a single RNA molecule. The sgRNA may be a crRNA and tracrRNA linked together. For example, the 3′ end of the crRNA may be linked to the 5′ end of the tracrRNA. A crRNA and a tracrRNA may be joined into a single unimolecular or chimeric gRNA, for example, by means of a four nucleotide (e.g., GAAA) “tetraloop” or “linker” sequence bridging complementary regions of the crRNA (at its 3′ end) and the tracrRNA (at its 5′ end).

As used herein, a “repeat” sequence or region is a nucleotide sequence at or near the 3′ end of the crRNA which is complementary to an anti-repeat sequence of a tracrRNA.

As used herein, an “anti-repeat” sequence or region is a nucleotide sequence at or near the 5′ end of the tracrRNA which is complementary to the repeat sequence of a crRNA.

Additional details regarding guide RNA structure and function, including the gRNA/Cas9 complex for genome editing may be found in, at least, Mali et al. Science, 339(6121), 823-826 (2013); Jiang et al. Nat. Biotechnol. 31(3). 233-239 (2013); and Jinek et al. Science, 337(6096), 816-821 (2012); which are incorporated by reference herein.

As used herein, a “guide sequence” or “targeting sequence” refers to the nucleotide sequence of a gRNA, whether unimolecular or modular, that is fully or partially complementary to a target domain or target polynucleotide within a DNA sequence in the genome of a cell where editing is desired. Guide sequences are typically 10-30 nucleotides in length, preferably 16-24 nucleotides in length (for example, 16, 17, 18, 19, 20, 21, 22, 23 or 24 nucleotides in length), and are at or near the 5′ terminus of a Cas9 gRNA.

As used herein, a “target domain” or “target polynucleotide sequence” or “target sequence” is the DNA sequence in a genome of a cell that is complementary to the guide sequence of the gRNA.

In the context of formation of a CRISPR complex, “target sequence” refers to a sequence to which a guide sequence is designed to have some complementarity, where hybridization between a target sequence and a guide sequence promotes the formation of a CRISPR complex. Full complementarity is not necessarily required, provided there is sufficient complementarity to cause hybridization and promote formation of a CRISPR complex. A target sequence may comprise any polynucleotide, such as DNA or RNA polynucleotides. In certain embodiments, a target sequence is located in the nucleus or cytoplasm of a cell. In other embodiments, the target sequence may be within an organelle of a eukaryotic cell, for example, mitochondrion or nucleus. Typically, in the context of a CRISPR system, formation of a CRISPR complex (comprising a guide sequence hybridized to a target sequence and complexed with one or more Cas proteins) results in cleavage of one or both strands in or near (e.g., within about 1, 2, 3, 4, 5, 6, 7, 8, 9, 10, 20, 50 or more base pairs) the target sequence. As with the target sequence, it is believed that complete complementarity is not needed, provided this is sufficient to be functional.

In certain embodiments, one or more vectors driving expression of one or more elements of a CRISPR system are introduced into a host cell, such that expression of the elements of the CRISPR system direct formation of a CRISPR complex at one or more target sites. For example, a Cas nuclease, a crRNA, and a tracrRNA could each be operably linked to separate regulatory elements on separate vectors. Alternatively, two or more of the elements expressed from the same or different regulatory elements may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In certain embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g., each in a different intron, two or more in at least one intron, or all in a single intron).

In certain embodiments, the CRISPR enzyme is part of a fusion protein comprising one or more heterologous protein domains (e.g. about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Additional domains that may form part of a fusion protein comprising a CRISPR enzyme are described in U.S. Patent Appl. Publ. No. US20110059502, incorporated herein by reference. In certain embodiments, a tagged CRISPR enzyme is used to identify the location of a target sequence.

Conventional viral and non-viral based gene transfer methods can be used to introduce nucleic acids in mammalian and non-mammalian cells or target tissues. Such methods can be used to administer nucleic acids encoding components of a CRISPR system to cells in culture, or in a host organism. Non-viral vector delivery systems include DNA plasmids, RNA (e.g., a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell (Anderson, 1992, Science 256:808-813; and Yu, et al., 1994, Gene Therapy 1:13-26).

In some embodiments, the CRISPR/Cas is derived from a type II CRISPR/Cas system. In other embodiments, the CRISPR/Cas sytem is derived from a Cas9 nuclease. Exemplary Cas9 nucleases that may be used in the present disclosure include, but are not limited to, S. pyogenes Cas9 (SpCas9), S. aureus Cas9 (SaCas9), S. thermophilus Cas9 (StCas9), N. meningitidis Cas9 (NmCas9), C. jejuni Cas9 (CjCas9), and Geobacillus Cas9 (GeoCas9).

In general, Cas proteins comprise at least one RNA recognition and/or RNA binding domain. RNA recognition and/or RNA binding domains interact with the guiding RNA. Cas proteins can also comprise nuclease domains (i.e., DNase or RNase domains), DNA binding domains, helicase domains, RNAse domains, protein-protein interaction domains, dimerization domains, as well as other domains. The Cas proteins can be modified to increase nucleic acid binding affinity and/or specificity, alter an enzymatic activity, and/or change another property of the protein. In certain embodiments, the Cas-like protein of the fusion protein can be derived from a wild type Cas9 protein or fragment thereof. In other embodiments, the Cas can be derived from modified Cas9 protein. For example, the amino acid sequence of the Cas9 protein can be modified to alter one or more properties (e.g., nuclease activity, affinity, stability, and so forth) of the protein. Alternatively, domains of the Cas9 protein not involved in RNA-guided cleavage can be eliminated from the protein such that the modified Cas9 protein is smaller than the wild type Cas9 protein. In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-stranded break in DNA. (Jinek, et al., 2012, Science, 337:816-821). In certain embodiments, the Cas9-derived protein can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the nuclease activity is absent). In some embodiments in which one of the nuclease domains is inactive, the Cas9-derived protein is able to introduce a nick into a double-stranded nucleic acid (such protein is termed a “nickase”), but not cleave the double-stranded DNA. In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In one non-limiting embodiment, a vector drives the expression of the CRISPR system. The art is replete with suitable vectors that are useful in the present disclosure. The vectors to be used are suitable for replication and, optionally, integration in eukaryotic cells. Typical vectors contain transcription and translation terminators, initiation sequences, and promoters useful for regulation of the expression of the desired nucleic acid sequence. The vectors of the present disclosure may also be used for nucleic acid standard gene delivery protocols. Methods for gene delivery are known in the art (U.S. Pat. Nos. 5,399,346, 5,580,859 & 5,589,466, incorporated by reference herein in their entireties).

Further, the vector may be provided to a cell in the form of a viral vector. Viral vector technology is well known in the art and is described, for example, in Sambrook et al. (4th Edition, Molecular Cloning: A Laboratory Manual, Cold Spring Harbor Laboratory, New York, 2012), and in other virology and molecular biology manuals. Viruses, which are useful as vectors include, but are not limited to, retroviruses, adenoviruses, adeno-associated viruses, herpes viruses, Sindbis virus, gammaretrovirus and lentiviruses. In general, a suitable vector contains an origin of replication functional in at least one organism, a promoter sequence, convenient restriction endonuclease sites, and one or more selectable markers (e.g., WO 01/96584; WO 01/29058; and U.S. Pat. No. 6,326,193).

In some embodiments, guide RNA(s) and Cas9 can be delivered to a cell as a ribonucleoprotein (RNP) complex (e.g., a Cas9/RNA-protein complex). RNPs are comprised of purified Cas9 protein complexed with gRNA and are well known in the art to be efficiently delivered to multiple types of cells, including but not limited to stem cells and immune cells (Addgene, Cambridge, MA, Mirus Bio LLC, Madison, WI). In some embodiments, the Cas9/RNA-protein complex is delivered into a cell by electroporation.

In some embodiments, a modified cell of the present disclosure is edited using CRISPR/Cas9 to disrupt an endogenous gene locus encoding Fli1. Suitable gRNAs for use in disrupting Fli1 are set forth herein (see Table 1 and Table 2) and include but are not limited to SEQ ID NOs: 152-156 and SEQ ID NOs: 676-713. It will be understood to those of skill in the art that guide RNA sequences may be recited with a thymidine (T) or a uridine (U) nucleotide.

TABLE 2
Human Fli1 sgRNAs

| sgRNA name | sgRNA sequence | SEQ ID NO: |
|---|---|---|
| FLI1_ETS_843 | GAAGCGGGCAGATCCAGCTG | SEQ ID NO: 676 |
| FLI1_ETS_851 | CCAGGAGGAATTGCCACAGC | SEQ ID NO: 677 |
| FLI1_ETS_856 | CCAGCTGTGGCAATTCCTCC | SEQ ID NO: 678 |
| FLI1_ETS_866 | TGTCGGAGAGCAGCTCCAGG | SEQ ID NO: 679 |
| FLI1_ETS_869 | CGCTGTCGGAGAGCAGCTCC | SEQ ID NO: 680 |
| FLI1_ETS_883 | ACAGCTGGCGTTGGCGCTGT | SEQ ID NO: 681 |
| FLI1_ETS_892 | CCAGGTGATACAGCTGGCGT | SEQ ID NO: 682 |
| FLI1_ETS_897 | CCAACGCCAGCTGTATCACC | SEQ ID NO: 683 |
| FLI1_ETS_898 | CCCCTCCCAGGTGATACAGC | SEQ ID NO: 684 |
| FLI1_ETS_898 | CAACGCCAGCTGTATCACCT | SEQ ID NO: 685 |
| FLI1_ETS_901 | CGCCAGCTGTATCACCTGGG | SEQ ID NO: 686 |
| FLI1_ETS_902 | GCCAGCTGTATCACCTGGGA | SEQ ID NO: 687 |
| FLI1_ETS_903 | CCAGCTGTATCACCTGGGAG | SEQ ID NO: 688 |
| FLI1_ETS_910 | CTCCCCGTTGGTCCCCTCCC | SEQ ID NO: 689 |
| FLI1_ETS_911 | ATCACCTGGGAGGGGACCAA | SEQ ID NO: 690 |
| FLI1_ETS_912 | TCACCTGGGAGGGGACCAAC | SEQ ID NO: 691 |
| FLI1_ETS_913 | CACCTGGGAGGGGACCAACG | SEQ ID NO: 692 |
| FLI1_ETS_922 | CGTCATTTTGAACTCCCCGT | SEQ ID NO: 693 |
| FLI1_ETS_928 | CAACGGGGAGTTCAAAATGA | SEQ ID NO: 694 |
| FLI1_ETS_940 | CAAAATGACGGACCCCGATG | SEQ ID NO: 695 |
| FLI1_ETS_943 | AATGACGGACCCCGATGAGG | SEQ ID NO: 696 |
| FLI1_ETS_947 | AGCGCCTGGCCACCTCATCG | SEQ ID NO: 697 |
| FLI1_ETS_948 | CGGACCCCGATGAGGTGGCC | SEQ ID NO: 698 |
| FLI1_ETS_948 | CAGCGCCTGGCCACCTCATC | SEQ ID NO: 699 |
| FLI1_ETS_949 | CCAGCGCCTGGCCACCTCAT | SEQ ID NO: 700 |
| FLI1_ETS_954 | CCGATGAGGTGGCCAGGCGC | SEQ ID NO: 701 |
| FLI1_ETS_955 | CGATGAGGTGGCCAGGCGCT | SEQ ID NO: 702 |
| FLI1_ETS_956 | GATGAGGTGGCCAGGCGCTG | SEQ ID NO: 703 |
| FLI1_ETS_961 | TTTCCGCTCGCCCCAGCGCC | SEQ ID NO: 704 |
| FLI1_ETS_963 | TGGCCAGGCGCTGGGGCGAG | SEQ ID NO: 705 |
| FLI1_ETS_990 | AGCTTGTCGTAATTCATGTT | SEQ ID NO: 706 |
| FLI1_ETS_1016 | CATAGTAATAACGGAGGGCC | SEQ ID NO: 707 |
| FLI1_ETS_1021 | TTTATCATAGTAATAACGGA | SEQ ID NO: 708 |
| FLI1_ETS_1022 | TTTTATCATAGTAATAACGG | SEQ ID NO: 709 |
| FLI1_ETS_1025 | TGTTTTTATCATAGTAATAA | SEQ ID NO: 710 |
| FLI1_ETS_1049 | AACATTATGACCAAAGTGCA | SEQ ID NO: 711 |
| FLI1_ETS_1054 | ATATCTTTTGCCGTGCACTT | SEQ ID NO: 712 |
| FLI1_ETS_1082 | GCTTACAAATTTGACTTCCA | SEQ ID NO: 713 |

Non-limiting types of CRISPR-mediated modifications include a substitution, an insertion, a deletion, and an insertion/deletion (INDEL). The modification can be located in any part of the endogenous gene locus encoding Fli1, including but not limited to an exon, a splice donor, or a splice acceptor.

In certain embodiments, the guide RNA comprises a guide sequence that is sufficiently complementary with a target sequence in the endogenous gene locus encoding Fli1. In certain embodiments, the guide RNA comprises a guide sequence that is sufficiently complementary with a target sequence in the endogenous gene locus encoding Fli1, such as, for example, a guide sequence comprising any one of the sequences set forth in SEQ ID NOs: 152-156 or SEQ ID NOs: 676-713.

In certain embodiments, the modified cell is resistant to cell dysfunction. In certain embodiments, the modified cell is resistant to cell exhaustion. In certain embodiments, the modified cell is an autologous cell. In certain embodiments, the modified cell is a cell isolated from a human subject. In certain embodiments, the modified cell is a modified immune cell. In certain embodiments, the modified cell is a modified T cell. In certain embodiments, the modified cell is a modified T cell resistant to T cell exhaustion. In certain embodiments, the modified cell is a modified T cell resistant to T cell dysfunction.

In some aspects, the provided compositions and methods include those in which at least or greater than about 50%, 60%, 65%, 70%, 75%, 80%, 85%, 90% or 95% of immune cells in a composition of immune cells contain the desired genetic modification. For example, about 50%, 60%, 65%, 70%, 75%, 80%, 85%, 90% or 95% of immune cells in a composition of cells into which an agent (e.g. gRNA/Cas9) for knockout or genetic disruption of endogenous gene (e.g., Fli1) was introduced contain the genetic disruption; do not express the targeted endogenous polypeptide, or do not contain a contiguous and/or functional copy of the targeted gene. In some embodiments, the methods, compositions and cells according to the present disclosure include those in which at least or greater than about 50%, 60%, 65%, 70%, 75%, 80%, 85%, 90% or 95% of cells in a composition of cells into which an agent (e.g. gRNA/Cas9) for knockout or genetic disruption of a targeted gene was introduced do not express the targeted polypeptide, such as on the surface of the immune cells. In some embodiments, at least or greater than about 50%, 60%, 65%, 70%, 75%, 80%, 85%, 90% or 95% of cells in a composition of cells into which an agent (e.g. gRNA/Cas9) for knockout or genetic disruption of the targeted gene was introduced are knocked out in both alleles, i.e. comprise a biallelic deletion, in such percentage of cells.

In some embodiments, provided are compositions and methods in which the Cas9-mediated cleavage efficiency (% indel) in or near the targeted gene (e.g. within or about within 100 base pairs, within or about within 50 base pairs, or within or about within 25 base pairs or within or about within 10 base pairs upstream or downstream of the cut site) is at least or greater than about 50%, 60%, 65%, 70%, 75%, 80%, 85%, 90% or 95% in cells of a composition of cells into which an agent (e.g. gRNA/Cas9) for knockout or genetic disruption of a targeted gene has been introduced.

In some embodiments, the provided cells, compositions and methods results in a reduction or disruption of signals delivered via the endogenous in at least or greater than about 50%, 60%, 65%, 70%, 75%, 80%, 85%, 90% or 95% of cells in a composition of cells into which an agent (e.g. gRNA/Cas9) for knockout or genetic disruption of a targeted gene was introduced.

In some embodiments, compositions according to the provided disclosure that comprise cells engineered with a recombinant receptor and comprise the reduction, deletion, elimination, knockout or disruption in expression of an endogenous gene (e.g. genetic disruption of Fli1) retain the functional property or activities of the receptor compared to the receptor expressed in engineered cells of a corresponding or reference composition comprising the receptor but do not comprise the genetic disruption of a gene or express the polypeptide when assessed under the same conditions. In some embodiments, the engineered cells of the provided compositions retain a functional property or activity compared to a corresponding or reference composition comprising engineered cells in which such are engineered with the recombinant receptor but do not comprise the genetic disruption or express the targeted polypeptide when assessed under the same conditions. In some embodiments, the cells retain cytotoxicity, proliferation, survival or cytokine secretion compared to such a corresponding or reference composition.

In some embodiments, the immune cells in the composition retain a phenotype of the immune cell or cells compared to the phenotype of cells in a corresponding or reference composition when assessed under the same conditions. In some embodiments, cells in the composition include naïve cells, effector memory cells, central memory cells, stem central memory cells, effector memory cells, and long-lived effector memory cells. In some embodiments, the percentage of T cells comprising the genetic disruption of a targeted gene (e.g., Fli1) exhibit a non-activated, long-lived memory or central memory phenotype that is the same or substantially the same as a corresponding or reference population or composition of cells not containing the genetic disruption. In some embodiments, such property, activity or phenotype can be measured in an in vitro assay. In some embodiments, any of the assessed activities, properties or phenotypes can be assessed at various days following electroporation or other introduction of the agent, such as after or up to 3, 4, 5, 6, 7 days. In some embodiments, such activity, property or phenotype is retained by at least 80%, 85%, 90%, 95% or 100% of the cells in the composition compared to the activity of a corresponding composition containing cells not comprising the genetic disruption of the targeted gene when assessed under the same conditions.

As used herein, reference to a “corresponding composition” or a “corresponding population of immune cells” (also called a “reference composition” or a “reference population of cells”) refers to immune cells (e.g., T cells) obtained, isolated, generated, produced and/or incubated under the same or substantially the same conditions, except that the immune cells or population of immune cells were not introduced with the agent. In some aspects, except for not containing introduction of the agent, such immune cells are treated identically or substantially identically as immune cells that have been introduced with the agent, such that any one or more conditions that can influence the activity or properties of the cell, including the upregulation or expression of the inhibitory molecule, is not varied or not substantially varied between the cells other than the introduction of the agent.

Methods and techniques for assessing the expression and/or levels of T cell markers are known in the art. Antibodies and reagents for detection of such markers are well known in the art, and readily available. Assays and methods for detecting such markers include, but are not limited to, flow cytometry, including intracellular flow cytometry, ELISA, ELISPOT, cytometric bead array or other multiplex methods, Western Blot and other immunoaffinity-based methods. In some embodiments, cells can be detected by flow cytometry or other immunoaffinity based method for expression of a marker unique to such cells, and then such cells can be co-stained for another cell surface marker or markers.

In some embodiments, the cells, compositions and methods provide for the deletion, knockout, disruption, or reduction in expression of the target gene in immune cells (e.g. T cells) to be adoptively transferred. In some embodiments, the methods are performed ex vivo on primary cells, such as primary immune cells (e.g. T cells) from a subject. In some aspects, methods of producing or generating such genetically engineered T cells include introducing into a population of cells containing immune cells (e.g. T cells) an agent or agents that is capable of disrupting, a gene (e.g. Fli1) to be targeted. As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The population of cells containing T cells can be cells that have been obtained from a subject, such as obtained from a peripheral blood mononuclear cells (PBMC) sample, an unfractionated T cell sample, a lymphocyte sample, a white blood cell sample, an apheresis product, or a leukapheresis product. In some embodiments, T cells can be separated or selected to enrich T cells in the population using positive or negative selection and enrichment methods. In some embodiments, the population contains CD4⁺, CD8⁺ or CD4⁺ and CD8⁺T cells. In some embodiments, the step of introducing the nucleic acid encoding a genetically engineered antigen receptor and the step of introducing the agent (e.g. Cas9/gRNA RNP) can occur simultaneously or sequentially in any order. In some embodiments, subsequent to introduction of the exogenous receptor and one or more gene editing agents (e.g. Cas9/gRNA RNP), the cells are cultured or incubated under conditions to stimulate expansion and/or proliferation of cells.

Thus, provided are cells, compositions and methods that enhance immune cell, such as T cell, function in adoptive cell therapy, including those offering improved efficacy, such as by increasing activity and potency of administered genetically engineered cells, while maintaining persistence or exposure to the transferred cells over time. In some embodiments, the genetically engineered cells, exhibit increased expansion and/or persistence when administered in vivo to a subject, as compared to certain available methods. In some embodiments, the provided immune cells exhibit increased persistence when administered in vivo to a subject. In some embodiments, the persistence of genetically engineered immune cells, in the subject upon administration is greater as compared to that which would be achieved by alternative methods, such as those involving administration of cells genetically engineered by methods in which T cells were not introduced with an agent that reduces expression of or disrupts a gene encoding an endogenous receptor. In some embodiments, the persistence is increased at least or about at least 1.5-fold, 2-fold, 3-fold, 4-fold, 5-fold, 6-fold, 7-fold, 8-fold, 9-fold, 10-fold, 20-fold, 30-fold, 50-fold, 60-fold, 70-fold, 80-fold, 90-fold, 100-fold or more.

In some embodiments, the degree or extent of persistence of administered cells can be detected or quantified after administration to a subject. For example, in some aspects, quantitative PCR (qPCR) is used to assess the quantity of cells in the blood or serum or organ or tissue (e.g., disease site) of the subject. In some aspects, persistence is quantified as copies of DNA or plasmid encoding the exogenous receptor per microgram of DNA, or as the number of receptor-expressing cells per microliter of the sample, e.g., of blood or serum, or per total number of peripheral blood mononuclear cells (PBMCs) or white blood cells or T cells per microliter of the sample. In some embodiments, flow cytometric assays detecting cells generally using antibodies specific for the cells also can be performed. Cell-based assays may also be used to detect the number or percentage of functional cells, such as cells capable of binding to and/or neutralizing and/or inducing responses, e.g., cytotoxic responses, against cells of the disease or condition or expressing the antigen recognized by the receptor. In any of such embodiments, the extent or level of expression of another marker associated with cells can be used to distinguish the administered cells from endogenous cells in a subject.

### C. Sources of Immune Cells

In certain embodiments, a source of immune cells is obtained from a subject for ex vivo manipulation. Sources of target cells for ex vivo manipulation may also include, e.g., autologous or heterologous donor blood, cord blood, or bone marrow. For example the source of immune cells may be from the subject to be treated with the modified immune cells of the disclosure, e.g., the subject's blood, the subject's cord blood, or the subject's bone marrow. Non-limiting examples of subjects include humans, dogs, cats, mice, rats, and transgenic species thereof. Preferably, the subject is a human.

Immune cells can be obtained from a number of sources, including blood, peripheral blood mononuclear cells, bone marrow, lymph node tissue, spleen tissue, umbilical cord, lymph, or lymphoid organs. Immune cells are cells of the immune system, such as cells of the innate or adaptive immunity, e.g., myeloid or lymphoid cells, including lymphocytes, typically T cells and/or NK cells. Other exemplary cells include stem cells, such as multipotent and pluripotent stem cells, including induced pluripotent stem cells (iPSCs). In some aspects, the cells are human cells. With reference to the subject to be treated, the cells may be allogeneic and/or autologous. The cells typically are primary cells, such as those isolated directly from a subject and/or isolated from a subject and frozen.

In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In some embodiments, the cells are monocytes or granulocytes, e.g., myeloid cells, macrophages, neutrophils, dendritic cells, mast cells, eosinophils, and/or basophils. In an embodiment, the target cell is an induced pluripotent stem (iPS) cell or a cell derived from an iPS cell, e.g., an iPS cell generated from a subject, manipulated to alter (e.g., induce a mutation in) or manipulate the expression of one or more target genes, and differentiated into, e.g., a T cell, e.g., a CD8+ T cell (e.g., a CD8+ naive T cell, central memory T cell, or effector memory T cell), a CD4+ T cell, a stem cell memory T cell, a lymphoid progenitor cell or a hematopoietic stem cell.

In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen- specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells. In certain embodiments, any number of T cell lines available in the art, may be used.

In some embodiments, the methods include isolating immune cells from the subject, preparing, processing, culturing, and/or engineering them. In some embodiments, preparation of the engineered cells includes one or more culture and/or preparation steps. The cells for engineering as described may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, and pig. In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps.

In some embodiments, the cells are washed with phosphate buffered saline (PBS). In some aspects, a washing step is accomplished by tangential flow filtration (TFF) according to the manufacturer's instructions. In some embodiments, the cells are resuspended in a variety of biocompatible buffers after washing. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In one embodiment, immune are obtained cells from the circulating blood of an individual are obtained by apheresis or leukapheresis. The apheresis product typically contains lymphocytes, including T cells, monocytes, granulocytes, B cells, other nucleated white blood cells, red blood cells, and platelets. The cells collected by apheresis may be washed to remove the plasma fraction and to place the cells in an appropriate buffer or media, such as phosphate buffered saline (PBS) or wash solution lacks calcium and may lack magnesium or may lack many if not all divalent cations, for subsequent processing steps. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In some embodiments, the isolation methods include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some embodiments, one or more of the T cell populations is enriched for or depleted of cells that are positive for (marker+) or express high levels (marker^{high}) of one or more particular markers, such as surface markers, or that are negative for (marker −) or express relatively low levels (marker^{low}) of one or more markers. For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some cases, such markers are those that are absent or expressed at relatively low levels on certain populations of T cells (such as non-memory cells) but are present or expressed at relatively higher levels on certain other populations of T cells (such as memory cells). In one embodiment, the cells (such as the CD8+ cells or the T cells, e.g., CD3+ cells) are enriched for (i.e., positively selected for) cells that are positive or expressing high surface levels of CD45RO, CCR7, CD28, CD27, CD44, CD 127, and/or CD62L and/or depleted of (e.g., negatively selected for) cells that are positive for or express high surface levels of CD45RA. In some embodiments, cells are enriched for or depleted of cells positive or expressing high surface levels of CD 122, CD95, CD25, CD27, and/or IL7-Ra (CD 127). In some examples, CD8+ T cells are enriched for cells positive for CD45RO (or negative for CD45RA) and for CD62L. For example, CD3+, CD28+ T cells can be positively selected using CD3/CD28 conjugated magnetic beads (e.g., DYNABEADS® M-450 CD3/CD28 T Cell Expander).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations. In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. In some embodiments, combining TCM-enriched CD8+ T cells and CD4+ T cells further enhances efficacy.

In some embodiments, memory T cells are present in both CD62L+ and CD62L− subsets of CD8+ peripheral blood lymphocytes. PBMC can be enriched for or depleted of CD62L-CD8+ and/or CD62L+CD8⁺fractions, such as using anti-CD8 and anti-CD62L antibodies. In some embodiments, a CD4+ T cell population and a CD8+ T cell sub-population, e.g., a sub-population enriched for central memory (TCM) cells. In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD 14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD 14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4− based separation are retained and used in subsequent steps of the methods, optionally following one or more further positive or negative selection steps.

CD4+ T helper cells are sorted into naive, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, CD4+ T cells. In some embodiments, central memory CD4+ cells are CD62L+ and CD45RO+. In some embodiments, effector CD4⁺ cells are CD62L- and CD45RO.

In one example, to enrich for CD4⁺ cells by negative selection, a monoclonal antibody cocktail typically includes antibodies to CD14, CD20, CD1 1b, CD16, HLA-DR, and CD8. In some embodiments, the antibody or binding partner is bound to a solid support or matrix, such as a magnetic bead or paramagnetic bead, to allow for separation of cells for positive and/or negative selection.

In some embodiments, the cells are incubated and/or cultured prior to or in connection with genetic engineering. The incubation steps can include culture, cultivation, stimulation, activation, and/or propagation. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells. In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell. Such agents can include antibodies, such as those specific for a TCR component and/or costimulatory receptor, e.g., anti-CD3, anti-CD28, for example, bound to solid support such as a bead, and/or one or more cytokines. Optionally, the expansion method may further comprise the step of adding anti-CD3 and/or anti CD28 antibody to the culture medium (e.g., at a concentration of at least about 0.5 ng/ml). In some embodiments, the stimulating agents include IL-2 and/or IL-15, for example, an IL-2 concentration of at least about 10 units/mL.

In another embodiment, T cells are isolated from peripheral blood by lysing the red blood cells and depleting the monocytes, for example, by centrifugation through a PERCOLL™ gradient. Alternatively, T cells can be isolated from an umbilical cord. In any event, a specific subpopulation of T cells can be further isolated by positive or negative selection techniques.

The cord blood mononuclear cells so isolated can be depleted of cells expressing certain antigens, including, but not limited to, CD34, CD8, CD14, CD19, and CD56. Depletion of these cells can be accomplished using an isolated antibody, a biological sample comprising an antibody, such as ascites, an antibody bound to a physical support, and a cell bound antibody.

Enrichment of a T cell population by negative selection can be accomplished using a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4⁺ cells by negative selection, a monoclonal antibody cocktail typically includes antibodies to CD14, CD20, CD11b, CD16, HLA-DR, and CD8.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion.

T cells can also be frozen after the washing step, which does not require the monocyte-removal step. While not wishing to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, in a non-limiting example, one method involves using PBS containing 20% DMSO and 8% human serum albumin, or other suitable cell freezing media. The cells are then frozen to −80° C. at a rate of 1° C. per minute and stored in the vapor phase of a liquid nitrogen storage tank. Other methods of controlled freezing may be used as well as uncontrolled freezing immediately at −20° C. or in liquid nitrogen.

In one embodiment, the population of T cells is comprised within cells such as peripheral blood mononuclear cells, cord blood cells, a purified population of T cells, and a T cell line. In another embodiment, peripheral blood mononuclear cells comprise the population of T cells. In yet another embodiment, purified T cells comprise the population of T cells.

In certain embodiments, T regulatory cells (Tregs) can be isolated from a sample. The sample can include, but is not limited to, umbilical cord blood or peripheral blood. In certain embodiments, the Tregs are isolated by flow-cytometry sorting. The sample can be enriched for Tregs prior to isolation by any means known in the art. The isolated Tregs can be cryopreserved, and/or expanded prior to use. Methods for isolating Tregs are described in U.S. Pat. Nos. 7,754,482, 8,722,400, and 9,555,105, and U.S. patent application Ser. No. 13/639,927, contents of which are incorporated herein in their entirety.

### D. Methods of Producing Modified Immune Cells

The present disclosure provides methods for producing or generating a modified immune cell or precursor thereof (e.g., a T cell).

In certain embodiments, the disclosure provides a method for generating a modified immune cell or precursor cell thereof, comprising introducing into an immune or precursor cell a CRISPR system comprising one or more polypeptides and/or nucleic acids capable of downregulating gene expression of endogenous Fli1.

In some embodiments, a nucleic acid is introduced into a cell by an expression vector. Suitable expression vectors include lentivirus vectors, gamma retrovirus vectors, foamy virus vectors, adeno associated virus (AAV) vectors, adenovirus vectors, engineered hybrid viruses, naked DNA, including but not limited to transposon mediated vectors, such as Sleeping Beauty, Piggybak, and Integrases such as Phi31. Some other suitable expression vectors include Herpes simplex virus (HSV) and retrovirus expression vectors.

In certain embodiments, the nucleic acid is introduced into the cell via viral transduction. In certain embodiments, the viral transduction comprises contacting the immune or precursor cell with a viral vector comprising the nucleic acid. In certain embodiments, the viral vector is an adeno-associated viral (AAV) vector. In certain embodiments, the AAV vector comprises a 5′ ITR and a 3′ITR. In certain embodiments, the AAV vector comprises a Woodchuck Hepatitis Virus post-transcriptional regulatory element (WPRE). In certain embodiments, the AAV vector comprises a polyadenylation (polyA) sequence. In certain embodiments, the polyA sequence is a bovine growth hormone (BGH) polyA sequence.

Adenovirus expression vectors are based on adenoviruses, which have a low capacity for integration into genomic DNA but a high efficiency for transfecting host cells. Adenovirus expression vectors contain adenovirus sequences sufficient to: (a) support packaging of the expression vector and (b) to ultimately express the target sequence in the host cell. In some embodiments, the adenovirus genome is a 36 kb, linear, double stranded DNA, where a foreign DNA sequence may be inserted to substitute large pieces of adenoviral DNA in order to make the expression vector of the present disclosure (see, e.g., Danthinne and Imperiale, Gene Therapy (2000) 7(20): 1707-1714).

Another expression vector is based on an adeno associated virus (AAV), which takes advantage of the adenovirus coupled systems. This AAV expression vector has a high frequency of integration into the host genome. It can infect nondividing cells, thus making it useful for delivery of genes into mammalian cells, for example, in tissue cultures or in vivo. The AAV vector has a broad host range for infectivity. Details concerning the generation and use of AAV vectors are described in U.S. Pat. Nos. 5,139,941 and 4,797,368.

Retrovirus expression vectors are capable of integrating into the host genome, delivering a large amount of foreign genetic material, infecting a broad spectrum of species and cell types and being packaged in special cell lines. The retroviral vector is constructed by inserting a nucleic acid into the viral genome at certain locations to produce a virus that is replication defective. Though the retroviral vectors are able to infect a broad variety of cell types, integration and stable expression of the gene/protein requires the division of host cells.

Lentiviral vectors are derived from lentiviruses, which are complex retroviruses that, in addition to the common retroviral genes gag, pol, and env, contain other genes with regulatory or structural function (see, e.g., U.S. Pat. Nos. 6,013,516 and 5,994,136). Some examples of lentiviruses include the Human Immunodeficiency Viruses (HIV-1, HIV-2) and the Simian Immunodeficiency Virus (SIV). Lentiviral vectors have been generated by multiply attenuating the HIV virulence genes, for example, the genes env, vif, vpr, vpu and nef are deleted making the vector biologically safe. Lentiviral vectors are capable of infecting non-dividing cells and can be used for both in vivo and ex vivo gene transfer and expression (see, e.g., U.S. Pat. No. 5,994,136).

Expression vectors including a nucleic acid of the present disclosure can be introduced into a host cell by any means known to persons skilled in the art. The expression vectors may include viral sequences for transfection, if desired. Alternatively, the expression vectors may be introduced by fusion, electroporation, biolistics, transfection, lipofection, or the like. The host cell may be grown and expanded in culture before introduction of the expression vectors, followed by the appropriate treatment for introduction and integration of the vectors. he host cells are then expanded and may be screened by virtue of a marker present in the vectors. Various markers that may be used are known in the art, and may include hprt, neomycin resistance, thymidine kinase, hygromycin resistance, etc. As used herein, the terms “cell,” “cell line,” and “cell culture” may be used interchangeably. In some embodiments, the host cell an immune cell or precursor thereof, e.g., a T cell, an NK cell, or an NKT cell.

The present disclosure also provides genetically engineered cells wherein the endogenous Fli1 is disrupted. In some embodiments, the genetically engineered cells are genetically engineered T-lymphocytes (T cells), naive T cells (TN), memory T cells (for example, central memory T cells (TCM), effector memory cells (TEM)), natural killer cells (NK cells), and macrophages capable of giving rise to therapeutically relevant progeny. In certain embodiments, the genetically engineered cells are autologous cells. In certain embodiments, the modified cell is resistant to T cell exhaustion. In certain embodiments, the modified cell is resistant to T cell dysfunction.

Modified cells may be produced by stably transfecting host cells with an expression vector including a nucleic acid of the present disclosure. Additional methods for generating a modified cell of the present disclosure include, without limitation, chemical transformation methods (e.g., using calcium phosphate, dendrimers, liposomes and/or cationic polymers), non-chemical transformation methods (e.g., electroporation, optical transformation, gene electrotransfer and/or hydrodynamic delivery) and/or particle-based methods (e.g., impalefection, using a gene gun and/or magnetofection). Transfected cells of the present disclosure may be expanded ex vivo.

Physical methods for introducing an expression vector into host cells include calcium phosphate precipitation, lipofection, particle bombardment, microinjection, electroporation, and the like. Methods for producing cells including vectors and/or exogenous nucleic acids are well-known in the art. See, e.g., Sambrook et al. (2001), Molecular Cloning: A Laboratory Manual, Cold Spring Harbor Laboratory, New York. Chemical methods for introducing an expression vector into a host cell include colloidal dispersion systems, such as macromolecule complexes, nanocapsules, microspheres, beads, and lipid-based systems including oil-in-water emulsions, micelles, mixed micelles, and liposomes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform may be used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). Compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present disclosure, in order to confirm the presence of the nucleic acids in the host cell, a variety of assays may be performed. Such assays include, for example, molecular biology assays well known to those of skill in the art, such as Southern and Northern blotting, RT-PCR and PCR; biochemistry assays, such as detecting the presence or absence of a particular peptide, e.g., by immunological means (ELISAs and Western blots) or by assays described herein to identify agents falling within the scope of the disclosure.

In one embodiment, the nucleic acids introduced into the host cell are RNA. In another embodiment, the RNA is mRNA that comprises in vitro transcribed RNA or synthetic RNA. The RNA may be produced by in vitro transcription using a polymerase chain reaction (PCR)-generated template. DNA of interest from any source can be directly converted by PCR into a template for in vitro mRNA synthesis using appropriate primers and RNA polymerase. The source of the DNA may be, for example, genomic DNA, plasmid DNA, phage DNA, cDNA, synthetic DNA sequence or any other appropriate source of DNA.

PCR may be used to generate a template for in vitro transcription of mRNA which is then introduced into cells. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary. Substantially complementary sequences are able to anneal or hybridize with the intended DNA target under annealing conditions used for PCR. The primers can be designed to be substantially complementary to any portion of the DNA template. For example, the primers can be designed to amplify the portion of a gene that is normally transcribed in cells (the open reading frame), including 5′ and 3′ UTRs. The primers may also be designed to amplify a portion of a gene that encodes a particular domain of interest. In one embodiment, the primers are designed to amplify the coding region of a human cDNA, including all or portions of the 5′ and 3′ UTRs. Primers useful for PCR are generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Chemical structures that have the ability to promote stability and/or translation efficiency of the RNA may also be used. The RNA preferably has 5′ and 3′ UTRs. In one embodiment, the 5′ UTR is between zero and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the gene of interest. Alternatively, UTR sequences that are not endogenous to the gene of interest can be added by incorporating the UTR sequences into the forward and reverse primers or by any other modifications of the template. The use of UTR sequences that are not endogenous to the gene of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous gene. Alternatively, when a 5′ UTR that is not endogenous to the gene of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be derived from an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In one embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The polyA/T segment of the transcriptional DNA template can be produced during PCR by using a reverse primer containing a polyT tail, such as 100T tail (size can be 50-5000 T), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines.

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps also provide stability to RNA molecules. In a preferred embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

In some embodiments, the RNA is electroporated into the cells, such as in vitro transcribed RNA. Any solutes suitable for cell electroporation, which can contain factors facilitating cellular permeability and viability such as sugars, peptides, lipids, proteins, antioxidants, and surfactants can be included.

The disclosed methods can be applied to the modulation of T cell activity in basic research and therapy, in the fields of cancer, stem cells, acute and chronic infections, and autoimmune diseases, including the assessment of the ability of the genetically modified T cell to kill a target cancer cell.

The methods also provide the ability to control the level of expression over a wide range by changing, for example, the promoter or the amount of input RNA, making it possible to individually regulate the expression level. Furthermore, the PCR-based technique of mRNA production greatly facilitates the design of the mRNAs with different structures and combination of their domains.

One advantage of RNA transfection methods of the disclosure is that RNA transfection is essentially transient and a vector-free. An RNA transgene can be delivered to a lymphocyte and expressed therein following a brief in vitro cell activation, as a minimal expressing cassette without the need for any additional viral sequences. Under these conditions, integration of the transgene into the host cell genome is unlikely. Cloning of cells is not necessary because of the efficiency of transfection of the RNA and its ability to uniformly modify the entire lymphocyte population.

Genetic modification of T cells with in vitro-transcribed RNA (IVT-RNA) makes use of two different strategies both of which have been successively tested in various animal models. Cells are transfected with in vitro-transcribed RNA by means of lipofection or electroporation. It is desirable to stabilize IVT-RNA using various modifications in order to achieve prolonged expression of transferred IVT-RNA.

Some IVT vectors are known in the literature which are utilized in a standardized manner as template for in vitro transcription and which have been genetically modified in such a way that stabilized RNA transcripts are produced. Currently protocols used in the art are based on a plasmid vector with the following structure: a 5′ RNA polymerase promoter enabling RNA transcription, followed by a gene of interest which is flanked either 3′ and/or 5′ by untranslated regions (UTR), and a 3′ polyadenyl cassette containing 50-70 A nucleotides. Prior to in vitro transcription, the circular plasmid is linearized downstream of the polyadenyl cassette by type II restriction enzymes (recognition sequence corresponds to cleavage site). The polyadenyl cassette thus corresponds to the later poly(A) sequence in the transcript. As a result of this procedure, some nucleotides remain as part of the enzyme cleavage site after linearization and extend or mask the poly(A) sequence at the 3′ end. It is not clear, whether this nonphysiological overhang affects the amount of protein produced intracellularly from such a construct.

In another aspect, the RNA construct is delivered into the cells by electroporation. See, e.g., the formulations and methodology of electroporation of nucleic acid constructs into mammalian cells as taught in US 2004/0014645, US 2005/0052630A1, US 2005/0070841A1, US 2004/0059285A1, US 2004/0092907A1. The various parameters including electric field strength required for electroporation of any known cell type are generally known in the relevant research literature as well as numerous patents and applications in the field. See e.g., U.S. Pat. Nos. 6,678,556, 7,171,264, and 7,173,116. Apparatus for therapeutic application of electroporation are available commercially, e.g., the MedPulser™ DNA Electroporation Therapy System (Inovio/Genetronics, San Diego, Calif.), and are described in patents such as U.S. Pat. Nos. 6,567,694; 6,516,223, 5,993,434, 6,181,964, 6,241,701, and 6,233,482; electroporation may also be used for transfection of cells in vitro as described e.g. in US20070128708A1. Electroporation may also be utilized to deliver nucleic acids into cells in vitro. Accordingly, electroporation-mediated administration into cells of nucleic acids including expression constructs utilizing any of the many available devices and electroporation systems known to those of skill in the art presents an exciting new means for delivering an RNA of interest to a target cell.

In some embodiments, the immune cells (e.g. T cells) can be incubated or cultivated prior to, during and/or subsequent to introducing the nucleic acid molecule encoding the gene editing agent (e.g. Cas9/gRNA RNP). In some embodiments, the method includes activating or stimulating cells with a stimulating or activating agent (e.g. anti-CD3/anti-CD28 antibodies) prior to introducing the the gene editing agent, e.g. Cas9/gRNA RNP. In some embodiments, prior to the introducing of the agent, the cells are allowed to rest, e.g. by removal of any stimulating or activating agent. In some embodiments, prior to introducing the agent, the stimulating or activating agent and/or cytokines are not removed.

E. Methods of Treatment with Modified Cells

The modified cells (e.g., T cells) described herein may be included in a composition for immunotherapy. The composition may include a pharmaceutical composition and further include a pharmaceutically acceptable carrier. A therapeutically effective amount of the pharmaceutical composition comprising the modified T cells may be administered.

In one aspect, a method for adoptive cell transfer therapy is provided herein, which comprises administering to a subject in need thereof a modified cell of the present disclosure. In another aspect, a method of treating a disease or condition in a subject is provided herein, which comprises administering to a subject in need thereof a population of modified cells. Also included is a method of treating a disease or condition in a subject in need thereof comprising administering to the subject a genetically edited modified cell (e.g., comprising downregulated expression of endogenous Fli1).

Methods for administration of immune cells for adoptive cell therapy are known and may be used in connection with the provided methods and compositions. For example, adoptive T cell therapy methods are described, e.g., in US Patent Application Publication No. 2003/0170238 to Gruenberg et al; U.S. Pat. No. 4,690,915 to Rosenberg; Rosenberg (2011) Nat Rev Clin Oncol. 8(10):577-85). See, e.g., Themeli et al. (2013) Nat Biotechnol. 31(10): 928-933; Tsukahara et al. (2013) Biochem Biophys Res Commun 438(1): 84-9; Davila et al. (2013) PLoS ONE 8(4): e61338. In some embodiments, the cell therapy, e.g., adoptive T cell therapy is carried out by autologous transfer, in which the cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive T cell therapy, is carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the subject has been treated with a therapeutic agent targeting the disease or condition, e.g. the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at a high risk of relapse, and thus the cells are administered prophylactically, e.g., to reduce the likelihood of or prevent relapse. In some aspects, the subject has not received prior treatment with another therapeutic agent.

In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

The modified immune cells of the present disclosure can be administered to an animal, preferably a mammal, even more preferably a human, to treat a cancer. In addition, the cells of the present disclosure can be used for the treatment of any condition related to a cancer, especially a cell-mediated immune response against a tumor cell(s), where it is desirable to treat or alleviate the disease. The types of cancers to be treated with the modified cells or pharmaceutical compositions of the disclosure include, carcinoma, blastoma, and sarcoma, and certain leukemia or lymphoid malignancies, benign and malignant tumors, and malignancies e.g., sarcomas, carcinomas, and melanomas. Other exemplary cancers include but are not limited breast cancer, prostate cancer, ovarian cancer, cervical cancer, skin cancer, pancreatic cancer, colorectal cancer, renal cancer, liver cancer, brain cancer, lymphoma, leukemia, lung cancer, thyroid cancer, and the like. The cancers may be non-solid tumors (such as hematological tumors) or solid tumors. Adult tumors/cancers and pediatric tumors/cancers are also included. In one embodiment, the cancer is a solid tumor or a hematological tumor. In one embodiment, the cancer is a carcinoma. In one embodiment, the cancer is a sarcoma. In one embodiment, the cancer is a leukemia. In one embodiment the cancer is a solid tumor.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastases).

Carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma.

Sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

In certain exemplary embodiments, the modified immune cells of the disclosure are used to treat a myeloma, or a condition related to myeloma. Examples of myeloma or conditions related thereto include, without limitation, light chain myeloma, non-secretory myeloma, monoclonal gamopathy of undertermined significance (MGUS), plasmacytoma (e.g., solitary, multiple solitary, extramedullary plasmacytoma), amyloidosis, and multiple myeloma. In one embodiment, a method of the present disclosure is used to treat multiple myeloma. In one embodiment, a method of the present disclosure is used to treat refractory myeloma. In one embodiment, a method of the present disclosure is used to treat relapsed myeloma.

In certain exemplary embodiments, the modified immune cells of the disclosure are used to treat a melanoma, or a condition related to melanoma. Examples of melanoma or conditions related thereto include, without limitation, superficial spreading melanoma, nodular melanoma, lentigo maligna melanoma, acral lentiginous melanoma, amelanotic melanoma, or melanoma of the skin (e.g., cutaneous, eye, vulva, vagina, rectum melanoma). In one embodiment, a method of the present disclosure is used to treat cutaneous melanoma. In one embodiment, a method of the present disclosure is used to treat refractory melanoma. In one embodiment, a method of the present disclosure is used to treat relapsed melanoma.

In yet other exemplary embodiments, the modified immune cells of the disclosure are used to treat a sarcoma, or a condition related to sarcoma. Examples of sarcoma or conditions related thereto include, without limitation, angiosarcoma, chondrosarcoma, Ewing's sarcoma, fibrosarcoma, gastrointestinal stromal tumor, leiomyosarcoma, liposarcoma, malignant peripheral nerve sheath tumor, osteosarcoma, pleomorphic sarcoma, rhabdomyosarcoma, and synovial sarcoma. In one embodiment, a method of the present disclosure is used to treat synovial sarcoma. In one embodiment, a method of the present disclosure is used to treat liposarcoma such as myxoid/round cell liposarcoma, differentiated/dedifferentiated liposarcoma, and pleomorphic liposarcoma. In one embodiment, a method of the present disclosure is used to treat myxoid/round cell liposarcoma. In one embodiment, a method of the present disclosure is used to treat a refractory sarcoma. In one embodiment, a method of the present disclosure is used to treat a relapsed sarcoma.

The cells of the disclosure to be administered may be autologous, with respect to the subject undergoing therapy.

In certain exemplary embodiments, the modified immune cells of the disclosure are used to treat an infection. In certain embodiments, the infection is an acute infection. In certain embodiments, the infection is a chronic infection. In certain embodiments, the infection is a viral infection. In certain embodiments, a method of the present disclosure is used to treat a disease, disorder, or infection selected from the group consisting of LCMV, HIV, Hepatitis B, Hepatitis C, malaria, or tuberculosis.

The administration of the cells of the disclosure may be carried out in any convenient manner known to those of skill in the art. The cells of the present disclosure may be administered to a subject by aerosol inhalation, injection, ingestion, transfusion, implantation or transplantation. The compositions described herein may be administered to a patient transarterially, subcutaneously, intradermally, intratumorally, intranodally, intramedullary, intramuscularly, by intravenous (i.v.) injection, or intraperitoneally. In other instances, the cells of the disclosure are injected directly into a site of inflammation in the subject, a local disease site in the subject, a lymph node, an organ, a tumor, and the like.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight) and a desired ratio of the individual populations or sub-types, such as the CD4⁺to CD8⁺ratio. In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations or sub-types of cells, such as CD8⁺ and CD4⁺T cells, are administered at or within a tolerated difference of a desired dose of total cells, such as a desired dose of T cells. In some aspects, the desired dose is a desired number of cells or a desired number of cells per unit of body weight of the subject to whom the cells are administered, e.g., cells/kg. In some aspects, the desired dose is at or above a minimum number of cells or minimum number of cells per unit of body weight. In some aspects, among the total cells, administered at the desired dose, the individual populations or sub-types are present at or near a desired output ratio (such as CD4⁺to CD8⁺ratio), e.g., within a certain tolerated difference or error of such a ratio.

In some embodiments, the cells are administered at or within a tolerated difference of a desired dose of one or more of the individual populations or sub-types of cells, such as a desired dose of CD4⁺ cells and/or a desired dose of CD8⁺ cells. In some aspects, the desired dose is a desired number of cells of the sub-type or population, or a desired number of such cells per unit of body weight of the subject to whom the cells are administered, e.g., cells/kg. In some aspects, the desired dose is at or above a minimum number of cells of the population or subtype, or minimum number of cells of the population or sub-type per unit of body weight. Thus, in some embodiments, the dosage is based on a desired fixed dose of total cells and a desired ratio, and/or based on a desired fixed dose of one or more, e.g., each, of the individual sub-types or sub-populations. Thus, in some embodiments, the dosage is based on a desired fixed or minimum dose of T cells and a desired ratio of CD4⁺ to CD8⁺ cells, and/or is based on a desired fixed or minimum dose of CD4⁺ and/or CD8⁺ cells.

In certain embodiments, the cells, or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 1×10⁵ cells/kg to about 1×10¹¹ cells/kg 10⁴ and at or about 10¹¹ cells/kilograms (kg) body weight, such as between 10⁵ and 10⁶ cells/kg body weight, for example, at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ T cells/kilograms (kg) body weight, such as between 10⁵ and 10⁶ T cells/kg body weight, for example, at or about 1×10⁵ T cells/kg, 1.5×10⁵ T cells/kg, 2×10⁵ T cells/kg, or 1×10⁶ T cells/kg body weight. In other exemplary embodiments, a suitable dosage range of modified cells for use in a method of the present disclosure includes, without limitation, from about 1×10⁵ cells/kg to about 1×10⁶ cells/kg, from about 1×10⁶ cells/kg to about 1×10⁷ cells/kg, from about 1×10⁷ cells/kg about 1×10⁸ cells/kg, from about 1×10⁸ cells/kg about 1×10⁹ cells/kg, from about 1×10⁹ cells/kg about 1×10¹⁰ cells/kg, from about 1×10¹⁰ cells/kg about 1×10¹¹ cells/kg. In an exemplary embodiment, a suitable dosage for use in a method of the present disclosure is about 1×10⁸ cells/kg. In an exemplary embodiment, a suitable dosage for use in a method of the present disclosure is about 1×10⁷ cells/kg. In other embodiments, a suitable dosage is from about 1×10⁷ total cells to about 5×10⁷ total cells. In some embodiments, a suitable dosage is from about 1×10⁸ total cells to about 5×10⁸ total cells. In some embodiments, a suitable dosage is from about 1.4×10⁷ total cells to about 1.1×10⁹ total cells. In an exemplary embodiment, a suitable dosage for use in a method of the present disclosure is about 7×10⁹ total cells.

In some embodiments, the cells are administered at or within a certain range of error of between at or about 10⁴ and at or about 10⁹ CD4⁺ and/or CD8⁺ cells/kilograms (kg) body weight, such as between 10⁵ and 10⁶ CD4⁺ and/or CD8⁺ cells/kg body weight, for example, at or about 1×10⁵ CD4⁺ and/or CD8⁺ cells/kg, 1.5×10⁵ CD4⁺ and/or CD8⁺ cells/kg, 2×10⁵ CD4⁺ and/or CD8⁺ cells/kg, or 1×10⁶ CD4⁺and/or CD8⁺ cells/kg body weight. In some embodiments, the cells are administered at or within a certain range of error of, greater than, and/or at least about 1×10⁶, about 2.5×10⁶, about 5×10⁶, about 7.5×10⁶, or about 9×10⁶ CD4⁺ cells, and/or at least about 1×10⁶, about 2.5×10⁶, about 5×10⁶, about 7.5×10⁶, or about 9×10⁶ CD8⁺ cells, and/or at least about 1×10⁶, about 2.5×10⁶, about 5×10⁶, about 7.5×10⁶, or about 9×10⁶ T cells. In some embodiments, the cells are administered at or within a certain range of error of between about 108 and 10¹² or between about 10¹⁰ and 10¹¹ T cells, between about 10′ and 10¹² or between about 10¹⁰ and 10¹¹ CD4⁺ cells, and/or between about 108 and 10¹² or between about 10¹⁰ and 10¹¹ CD8⁺ cells.

In some embodiments, the cells are administered at or within a tolerated range of a desired output ratio of multiple cell populations or sub-types, such as CD4⁺ and CD8⁺ cells or sub-types. In some aspects, the desired ratio can be a specific ratio or can be a range of ratios, for example, in some embodiments, the desired ratio (e.g., ratio of CD4⁺to CD8⁺ cells) is between at or about 5:1 and at or about 5:1 (or greater than about 1:5 and less than about 5:1), or between at or about 1:3 and at or about 3:1 (or greater than about 1:3 and less than about 3:1), such as between at or about 2:1 and at or about 1:5 (or greater than about 1:5 and less than about 2:1, such as at or about 5:1, 4.5:1, 4:1, 3.5:1, 3:1, 2.5:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9: 1:2, 1:2.5, 1:3, 1:3.5, 1:4, 1:4.5, or 1:5. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

In some embodiments, a dose of modified cells is administered to a subject in need thereof, in a single dose or multiple doses. In some embodiments, a dose of modified cells is administered in multiple doses, e.g., once a week or every 7 days, once every 2 weeks or every 14 days, once every 3 weeks or every 21 days, once every 4 weeks or every 28 days. In an exemplary embodiment, a single dose of modified cells is administered to a subject in need thereof. In an exemplary embodiment, a single dose of modified cells is administered to a subject in need thereof by rapid intravenous infusion.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

In certain embodiments, the modified cells of the disclosure (e.g., a modified cell comprising modified endogenous Fli1) may be administered to a subject in combination with an immune checkpoint antibody (e.g., an anti-PDi, anti-CTLA-4, or anti-PDL1 antibody). For example, the modified cell may be administered in combination with an antibody or antibody fragment targeting, for example, PD-1 (programmed death 1 protein). Examples of anti-PD-1 antibodies include, but are not limited to, pembrolizumab (KEYTRUDA®, formerly lambrolizumab, also known as MK-3475), and nivolumab (BMS-936558, MDX-1106, ONO-4538, OPDIVA®) or an antigen-binding fragment thereof. In certain embodiments, the modified cell may be administered in combination with an anti-PD-L1 antibody or antigen-binding fragment thereof. Examples of anti-PD-L1 antibodies include, but are not limited to, BMS-936559, MPDL3280A (TECENTRIQ®, Atezolizumab), and MEDI4736 (Durvalumab, Imfinzi). In certain embodiments, the modified cell may be administered in combination with an anti-CTLA-4 antibody or antigen-binding fragment thereof. An example of an anti- CTLA-4 antibody includes, but is not limited to, Ipilimumab (trade name Yervoy). Other types of immune checkpoint modulators may also be used including, but not limited to, small molecules, siRNA, miRNA, and CRISPR systems. Immune checkpoint modulators may be administered before, after, or concurrently with the modified cell. In certain embodiments, combination treatment comprising an immune checkpoint modulator may increase the therapeutic efficacy of a therapy comprising a modified cell of the present disclosure.

Following administration of the cells, the biological activity of the engineered cell populations in some embodiments is measured, e.g., by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells is measured by assaying expression and/or secretion of one or more cytokines, such as CD 107a, IFNγ, IL-2, and TNF. In some aspects the biological activity is measured by assessing clinical outcome, such as reduction in tumor burden or load.

In certain embodiments, the subject is provided a secondary treatment. Secondary treatments include but are not limited to chemotherapy, radiation, surgery, and medications.

In some embodiments, the subject can be administered a conditioning therapy prior to being administered the modified cell. In some embodiments, the conditioning therapy comprises administering an effective amount of cyclophosphamide to the subject. In some embodiments, the conditioning therapy comprises administering an effective amount of fludarabine to the subject. In preferred embodiments, the conditioning therapy comprises administering an effective amount of a combination of cyclophosphamide and fludarabine to the subject. Administration of a conditioning therapy prior to the modified cell may increase the efficacy of the modified cell. Methods of conditioning patients for T cell therapy are described in U.S. Pat. No. 9,855,298, which is incorporated herein by reference in its entirety.

In some embodiments, a specific dosage regimen of the present disclosure includes a lymphodepletion step prior to the administration of the modified T cells. In an exemplary embodiment, the lymphodepletion step includes administration of cyclophosphamide and/or fludarabine.

Cells of the disclosure can be administered in dosages and routes and at times to be determined in appropriate pre-clinical and clinical experimentation and trials. Cell compositions may be administered multiple times at dosages within these ranges. Administration of the cells of the disclosure may be combined with other methods useful to treat the desired disease or condition as determined by those of skill in the art.

In certain embodiments, following administration of the modified cell, the subject may be administered a treatment of cytokine release syndrome (CRS), immune activation resulting in elevated inflammatory cytokines. Accordingly, the disclosure provides for, following the diagnosis of CRS, appropriate CRS management strategies to mitigate the physiological symptoms of uncontrolled inflammation without dampening the antitumor efficacy of the modified cells. CRS management strategies are known in the art. For example, systemic corticosteroids may be administered to rapidly reverse symptoms of sCRS (e.g., grade 3 CRS) without compromising initial antitumor response. In some embodiments, an anti-IL-6R antibody may be administered. An example of an anti-IL-6R antibody is the Food and Drug Administration-approved monoclonal antibody tocilizumab, also known as atlizumab (marketed as Actemra, or RoActemra). Tocilizumab is a humanized monoclonal antibody against the interleukin-6 receptor (IL-6R). Administration of tocilizumab has demonstrated near-immediate reversal of CRS.

The modified immune cells of the present disclosure may be used in a method of treatment as described herein. In some embodiments, the modified immune cells comprise an insertion and/or deletion in a Fli1 gene locus that is capable of downregulating gene expression of Fli1. In some embodiments, when Fli1 is downregulated, the function of the immune cell is enhanced. For example, without limitation, when downregulated, Fli1 enhances tumor infiltration, tumor killing, and/or resistance to immunosuppression of the immune cell. In some embodiments, when Fli1is downregulated, T cell exhaustion is reduced or eliminated. In some embodiments, when Fli1is downregulated, T cell dysfunction is reduced or eliminated.

In one aspect, the disclosure includes a method of treating cancer in a subject in need thereof, comprising administering to the subject any one of the modified immune or precursor cells disclosed herein. Yet another aspect of the disclosure includes a method of treating cancer in a subject in need thereof, comprising administering to the subject a modified immune or precursor cell generated by any one of the methods disclosed herein.

Still another aspect of the disclosure includes a method of treating a disease or disorder in a subject in need thereof, comprising administering to the subject a modified cell comprising: a CRISPR-mediated modification in an endogenous gene locus encoding Fli1, wherein the modification is capable of downregulating gene expression of endogenous Fli1.

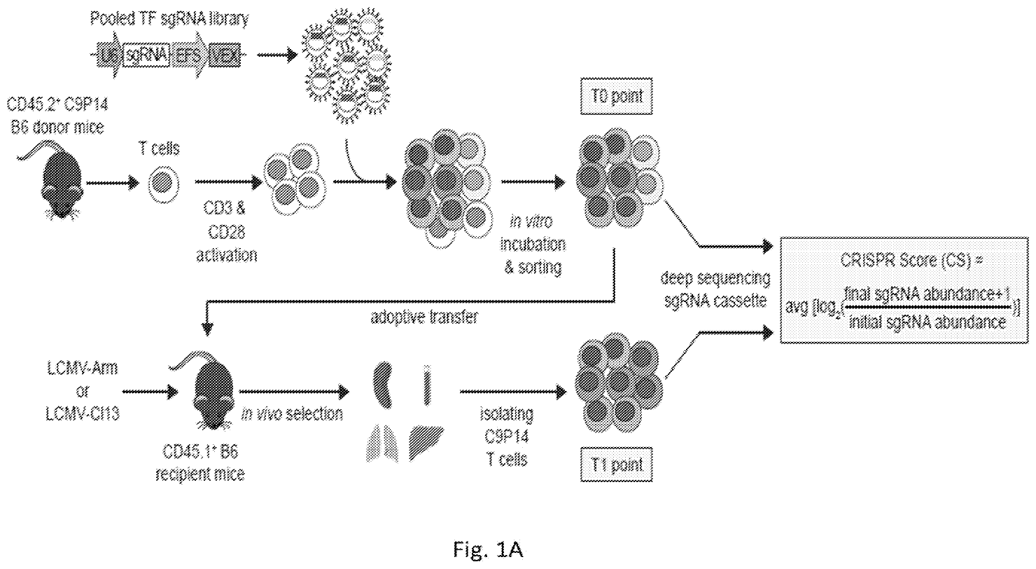

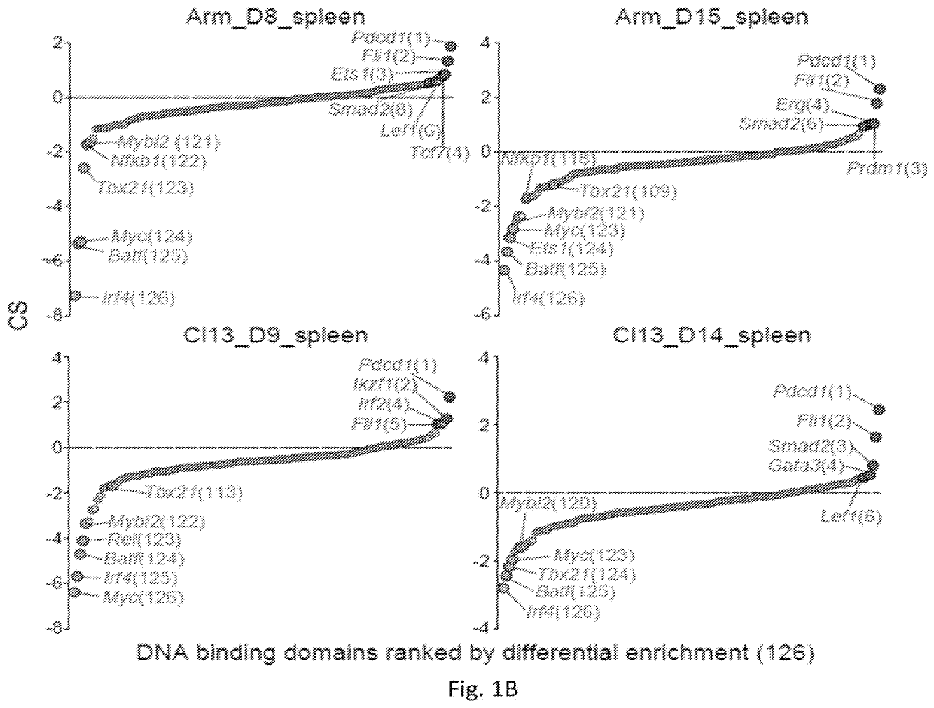

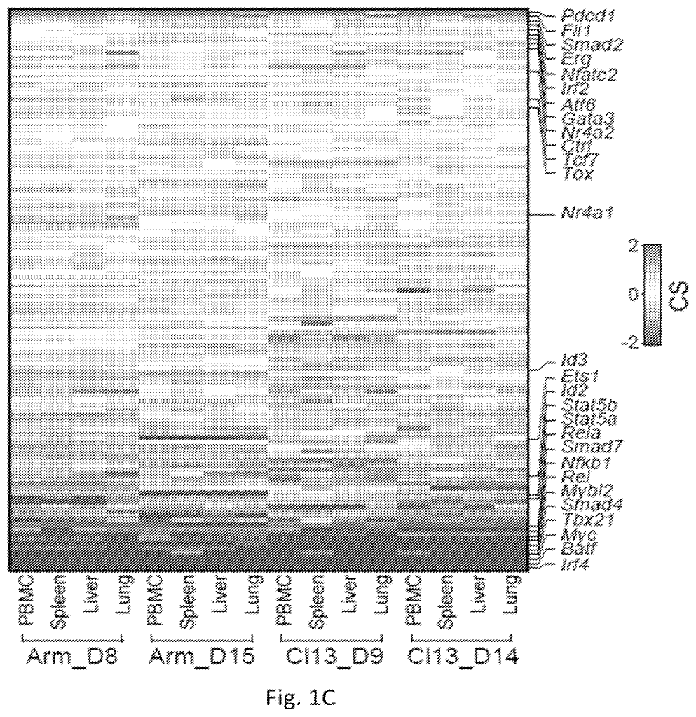

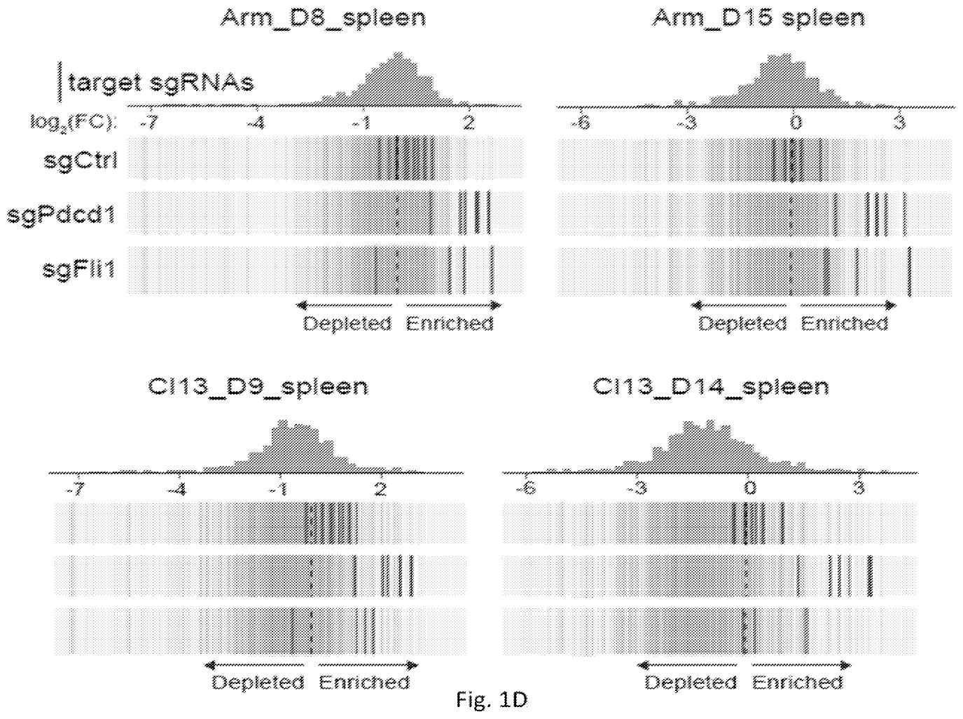

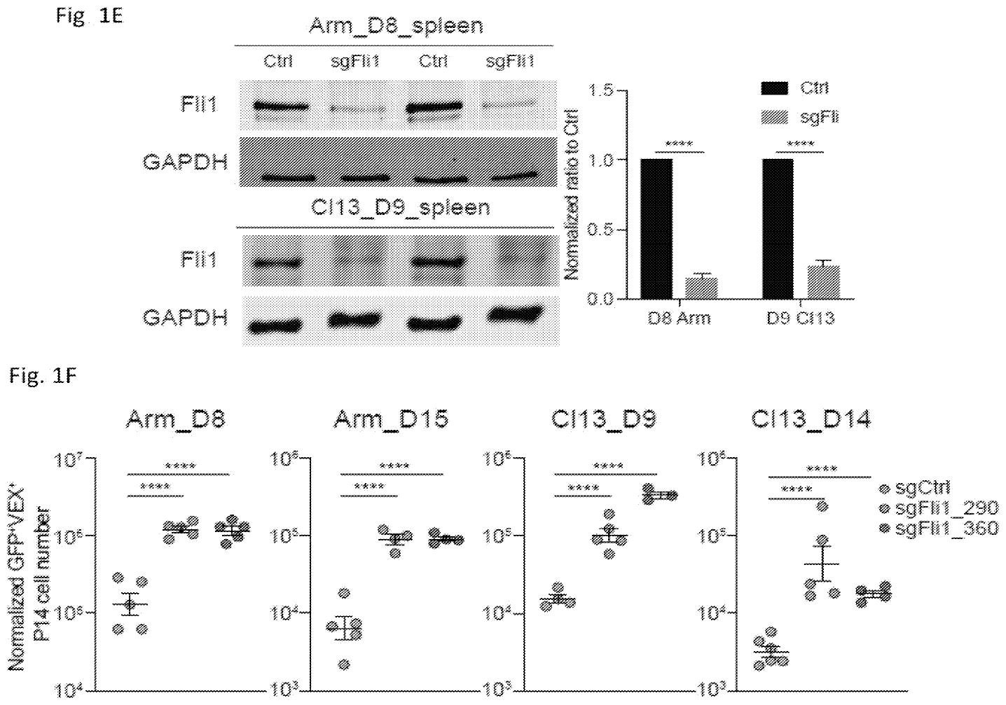

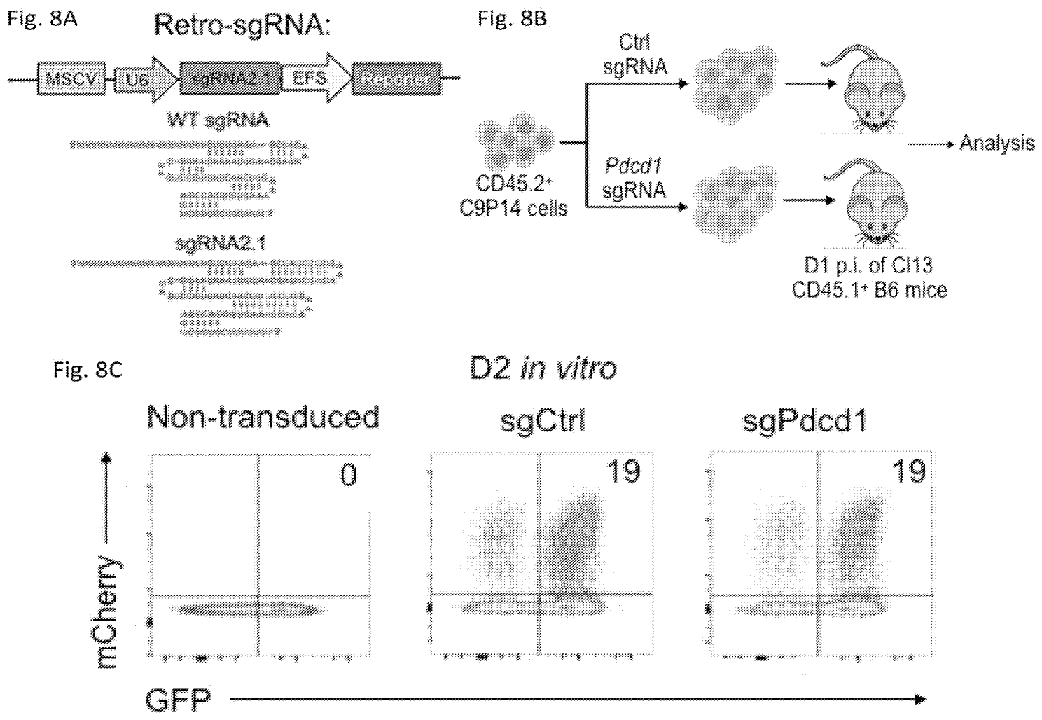

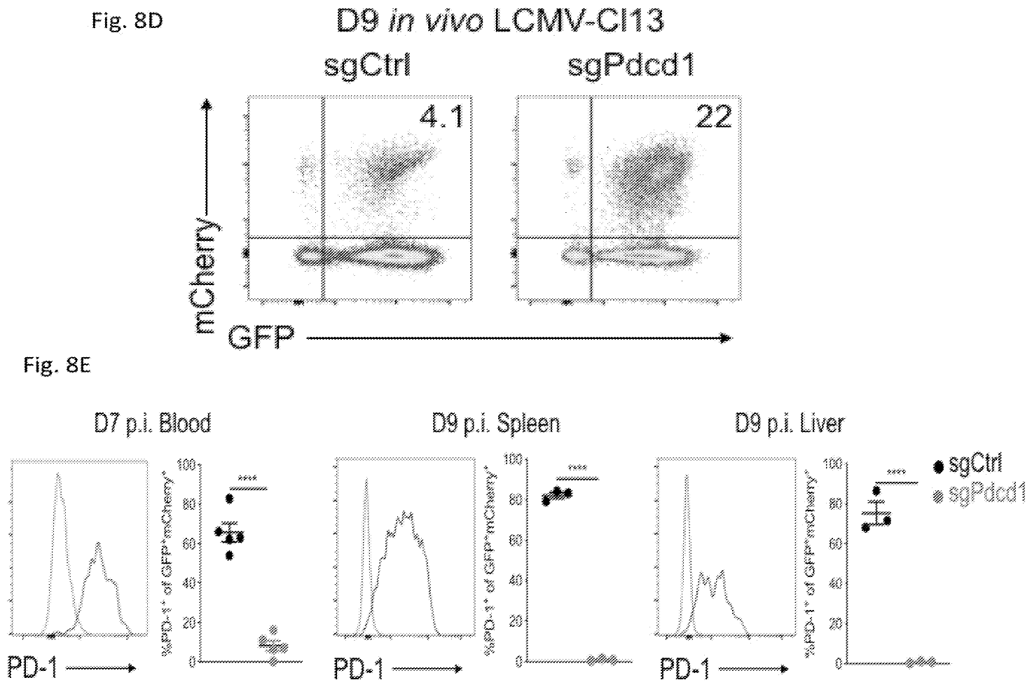

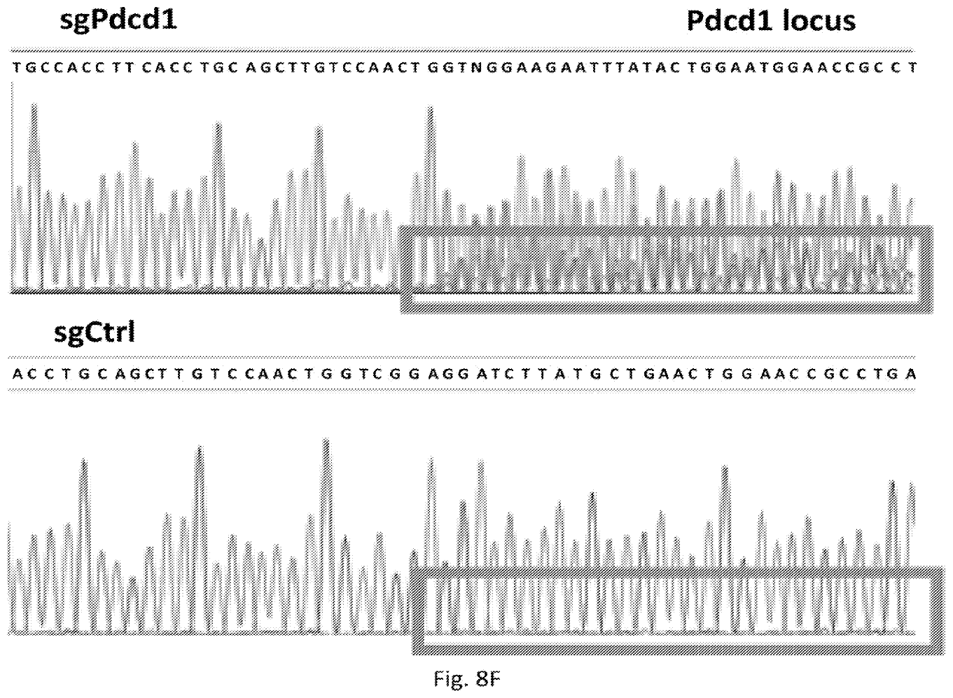

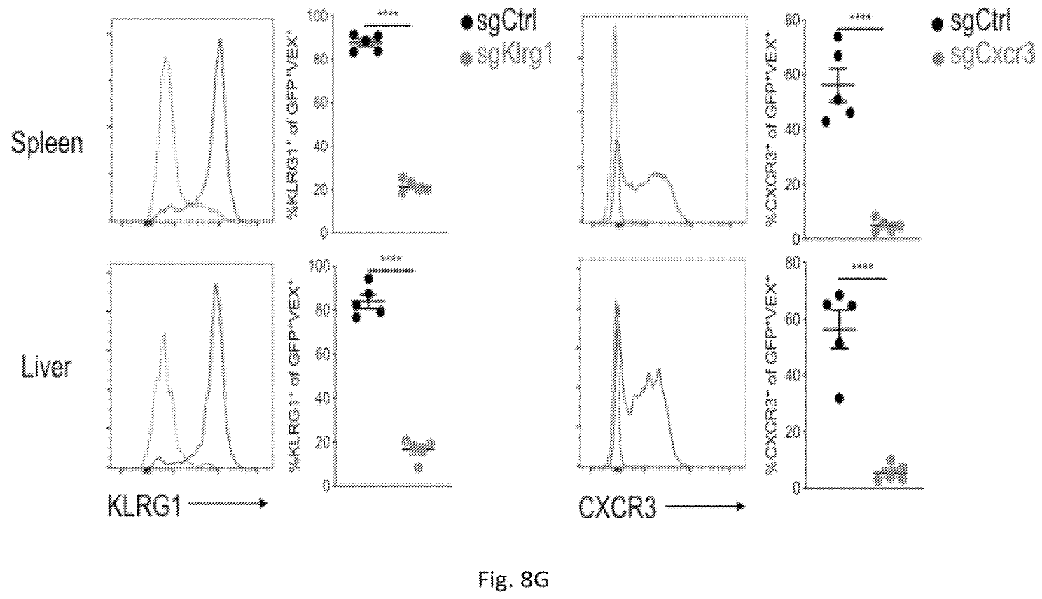

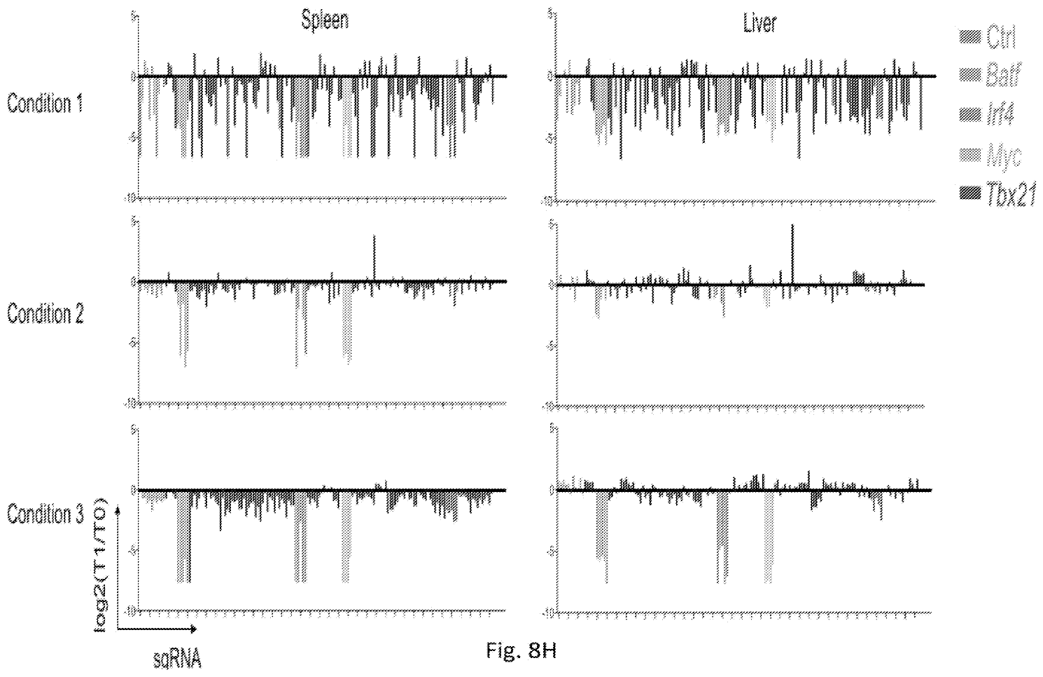

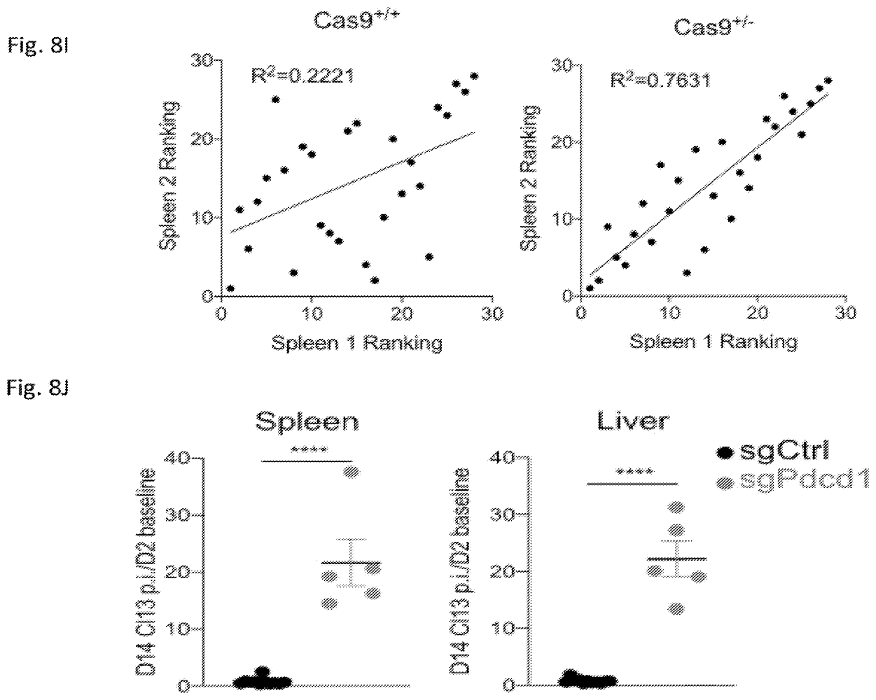

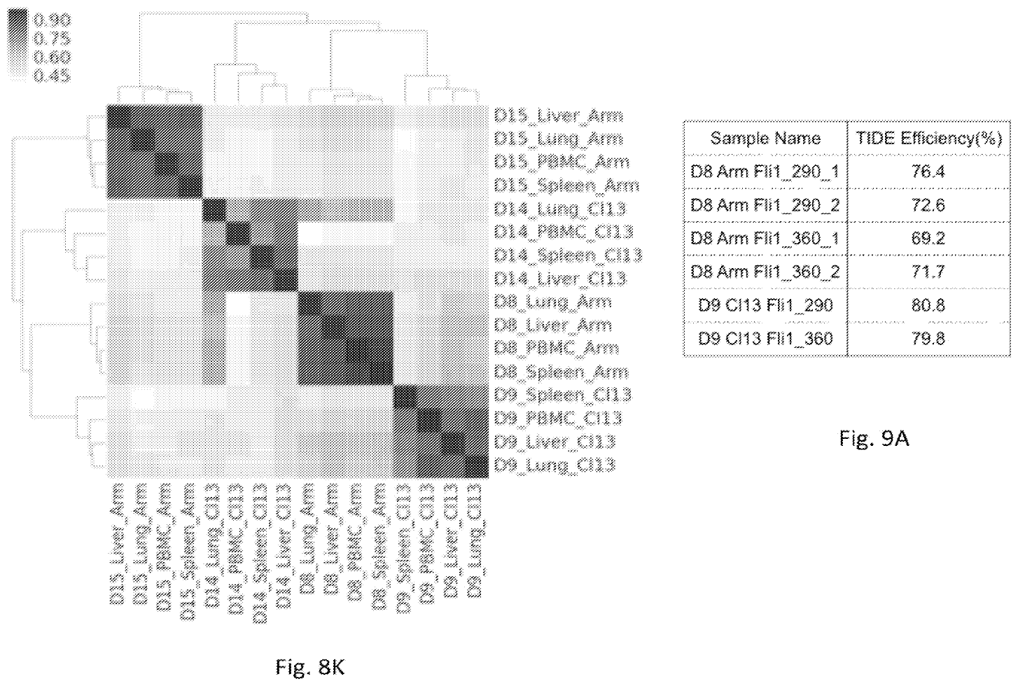

F. Methods of screening cells The present disclosure provides methods for screening cells (e.g. T cells), such as the Qptimized T cell In vivo CRISPR Screening (OpTICS) method, as exemplified in FIGS. 1A, 8A, and 8B.

In one aspect, the disclosure provides a method of screening a cell comprising: i) introducing into an activated cell a Cas enzyme (or a nucleic acid encoding Cas) and an sgRNA library, ii) administering the cell into an infected or tumor-bearing mouse, iii) isolating the cell from the infected mouse, and iv) analyzing the cell.

In one aspect, the disclosure provides a method of screening a T cell comprising: i) introducing into an activated T cell a Cas enzyme and an sgRNA library, ii) administering the T cell into an infected or tumor-bearing mouse, iii) isolating the T cell from the infected mouse, and iv) analyzing the T cell.

The sgRNA library should be construed to contain any number of sgRNAs targeting any number of genes of interest, including but not limited to, any and all genes with annotated functional domains.

In certain embodiments, the sgRNA library comprises a plurality of sgRNAs that target a plurality of transcription factors. In certain embodiments, the plurality of transcription factors comprise any of the transcription factors listed in Table 1. In certain embodiments, each sgRNAs targets the DNA binding domain of each transcription factor. In certain embodiments, the sgRNA library comprises at least one sequence selected from the group consisting of SEQ ID NOs: 1-675. In certain embodiments, the sgRNA library consists of the nucleotide sequences set forth in SEQ ID NOs: 1-675. The library should be construed to contain any and all numbers of sgRNAs selected from the group consisting of SEQ ID NO: 1-675. For example, the number of sgRNAs in the sgRNA library can be 1, 10, 20, 50, 100, 200, 300, 400, 500, 600, 675, or any and all numbers in between 1-675.

In certain embodiments, the method screens a T cell to assesses T cell exhaustion. In certain embodiments, the method identifies novel transcription factors governing T_{EFF} and T_{EX} cell differentiation.

In certain embodiments, the method is used for screening in a tumor system, i.e. for identifying genes of interest in a tumor/cancer. In certain embodiments, the method screens B cells to assess memory B cell and plasma cell formation. In certain embodiments, the method screens hematopoietic or tissue stem cells to assess stem cell and related lineages.

In certain embodiments, analyzing the cell comprises a method selected from the group consisting of sequencing, PCR, MACS, and FACS. In certain embodiments, sequencing reveals a target of interest. In certain embodiments, a drug is designed against the target of interest. In certain embodiments, when the drug is administered to the T cell, at least one T cell response is increased or elicited. In certain embodiments, during the analysis the CRISPR Score (CS) is calculated, e.g. as shown in FIG. 1A.

In certain embodiments, around 1×10⁵ T cells are administered to the infected mouse.

### G. Pharmaceutical Compositions and Formulations

Also provided are populations of immune cells of the disclosure, and compositions containing such cells and/or enriched for such cells. Among the compositions are pharmaceutical compositions and formulations for administration, such as for adoptive cell therapy. Also provided are therapeutic methods for administering the cells and compositions to subjects, e.g., patients.

Also provided are compositions including the cells for administration, including pharmaceutical compositions and formulations, such as unit dose form compositions including the number of cells for administration in a given dose or fraction thereof. The pharmaceutical compositions and formulations generally include one or more optional pharmaceutically acceptable carrier or excipient. In some embodiments, the composition includes at least one additional therapeutic agent.

The term “pharmaceutical formulation” refers to a preparation which is in such form as to permit the biological activity of an active ingredient contained therein to be effective, and which contains no additional components which are unacceptably toxic to a subject to which the formulation would be administered. A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative. In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine. The pharmaceutical composition in some embodiments contains the cells in amounts effective to treat or prevent the disease or condition, such as a therapeutically effective or prophylactically effective amount.

Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

Formulations include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection. Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyoi (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility may be readily accomplished, e.g., by filtration through sterile filtration membranes.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other physical and electronic documents.

While the present disclosure has been described with reference to the specific embodiments thereof, it should be understood by those skilled in the art that various changes may be made and equivalents may be substituted without departing from the true spirit and scope of the invention. It will be readily apparent to those skilled in the art that other suitable modifications and adaptations of the methods described herein may be made using suitable equivalents without departing from the scope of the embodiments disclosed herein. In addition, many modifications may be made to adapt a particular situation, material, composition of matter, process, process step or steps, to the objective, spirit and scope of the present disclosure. All such modifications are intended to be within the scope of the claims appended hereto. Having now described certain embodiments in detail, the same will be more clearly understood by reference to the following examples, which are included for purposes of illustration only and are not intended to be limiting.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Materials and Methods

Mice: CD4CRE, LSL-Cas9-GFP and Constitutive-Cas9-GFP mice were purchased from Jackson Laboratory. LSL-Cas9-GFP mice were bred to CD4CRE mice and TCR transgenic P14 C57BL/6 mice (TCR specific for LCMV DbGP33-41) and back crossed for more than 6 generations before use. Constitutive-Cas9-GFP mice were bred to TCR transgenic P14 C57BL/6 mice. Constitutive-Cas9-GFP mice for recipient use were bred in house. 6-8 week-old C57BL/6 Ly5.2CR (CD45.1) or C57BL/6 (CD45.2) mice were purchased from NCI. 6 week-old C57BL/6 (CD45.2) mice were purchased from Jackson Laboratory. 5-7 week-old Rag2−/−C57BL/6 mice were purchased from Jackson Laboratory. Recipient mice for LCMV challenge were from NCI unless otherwise noted in the figure legends. Both male and female mice were used. All mice were used in accordance with Institutional Animal Care and Use Committee guidelines for the University of Pennsylvania.

### Experimental Models

LCMV Infection: Mice were infected intraperitoneally (i.p.) with 2×10⁵ plaque-forming units (PFU) Arm or intravenously (i.v.) with 4×10⁶ PFU C113. Plaque assay for LCMV C113 to detect viral load was processed as previously described (Pauken et al., 2016). Basically, tissues were homogenized, and either 1:10, 1:10², 1:103 dilution of serum or 1:10, 1:10², 1:103, 1:104, 1:10⁵ and 1:10⁶ dilution of homogenized tissue-contained media were incubated on adherent Vero cells for 1 hr. Cells were overlaid with a 1:1 mixture of medium and 1% agarose and cultured for 4 days. Plaques (PFUs) were counted after overlaying with a 9.5:9.5:1 mixture of medium:1% agarose:neutral red for 16 hr.

Listeria monocytogenes (LM) infection: LM expressing DbGP33(LM-gp33) concentration was measured by optical density (OD) after overnight culture in brain heart infusion (BHI) media (1 OD refers to 8×10⁸ LM-gp33). Each recipient mouse was infected intravenously (i.v.) with 1×10⁵ CFU LM-gp33. Adjusted survival was based on mice remaining above the mandatory Institutional Animal Care and Use Committee (IACUC) euthanasia cut off of 30% weight loss. LM-gp33 colony formation per unit calculation for bacteria load was calculated using 2% agarose plate with complete BHI media. Infected organs are smashed in 1 ml BHI media and 20 ul (2%) of the organs are taken out. 1:10, 1:102, 1:103, 1:104, 1:105 and 1:106 dilution of the infected organ BHI media are made and plated on the BHI agarose plates. Colonies are counted after 16 hours incubation of the plates in 37° C. incubator.

Influenza PR8 infection: Mice were infected intranasally (i.n.) with PR8 strain expressing DbGP33 (PR8-gp33) at a dose of 3.0 LD50. Mice were anesthetized before i.n. infection. PR8 viral qPCR detection for viral RNA amount was calculated as previously described (Laidlaw et al., (2013) PLOS Pathogens 9, e1003207). Total RNA (including host and viral RNA) was purified from lungs of PR8-GP33 infected mice as well as the paired spleen, followed by reverse transcriptions with random primers. Real-time quantitative PCR was performed on cDNA targeting the influenza PA protein with technical triplicate. Influenza viral RNA amount was standardized using influenza PA protein cDNA standards.

Tumor transfer: B16F10 melanoma cells expressing DbGP33 (B16F10-gp33,(Prévost-Blondel et al., (1998) The Journal of Immunology 161, 2187-2194) were maintained at 37° C. in DMEM medium supplemented with 10% FBS, penicillin, streptomycin and L-glutamine. Tumor cells were injected subcutaneously into the flanks of Rag2−/−mice at 1×10⁵ cells/recipient and of Cas9+ B6 mice at 2×10⁵ cells/recipient. Activated sgRNA+C9P14 cells are sorted and transferred into recipient mice at a dose of 1×10⁶ cells/recipient (for Rag2−/−) or 3×10⁶ cells/recipient (for Cas9⁺). Tumor size was measured using digital calipers every 2-3 days after inoculation.

Vector construction and sgRNA cloning: In this study, SpCas9 sgRNA was expressed using pSL21-VEX or pSL21-mCherry (U6-sgRNA-EFS-VEX or U6-sgRNA-EFS-mCherry). To generate the pSL21-VEX or pSL21-mCherry, U6-sgRNA expression cassette was PCR cloned from LRG2.1 into a retroviral vector MSCV-Neo, followed by swapping the Neo selection marker with a VEX or mCherry fluorescence reporter. sgRNAs were cloned by annealing two DNA oligos and T4 DNA ligation into a Bbs1- digested pSL21-VEX or pSL21-mCherry vector. To improve U6 promoter transcription efficiency, an additional 5′ G nucleotide was added to all sgRNA oligo designs that did not already start with a 5′ G. Runx1 and Runx3 constructs are built on the MIGR or MSCV-mCherry constructs, empty MIGR or MSCV-mCherry are used as the controls for these vectors.

Cell culture and in vitro stimulation: CD8 T cells were purified from spleens by negative selection using EasySep Mouse CD8+ T Cell Isolation Kit (STEMCELL Technologies) according to manufacturer's instructions. Cells were stimulated with 100 U/mL recombinant human IL-2, 1 μg/mL anti-mouse CD3R, and 5 μg/mL anti-mouse CD28 in RPMI-1640 medium with 10% fetal bovine serum (FBS), 10 mM HEPES, 100 μM non-essential amino acids (NEAA), 50 U/mL penicillin, 50 μg/mL streptomycin, and 50 μM β-mercaptoethanol.

Retroviral vector (RV) experiments: RVs were produced in 293T cells with MSCV and pCL-Eco plasmids using Lipofectamine 3000. RV transduction was performed as described (Kurachi et al., (2017) Nature Protocols, 12:9 12, 1980-1998). Briefly, CD8+ T cells were purified from spleens of P14 mice using EasySep™ Mouse CD8⁺T Cell Isolation Kit. After 18-24 hrs of in vitro stimulation, P14 cells were transduced with RV in the presence of polybrene (0.5 μg/ml) during spin infection (2,000 g for 60 min at 32° C.) following incubation at 37° C. for 6 hrs for single RV and sgRNA library, or 12 hrs for double RV. RV-transduced P14 cells were adoptively transferred into recipient mice that were infected 24-48 hrs prior to transfer.

Flow cytometry and sorting: For mouse experiments, tissues were processed, single cell suspensions obtained, and cells were stained as described (Wherry et al., (2003) Nature Immunology 2006 7:12 4, 225-234). Mouse cells were stained with LIVE/DEAD cell stain (Invitrogen) and antibodies targeting surface or intracellular proteins. Intracellular cytokine staining was performed after 5 hrs ex vivo stimulation with GP33-41 peptide in the presence of GolgiPlug, GolgiStop and anti-CD107a. After stimulation, cells were stained with surface antibodies, followed by fixation with Fixation/Permeabilization Buffer and then stained with intracellular antibodies for TNF, IFN-7 and MIP1α using Permeabilization Wash Buffer according to manufacturer's instructions. Flow cytometry was performed with an LSRII. Cell sorting experiments were performed with a BD-Aria sorter, with 70-micron nozzle and a 4° C. circulating cool-down system for sequencing, western and TIDE assays.

For sorting RV+ cells optimized sorting in the transfer experiments, the BD Aria Sorter was set at 37° C. and 100-micron nozzle, with a flow rate lower than 3.0. 3×10⁶ Cells were concentrated in 300ul 10% complete RPMI with 100 U/mL recombinant human IL-2 during sorting. 37° C. pre-warmed collection tubes with 10% complete RPMI (100 U/ml IL-2) were used. Sorted cells were washed by 37° C. warm pure RPMI before transferring into recipients.

TIDE Assay: At least 1×10⁴ Cas9⁺sgRNA+ T cell pellets were frozen down. Genomic DNA was isolated from these samples using QIAmp DNA Mini Kit. A TIDE PCR, using 2×Phusion Flash High-Fidelity PCR Master Mix and primers designed around the genome region of the sgRNA target part was run for each sample to extract the guide region from the genome DNA; the resulting products were then gel verified, PCR purified, and sent for Sanger sequencing.

Western Blot: 2×10⁵ T cells were sorted using FACS machine and the pellets were frozen down. Protein from these samples was extracted and denatured by boiling at 95° C. in 2×working loading sample buffer (1M Tris-HCl, 10% SDS, Glycerol, 10% Bromophenol blue). Lysate was run on a 10% SDS-PAGE gel and then transferred to a nitrocellulose membrane. Primary Fli1 (1:200) and GAPDH (1:1000) antibodies were staining over night, followed 1:5000 secondary antibody staining on the next day.

### OpTICS Screening

sgRNA candidate selection: 271 TFs that met the following criteria were selected 1) Among the top 50 differentially expressed across (Doering et al., (2012) Immunity 37, 1130-1144) and (Philip et al., (2017) Nature 2017 545:7652 545, 452-456), 2) Among the top 10 differentially open TF motifs across Naive, D8 Arm and D8 C113 in the previous described (Sen et al., (2016) Science 354, 1165-1169), 3) Involved in the top immune-regulatory families, such as IRF and STAT proteins. 120 TFs were manually chosen to be included in the TF library with the following principles: 1) TFs with known functions in CD8 T cells; 2) TF family members of those with related to immune functions, e.g. IRFs, STATs and Smads; 3) TF with the most significant differential RNA expression across published CD8 T cell datasets; 4) TF with the highest motif enrichment in ATAC-seq data from previous CD8 T cell data sets.

Library construction: 4-5 sgRNA were designed against individual DNA binding domains or other functional domains of each TF based on the domain sequence information retrieved from NCBI Conserved Domains Database. All of the sgRNA oligos, including positive and negative control sgRNAs, were synthesized by Integrated DNA Technologies (IDT) and pooled in equal molarity. The pooled sgRNA oligos were then amplified by PCR and cloned into BsmBI-digested SL21 vector using Gibson Assembly Kit. To verify the identity and relative representation of sgRNAs in the pooled plasmids, a deep-sequencing analysis was performed on a MiSeq instrument. We confirmed that 100% of the designed sgRNAs were cloned in the SL21 vector and the abundance of >95% of individual sgRNA constructs was within 5-fold of the mean.

Mouse Experimental Workflow: On day 0, C9P14 cells were isolated from the spleens and lymph nodes of CD45.2⁺ C9P14 mice and processed to standard T cell activation protocol using anti-CD3/CD28 and IL-2; on the same day, naive CD45.1V recipient mice were infected by LCMV. On D1 p.i., activated C9P14 cells were transduced by RV-sgRNA library and incubated for 6 hours before washing out the RV supernatant. 18-24 hours later, the transduced sgRNA⁺Cas9⁺ cells were sorted. Then, 10% of the sgRNA+Cas9⁺T cells were frozen down as a D2 baseline (TO time point) control prior to any selection, while 90% of the cells are transfer to the infected recipients (maximum 1×10⁵ cells/recipient). On the T1 time point (D8 in the graph), sgRNA⁺Cas9⁺ CD45.2⁺ T cells were sorted out from multiple organs of the recipients.

Isolated library construction and MiSeqprocessing: To quantify the sgRNA abundance of reference and end time points, the sgRNA cassette was PCR amplified from genomic DNA using high-fidelity polymerase. The PCR product was end-repaired by T4 DNA polymerase, DNA Polymerase I, Large (Klenow) Fragment, and T4 polynucleotide kinase. Next, a 3′ A-overhang was then added to the ends of blunted DNA fragments with Klenow Fragment (3′-5′ exo-). The DNA fragments were ligated to diversity-increased custom barcodes with Quick ligation kit. Illumina paired-end sequencing adaptors were attached to the barcoded ligated products through PCR reaction with high-fidelity polymerase. The final product was quantified by Bioanalyzer Agilent DNA 1000 and pooled together in equal molar ratio and pair-end sequenced by using MiSeq (Illumina) with MiSeq Reagent Kit V3 150-cycle (Illumina).

Dataprocessing: The sequencing data was de-multiplexed and trimmed to contain only the sgRNA sequence cassettes. The read count of each individual sgRNA was calculated with no mismatches and compared to the sequence of reference sgRNA as described previously (Shi et al., (2015) Nature Biotechnology 2015 33:6 33, 661-667). Data of each sample were normalized to the same read account. Waterfall plots (FIG. 1B): For each gene, mean of the log 2 fold changes from multiple sgRNA was computed. Heatmap (FIG. 1C): For each gene, mean of the log 10 fold changes from multiple sgRNA was computed. In the matrix of genes by conditions, quantile normalization was performed across conditions such that each condition has the same distribution of values. Genes were ordered by the mean value of each row. Histogram (FIG. 1D): For each condition, the background (grey bars and the histogram) was plotted for sgRNAs of all the genes. The 5% and 95% intervals were extracted by using the 5th percentile and 95th percentile of background values. The red bars show the log folds change for sgRNAs for one gene (or the control)

### RNA-Sequencing

Experiment workflow: At D8 p.i. with C113, CD8 T cells were isolated from spleens of infected recipients. VEX⁺GFP⁺ cells are sorted using FACS with >95% purity. RNA were isolated using the QIAGEN RNeasy Micro Kit with 2×10⁴ cell per sample. cDNA libraries were generated using SMARTSeq V4 Ultra Low kit. Libraries were quantified by qPCR using a KAPA Library Quant Kit (KAPA Biosystems). Normalized libraries were pooled, diluted to 1.8 μg/ml loaded onto a TG NextSeq 500/550 Mid Output Kit v2 (150 cycles, 130M reads, Illumina) and paired-end sequencing was performed on a NextSeq 550 (Illumina). The estimated read depth per sample is 15M reads.

Data processing: Raw FASTQ files from RNAseq paired-end sequencing were aligned to the GRCm38/mm10 reference genome using Kallisto (https://pachterlab.github.io/kallisto/). Sequencing reads were read in for 19357 genes and 8 samples. Genes with zero read count in more than three conditions were filtered out. 13628 genes remained after this step. Then, differential expression analysis was run using DESeq 2 package.

The expression of 1440 genes were found to significantly differ between the two conditions at a BH corrected P-value <0.05. GO enrichment analysis was performed using ClusterProfiler. The top 20 most enriched pathways are shown in the plot. GSEA was performed using the Broad Institute software (https://www.broadinstitute.org/gsea/index.jsp). Enrichment scores were calculated by comparing sgCtrl to sgFli1 groups. T_{EX} precursor gene signature was 25 from (Chen et al., (2019) Immunity, 51 6, 970-972). T_{EFF} gene signature was from (Bengsch et al., (2018) Immunity 48, 1029-1045.e5).

### ATAC-Sequencing

Experimental Workflow: ATACseq sample preparation was performed as described with minor modifications (Buenrostro et al., 2013). VEX⁺GFP+ cells were sorted using FACS with >95% purity. Sorted cells (2.5×10⁴) were washed twice in cold PBS and resuspended in 50 μl of cold lysis buffer (10 nM Tris-HCl, pH 7.4, 10 mM NaCl, 3 mM MgCl2, 0.1% Tween). Lysates were centrifuge (750xg, 10 min, 4° C.) and nuclei were resuspended in 50 μl of transposition reaction mix (TD buffer [25 μl], Tn5 Transposase [2.5 μl], nuclease-free water [22.5 μl]; (Illumina)) and incubated for 30 min at 37° C. Transposed DNA fragments were purified using a Qiagen Reaction MiniElute Kit, barcoded with NEXTERA dual indexes (Illumina) and amplified by PCR for 11 cycles using NEBNext High Fidelity 2×PCR Master Mix (New England Biolabs). PCR products were purified using a PCR Purification Kit (Qiagen) and amplified fragment sizes were verified on a 2200 TapeStation (Agilent Technologies) using High Sensitivity D1000 ScreenTapes (Agilent Technologies).

Libraries were quantified by qPCR using a KAPA Library Quant Kit (KAPA Biosystems). Normalized libraries were pooled, diluted to 1.8 μg/ml loaded onto a TG NextSeq 500/550 Mid Output Kit v2 (150 cycles, 130M reads, Illumina) and paired-end sequencing was performed on a NextSeq 550 (Illumina). Estimation read depth per sample is 10M reads.

Data processing: Raw ATACseq FASTQ files from paired-end sequencing were processed using the script available at the following repository (https://github.com/wherrylab/jogiles_ATAC). Samples were aligned to the GRCm38/mm10 reference genome using Bowtie2. Samtools was used to remove unmapped, unpaired, mitochondrial reads. ENCODE blacklist regions were also removed (https://sites.google.com/site/anshulkundaje/projects/blacklists). PCR duplicates were removed using Picard. Peak calling was performed using MACS v2 (FDR q-value 0.01). For each experiment, peaks of all samples were combined to create a union peak list and overlapping peaks were merged with BedTools merge. The number of reads in each peak was determined using BedTools coverage. Differentially accessible regions were identified following DESeq2 normalization using an FDR cut-off <0.05 unless otherwise indicated. Motif enrichment was calculated using HOMER (default parameters) on peaks differentially accessible across sgCtrl group and sgFli1 group. Transcription binding site prediction analysis was performed using known motif discovery strategy.

### Cut&Run

Experimental Workflow: CUT&RUN experiments were performed as previously described (Skene et al., (2018) Nature Protocols 2017 12:9 13, 1006-1019) with modifications. Briefly, 2×10⁵ sorted cells were washed twice with 1 ml of cold wash buffer (20 mM HEPES-NaOH pH 7.5, 150 mM NaCl, 0.5 mM Spermidine, and protease inhibitor cocktails from Sigma) in 1.5 ml tubes. Cells were then resuspended in 1 ml of cold wash buffer and incubated with 10 l of BioMagPlus Concanavalin A (Bangs laboratories) by rotating at 4° C. for 25 min to allow the cells to bind. Tubes were placed on a magnetic stand and liquid was removed after the solution turned clear. Primary antibody in 250 μl of cold antibody buffer (20 mM HEPES-NaOH pH 7.5, 150 mM NaCl, 0.5 mM Spermidine, 2 mM EDTA, 0.1% digitonin, and protease inhibitor cocktails from Sigma) was added to the tubes and rotated at 4° C. overnight. The next day, after washing cells once with 1 ml of cold wash buffer, protein A-MNase (pA-MN) in 250 μl of cold digitonin buffer (20 mM HEPES-NaOH pH 7.5, 150 mM NaCl, 0.5 mM Spermidine, 0.1% digitonin, and protease inhibitor cocktails from Sigma) was added to the tube and rotate at 4° C. for 1 h. To wash away unbound pA-MN, cells were washed twice with 1 ml of cold digitonin buffer, and then resuspended in 150 μl of cold digitonin buffer. The tubes were placed on a pre-cooled metal block. To initiate pA-MN digestion, 3 μl of 0.1 M CaCl₂) was mixed with cells in 150 μl cold digitonin buffer by gently flicking the tubes 10 times. Tubes were immediately placed back in the metal block. After 30 min incubation, the digestion was stopped by adding 150 μl of 2× stop buffer (340 mM NaCl, 20 mM EDTA, 4 mM EGTA, 0.02% Digitonin, 50 μg/ml RNase A, 50 μg/ml Glycogen, and 4 μg/ml yeast heterologous spike-in DNA). Target chromatin was released by incubating the tubes on a heat block at 37° C. for 10 min. Supernatant was spun at 16,000 g for 5 min at 4° C. and transferred to a new tube. Chromatin was incubated with 3 μl of 10% SDS and 2.5 μl of 20 mg/ml proteinase K at 70° C. for 10 min, followed by phenol:chloroform:isoamyl alcohol extraction. Upper phase containing DNA was mixed with 20 μg of glycogen and incubated with 750 μl of cold 100% ethanol at −20° C. overnight. DNA was precipitated by centrifugation at 20,000 g for 30 min at 4° C. DNA pellets was washed once by cold 100% ethanol, air-dried, and stored at −20° C. for library preparation. Protein A-MNase (batch 6, use at 1:200) and yeast heterologous spike-in DNA were kindly provided by Dr. Steve Henikoff. The antibodies used were: Fli1, ab15289, used at 1:50 (abcam) and guinea pig anti-rabbit IgG, used at 1:100, ABIN101961 (antibodies-online).

CUT&RUN DNA library was prepared as previously descried(Liu et al., 2018) with slight modifications. Briefly, all DNA precipitated from pA-MN digestion was used for library preparation using NEBNext Ultra II DNA Library Prep Kit (NEB). The adaptor was diluted to 1:25 for adaptor ligation. DNA was barcoded and amplified for 14 PCR cycle, and DNA library was cleaned up by AMPure XP beads(Liu et al., 2018). The library quality was checked with Qubit and bioanalyzer, and the quantity of the library was determined by qPCR using NEBNext Library Quant Kit for Illumina (NEB) according to manufacture's instruction. Eighteen barcoded libraries were pooled at equal molarity and sequenced in the NextSeq 550 platform with NextSeq 500/550 High Output Kit (75 cycles) v2.5 kit. Paired-end sequencing was carried out (42:6:0:42).

Dataprocessing: Paired-end reads were aligned to mm10 reference genome using Bowtie2 v2.3.4.1 with options suggested by Henikoff (Skene et al., (2018) Nature Protocols 2017 12:9 13, 1006-1019). Picard tools vl.96 was used to remove presumed PCR duplicates using the MarkDuplicates command. Bam files containing uniquely mapped reads were created using Samtools vi.1. For downstream analysis, biological replicates (3 per condition) were merged at this step. Bedtools v2.28.0 was used to generate fragment BED files with size 40 bp-500 bp. Blacklist regions, random chromosomes, and mitochondria were removed. Filtered BED files were used for downstream analysis.

Read per million (RPM) normalized bigwig files were created using bedGraphToBigWig (UCSC) and were used to visualize binding signals. Peaks were called using MACS v2.1 using the broadPeak setting with p-value cutoff of 1e-8, -f BEDPE and IgG as controls. Genes proximal to peaks were annotated against the mm10 genome using annotatePeaks.pl from HOMER v4. Fli1 binding motifs were identified using findMotifsGenome.pl from HOMER v4. Venn diagram of comparison with ATAC-Seq peaks was plotted using Bioconductor package ChIPpeakAnno. Heatmap was generated using Bioconductor package ComplexHeatmap.

Statistical analysis: Statistical significance was calculated with unpaired two-tailed student's t-test or one-way ANOVA with Tukey's multiple comparisons test by Prism 7 (GraphPad Software). P values are reported in the figure legends.

The results of the experiments are now described:

### Example 1: Optimized CRISPR-Cas9 for Gene Editing in Mouse Primary T Cells In Vivo

To enable gene editing in antigen specific primary CD8 T cells, LSL-Cas9⁺mice (Platt et al., (2014) Cell 159, 440-455) were crossed to CD4^{CRE+}P14⁺mice bearing CD8 T cells specific for the LCMV D^{b}GP₃₃-4₁ epitope (termed Cas9⁺P14, or C9P14). The backbone-optimized Cas9 single guide RNA (sgRNA) (Grevet et al., (2018) Science 361, 285-290) was expressed with a fluorescence marker in a retroviral (RV) vector (FIG. 8A). To evaluate gene editing efficiency in vivo, C9P14 cells were transduced ex vivo with either negative control sgRNA (sgCtrl1: AGTGGAAGCCATTGCTCTCG (SEQ ID NO: 714); sgCtrl_2: AATGGCAACTGGTCCCCTTC (SEQ ID NO: 715); sgCtrl_3 GAAGATGGGCGGGAGTC TTC (SEQ ID NO: 716)) or sgRNA targeting Pdcd1 (Encoding PD-1, sgPdcd1: CAGCTTGTCCAACTGGTCGG (SEQ ID NO: 717)) using an optimized RV transduction protocol (Kurachi et al., (2017) Nature Protocols, 12:9 12, 1980-1998) (FIG. 8B). The double-positive populations of sgRNA (mCherry+) and Cas9 (GFP+) CD8 T cells (FIG. 8C) were adoptively transferred into congenic recipient mice infected with the chronic strain of LCMV (clonel3; C113) (FIG. 8B). On day 9 post infection (p.i.), sgRNA⁺C9P14 cells were isolated and evaluated. As expected, sgPdcd1 induced antigen specific CD8 T cell expansion 5 fold greater than the control sgRNA (FIG. 8D), consistent with the genetic knockout of Pdcd1. The sgPdcd1 also resulted in a robust decrease of PD-1 protein expression (FIG. 8E) and indel mutations in the Pdcd1 locus (FIG. 8F). Furthermore, the high gene editing efficiency of the system was confirmed by designing sgRNAs targeting Klrgi (sgKlrgl: CCAAAGCCACCATT GCAAAG (SEQ ID NO: 718)) and Cxcr3 (sgCxcr3: GAACATCGGCTACAGCCAGG (SEQ ID NO: 719)) (FIG. 8G). Collectively, this in vitro sgRNA RV transduction in C9P14 followed by in vivo adoptive transfer system provides a robust platform to investigate genetic regulatory network of mouse CD8 T cells in vivo.

### Example 2: OpTICS Enables Pooled Genetic Screening in CD8 T Cells In Vivo

To enable in vivo pooled genetic screening in the LCMV infection system, the C9P14 and RV sgRNA platform (FIG. 1A) was further optimized. First, a physiological number of adoptively transferred CD8 T cells for screening was determined; as the number of T cells transferred can influence progression of cancer in tumor models or the outcome of infection in vivo. The number of adoptively transferred RV-transduced CD8 T cells was therefore limited to 1×10⁵ per mouse (˜1×10⁴ after take), a number previously optimized in chronic infection. Next, the performance of the system was evaluated by targeting a focused set of 29 TFs in CD8 T cells in vivo using the LCMV model. Previously, it was found that sgRNAs targeting functionally important protein-coding domains can substantially improve genetic screening efficiency, as both in frame and frameshift mutations contribute to generating loss-of-function alleles. A sgRNA library targeting the DNA-binding domains of 29 TFs and other control genes (e.g. non-selected control sgRNAs and Pdcd1) was designed and cloned, with 4-5 sgRNAs per target. An average input coverage of ˜400 cells per sgRNA after CD8 T cell engraftment improved the signal-to-noise ratio, and successfully identified hits compared to 100 cells per sgRNA (FIG. 8H). Third, the performance of the in vivo screen was assessed using P14 cells expressing heterozygous versus homozygous alleles of the LSL-Cas9 transgene. Cas9 heterozygous P14 cells outperformed Cas9 homozygous P14 cells in terms of signal-to-noise and consistency between independent screens (FIG. 8H-8I) perhaps due to reduced off-target DNA damage in the heterozygous setting. From these preliminary optimization screens, Batf; Irf4, and Myc we identified as essential for early T cell activation because genetic targeting of these genes potently inhibited T cell activation in vivo (FIG. 8H), consistent with known roles for Batf, Irf4, and Myc in T_{EFF} biology. This system was highly efficient with up to ˜100-fold enrichment for genes essential for CD8 T cell responses (FIG. 8H) and nearly 20-fold enrichment for genes that repress T cell activation and differentiation (FIG. 8J).

### Example 3: OpTICS Identifies Novel TF Involved in T_{EFF} and T_{EX} Cell Differentiation

To identify new TFs governing T_{EFF} and T_{EX} cell differentiation, another domain-focused sgRNA library was constructed against 120 TFs (Table 1). This library has 675 sgRNAs total, including 4-5 sgRNAs per DNA-binding domain, positive selection controls (sgPdcd1), and non-selection controls (e.g. sgAno9, sgRosa26, etc.) (FIG. 1A). With this 120-TF targeting sgRNA library, in vivo selection and sgRNA enrichment was interrogated at 1 or 2 weeks p.i. with acutely resolving LCMV Arm (Arm) or chronic C113 (FIG. 1A). C9P14 cells from different organs (PBMC, spleen, liver and lung) were examined to identify TFs broadly important for CD8 T cell responses to infection. In general, groups clustered by time point and infection, rather than anatomical location (FIG. 8K). At 2 weeks p.i. the data for Arm and C113 diverged (FIG. 8K), consistent with distinct trajectories of T cell differentiation during acutely resolved versus chronic infections. Focusing on the spleen, Batf; Irf4 and Myc emerged as some of the strongest negatively selected hits at both time points in both infections (FIG. 1B-1C). Several other known effector-driving TFs were confirmed including Tbx21 (encoding T-bet), Id2, Stat5a, Stat5b, and components of the NF-κB complex (FIG. 1B-1C). In addition, several TFs with potential novel roles in T cell activation and differentiation were revealed including Smad4, Smad7 andMybl2 (FIG. 1B-1C).

The OpTICS system was also used as an “UP” screen (Kaelin, (2017) Nature Reviews Cancer 2012 13:1 17, 441-450) to identify genes that repressed optimal T cell activation and T_{EFF} cell differentiation. Such genes, like Pdcd1, represent potential immunotherapy targets for improving T cell responses in cancer or infections. PD-1 served as a prototypical positive control where, as expected, Pdcd1-sgRNAs were strongly positively selected across infections, time points and in all tissues (FIG. 1B-1D). This screen also identified TFs that antagonized robust CD8 T cell responses (FIG. 1B-1C). Among these, Smad2 has been shown to limit T_{EFF} cell responses during both acute and chronic infection. Nfatc2 and Nr4a2 were also identified (FIG. 1C), both of which have been implicated in fostering T cell exhaustion and thus limiting T_{EFF} responses. In addition, Gata3 identified here has been implicated in driving T cell dysfunction and inhibiting T_{EFF} cell response. Thus, this screen identified key TFs known to restrain T_{EFF} differentiation and, in some cases, promote exhaustion.

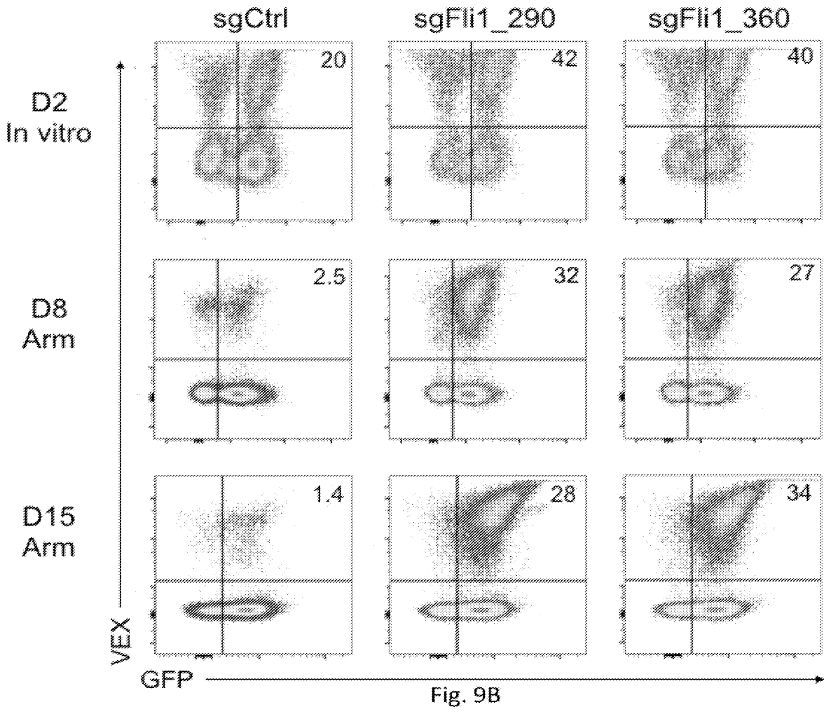

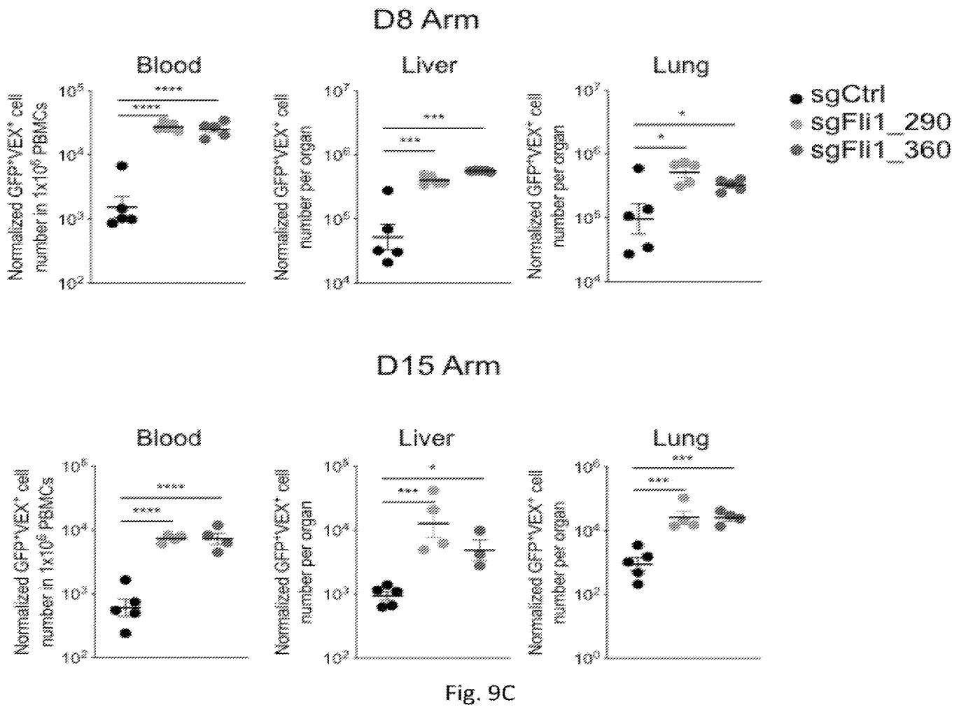

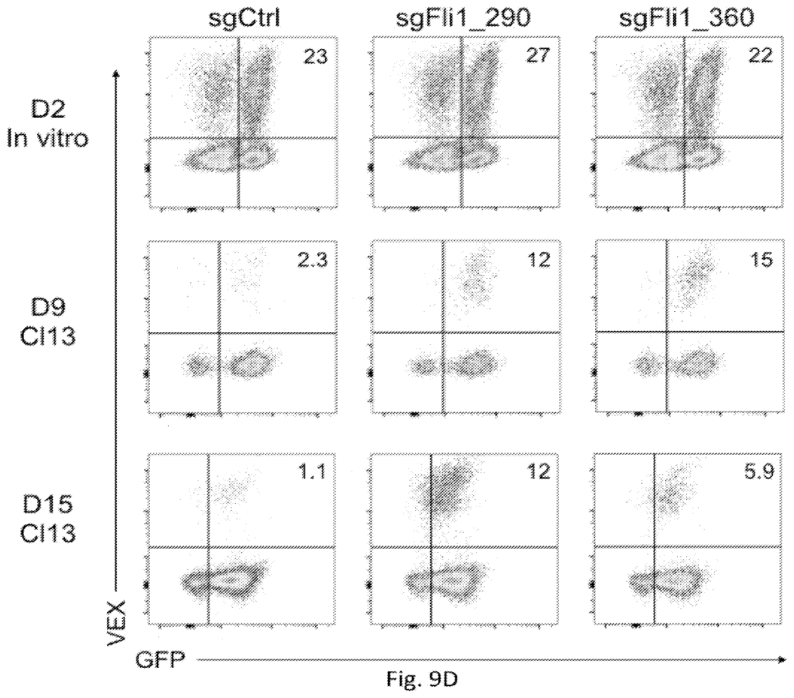

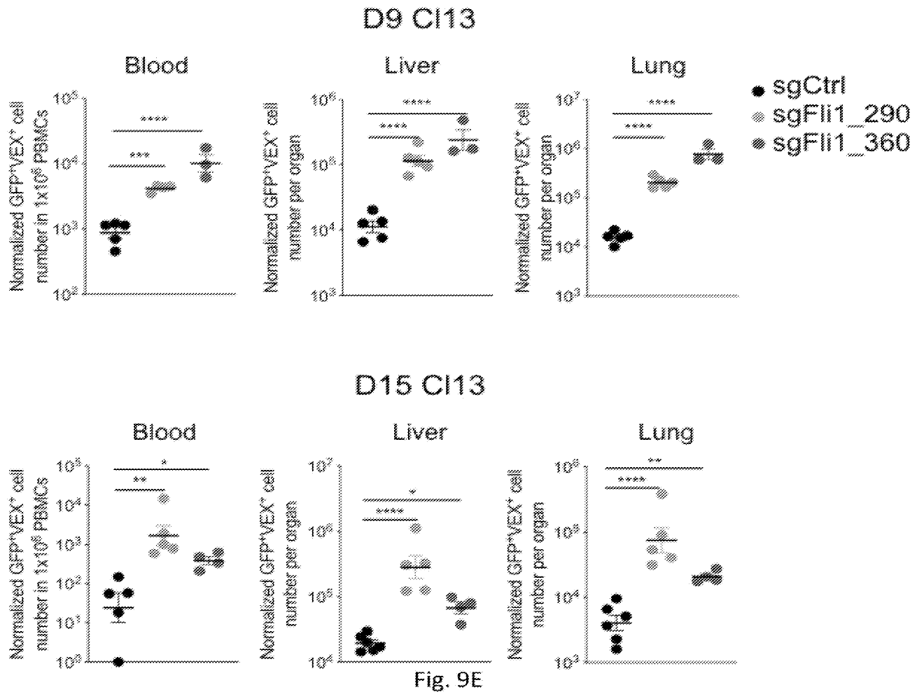

This OpTICS screen also identified novel TFs that restrained optimal T_{EFF} differentiation. This set of genes included Atf6, Irf2, Erg and Fli1, with Fli1 among the strongest hits in repressing T_{EFF} differentiation. The identification of Fli1 as a repressor of T_{EFF} differentiation occurred similarly in Arm and C113 infections (FIG. 1B-1D) indicating a common role for this TF in restraining T_{EFF} biology. Two Fli1-sgRNAs (sgFli1_290: CGCTGTCGGACAGTAGT TCC (SEQ ID NO: 720) and sgFli1_360: GCCATGGAAGTCAAACTTGT (SEQ ID NO: 721)) were selected and it was confirmed that these sgRNAs effectively edited the Fli1 gene (70%-80% editing; FIG. 9A) leading to reduced protein expression (FIG. 1E). Targeting Fli1 using these individual Fli1-sgRNAs in C9P14 cells in vivo resulted in 5-20 fold greater expansion at 1 and 2 weeks p.i. with either Arm or C113 (FIG. 1F and 9B-9E). These data indicate2 repression of robust CD8 T cell expansion by Fli1 in acutely resolving or developing chronic infection.

### Example 4: Genetic Deletion of Fli1 Promotes Robust T_{EFF}-Differentiation During Acutely Resolved Infection

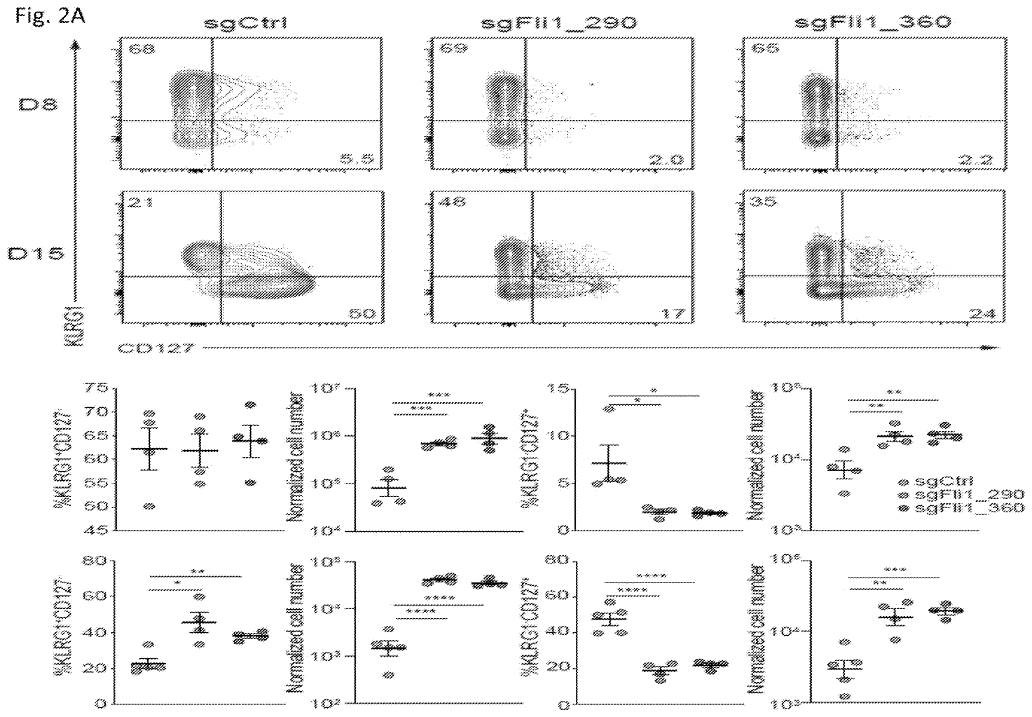

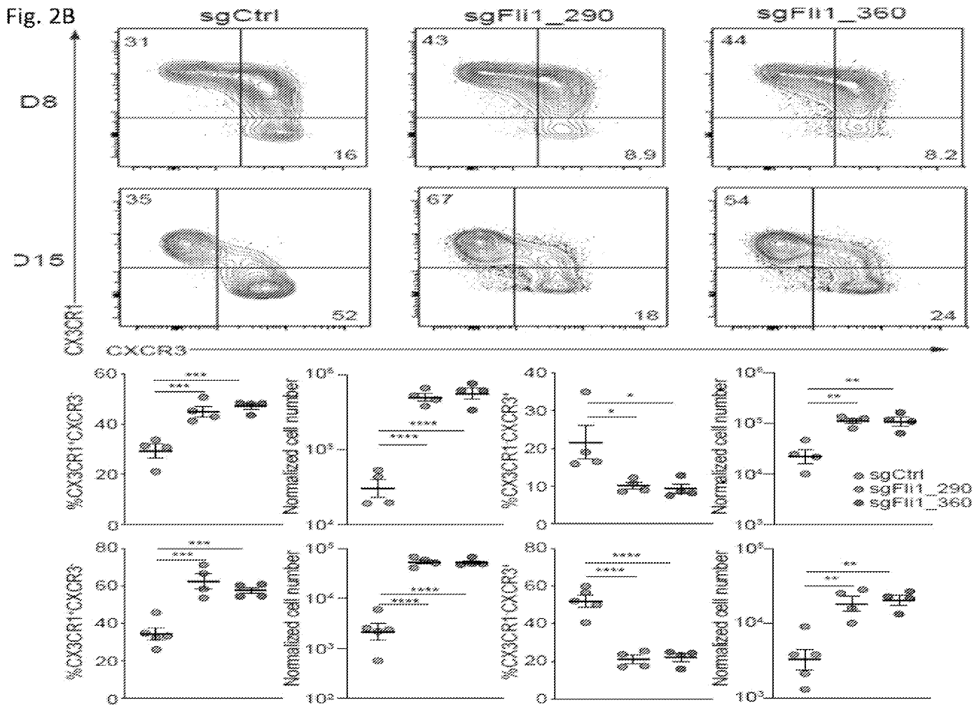

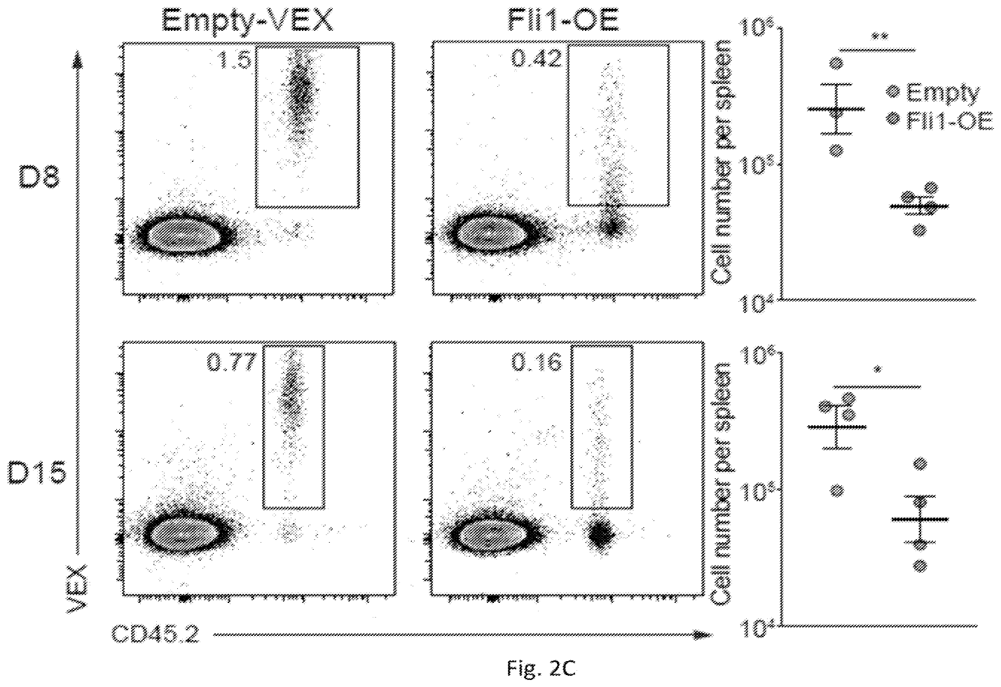

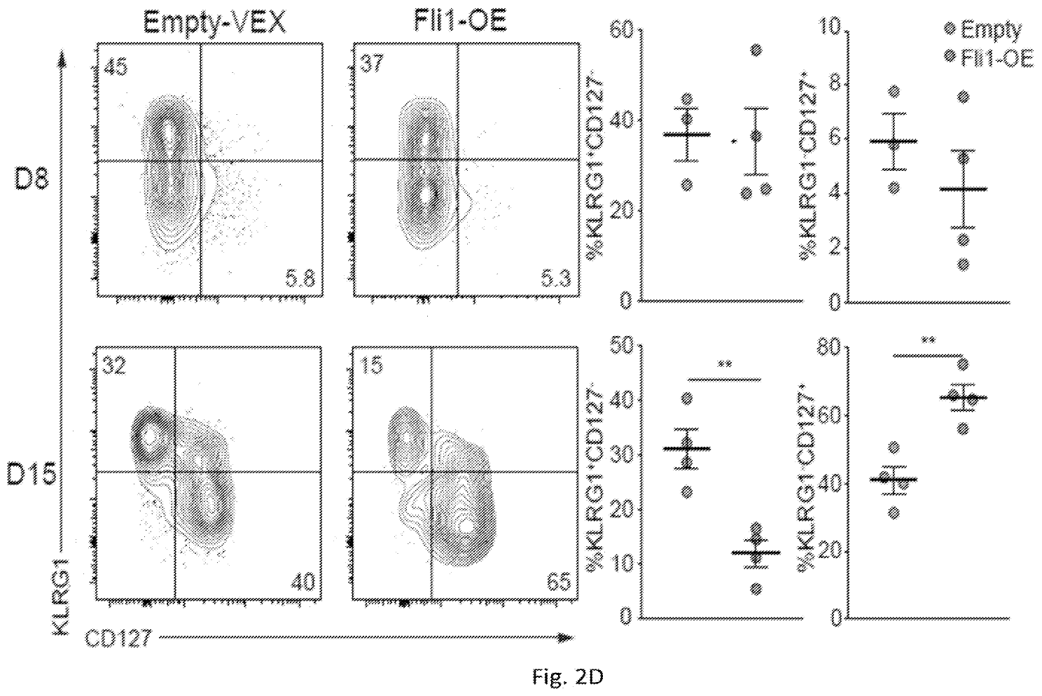

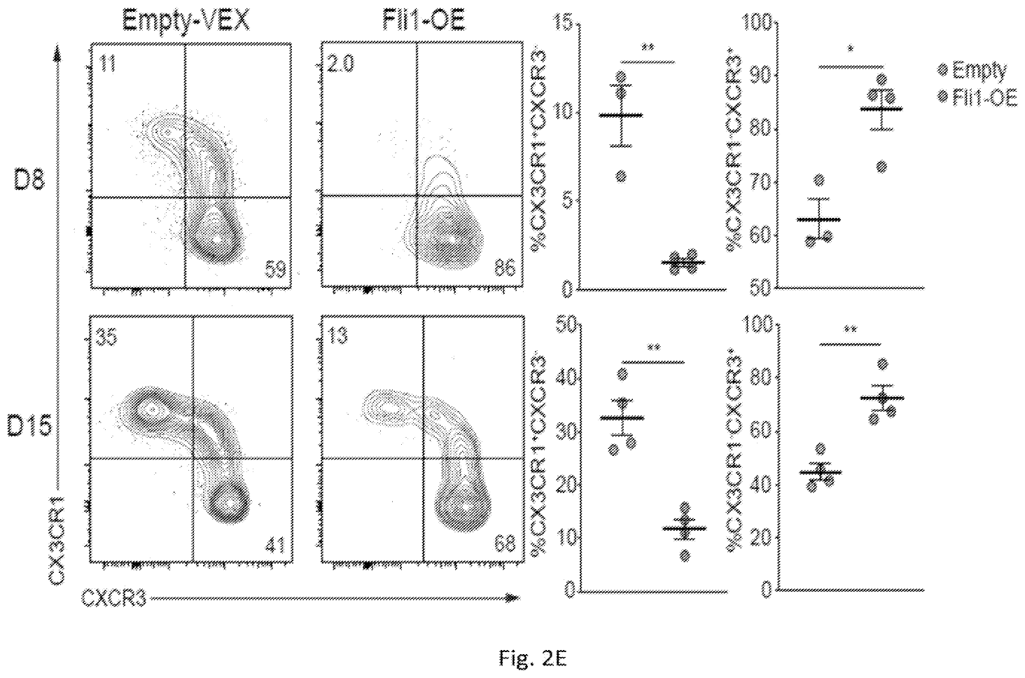

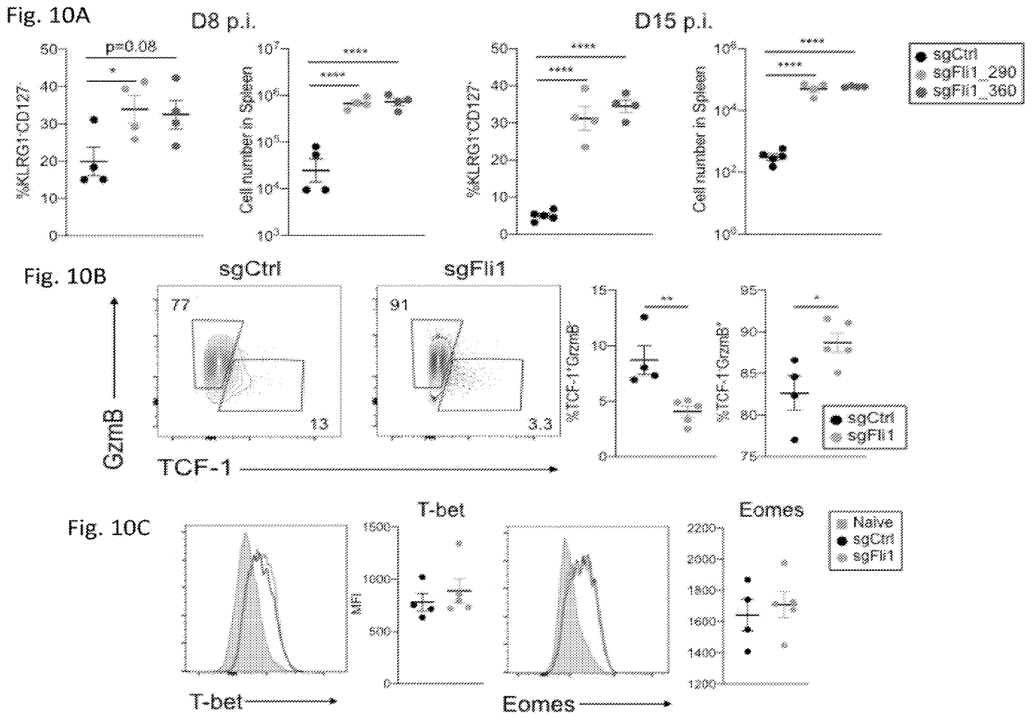

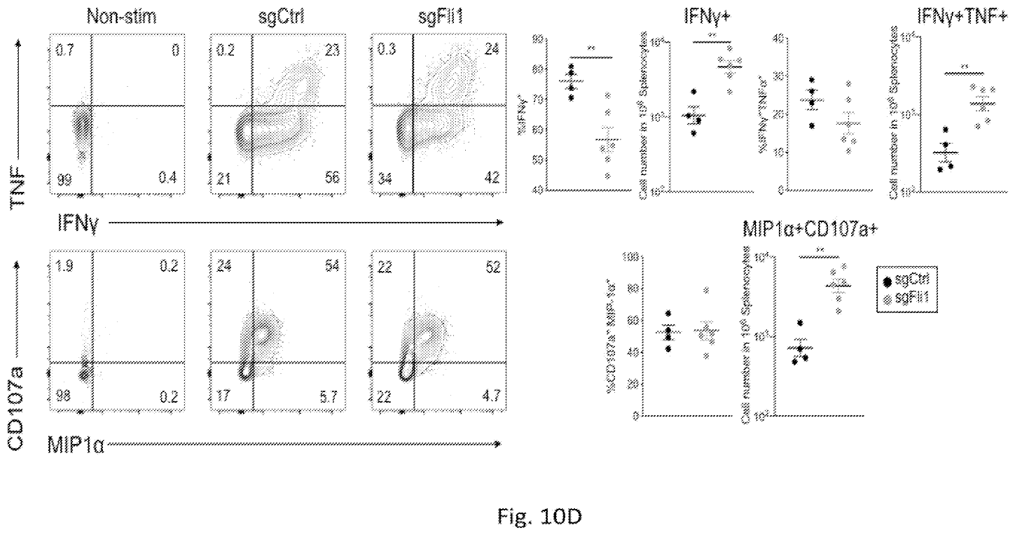

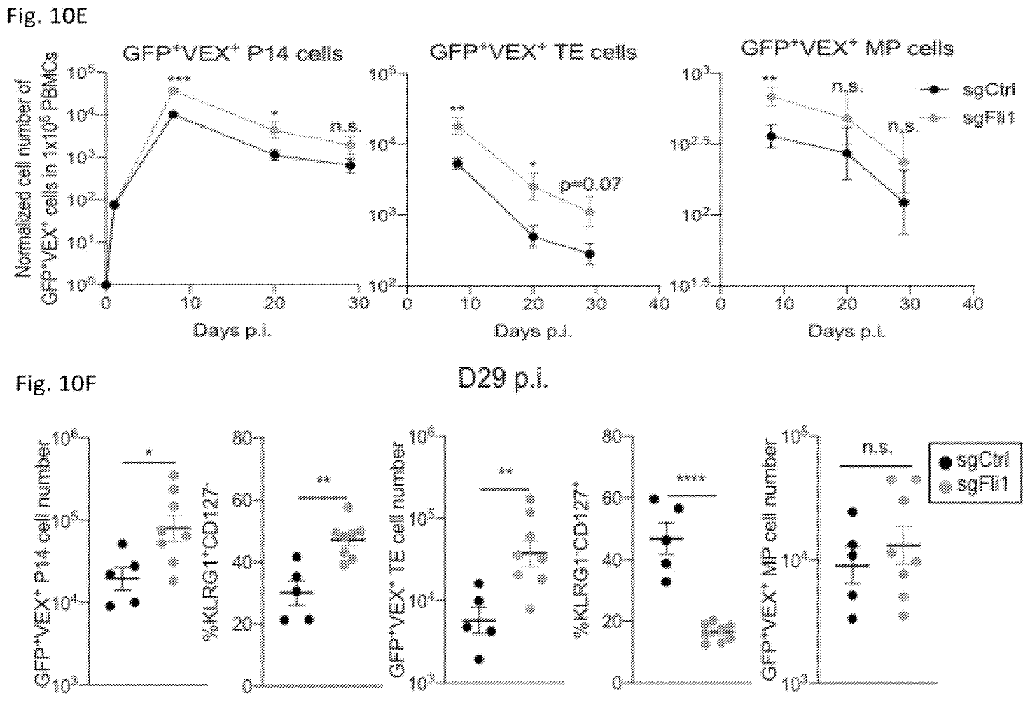

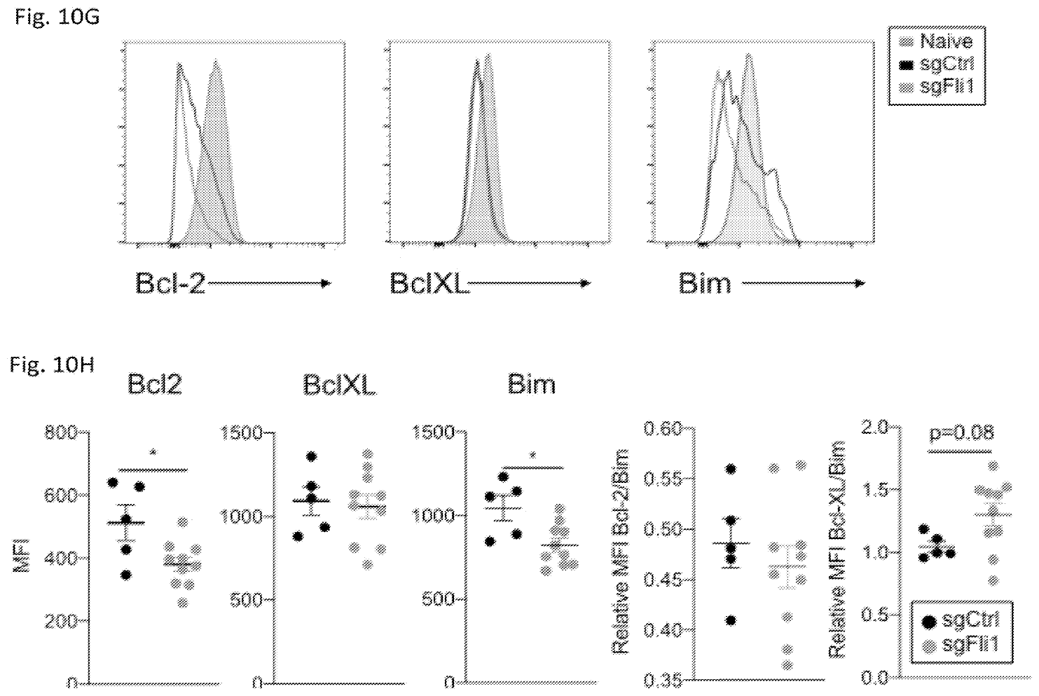

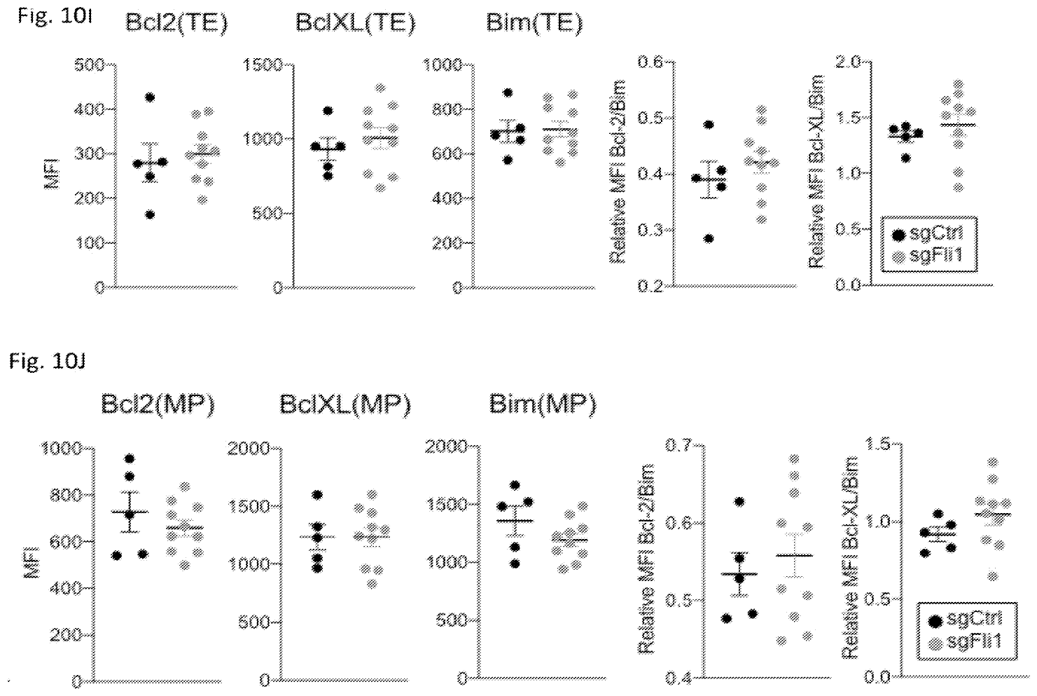

The differentiation state of Fli1-sgRNA (sgFli1) or Ctrl-sgRNA (sgCtrl) transduced C9P14 cells was next interrogated during acutely resolved infection. On Day 8 p.i., Fli1 deletion reduced the proportion of the CD127^{Hi} memory precursors (TMw), whereas the frequency KLRG1^{Hi} terminal effector (T_{EFF}) population remained unchanged and the CD127^{Lo}KLRG1^{Lo}population slightly increased (FIGS. 2A and 10A). These effects were more dramatic at Day 15 p.i. when the frequency of the CD127^{Hi} T_{MP} population was reduced and the KLRG1^{Hi} T_{EFF} cell population increased (FIG. 2A). However, at both time points the absolute number of both T_{MP} and T_{EFF} was increased ˜2-10-fold due to the proliferative expansion of Fli1-deficient CD8 T cells (FIG. 2A). This skewing of T cell differentiation towards T_{EFF}-like populations was also observed using CX3CR1 and CXCR3, with sgFli1 significantly enriching for the CX3CR1⁺CXCR3⁻ T_{EFF} population compared to the CX3CR1⁻CXCR3⁺early T_{MEM} subset (FIG. 2B). C9P14 cells with increased cytotoxic potential (GzmB⁺TCF-1⁻) were also increased in the sgFli1⁺group (FIG. 10B), though the proportion of cells expressing T-bet or Eomes was similar between the groups (FIG. 10C). The frequency of C9P14 cells producing IFN-γ after ex vivo peptide stimulation was slightly lower in the sgFli1⁺versus sgCtrl⁺ group, but the absolute number of IFN-γ producing cells, or cells co-producing multiple effector molecules simultaneously was increased (FIG. 10D).

One concern with fostering an increase in T_{EFF} is preventing the formation of T_{MEM}. Thus, formation of Fli1-deficient T_{MEM} was examined 1 month p.i. Indeed, the number of KLRG1^{Hi} effector memory C9P14 cells remained higher in the sgFli1⁺C9P14 group compared to the sgCtrl⁺ group on Day 29 p.i. The number of CD127^{Hi} T_{MEM} at this time point was similar between the groups (FIG. 10E-10F), indicating that the robust increase in the KRLG1^{H}I T_{EFF} populations generated in the absence of Fli1 did not compromise the formation of T_{MEM}. To further interrogate the effects of Fli1 deficiency on T_{MEM}, expression of pro- and anti-apoptotic molecules, Bcl-2, Bcl-XL and Bim, were examined. On Day 15 p.i. sgFli1⁺C9P14 cells had lower expression of both Bcl-2 and Bim but no change in Bcl-XL expression compared to the 20 sgCtrl⁺ group (FIG. 10G-10H), resulting in a trend to a higher BclXL/Bim ratio in the sgFli1⁺ C9P14 group (FIG. 10H). There were no differences in expression of Bcl-2, Bcl-XL or Bim in T_{EFF} or T_{MP} subsets (FIG. 10I-10J) suggesting that the differences in total C9P14 populations reflect, in part, different proportions of terminal T_{EFF} and T_{MP}. These observations may be consistent with the observation that the Bcl-XL/Bim ratio may be more important in terminal T_{EFF} survival than the Bcl-2/Bim ratio.

Fli1 expression was enforced in WT LCMV-specific P14 cells using an RV based overexpression (OE) system (Kurachi et al., (2017) Nature Protocols, 12:9 12, 1980-1998). A ˜5-fold reduction in responding Fli1-OE-RV transduced P14 cells was observed on Day 8 and Day 16 p.i. compared to the empty vector control (FIG. 2C). Furthermore, enforced Fli1 expression diverted the responding P14 cells towards T_{MP} differentiation (FIG. 2D-2E). Together, these data reveal a role for Fli1 in restraining T_{EFF} differentiation and promoting T_{MP} development during acute infection.

### Example 5: Fli1 Antagonizes T_{EFF}-Like Differentiation During Chronic Infection

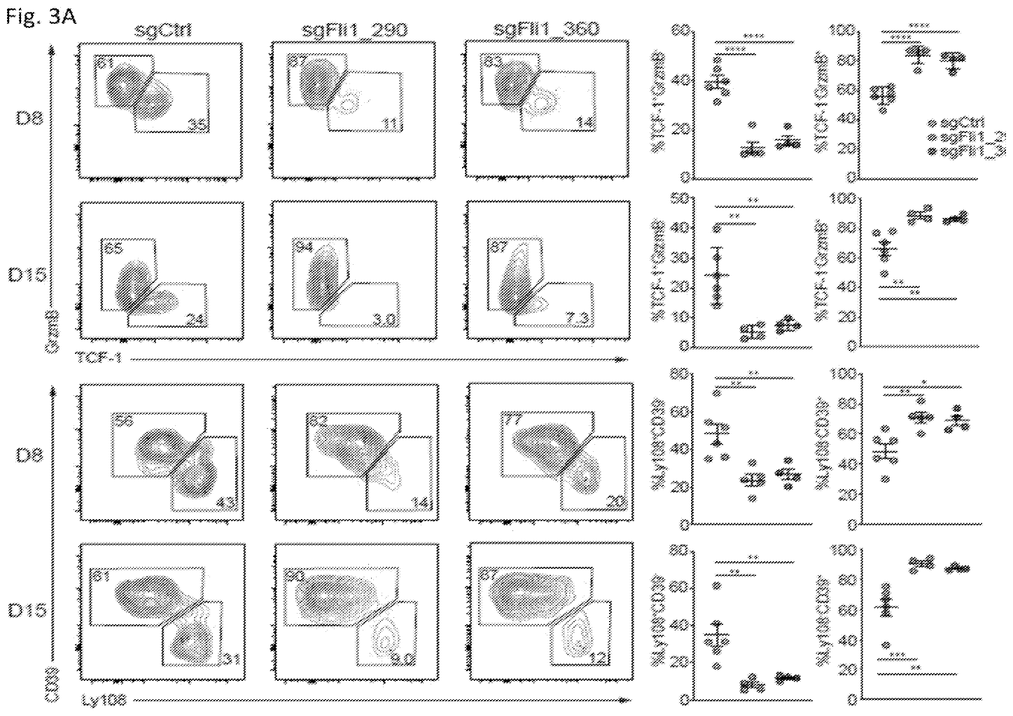

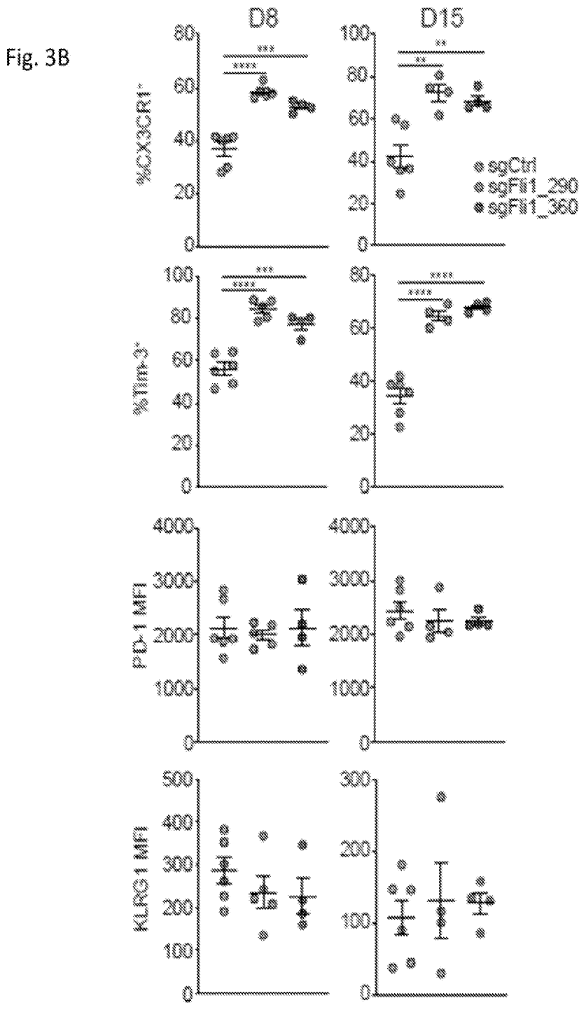

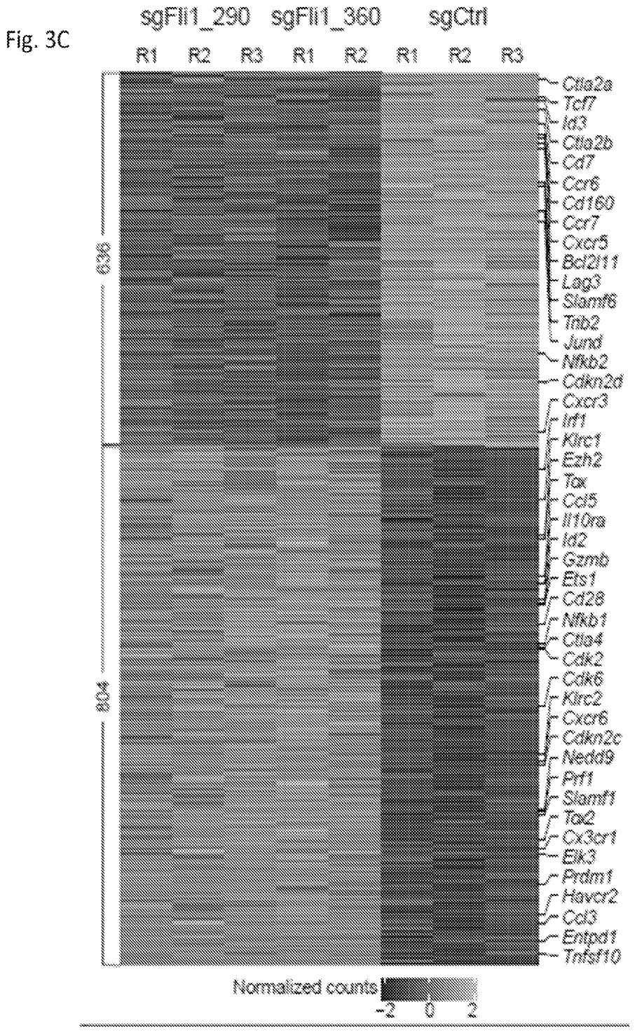

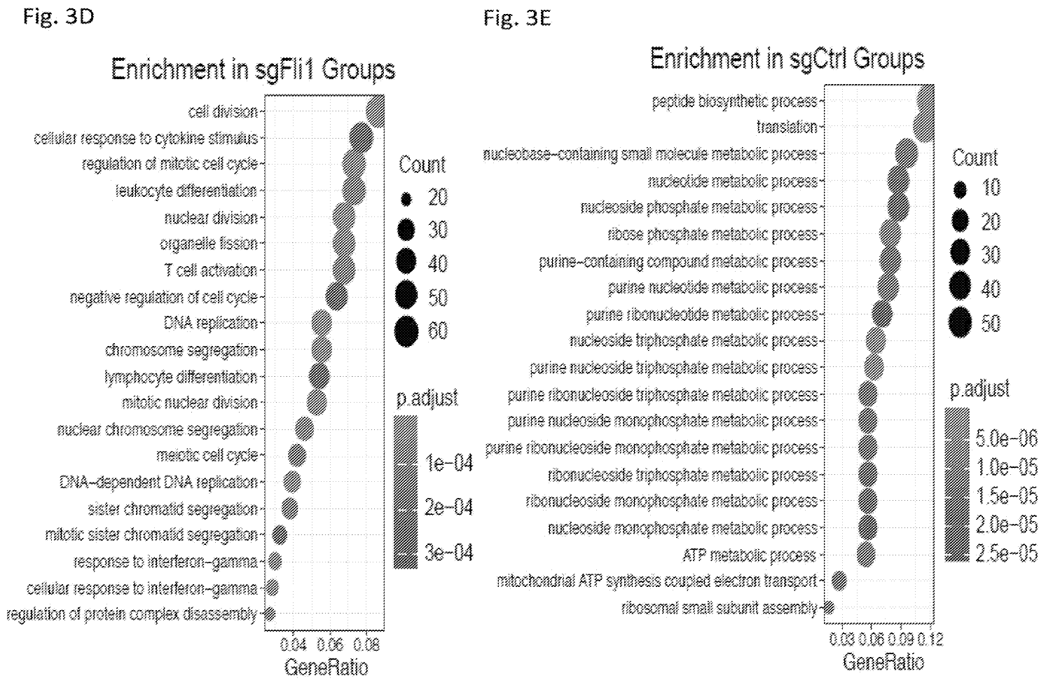

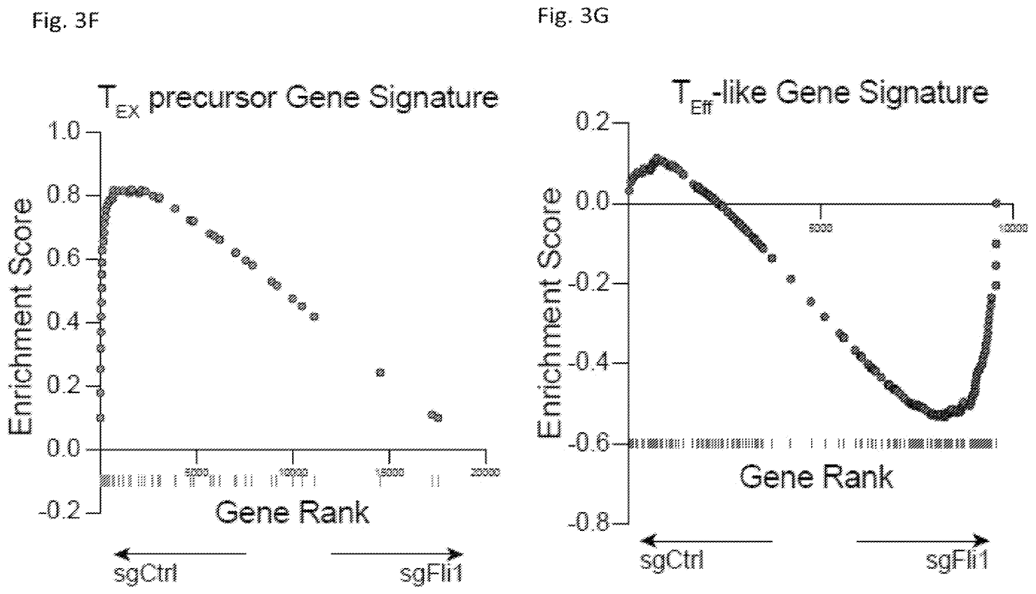

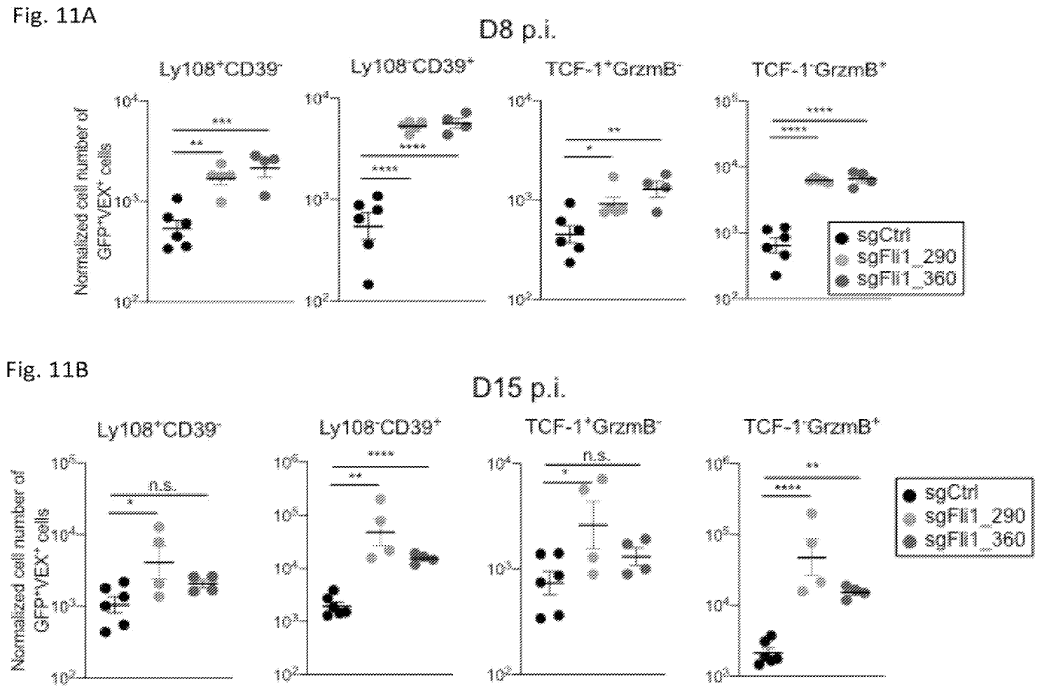

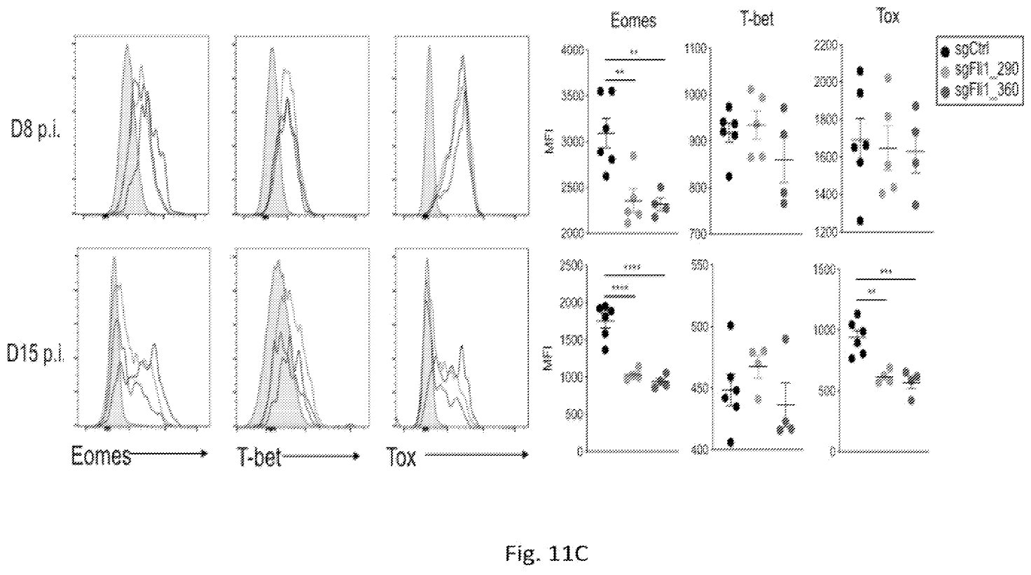

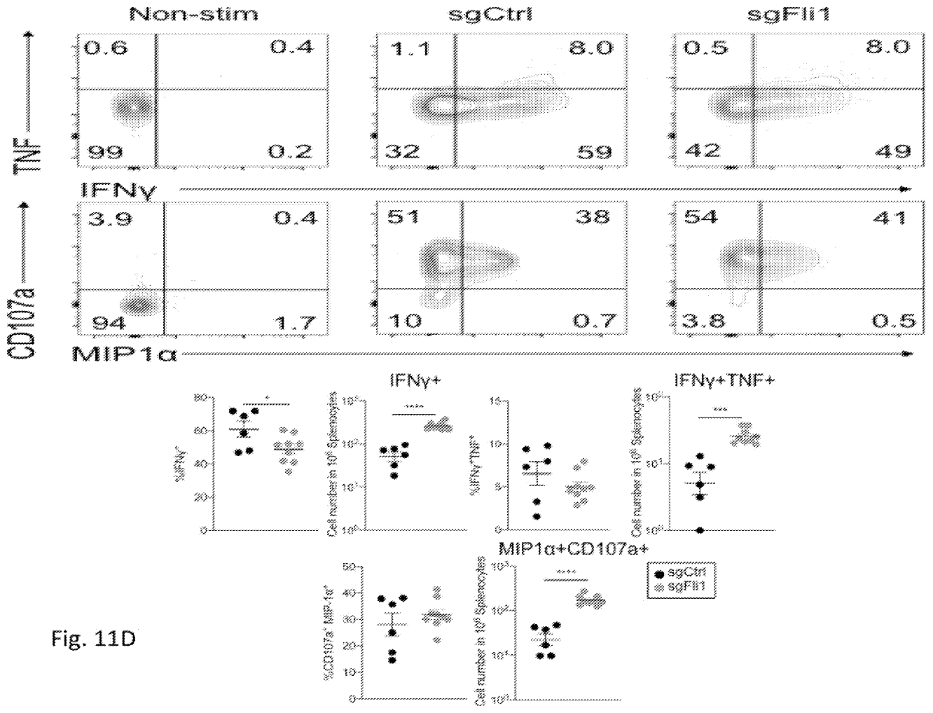

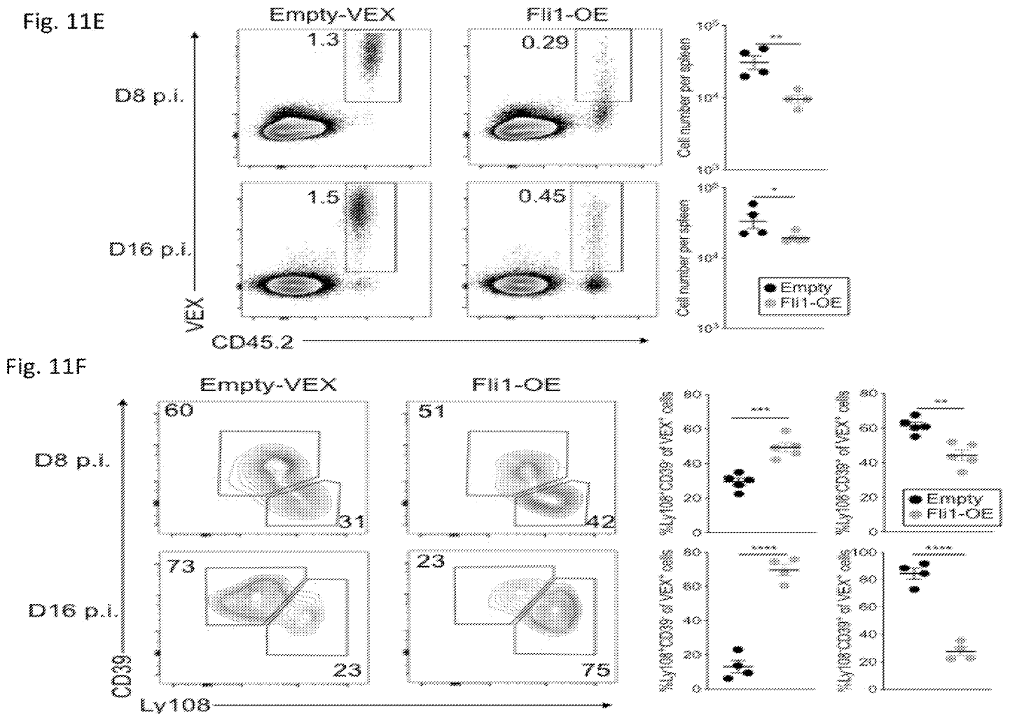

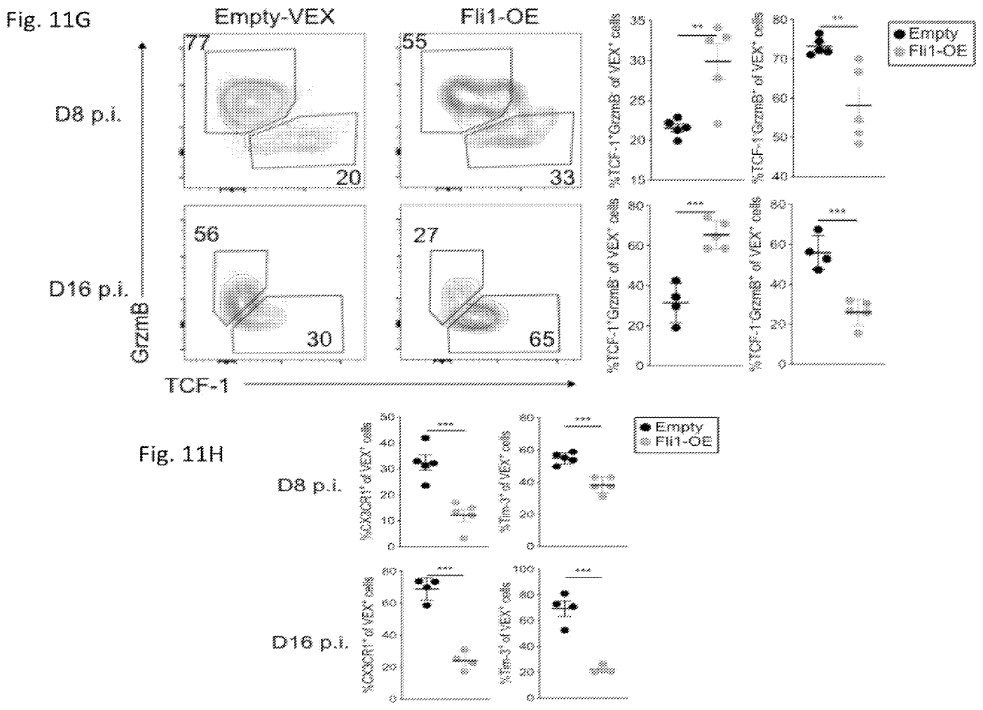

During chronic viral infection, there is an early fate bifurcation for CD8 T cell responses where antiviral CD8 T cells develop into either terminal T_{EFF}-like cells or form T_{EX} precursors that ultimately seed the mature T_{EX} population. Therefore, the role of Fli1 in this cell fate decision was investigated early during chronic infection. As in acutely resolving infection, genetic perturbation of Fli1 skewed the virus-specific CD8 T cell response towards the T_{EFF} pathway, defined as TCF-1-GrzmB+or Ly108-CD39⁺ cells (FIG. 3A). Due to the 5-10-fold increase in total Fli1-deficient cells, the cell numbers of both the T_{EFF}-like and T_{EX} precursor populations were increased (FIG. 11A-11B). The TF circuitry known to be involved in early T_{EX} formation was also investigated. TCF-1 at this early time point drives expression of Eomes, another TF central to T_{EX} formation and, indeed, Eomes was reduced in the absence of Fli1 (FIG. 11C). Expression of T-bet, another T-box TF, was similar between sgCtrl⁺ and sgFli1⁺groups (FIG. 11C). Loss of Fli1 was also associated with lower expression of the T_{EX} master regulator Tox on Day 15 p.i. with C113, suggesting that Fli1-deficiency antagonized the development of T_{EX} and instead fostered T_{EFF}-like differentiation (FIG. 11C). This skewing towards the T_{EFF} fate was associated with increased expression of cytotoxic molecules (FIG. 3A) and higher cell numbers that translated to increased numbers of cytokine producing cells (FIG. 11D). Moreover, genetic perturbation of Fli1 resulted in an increased proportion of CX3CR1⁺ and Tim3⁺T_{EFF}-like cells in chronic infection (FIG. 3B). In contrast, enforced Fli1 expression had the opposite effect and not only resulted in lower cell numbers (FIG. 11E), but also fostered formation of Ly108⁺CD39⁻ or TCF-1⁺GrzmB- T_{EX} precursors and fewer CX3CR1⁺cells (FIG. 11F-11H). Notably, PD-1 expression was unchanged and, unlike acutely resolved infection, KLRG1 expression was unaffected (FIG. 3B).

To dissect the underlying mechanisms, RNA-seq was performed on sorted sgCtrl⁺ or sgFli1⁺C9P14 cells on Day 9 of C113 infection. Both sgRNAs targeting Fli1 resulted in a similar transcriptional effect (FIG. 3C, 12A). The sgFli1⁺C9P14 cells were transcriptionally distinct from the sgCtrl⁺ C9P14 cells with over 1400 genes differentially expressed between the two conditions (FIG. 3C, 12A). Effector-associated genes such as Prfl, Gzmb, Cd28, Ccl3 and Prdm1 were robustly increased in the sgFli1⁺C9P14 cells, whereas the sgCtrl⁺ C9P14s were enriched in T_{EX} precursor genes such as Tcf7, Cxcr5, Slamf6 and Id3 (FIG. 3C). Gene Ontology enrichment analysis also identified cell division-associated and T cell activation-associated pathways in sgFli1⁺C9P14 cells (FIG. 3D), whereas sgCtrl⁺ C9P14s enriched in metabolic pathways, in particular nucleotide, nucleoside and purine biosynthesis (FIG. 3E). Gene set enrichment analysis (GSEA) was used to examine the early phase of chronic infection, when a divergence of differentiation into either T_{EFF}-like or T_{EX} precursor cell fates occurs. Indeed, the T_{EX} precursor signature was strongly enriched in sgCtrl⁺ C9P14 cells compared to the sgFli1V C9P14 population whereas a T_{EFF} gene signature was strongly enriched in the sgFli1V C9P14 population (FIG. 3F-3G). Thus, Fli1 repressed optimal T_{EFF} differentiation in both acutely resolving and chronic infection and loss of Fli1 antagonized the development of T_{EX} cells. However, although genetic perturbation of Fi drove an increase in expression of effector-associated genes at Day 9 of chronic infection, Tox, Tox2 and Cd28 were also increased. This effect may suggest that although loss of Fli1 might enhance T_{EFF}-like biology, this effect might not be at the expense of genes necessary to sustain responses in chronic infection of cancer.

### Example 6: Fli1 Remodels the Epigenetic Landscape of CD8 T Cells and Antagonizes T_{EFF}-Associated Gene Expression

In acute myeloid leukemia, FLIl co-localizes with the chromatin remodeler BRD4 and the EWS-Fli1 fusion oncoprotein driving Ewing's sarcoma can trigger de novo enhancer formation via chromatin remodeling and can inactivate existing enhancers by displacing ETS family members. It is unclear, however, how Fli1 affects epigenetic landscape changes in developing T_{EFF}, T_{MEM} or T_{EX} cells.

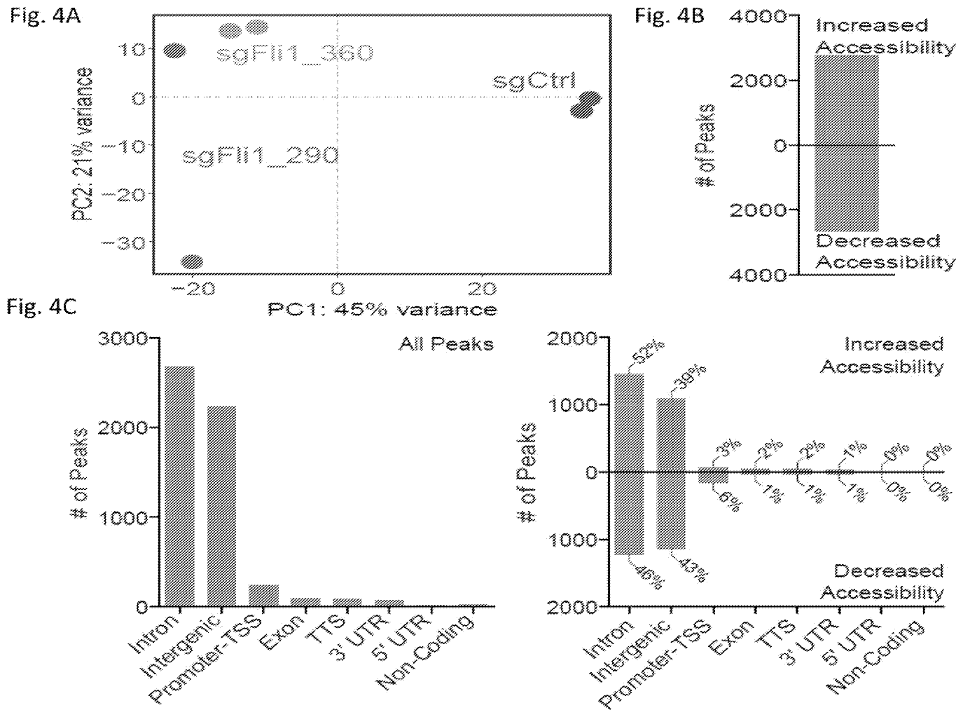

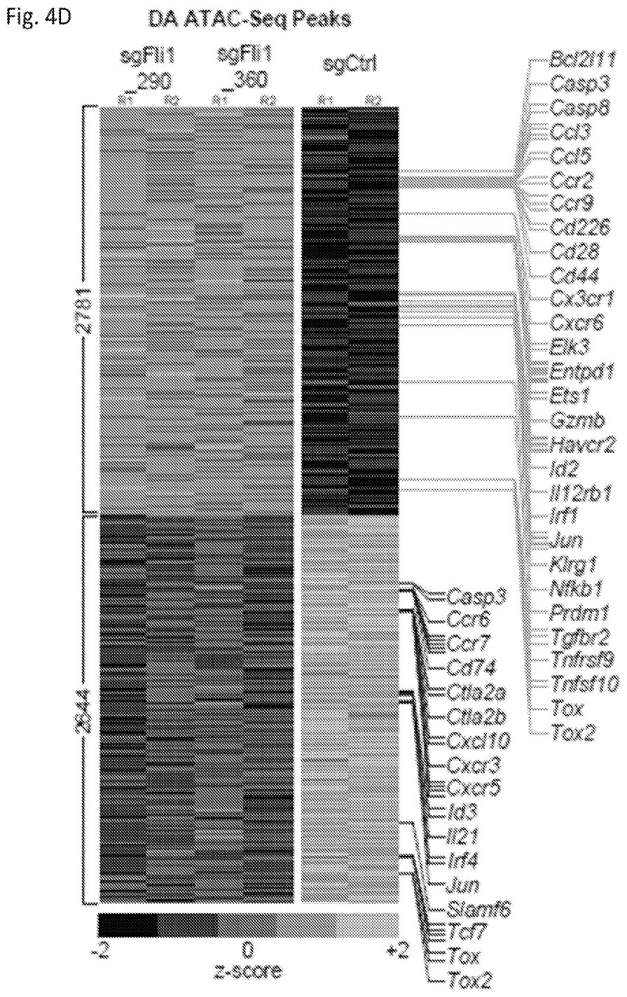

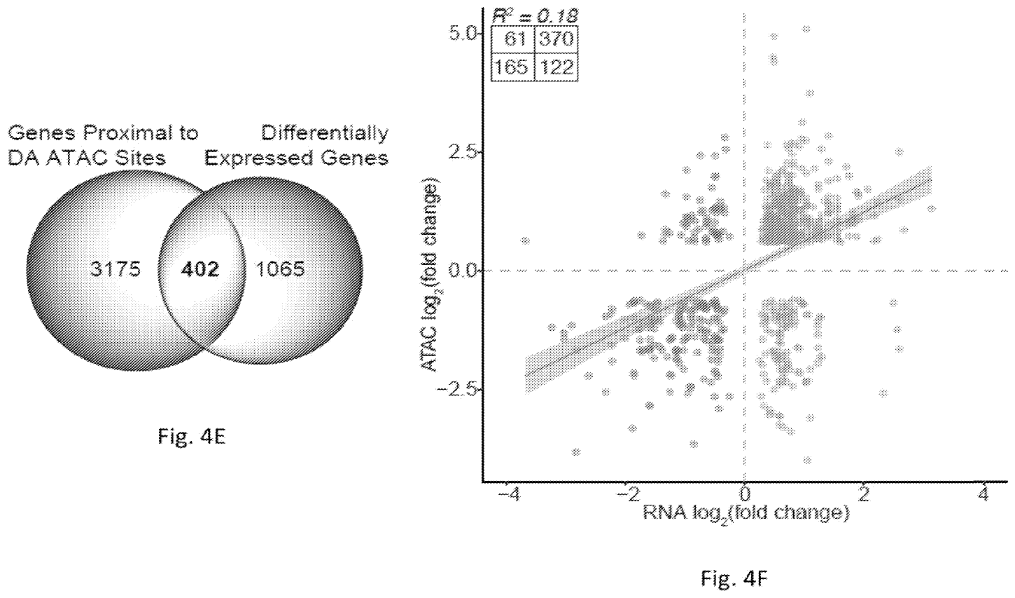

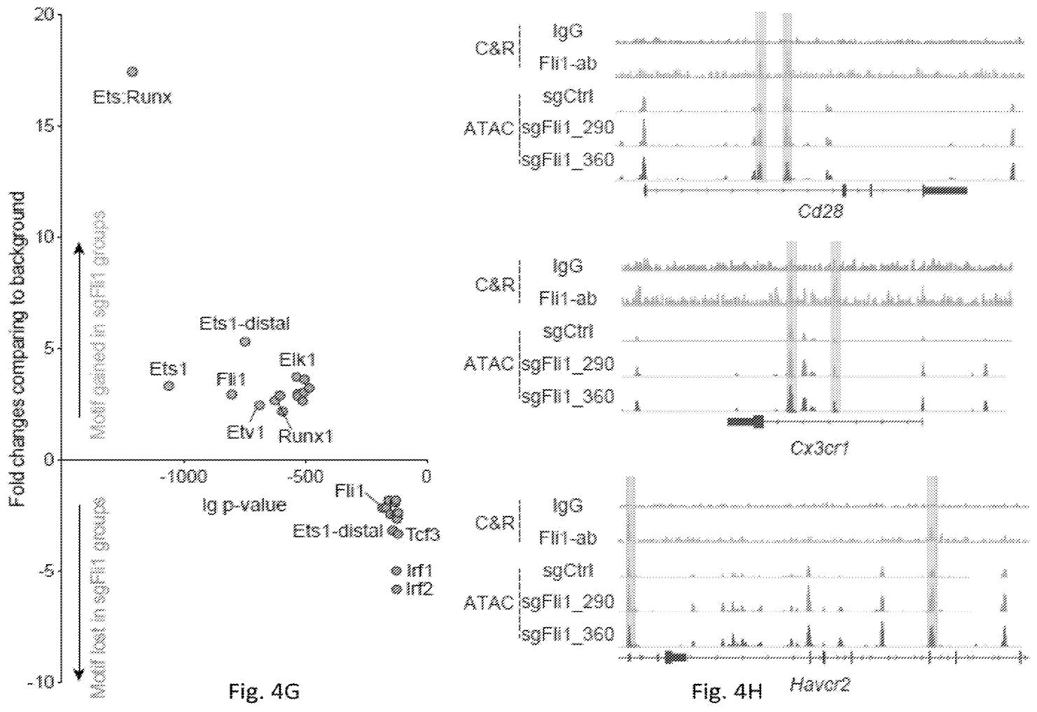

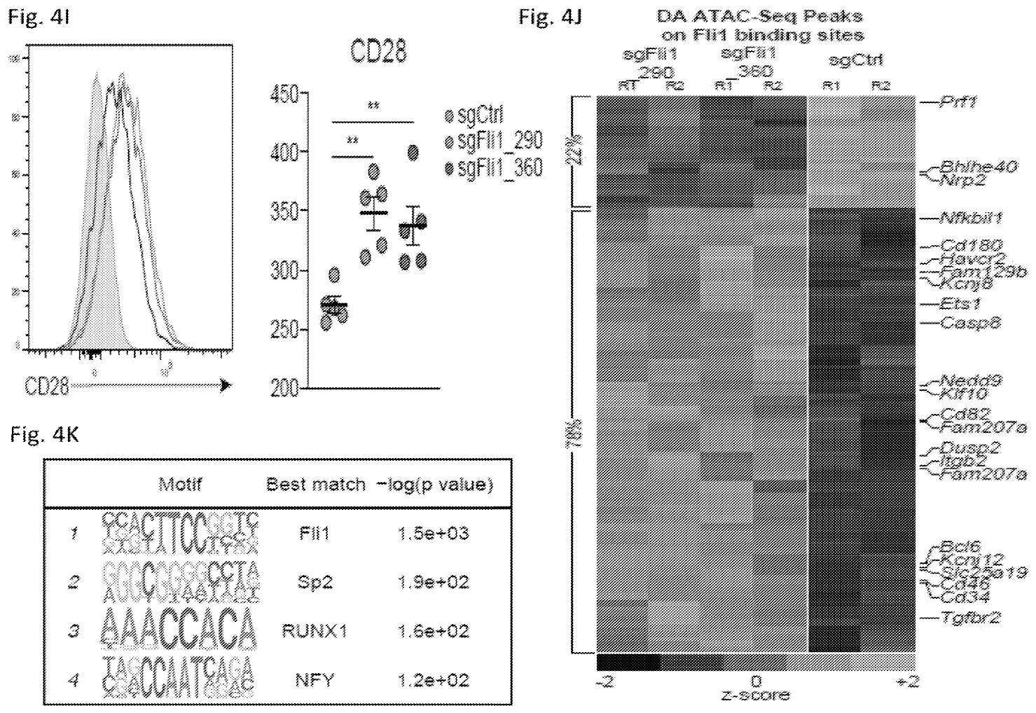

To examine the role of Fli1 in supporting the epigenetic landscape of CD8 T cells, ATAC-seq was performed on sgFli1V and sgCtrl⁺ C9P14 cells on Day 9 p.i. with C113. Compared to sgCtrl⁺ C9P14 cells, the sgFli1V group had considerable changes in chromatin accessibility (FIG. 4A). Over 5000 open chromatin regions (OCRs) differed between the control and Fli1-perturbed groups with approximately equal numbers of peaks gained or lost (FIG. 4B-4D). Most of these changes were located in intronic or intergenic regions consistent with cis-regulatory or enhancer elements (FIG. 4C).

Each OCR was assigned to the nearest gene to estimate genes that could be regulated by these cis-regulatory elements. T_{EFF}-associated genes, such as Ccl3, Ccl5, Cd28, Cx3cr1 and Prdm1, gained chromatin accessibility in the sgFli1V group (FIG. 4D), consistent with the RNA-seq data. In contrast, there was decreased chromatin accessibility near genes involved in T cell progenitor biology such as Tcf7, Slamf6, Id3 and Cxcr5 (FIG. 4D) in the sgFli1V group. Moreover, the sgFli1⁺C9P14 cells had altered accessibility in the Tox (and Tox2) locus, but these changes included both increased and decreased accessibility for different peaks (FIG. 4D). These changes in chromatin accessibility corresponded to changes in gene expression with −⅓ of the genes that changed transcriptionally associated with a differentially accessible chromatin region (402 out of 1467) (FIG. 4E). In general, increased accessibility correlated with increased transcription though there was a clear subset of regions where decreased accessibility corresponded to increased transcription (FIG. 4F).

Next, TF motifs present in the OCRs that were dependent on Fli1 for altered accessibility were defined. Among the OCRs that decreased in accessibility in the absence of Fli1, the most enriched TF motifs were for IRF1 and IRF2 (FIG. 4G), potentially linking Fli1 to IRF1 and IRF2 downstream of IFN signaling or to the regulation of cell cycle by these TFs. In the group of OCRs that increased in accessibility in the absence of Fli1, ETS and RUNX motifs were highly enriched (FIG. 4G). The composite ETS:RUNX motif was by far the most changed with 18-fold enrichment (FIG. 4G). These observations suggested that Fli1 may limit the activity of other ETS family members (e.g. ETS1, ETV1 or ELK1) or alter accessibility at ETS:RUNX binding sites (FIG. 4G). Runx3 is a central driver of T_{EFF} differentiation and functions by directly regulating effector gene expression, coordinating and enabling the effector gene regulation via T-bet and Eomes and antagonizing TCF-1 expression. Thus, a potential role for Fli1 in Runx3 biology would provide a mechanistic link between loss of Fli1 and improved T_{EFF} differentiation.

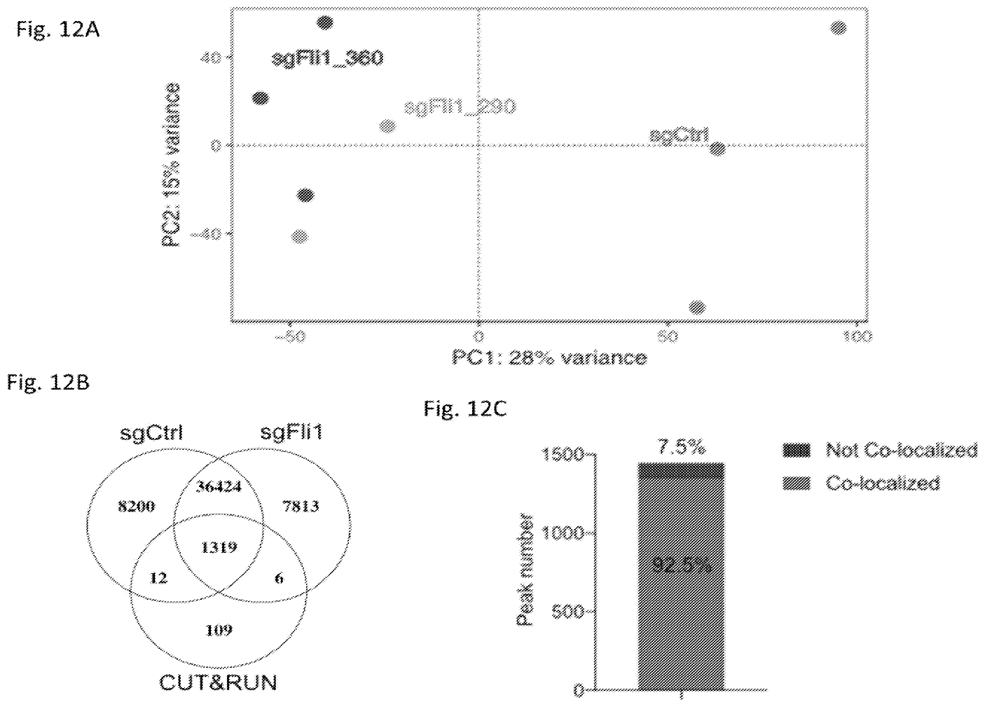

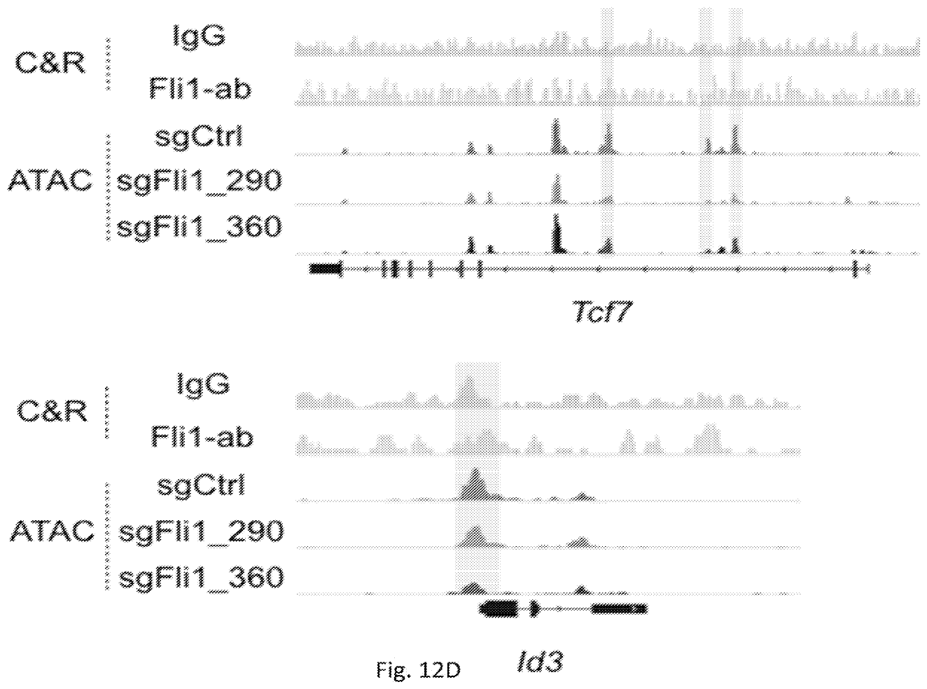

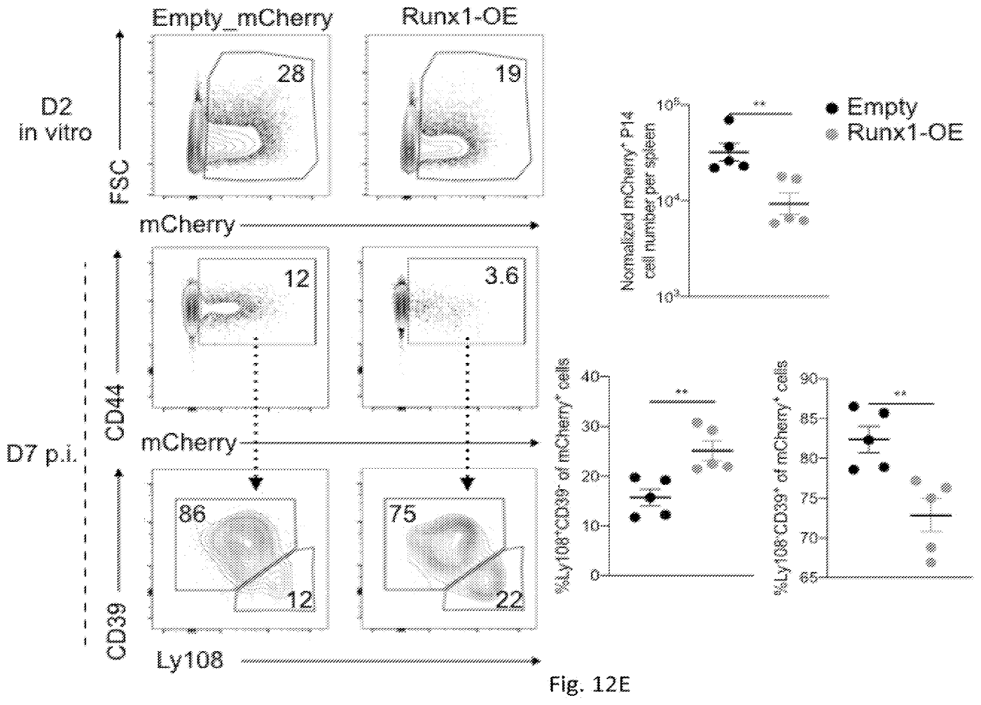

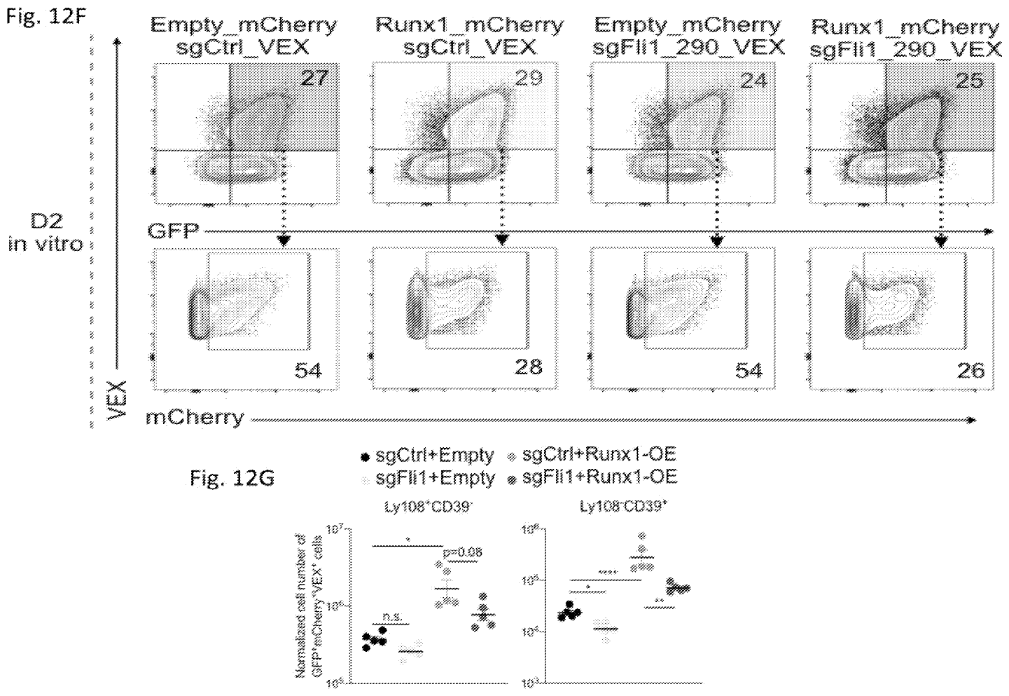

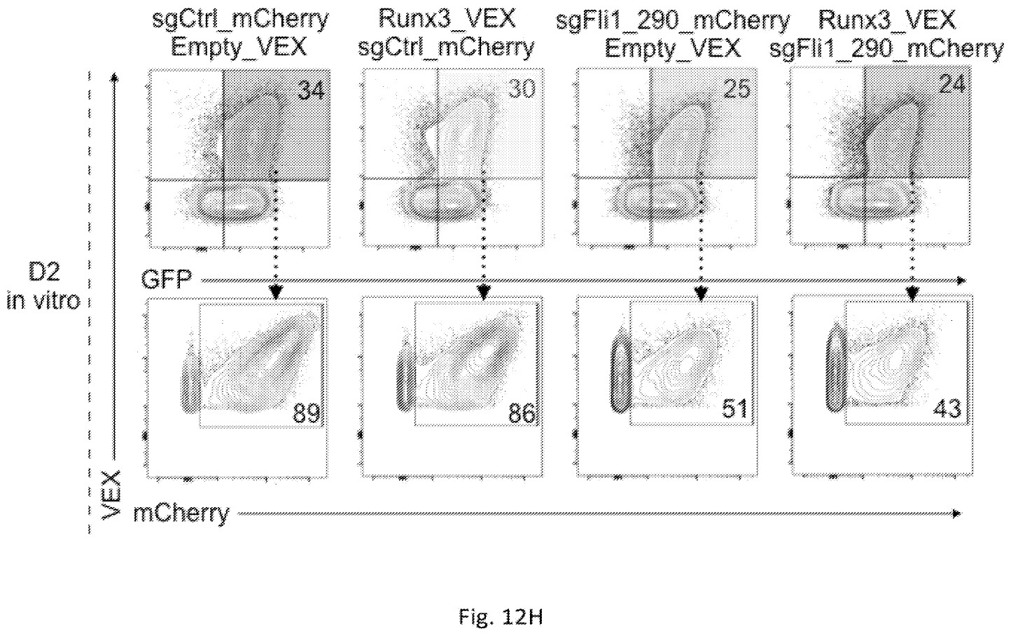

Fli1 CUT&RUN (Skene et al. (2017) Cdn.Elifesciences.org) was used to test how Fli1 genomic binding was related to changes in chromatin accessibility and T_{EFF} biology. At Day 9 p.i. with C113, >90% of the identified Fli1 binding sites were contained in OCRs detected by ATAC-seq (FIG. 12B-12D). Specifically, Fli1 bound to OCRs of T_{EFF}-like genes such as Cx3cr1, Cd28 and Havcr2. Chromatin accessibility increased at these locations upon Fli1 deletion (FIG. 4H), resulting in increased transcription (FIG. 3C) and protein expression (FIGS. 3B and 4I). In contrast, for genes involved in progenitor biology that were decreased in expression in the absence of Fli1 such as Tcf7 and Id3, direct binding of Fli1 was not observed (FIG. 12D), likely indicating that the major role of Fli1 is to safeguard against an overly robust T_{EFF} program rather directly enabling memory/progenitor biology. Furthermore, 78% of the sites where Fli1 was defined to bind by CUT&RUN increased in chromatin accessibility in the absence of Fli1; in contrast, 22% decreased in accessibility (FIG. 4J), suggesting that Fli1 mainly functions to repress chromatin accessibility. Analyzing the DNA binding motifs in the Fli1 CUT&RUN data revealed the expected FLIl motif However, SP2, NFY1 and RUNX1 motifs were also significantly enriched where Fli1 bound (FIG. 4K). Together with the increase in ETS:RUNX motifs in the Fli1-deficient ATAC-seq observations above, these data support a model where Fli1 coordinates with RUNX family members to control T_{EFF} differentiation.

### Example 7: Enforced Runx3 Expression Synergizes with Fli1-Deletion to Enhance T_{EFF} Responses

Unlike T_{EFF} biology, the roles of Runx1 and Runx3 in T_{EX} development are less clear. Because T_{EFF} and T_{EX} are opposing fates in chronic infection and ETS:RUNX motifs become more accessible in the absence of Fli1, it was hypothesized that a RUNX-Fli1 axis might influence T_{EFF} versus T_{EX} differentiation. Therefore, it was tested whether Runx1 or Runx3 expression in Fli1-deficient CD8 T cells would impact T_{EFF} differentiation in early chronic infection.

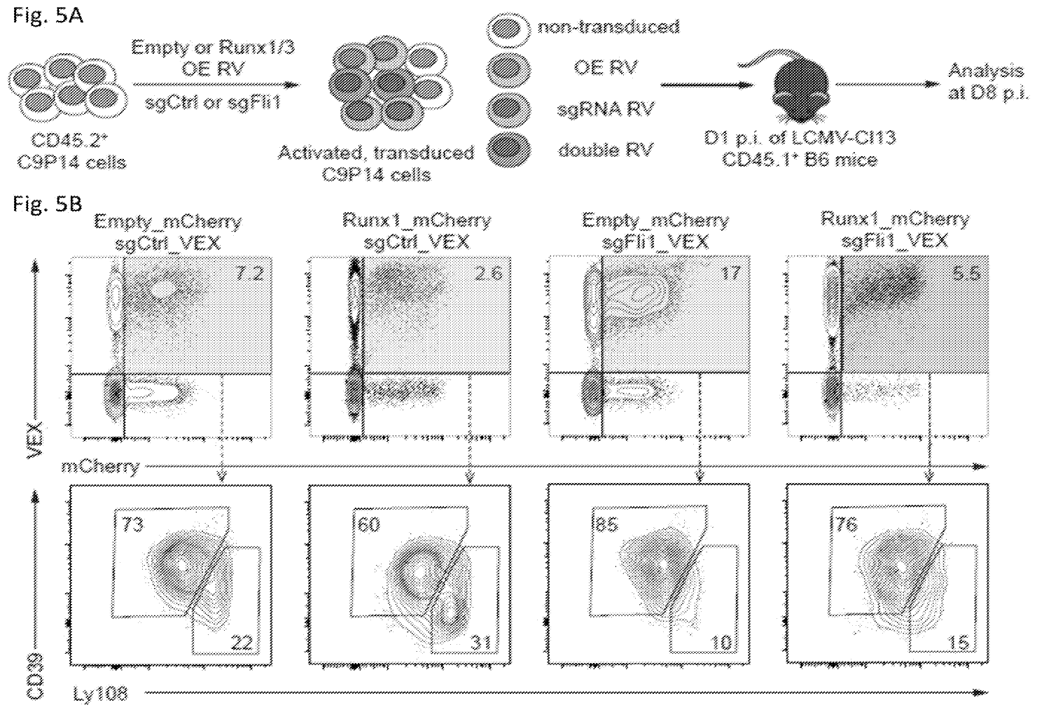

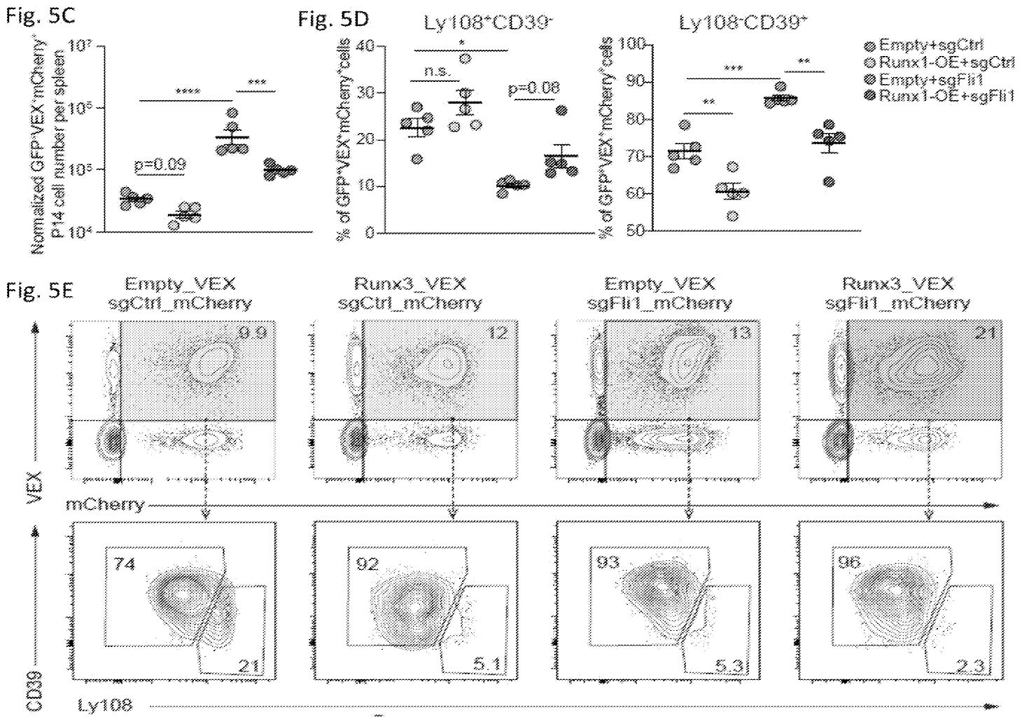

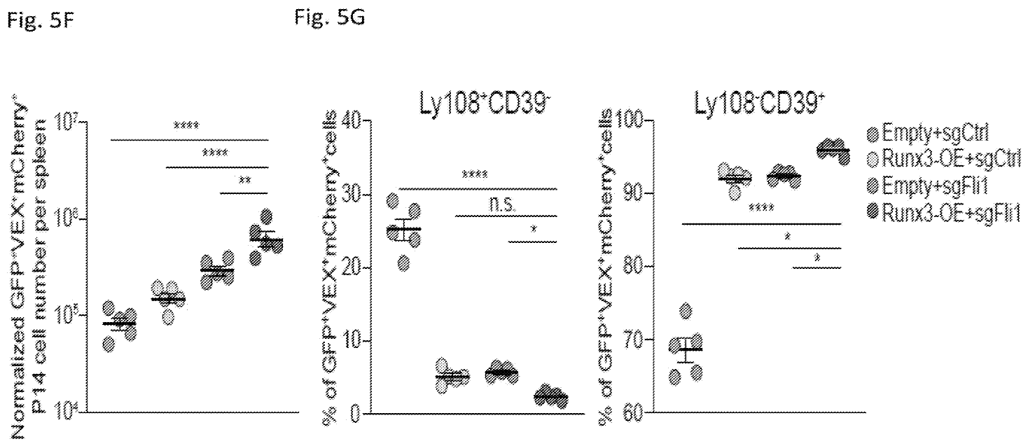

Enforced expression of Runx1 in WT P14 cells reduced cell numbers at Day 7 p.i. with C113 infection (FIG. 12E). Moreover, Runx1-OE fostered formation of Ly108⁺CD39⁻ T_{EX} precursors at the expense of the more T_{EFF}-like Ly108-CD39⁺population at this time point (FIG. 12E). To interrogate the impact of enforced Runx1 expression in the absence of Fli1, a dual RV transduction approach was used and control or Fli1 sgRNA RV transduction was combined with either empty or Runx1 expressing RVs. Singly versus dually transduced cells were distinguished using VEX (for sgRNA) and mCherry. C9P14 cells were efficiently singly or dually transduced (FIG. 12F) and adoptively transferred into C113-infected mice and the double transduced (i.e. GFP⁺VEX⁺mCherry⁺) C9P14 cells were analyzed on Day 8 p.i. (FIG. 5A). In the sgFli1⁺Runx1-OE group the number of GFP⁺VEX⁺mCherry⁺C9P14 cells was reduced and there were fewer Ly108-CD39′ cells. In contrast the sgFli1⁺Empty-RV group had an increase in the GFP⁺VEX⁺mCherry⁺ C9P14 cell population and these cells were skewed towards the Ly108⁻CD39⁺T_{EFF}-like fate (FIG. 5B-5D, 12G) as above. However, in the Fli1-deficient setting where there was enhanced CD8 T cell expansion, Runx1 overexpression reduced the magnitude of the response and partially reversed the skewing towards the Ly108⁻CD39⁺T_{EFF}-like fate caused by loss of Fli1 (FIG. 5B-5D).

In contrast to the effect of Runx1, enforced expression of Runx3 alone (in the sgCtrl⁺group), modestly increased the magnitude of the CD8 T cell response but robustly skewed the G GFP⁺VEX⁺mCherry⁺C9P14 population towards a CD39⁺Ly108- T_{EFF}-like population (FIG. 5A, 5E-5G). These effects were more dramatic in the absence of Fli1 with greater numerical amplification and even further skewing towards CD39⁺Ly108- T_{EFF}-like cells in the sgFli1⁺Runx3-OE enforced expression group (FIG. 5E-5G). Whereas the sgFli1⁺Runx3-OE group had lower frequency of the T_{EX} precursor population, the absolute number of this population remained unchanged compared to control (FIG. 5E, 5G, 12I). Taken together, these data support a model where loss of Fli1 reveals ETS:RUNX motifs that can be used by Runx1 and/or Runx3. However, whereas Runx3 drives a more T_{EFF}-like population, an effect amplified in the absence of Fli1, Runx1 appears to antagonize T_{EFF} generation, in agreement with the opposing functions of Runx1 and Runx3. Thus, Fli1 restrains the T_{EFF} promoting activity of Runx3 function by restricting genome access and protecting ETS:RUNX binding sites. These data reveal Fli1, Runx3 and perhaps Runx1 as key regulators of the fate choice between T_{EFF} and T_{EX} early after initial activation.

### Example 8: Augmented T_{EFF} Cell Responses in the Absence of Fli1 Improve Protective Immunity Against Pathogens

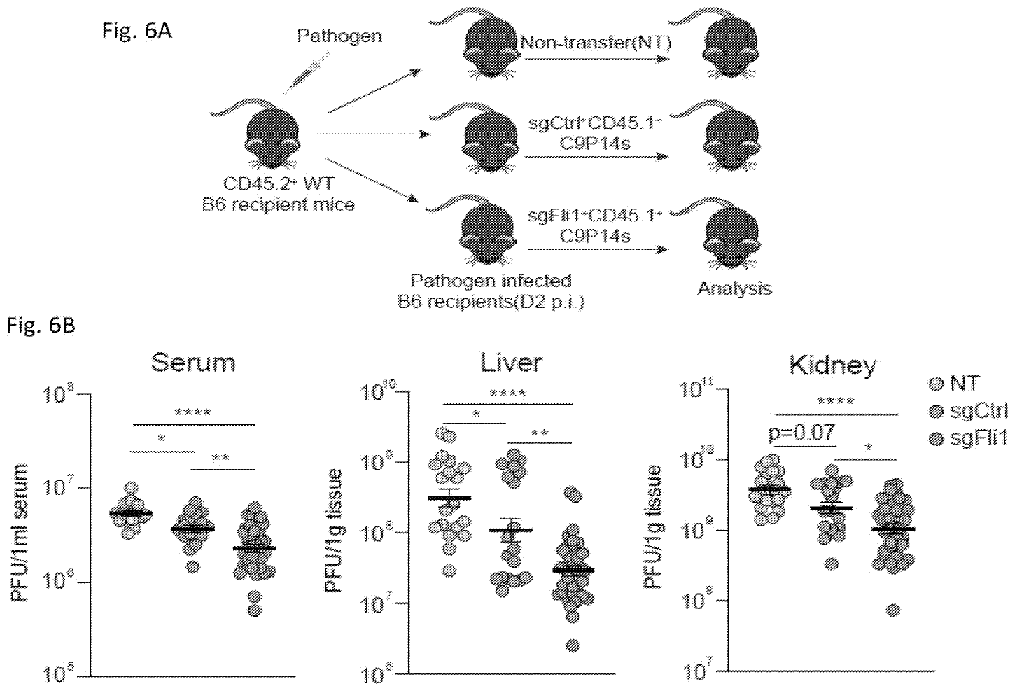

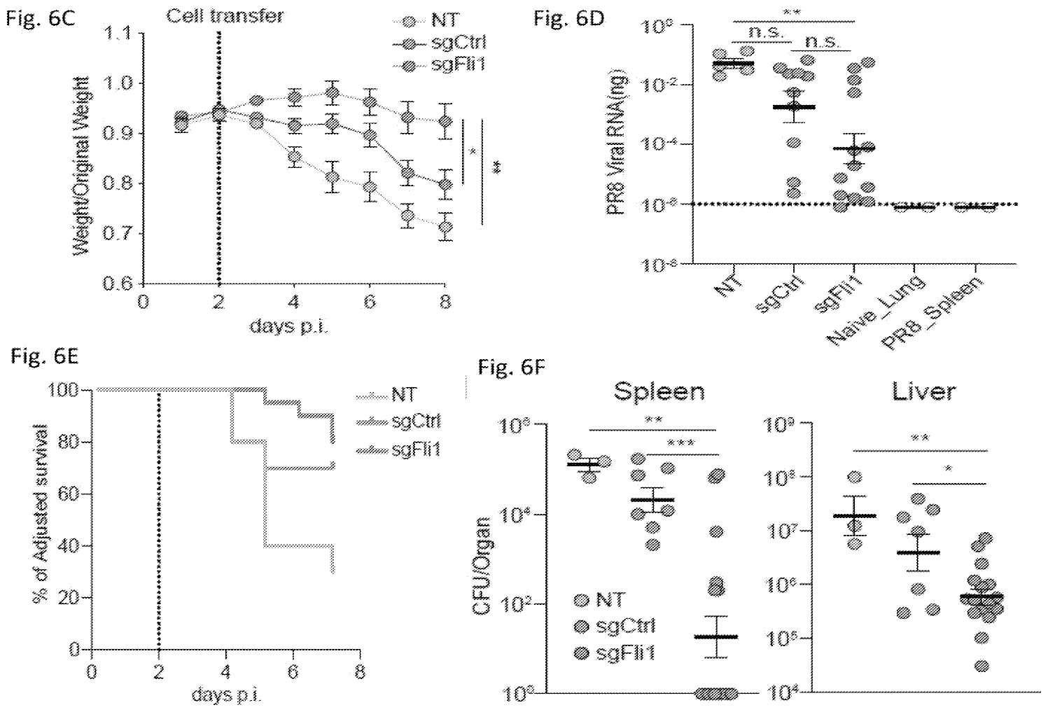

The data above provoke the question of whether loss of Fli1 would improve control of infections due to the augmented T_{EFF} differentiation. To test this idea, LCMV C113 was used to investigate protective immunity during chronic infection along with two models of acute infection: influenza virus (PR8) or Listeria monocytogenes (LM), each expressing the LCMV GP33-41 epitope (PR8GP33 and LMGP33) recognized by P14 cells (FIG. 6A).

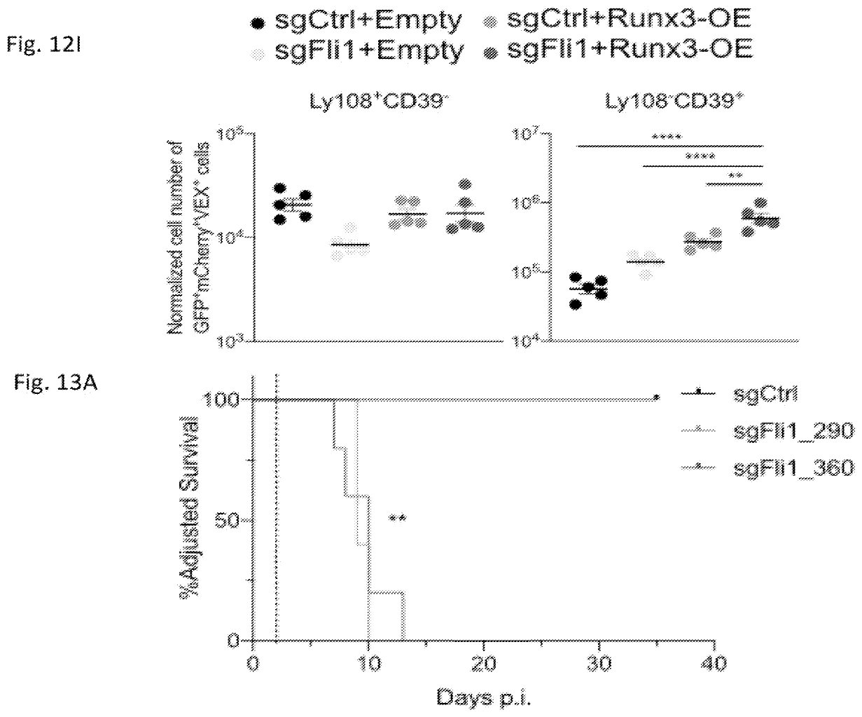

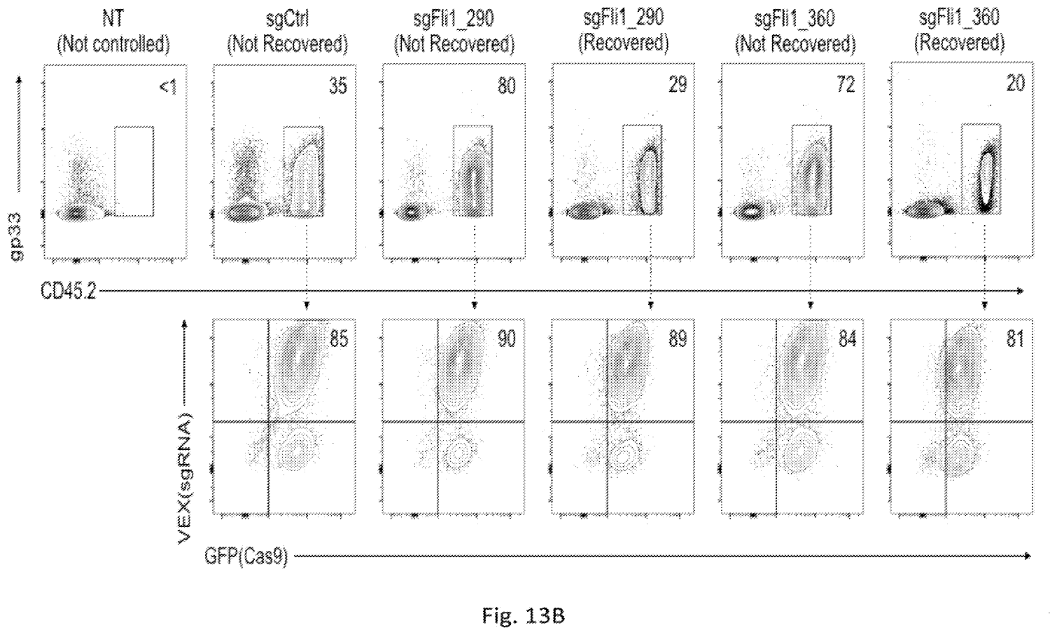

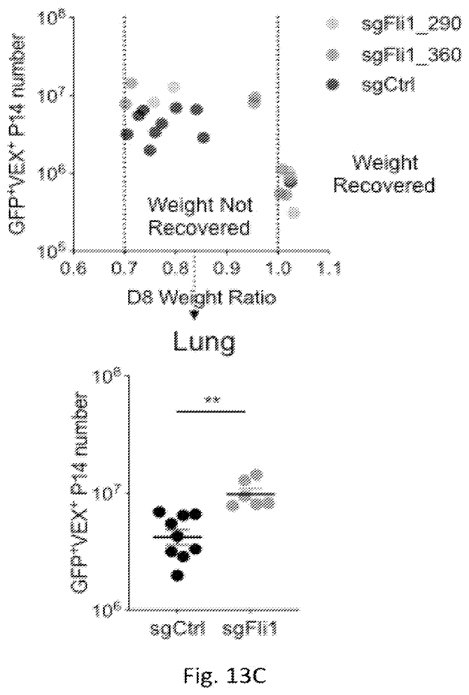

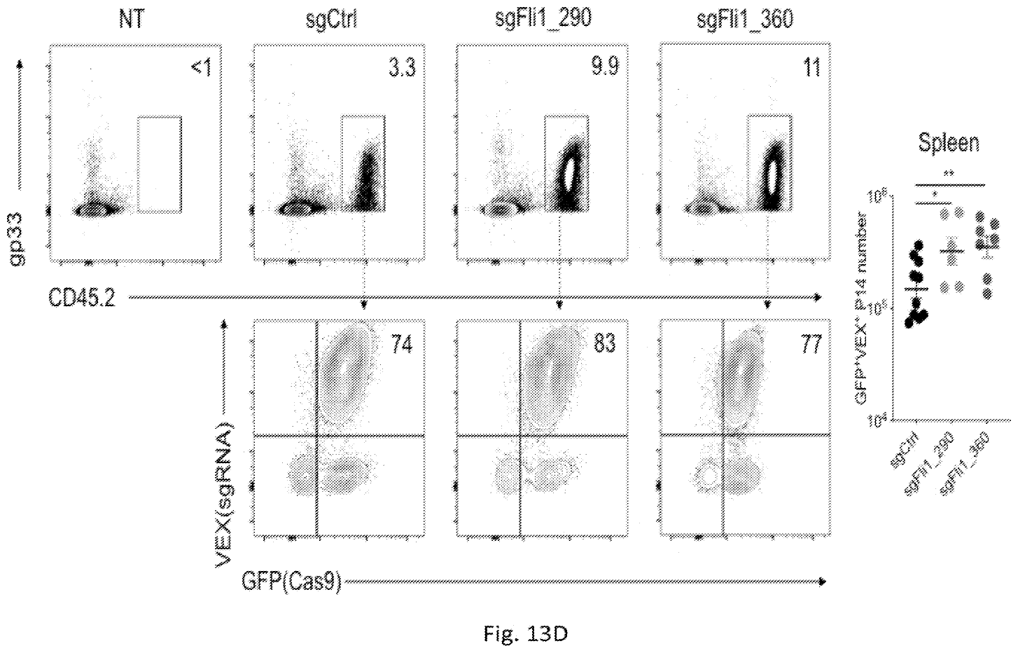

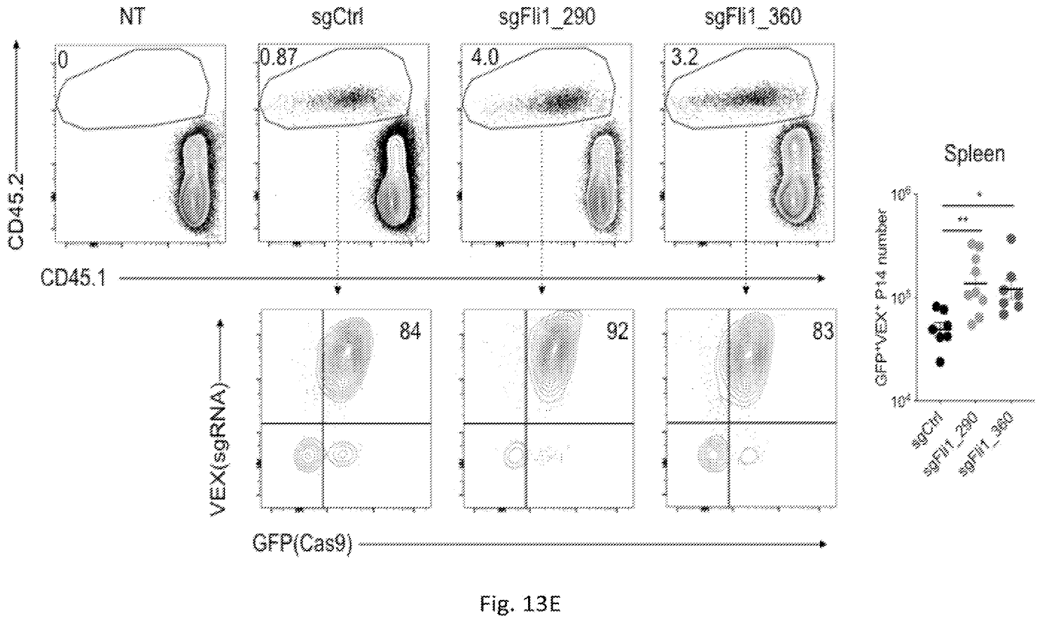

During C113 infection, adoptive transfer of sgCtrl⁺ C9P14 cells conferred a moderate degree of viral control compared to the no transfer condition (NT, FIG. 6B). However, sgFli1V C9P14 provided substantially improved control of viral replication compared to sgCtrl⁺ C9P14 cells at −2 weeks p.i. (FIG. 6B), demonstrating a benefit due to loss of Fli1 even in chronic viral infection where induction of exhaustion is a major barrier to effective protective immunity. Notably, in some experiments sgFli1⁺C9P14, but not sgCtrl⁺ C9P14 recipient mice experienced severe disease and had to be euthanized between D7-D13 p.i., (FIG. 13A), suggesting that Fli1 may safeguard from excessive T_{EFF}-like differentiation and limit T cell mediated immunopathology.

Next, the impact of loss of Fli1 during acutely resolving infections was evaluated. During influenza-PR8_{GP33} infection, mice receiving sgFli1⁺ C9P14 cells lost less weight than control non-transferred mice or mice receiving sgCtrl⁺ C9P14 cells (FIG. 6C). Decreased weight loss was associated with better control of viral replication in the lungs of mice receiving sgFli1⁺C9P14 cells (FIG. 6D). In this setting, there was variation in the magnitude of the sgFli1⁺C9P14 expansion in the lungs following PR8GP33 infection (FIG. 13B-13D). This heterogeneity in the T cell responses was associated with differences in viral control, with some mice nearly eliminating viral RNA by this time point (FIG. 6D) and recovering from weight loss. Indeed, the overall magnitude of the C9P14 response was lower in mice that had recovered, consistent with prolonged viral replication and higher antigen load driving increased T cell expansion in mice that had not yet controlled the infection (FIG. 13B-13C). Notably, 6 out of 12 of the mice receiving sgFli1⁺C9P14 cells had controlled disease by this time point, compared to only 1 out of 11 mice receiving sgCtrl⁺ C9P14 (FIG. 6D, 13C). In the group of mice still harboring viral RNA in the lungs, sgFli1⁺C9P14 cells had expanded to substantially higher numbers than sgCtrl⁺ C9P14 cells (FIG. 13C). A similar difference in T_{EFF} cell expansion was also observed in the spleen (FIG. 13D).

Loss of Fli1 conferred a similar advantage following LMGP33 infection. Although both sgCtrl⁺ and sgFli1⁺C9P14 cells improved survival following a high dose LMGP33 challenge (FIG. 6E), sgFli1⁺ C9P14 cells resulted in substantially better control of bacterial replication compared to the sgCtrl⁺ C9P14 cells at Day 7 p.i. (FIG. 6F). Consistent with the influenza virus model, this improved protective immunity was associated with greater numerical expansion of sgFli1³⁰ C9P14 cells compared to the sgCtrl⁺ group (FIG. 13E). Thus, deficiency in Fli1 confers a substantial benefit on T_{EFF} cell expansion and protective immunity during chronic C113 infection, respiratory influenza virus infection, and systemic infection with an intracellular bacterium.

### Example 9: Loss of Fli1 in CD8 T Cells Enhances Immunity to Tumors

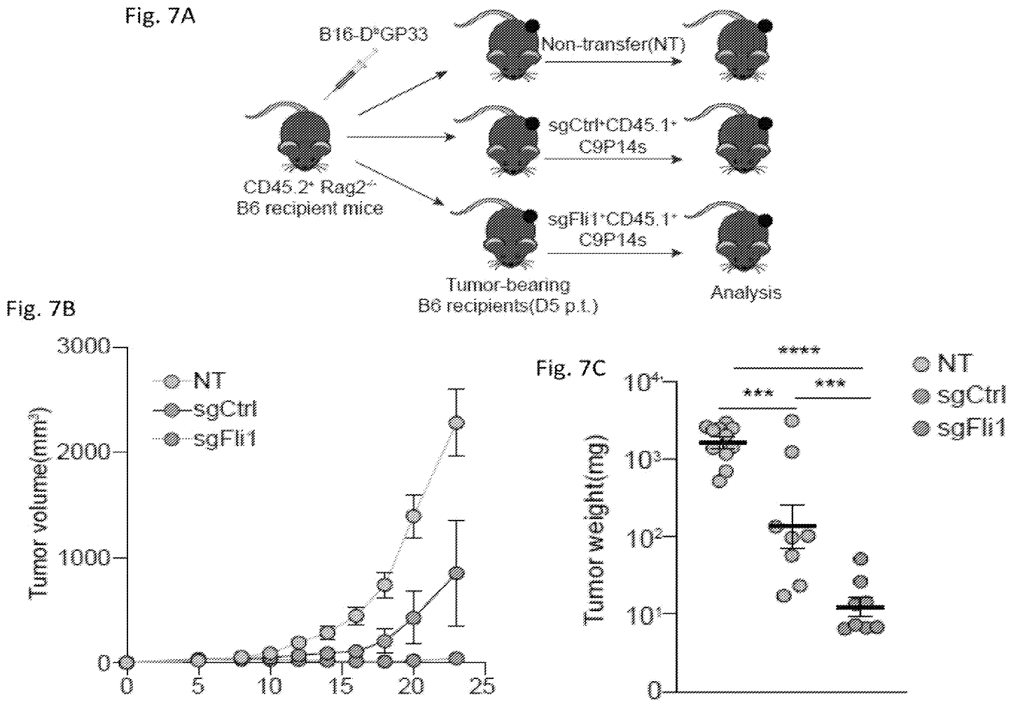

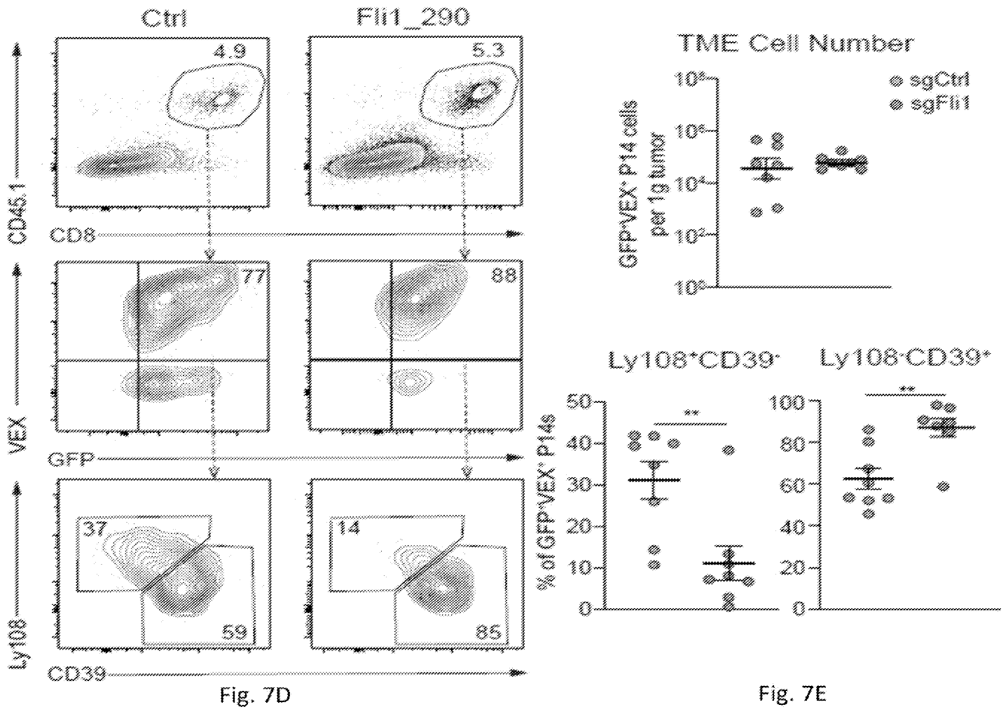

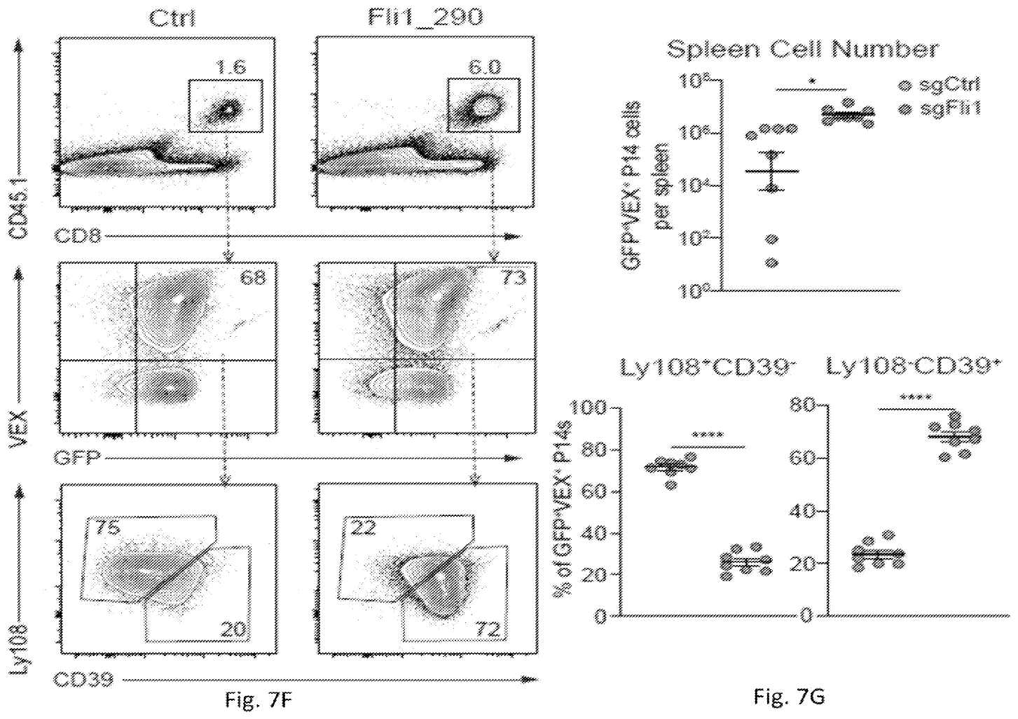

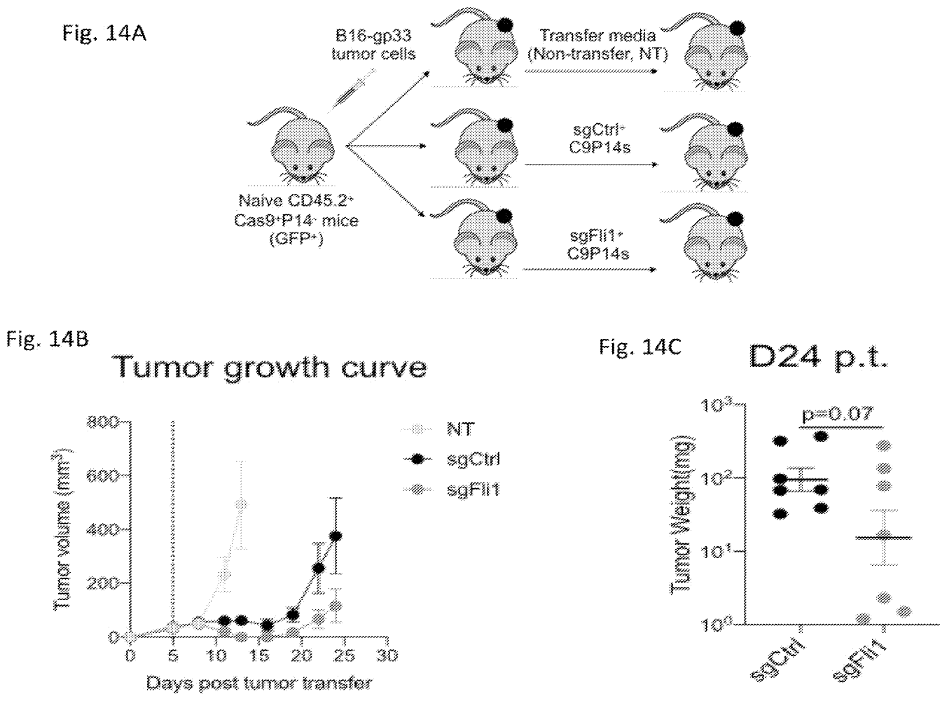

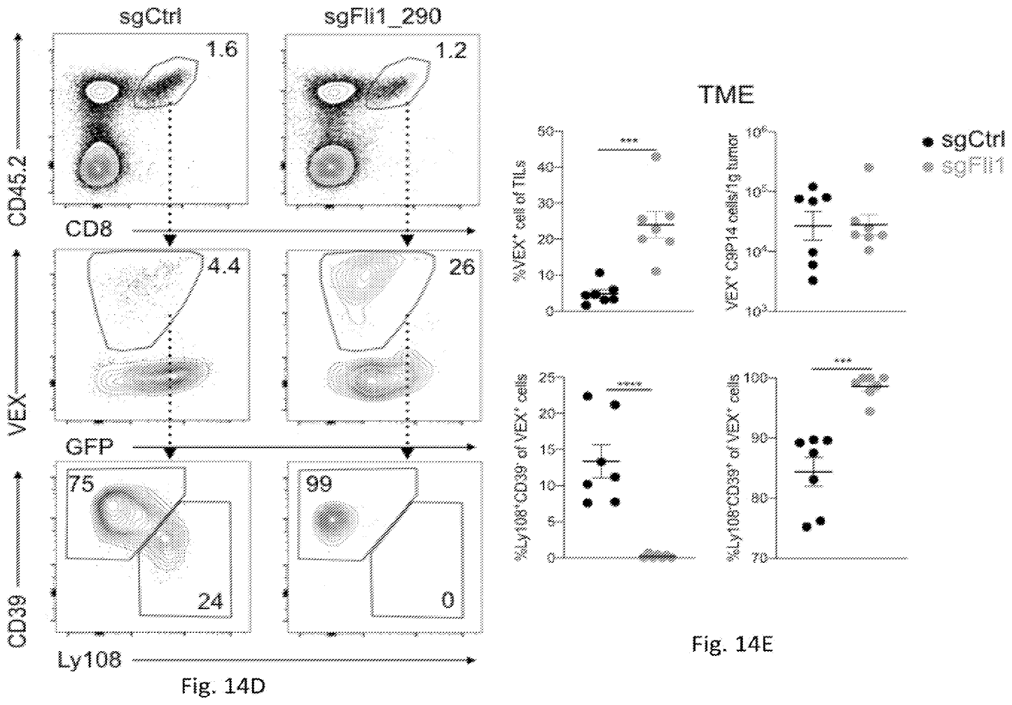

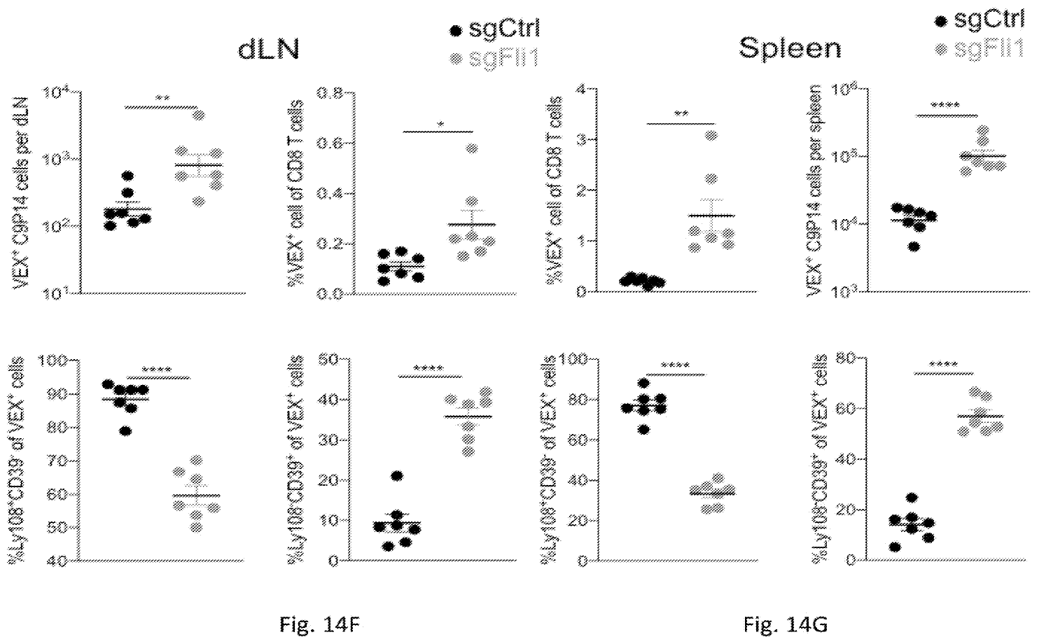

It was next asked whether Fli1 deficiency provided enhanced control of tumors. A subcutaneous B1⁶GP33 tumor model was employed. Tumor-bearing mice received equal numbers of sgCtrl⁺ or sgFli1⁺C9P14 cells on day 5 post tumor inoculation (p.t.) (FIG. 7A). Rag2−/− recipient mice were used to isolate the effects of sgCtrl⁺ versus sgFli1⁺C9P14 cells (FIG. 7A). In this setting, sgFli1⁺C9P14 cells robustly controlled tumor progression compared to non-transferred mice or the sgCtrl⁺ C9P14 group (FIG. 7B). Furthermore, tumor weight was significantly lower in the sgFli1⁺C9P14 group compared to either control group at endpoint (FIG. 7C). Although there was not a clear difference in the number of C9P14 cells/gram of tumor, this tumor control was associated with a significant increase in Ly108-CD39⁺donor C9P14 in the sgFli1⁺ group, consistent with the more T_{EFF}-like population (FIG. 7D-7E). In the spleen, however, there was a significant increase in sgFli1V C9P14 cell numbers, as well as the proportion of Ly108⁻CD39⁺ cells compared to the sgCtrl⁺ group (FIG. 7F-7G). These findings were extended to immune competent mice. Cas9⁺C57BL/6 recipient mice were used to prevent rejection of the C9P14 donor cells and allow responses to be analyzed for an extended period of time (FIG. 14A). In this setting, sgFli1⁺C9P14 cells again conferred substantial benefit on tumor control (FIG. 14B-14C) compared to sgCtrl⁺ C9P14 cells. Furthermore, this improved tumor control was associated with an increase in the Ly108-CD39⁺T_{EFF}-like population in the tumor, draining lymph node (dLN) and spleen, as well as increased C9P14 cell numbers in the dLN and spleen (FIG. 14D-14G). Thus, genetic deletion of Fli1 conferred a substantial benefit on tumor control indicating a central role for Fli1 in coordinating and restraining protective T_{EFF} responses during tumor progression. Together, these data show that loss of Fli1 results in improved protective immunity, in the setting of systemic and local, acutely resolving and chronic infections, as well as tumor progression.

### Example 10: Discussion

Efforts herein focused on improving immunotherapy for cancer and chronic infections through a better understanding of the biology of T_{EX} and T_{EFF} differentiation. An in vivo CRISPR screening approach was used to specifically interrogate the mechanisms governing T_{EX} versus T_{EFF} differentiation. Fli1 was identified as a key TF that safeguards the transcriptional and epigenetic commitment to full T_{EFF} differentiation. Mechanistically, Fli1 limited epigenetic accessibility to ETS:RUNX sites, preventing Runx3 from fully enabling an effector program. As a result, deleting Fli1 robustly improved protective immunity in multiple models of acute infection, chronic infection and cancer, thus identifying Fli1 as a novel regulator of the T_{EFF} versus T_{EX} differentiation programs and a target for future immunotherapy strategies.

Recent advances in CRISPR-based screening approaches have allowed the dissection of in vitro T cell activation and in vivo responses to infections and tumors. To better understand the fate commitment of T_{EFF} and T_{EX}, OpTICS was developed herein. OpTICS is an in vivo CRISPR system that, among other things, allows screening in mature CD8 T cells using physiological T cell numbers that preserve disease pathogenesis and normal CD8 T cell differentiation biology. In addition to identifying many known regulators of CD8 T cell responses, this approach identified several novel negative regulators of T_{EFF} differentiation, including Smad2, Erg and Fli1. Indeed, genetic loss of Fli1 improved protective immunity in multiple settings of acute or chronic infection and cancer. Moreover, unlike the effect seen with loss of the T_{EX}-driving TF Tox, where CD8 T cell responses cannot be sustained during chronic infection or cancer due to loss of T_{EX} progenitor cells, deficiency in Fli1 did not diminish the T_{EX} progenitor population.

Fli1 has a role in hematopoietic stem cell differentiation and co-localizes with other TFs such as Gatal/2 and Runx1. Herein, it was discovered that genetic perturbation of Fli1 significantly increased chromatin accessibility at ETS:RUNX motifs in antigen-specific CD8 T cells responding to viral infection. Moreover, the effect of enforced Runx3 expression is enhanced in the absence of Fli1. These observations suggest that Fli1 prevents accessibility to RUNX binding sites, restricting the activity of the effector-promoting TF Runx3. Moreover, Runx3 can coordinate epigenetic changes at loci encoding other effector-promoting TFs. Runx1 likely antagonizes Runx3 and vice versa, though Runx3 appears to dominate in settings of T cell activation. Data herein also suggest that Fli1 can cooperate with Runx1 to restrain T_{EFF} differentiation, perhaps with Fli1 and Runx1 co-binding at ETS-RUNX motifs. Together, these data suggest a model where Fli1, in combination with Runx1, prevents efficient genome accessibility or activity of Runx3 and thus restrain the full effector gene program that involves the positive feed-forward effector promoting activity of Runx3. Thus, genetic deletion of Fli1 de-represses T_{EFF} differentiation, at least partially by creating opportunities for more efficient Runx3 activity.

Recent work has begun to define the transcriptional circuitry that directs fate decisions between terminal T_{EFF}, T_{MEM} and T_{EX}. Many of these transcriptional mechanisms that promote one cell fate directly repress the opposing fate. For example, Tox promotes T_{EX} while repressing T_{EFF}, TCF-1 promotes T_{MEM} or TEx at the expense of T_{EFF} and Blimp-1, T-bet, Id2, and others drive T_{EFF} and repress T_{MEM}. The identification of Fli1 as a type of genomic “safeguard” against over-commitment to effector differentiation reveals several novel concepts. First, during chronic infection, loss of TCF-1 or Tox results in an ability to sustain responses later in infection due to commitment to terminally differentiated T_{EFF}. These observations provoke the question of whether fostering an increase in the T_{EFF} cell fate necessarily comes at the expense of losing the T_{MEM} or TEx lineage. Fli1 represents a distinct type of damper on an otherwise robust feed-forward effector transcriptional circuit. By restraining the Runx3 node in the effector wiring, Fli1 tempers a central step that not only directly controls expression of key effector genes but also positively reinforces other cooperating effector TFs. However, unlike TCF-1 and Tox, Fli1 is not required for progenitor biology and the number of both T_{EFF} cells and T_{MP} (in acutely resolved infections) or T_{EX} progenitor cells (in chronic infection) were increased in the absence of Fli1. Thus, by interrupting this “damper” in the circuit, rather than deleting the master switch of T_{MP} or T_{EX} differentiation it may be possible to augment beneficial aspects of short-term protective immunity without compromising long-term immunity. Second, data herein reveal a mechanism of competition for epigenetic access between Fli1 and other factors that bind at ETS:RUNX motifs. These effects may manifest because Fli1 occupies genomic locations that can be bound by ETS:RUNX family TFs that would catalyze chromatin accessibility changes. Alternatively, these effects may be due to chromatin changes coordinated by Fli1 itself. For example, the EWS-FLIl fusion recruits the BAF complex to initiate chromatin changes in cancer cells. Thus, the role of Fli1 in CD8 T cells likely involves a chromatin accessibility-based mechanism to restrain ETS:RUNX driven effector biology, though other effects through IRF1/IRF2 may also exist.

The current studies demonstrate a major beneficial effect of loss of Fli1 on protective immunity in multiple settings of infection and cancer. The absence of Fli1 consistently improved protective immunity across models. Of particular relevance for immunotherapy, deleting Fli1 improved control of both tumor growth and chronic LCMV infection where the induction of exhaustion typically limits immunity. Finally, given the ability to apply CRISPR-mediated genetic manipulations in cellular therapy settings clinical benefits can be achieved by targeting Fli1 or related pathways.

Thus, the OpTICS platform provides a highly robust in vivo platform to screen genes involved in regulating CD8 T cell differentiation as it relates to tumor immunotherapy. This highly focused and optimized platform allows for a 20˜100-fold enrichment of sgRNA detection and considerable resolution for gain-of-function screening. In addition to the novel role of Fli1 revealed here, many other potential targets for exploration exist from this screen. Moreover, using OpTICS to extend from this TF focused biology to other areas of cellular biology should provide a robust platform for future discovery.

### Enumerated Embodiments

The following enumerated embodiments are provided, the numbering of which is not to be construed as designating levels of importance.

Embodiment 1 provides a modified immune cell or precursor thereof, comprising a modification in an endogenous gene locus encoding Fli1.

Embodiment 2 provides the modified immune cell or precursor thereof of embodiment 1, wherein the endogenous Fli1 gene or protein is disrupted.

Embodiment 3 provides the modified immune cell or precursor thereof of embodiment 1 or 2, wherein the modification or disruption is made by a method selected from the group consisting of a CRISPR system, an antibody, an siRNA, a miRNA, an antagonist, a drug, a small molecule inhibitor, a PROTAC target, a TALEN, and a Zinc Finger Nuclease.

Embodiment 4 provides the modified immune cell or precursor thereof of embodiment 3, wherein the CRISPR system comprises at least one sgRNA comprising any one of SEQ ID NOs: 152-156 or SEQ ID NOs: 676-713.

Embodiment 5 provides the immune cell or precursor thereof of any of the preceding embodiments, wherein the cell is a human cell.

Embodiment 6 provides the immune cell or precursor thereof of any of the preceding embodiments, wherein the cell is a T cell.

Embodiment 7 provides the immune cell or precursor thereof of embodiment 6, wherein the T cell is resistant to T cell exhaustion.

Embodiment 8 provides a pharmaceutical composition comprising an inhibitor of Fli1.

Embodiment 9 provides the pharmaceutical composition of embodiment 8, wherein the inhibitor is selected from the group consisting of a CRISPR system, an antibody, an siRNA, a miRNA, an antagonist, a drug, a small molecule inhibitor, a PROTAC target, a TALEN, and a Zinc Finger Nuclease.

Embodiment 10 provides the pharmaceutical composition of embodiment 9, wherein the CRISPR system comprises at least one sgRNA comprising any one of SEQ ID NOs: 152-156 or SEQ ID NOs: 676-713.

Embodiment 11 provides a method of treating a disease or disorder in a subject in need thereof, the method comprising administering to the subject the cell of any one of embodiments 1-7 or the composition of any one of embodiments 8-10.

Embodiment 12 provides the method of embodiment 11, wherein the disease or disorder is an infection.

Embodiment 13 provides the method of embodiment 11, wherein the disease is cancer.

Embodiment 14 provides a method of screening a T cell, the method comprising: i) introducing into an activated T cell a Cas enzyme and an sgRNA library, ii) administering the T cell to an infected mouse, iii) isolating the T cell from the infected mouse, and iv) analyzing the T cell.

Embodiment 15 provides the method of embodiment 14, wherein the sgRNA library comprises a plurality of sgRNAs that target a plurality of transcription factors.

Embodiment 16 provides the method of embodiment 15, wherein the plurality of transcription factors comprise any of the transcription factors listed in Table 1.

Embodiment 17 provides the method of embodiment 15, wherein each sgRNA targets the DNA binding domain of each transcription factor.

Embodiment 18 provides the method of embodiment 14, wherein the sgRNA library comprises at least one sequence selected from the group consisting of SEQ ID NOs: 1-675.

Embodiment 19 provides the method of embodiment 14, wherein the sgRNA library consists of the nucleotide sequences set forth in SEQ ID NOs: 1-675.

Embodiment 20 provides the method of embodiment 14, wherein the screening assesses T cell exhaustion.

Embodiment 21 provides the method of embodiment 14, wherein analyzing the cell comprises a method selected from the group consisting of sequencing, PCR, MACS, and FACS.

Embodiment 22 provides the method of embodiment 14, wherein the sequencing reveals a target of interest.

Embodiment 23 provides the method of embodiment 22, wherein a drug is designed against the target of interest.

Embodiment 24 provides the method of embodiment 22, wherein when the drug is administered to the T cell, at least one T cell response is increased.

Embodiment 25 provides the method of embodiment 14, wherein 1×10⁵ T cells are administered to the infected mouse.

Embodiment 26 provides the method of embodiment 14, wherein the method identifies novel transcription factors governing T_{EFF} and T_{EX} cell differentiation.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

TABLE 1

| sgRNA name | sgRNA Sequence(No PAM) | SEQ ID NO: |
|---|---|---|
| sgCtrl_1 | AATGGCAACTGGTCCCCTTC | SEQ ID NO: 1 |
| sgCtrl_2 | GAAGATGGGCGGGAGTCTTC | SEQ ID NO: 2 |
| sgCtrl_3 | CATCGGCCCGGGGCAACTCG | SEQ ID NO: 3 |
| sgCtrl_4 | AAAGTCCTTCATCCGGCTTA | SEQ ID NO: 4 |
| sgCtrl_5 | CGTGTCCGAACTCTGCATCC | SEQ ID NO: 5 |
| sgCtrl_6 | CTGATCAACATGTACCTGCC | SEQ ID NO: 6 |
| sgCtrl_7 | GTGAGCGCACCGCAGTGCCC | SEQ ID NO: 7 |
| sgCtrl_8 | TAGTTGGGTGCGCCCGCGTA | SEQ ID NO: 8 |
| sgCtrl_9 | AACATGCGCGGCAGCGGCAC | SEQ ID NO: 9 |
| sgCtrl_10 | CGAGCAGCGGGTCACCGTCC | SEQ ID NO: 10 |
| sgCtrl_11 | AGTCTTGTCAAGCTTAACTC | SEQ ID NO: 11 |
| Ahr_HLH_58_5 | GCTTATTAATAACATCTTGC | SEQ ID NO: 12 |
| Ahr_HLH_61_4 | GCAAGATGTTATTAATAAGC | SEQ ID NO: 13 |
| Ahr_HLH_68_3 | TGGACAAACTCTCTGTTCTT | SEQ ID NO: 14 |
| Ahr_HLH_75_1 | GGCTCAGCGTCAGCTACCTG | SEQ ID NO: 15 |
| Ahr_HLH_75_2 | GCTCAGCGTCAGCTACCTGA | SEQ ID NO: 16 |
| Atf3_bZIP_106_5 | CAAGTGTCGAAACAAGAAAA | SEQ ID NO: 17 |
| Atf3_bZIP_127_4 | GAGTGTGAATGCTGAGCTGA | SEQ ID NO: 18 |
| Atf3_bZIP_131_3 | TGAGCTGAAGGCCCAGATTG | SEQ ID NO: 19 |
| Atf3_bZIP_148_2 | TATACATGCTCAACCTGCAC | SEQ ID NO: 20 |
| Atf3_bZIP_88_1 | CTGAAGAAGATGAGAGGAAA | SEQ ID NO: 21 |
| Atf4_DBD_19_4 | TGGATGAAGATCCAGATGAG | SEQ ID NO: 22 |
| Atf4_DBD_19_5 | AACCGCTGCCGCCGCTCATC | SEQ ID NO: 23 |
| Atf4_DBD_22_3 | ATGAAGATCCAGATGAGCGG | SEQ ID NO: 24 |
| Atf4_DBD_28_2 | ATCCAGATGAGCGGCGGCAG | SEQ ID NO: 25 |
| Atf4_DBD_67_1 | GAGCTGCAGCCTCTCGATGC | SEQ ID NO: 26 |
| Atf6_bZIP_297_3 | CTGTGCTGAGGAGACAGCAG | SEQ ID NO: 27 |
| Atf6_bZIP_306_4 | GCGACTGACAAGCAGACTCT | SEQ ID NO: 28 |
| Atf6_bZIP_317_5 | AAGAAGAAAGAGTATATGCT | SEQ ID NO: 29 |
| Atf6_bZIP_324_2 | AGGACTGGAGGCCAGGCTCA | SEQ ID NO: 30 |
| Atf6_bZIP_346_1 | CCTGAAGCGACAGCTGGACG | SEQ ID NO: 31 |
| Bach2_bZIP_641 | CAAGCTAACCTCAGAACAGT | SEQ ID NO: 32 |
| Bach2_bZIP_648 | TGGAGTTCATCCACGACATC | SEQ ID NO: 33 |
| Bach2_bZIP_650 | TCATCCACGACATCCGGAGG | SEQ ID NO: 34 |
| Bach2_bZIP_662 | GCTTCCTCTTTCGACAGCGC | SEQ ID NO: 35 |
| Bach2_bZIP_687 | CGAGAAGGAAAAACTGTTGT | SEQ ID NO: 36 |
| Batf_bZIP_30 | ATGATGTGAGGAAAGTTCAG | SEQ ID NO: 37 |
| Batf_bZIP_43 | CTGTCTCTGTCGGCTCTTCT | SEQ ID NO: 38 |
| Batf_bZIP_63 | GAGCTGCGTTCTGTTTCTCC | SEQ ID NO: 39 |
| Batf_bZIP_71 | TGAGCTGTTTGATCTCTTTG | SEQ ID NO: 40 |
| Batf_bZIP_75 | AGAGATCAAACAGCTCACCG | SEQ ID NO: 41 |
| Bcl6_DBD_592 | GAATTCGAGTGTGGGTCTTC | SEQ ID NO: 42 |
| Bcl6_DBD_596 | ACCCACACTCGAATTCACTC | SEQ ID NO: 43 |
| Bcl6_DBD_605 | CCCACAGGTTTCACATTTGT | SEQ ID NO: 44 |
| Bcl6_DBD_610 | CTGAACAAACCTGGCCCCAC | SEQ ID NO: 45 |
| Bcl6_DBD_620 | GGATGAGCACGTGGGCACGG | SEQ ID NO: 46 |
| Bhlhe40_HLH_56 | AAACTTACAAACTGCCGCAC | SEQ ID NO: 47 |
| Bhlhe40_HLH_59 | TTTTTCTCAATCAGCCGGTG | SEQ ID NO: 48 |
| Bhlhe40_HLH_65 | TTGAGAAAAAGAGACGTGAC | SEQ ID NO: 49 |
| Bhlhe40_HLH_69 | GGGCAATGCACTCGTTAATC | SEQ ID NO: 50 |
| Bhlhe40_HLH_74 | CGAGTGCATTGCCCAGCTGA | SEQ ID NO: 51 |
| Bhlhe41_HLH_104 | CTGATGCTGCTGCTCAGTTA | SEQ ID NO: 52 |
| Bhlhe41_HLH_47 | TAATCTGTGCGGTAACTTGT | SEQ ID NO: 53 |
| Bhlhe41_HLH_51 | TTCTTTTCTATTAATCTGTG | SEQ ID NO: 54 |
| Bhlhe41_HLH_61 | GAGCAATGCATTCATTAATT | SEQ ID NO: 55 |
| Bhlhe41_HLH_87 | TTTGGAGAAAGCAGTAGTCT | SEQ ID NO: 56 |
| Cebpa_bZIP_322_3 | ACAAGAACAGCAACGAGTAC | SEQ ID NO: 57 |
| Cebpa_bZIP_322_4 | CAAGAACAGCAACGAGTACC | SEQ ID NO: 58 |
| Cebpa_bZIP_324_5 | ACAGCAACGAGTACCGGGTA | SEQ ID NO: 59 |
| Cebpa_bZIP_375_1 | GCCGTGAACTGGACACGCTG | SEQ ID NO: 60 |
| Cebpa_bZIP_375_2 | CGTGAACTGGACACGCTGCG | SEQ ID NO: 61 |
| Cebpb_bZIP_215_3 | GTCCACCGTCTTCTTGGCCT | SEQ ID NO: 62 |
| Cebpb_bZIP_216_4 | GGCCAAGGCCAAGAAGACGG | SEQ ID NO: 63 |
| Cebpb_bZIP_217_5 | CAGCTTGTCCACCGTCTTCT | SEQ ID NO: 64 |
| Cebpb_bZIP_280_1 | GCTGCTTGAACAAGTTCCGC | SEQ ID NO: 65 |
| Cebpb_bZIP_280_2 | CTGCTTGAACAAGTTCCGCA | SEQ ID NO: 66 |
| Creb5_bZIP_226_3 | AATTTCCTCCGCCTCTCATC | SEQ ID NO: 67 |
| Creb5_bZIP_226_4 | AGGACCCAGATGAGAGGCGG | SEQ ID NO: 68 |
| Creb5_bZIP_229_5 | TGAGAGGCGGAGGAAATTTC | SEQ ID NO: 69 |
| Creb5_bZIP_240_2 | CTTCTGTCTGCAGCGGGTGG | SEQ ID NO: 70 |
| Creb5_bZIP_281_1 | TTAACAACAACTGCTTCAGC | SEQ ID NO: 71 |
| Crebl2_bZIP_25_3 | TTTCGCTTTCAAGTCGATTT | SEQ ID NO: 72 |
| Crebl2_bZIP_32_4 | AAGCGAAACTTGAGCGAAGC | SEQ ID NO: 73 |
| Crebl2_bZIP_36_5 | GGCATTCTCTCGCACTCTGC | SEQ ID NO: 74 |
| Crebl2_bZIP_49_2 | AAAGCTGAGGTACCAGTACT | SEQ ID NO: 75 |
| Crebl2_bZIP_79_1 | TGGTGCATGGCAATGGACCA | SEQ ID NO: 76 |
| Crem_bZIP_300_4 | CAGAAGAAGCAACTCGCAAG | SEQ ID NO: 77 |
| Crem_bZIP_300_5 | AGAAGAAGCAACTCGCAAGC | SEQ ID NO: 78 |
| Crem_bZIP_303_3 | CAACTCGCAAGCGGGAGCTG | SEQ ID NO: 79 |
| Crem_bZIP_332_2 | TGAGAGTCGAGTCGCAGTGC | SEQ ID NO: 80 |
| Crem_bZIP_341_1 | TCAGAACAAGAAGCTTATAG | SEQ ID NO: 81 |
| E2f1_TDP_128_3 | TTTGGTGGTCAGATTTAGTG | SEQ ID NO: 82 |
| E2f1_TDP_133_4 | CAGCTCCAAGAAGCGTTTGG | SEQ ID NO: 83 |
| E2f1_TDP_133_5 | TCTGACCACCAAACGCTTCT | SEQ ID NO: 84 |
| E2f1_TDP_179_2 | GATATGATTCTTGGACTTCT | SEQ ID NO: 85 |
| E2f1_TDP_183_1 | AGTCCAAGAATCATATCCAG | SEQ ID NO: 86 |
| E2f2_TDP_134_3 | ACACGCTATGACACGTCGCT | SEQ ID NO: 87 |
| E2f2_TDP_134_4 | GACACGCTATGACACGTCGC | SEQ ID NO: 88 |
| E2f2_TDP_135_5 | CACGCTATGACACGTCGCTG | SEQ ID NO: 89 |
| E2f2_TDP_192_1 | GTCCAAAAACAACATCCAGT | SEQ ID NO: 90 |
| E2f2_TDP_192_2 | AGTCCAAAAACAACATCCAG | SEQ ID NO: 91 |
| E2f3_TDP_173_3 | ACGCGGTATGATACGTCCCT | SEQ ID NO: 92 |
| E2f3_TDP_177_4 | ACTTCTTGGTGAGCAGACCG | SEQ ID NO: 93 |
| E2f3_TDP_177_5 | CTTCTTGGTGAGCAGACCGA | SEQ ID NO: 94 |
| E2f3_TDP_190_2 | TCCTGAGCCAGTCTCCTGAT | SEQ ID NO: 95 |
| E2f3_TDP_231_1 | AGTCTAAGAACAACGTCCAG | SEQ ID NO: 96 |
| E2f5_TDP_62_5 | GGAGGCGCAGGACGGCGTCC | SEQ ID NO: 97 |
| E2f5_TDP_74_4 | CTGCAGATACCTTGGCTGTG | SEQ ID NO: 98 |
| E2f5_TDP_87_2 | TGATATCACCAATGTCTTAG | SEQ ID NO: 99 |
| E2f5_TDP_87_3 | GATATCACCAATGTCTTAGA | SEQ ID NO: 100 |
| E2f5_TDP_88_1 | ATCAATTCCCTCTAAGACAT | SEQ ID NO: 101 |
| E2f6_TDP_108_3 | GTATGACATCACCAATGTCT | SEQ ID NO: 102 |
| E2f6_TDP_110_4 | TTCGATGCCATCCAAGACAT | SEQ ID NO: 103 |
| E2f6_TDP_113_5 | TGTCTTGGATGGCATCGAAC | SEQ ID NO: 104 |
| E2f6_TDP_97_2 | TTGCCACAAAACTGGGTGTT | SEQ ID NO: 105 |
| E2f6_TDP_99_1 | CAAAACTGGGTGTTCGGAAG | SEQ ID NO: 106 |
| E2f8_TDP_138_5 | CAGATGTCGTTATTCACAGC | SEQ ID NO: 107 |
| E2f8_TDP_141_4 | TGTGAATAACGACATCTGCC | SEQ ID NO: 108 |
| E2f8_TDP_143_3 | TAACGACATCTGCCTGGACG | SEQ ID NO: 109 |
| E2f8_TDP_145_2 | GCTCCTCGGCCACCTCGTCC | SEQ ID NO: 110 |
| E2f8_TDP_344_1 | GGGCCCGTCCATTTAAAAGC | SEQ ID NO: 111 |
| Egr2_C2H2_339_3 | CCTTCTGCTGGGCAGGGATA | SEQ ID NO: 112 |
| Egr2_C2H2_341_4 | ATCACAACCTTCTGCTGGGC | SEQ ID NO: 113 |
| Egr2_C2H2_341_5 | TCACAACCTTCTGCTGGGCA | SEQ ID NO: 114 |
| Egr2_C2H2_408_1 | GCGCTTCCTTTCGTCACTCC | SEQ ID NO: 115 |
| Egr2_C2H2_408_2 | AGTTTGCCAGGAGTGACGAA | SEQ ID NO: 116 |
| Elk3_ETS_13_3 | GGTCCAGCAGCAAGTGCAAG | SEQ ID NO: 117 |
| Elk3_ETS_14_4 | GTTCCTCTTGCACTTGCTGC | SEQ ID NO: 118 |
| Elk3_ETS_20_5 | AGATGAGGTGCTCATGTTTC | SEQ ID NO: 119 |
| Elk3_ETS_38_2 | TGGCCACTTCTTCCGCCTTG | SEQ ID NO: 120 |
| Elk3_ETS_86_1 | GTACAAGTTCGTCTCTTTCC | SEQ ID NO: 121 |
| Elk4_ETS_17_3 | GCTCATTCTGAGGCTCCTGC | SEQ ID NO: 122 |
| Elk4_ETS_21_4 | CAGATCATGTGCTCATTCTG | SEQ ID NO: 123 |
| Elk4_ETS_24_5 | AGAATGAGCACATGATCTGC | SEQ ID NO: 124 |
| Elk4_ETS_63_2 | CGTAGTAGTATCGCAGGGCT | SEQ ID NO: 125 |
| Elk4_ETS_65_1 | CTTTACGTAGTAGTATCGCA | SEQ ID NO: 126 |
| Eomes_TBOX_270 | TGCACAGGTAGACGTGGGCG | SEQ ID NO: 127 |
| Eomes_TBOX_274 | CCCACGTCTACCTGTGCAAC | SEQ ID NO: 128 |
| Eomes_TBOX_278 | GGAATTTGAGCCATAGGGGC | SEQ ID NO: 129 |
| Eomes_TBOX_286 | TGATCATCTCAGTTTGGTGC | SEQ ID NO: 130 |
| Eomes_TBOX_292 | TGAGATGATCATCACCAAAC | SEQ ID NO: 131 |
| Erg_ETS_319_3 | GGAACTGCCACAGCTGGATC | SEQ ID NO: 132 |
| Erg_ETS_319_4 | GTAGTGGCCAGATCCAGCTG | SEQ ID NO: 133 |
| Erg_ETS_321_5 | CGAGCAGGAACTGCCACAGC | SEQ ID NO: 134 |
| Erg_ETS_397_2 | CCCGTGGAAGTCAAACTTGT | SEQ ID NO: 135 |
| Erg_ETS_399_1 | CCTACAAGTTTGACTTCCAC | SEQ ID NO: 136 |
| Ets1_ETS_403_5 | CAAAAATATCATCCACAAGA | SEQ ID NO: 137 |
| Ets1_ETS_404_3 | AATATCATCCACAAGACGGC | SEQ ID NO: 138 |
| Ets1_ETS_404_4 | AAATATCATCCACAAGACGG | SEQ ID NO: 139 |
| Ets1_ETS_405_2 | AGCGCTTGCCCGCCGTCTTG | SEQ ID NO: 140 |
| Ets1_ETS_415_1 | TCTGCAGGTCGCACACAAAG | SEQ ID NO: 141 |
| Ets2_ETS_363_3 | GAAGCGGACCAATCCAGTTG | SEQ ID NO: 142 |
| Ets2_ETS_379_4 | CCAGCTGATGAAAGATTGAC | SEQ ID NO: 143 |
| Ets2_ETS_381_5 | CCTGTCAATCTTTCATCAGC | SEQ ID NO: 144 |
| Ets2_ETS_432_2 | AGCGCTTGCCCGAAGTCTTG | SEQ ID NO: 145 |
| Ets2_ETS_442_1 | TCTGCAGGTCACATACGAAA | SEQ ID NO: 146 |
| Etv3_DBD_105_5 | AGGATCCTTCATAAAACAAA | SEQ ID NO: 147 |
| Etv3_DBD_106_4 | GGATCCTTCATAAAACAAAA | SEQ ID NO: 148 |
| Etv3_DBD_108_3 | TTCATAAAACAAAAGGGAAA | SEQ ID NO: 149 |
| Etv3_DBD_44_2 | CTTCCTTCTGCAGCAGTTCC | SEQ ID NO: 150 |
| Etv3_DBD_53_1 | GCTGCCAGGCGATGACATGG | SEQ ID NO: 151 |
| Fli1_ETS_290_3 | CGCTGTCGGACAGTAGTTCC | SEQ ID NO: 152 |
| Fli1_ETS_295_4 | ACAGCTGGCGTTGGCGCTGT | SEQ ID NO: 153 |
| Fli1_ETS_298_5 | CCAGGTGATACAGCTGGCGT | SEQ ID NO: 154 |
| Fli1_ETS_353_2 | TGACCAAAGTGCATGGCAAA | SEQ ID NO: 155 |
| Fli1_ETS_360_1 | GCCATGGAAGTCAAACTTGT | SEQ ID NO: 156 |
| Fos_bZIP_142 | AAGAGAAACGGAGAATCCGA | SEQ ID NO: 157 |
| Fos_bZIP_145 | CCATCTTATTCCGTTCCCTT | SEQ ID NO: 158 |
| Fos_bZIP_155 | CTGCAGCCAAGTGCCGGAAT | SEQ ID NO: 159 |
| Fos_bZIP_187 | TTCCTTCTCTTTCAGCAGAT | SEQ ID NO: 160 |
| Fos_bZIP_195 | GGAAAAACTGGAGTTTATTT | SEQ ID NO: 161 |
| Fosb_bZIP_157_3 | CAGAAGAAGAAGAAAAGCGA | SEQ ID NO: 162 |
| Fosb_bZIP_162_4 | AGCGAAGGGTTCGCAGAGAG | SEQ ID NO: 163 |
| Fosb_bZIP_165_5 | TCGCAGAGAGCGGAACAAGC | SEQ ID NO: 164 |
| Fosb_bZIP_177_2 | GATCTGTCAGCTCCCTCCGA | SEQ ID NO: 165 |
| Fosb_bZIP_217_1 | AGCCCGGTTTGTGGGCCACC | SEQ ID NO: 166 |
| Fosl2_bZIP_129 | AGGAGAAGCGTCGAATCCGG | SEQ ID NO: 167 |
| Fosl2_bZIP_140 | AGCTAGCTGCAGCCAAGTGT | SEQ ID NO: 168 |
| Fosl2_bZIP_146 | TCTCTGTCAGCTCCCGGCGA | SEQ ID NO: 169 |
| Fosl2_bZIP_151 | GGAGCTGACAGAGAAGCTGC | SEQ ID NO: 170 |
| Fosl2_bZIP_159 | CTTCTCCTCTTCCAGCTCCT | SEQ ID NO: 171 |
| Foxm1_FH_238_3 | ATGGCCATATAAGAGTAGGG | SEQ ID NO: 172 |
| Foxm1_FH_238_4 | GAGGCCACCCTACTCTTATA | SEQ ID NO: 173 |
| Foxm1_FH_239_5 | ATCATGGCCATATAAGAGTA | SEQ ID NO: 174 |
| Foxm1_FH_259_2 | CCAAGTGTAGATGTCCTTCA | SEQ ID NO: 175 |
| Foxm1_FH_304_1 | CCAATGGCAAGGTCTCCTTC | SEQ ID NO: 176 |
| Foxo1_FH_162 | TGATGAGGTCGGCGTACGAC | SEQ ID NO: 177 |
| Foxo1_FH_172 | CTTCTCGGCTGAGCTCTCGA | SEQ ID NO: 178 |
| Foxo1_FH_177 | CGACAGGGTGAGCCTCTTCT | SEQ ID NO: 179 |
| Foxo1_FH_184 | CCCTGTCGCAGATCTACGAG | SEQ ID NO: 180 |
| Foxo1_FH_193 | GAAGAGCGTGCCCTACTTCA | SEQ ID NO: 181 |
| Foxo3_FH_158_3 | GGCATAGGACAGGTTCCCCC | SEQ ID NO: 182 |
| Foxo3_FH_161_4 | TGATCAGGTCGGCATAGGAC | SEQ ID NO: 183 |
| Foxo3_FH_163_5 | GCGGGTGATCAGGTCGGCAT | SEQ ID NO: 184 |
| Foxo3_FH_176_2 | GACAAAGTGAGCCGTTTGTC | SEQ ID NO: 185 |
| Foxo3_FH_231_1 | GCACGGGCAAGAGCTCTTGG | SEQ ID NO: 186 |
| Gata3_ZnF_277_2 | CTGTGGCGGCGAGATGGTAC | SEQ ID NO: 187 |
| Gata3_ZnF_296_5 | ATAAAATGAATGGGCAGAAC | SEQ ID NO: 188 |
| Gata3_ZnF_300_4 | GCTTGGGCTTGATAAGGGGC | SEQ ID NO: 189 |
| Gata3_ZnF_302_3 | CCTTCGCTTGGGCTTGATAA | SEQ ID NO: 190 |
| Gata3_ZnF_364_1 | ACATCTTCCGGTTTCGGGTC | SEQ ID NO: 191 |
| Hif1a_DBD_51_3 | CAAGATGTGAGCTCACATTG | SEQ ID NO: 192 |
| Hif1a_DBD_51_4 | AAGATGTGAGCTCACATTGT | SEQ ID NO: 193 |
| Hif1a_DBD_51_5 | AGATGTGAGCTCACATTGTG | SEQ ID NO: 194 |
| Hif1a_DBD_60_2 | TTGATAAAGCTTCTGTTATG | SEQ ID NO: 195 |
| Hif1a_DBD_67_1 | TCTCACACGTAAATAACTGA | SEQ ID NO: 196 |
| Hmbox1_DBD_320_5 | TCAGAGAGGTAACTCGTTCC | SEQ ID NO: 197 |
| Hmbox1_DBD_325_4 | CCAATTATATACTTTCAGAG | SEQ ID NO: 198 |
| Hmbox1_DBD_327_3 | CCTCTCTGAAAGTATATAAT | SEQ ID NO: 199 |
| Hmbox1_DBD_332_2 | ATAATTGGTTTGCTAATCGA | SEQ ID NO: 200 |
| Hmbox1_DBD_333_1 | TTGGTTTGCTAATCGACGGA | SEQ ID NO: 201 |
| Hmgb2_DBD_17_5 | CTGCACGAAGAAGGCGTACG | SEQ ID NO: 202 |
| Hmgb2_DBD_23_3 | CTTCGTGCAGACCTGCCGCG | SEQ ID NO: 203 |
| Hmgb2_DBD_33_2 | GAAGAAGCATCCCGACTCGT | SEQ ID NO: 204 |
| Hmgb2_DBD_35_1 | GCGAAGTTCACCGACGAGTC | SEQ ID NO: 205 |
| Hmgb2_DBD_35_4 | GGCGAAGTTCACCGACGAGT | SEQ ID NO: 206 |
| Hopx_DBD_11_3 | GAGCGGCCCCACGGAGGACC | SEQ ID NO: 207 |
| Hopx_DBD_11_4 | TCTCCACCTGGTCCTCCGTG | SEQ ID NO: 208 |
| Hopx_DBD_12_5 | ATCTCCACCTGGTCCTCCGT | SEQ ID NO: 209 |
| Hopx_DBD_32_2 | CGATGAGGCACAGCGTGGTG | SEQ ID NO: 210 |
| Hopx_DBD_60_1 | GCAGAGTGGCGGCGGTCAGA | SEQ ID NO: 211 |
| Id2_HLH_47 | CTACTCCAAGCTCAAGGAAC | SEQ ID NO: 212 |
| Id2_HLH_67 | TGTAATCGATGACGTGCTGC | SEQ ID NO: 213 |
| Id2_HLH_71 | GCACGTCATCGATTACATCT | SEQ ID NO: 214 |
| Id2_HLH_77 | CTTGGACCTGCAGATCGCCC | SEQ ID NO: 215 |
| Id2_HLH_81 | CGATAGTGGGATGCGAGTCC | SEQ ID NO: 216 |
| Id3_HLH_50 | ACCACTGCTACTCGCGCCTG | SEQ ID NO: 217 |
| Id3_HLH_53 | CTCCCGGCACCAGTTCCCGC | SEQ ID NO: 218 |
| Id3_HLH_58 | CTGGTGCCGGGAGTCCCGCG | SEQ ID NO: 219 |
| Id3_HLH_62 | TGGCTAAGCTGAGTGCCTCG | SEQ ID NO: 220 |
| Id3_HLH_68 | CACGCTGCAGGATTTCCACC | SEQ ID NO: 221 |
| Ikzf1_DBD_125_3 | ATCTGTGGGATCGTTTGCAT | SEQ ID NO: 222 |
| Ikzf1_DBD_132_4 | TTTGTGAACCATGAGCACAT | SEQ ID NO: 223 |
| Ikzf1_DBD_147_5 | CCACACTGGTTGCACTGGAA | SEQ ID NO: 224 |
| Ikzf1_DBD_210_2 | AGCTTCGCTGTTTATAGCTC | SEQ ID NO: 225 |
| Ikzf1_DBD_215_1 | TAAACAGCGAAGCTCTTTAG | SEQ ID NO: 226 |
| Ikzf2_DBD_143_5 | AAGCTCCGCACTGGTTACAG | SEQ ID NO: 227 |
| Ikzf2_DBD_146_4 | GGGTAAAAGAAGCTCCGCAC | SEQ ID NO: 228 |
| Ikzf2_DBD_153_2 | TCTCAGAAGGTTGCCCTTCT | SEQ ID NO: 229 |
| Ikzf2_DBD_153_3 | GTCTCAGAAGGTTGCCCTTC | SEQ ID NO: 230 |
| Ikzf2_DBD_182_1 | AGAAGAAGGGACGCTCTCAC | SEQ ID NO: 231 |
| Ikzf3_DBD_120_3 | AAGATGAACTGCGACGTGTG | SEQ ID NO: 232 |
| Ikzf3_DBD_121_4 | AGATGAACTGCGACGTGTGC | SEQ ID NO: 233 |
| Ikzf3_DBD_128_5 | CAAGACGTTGAAGCTAATGC | SEQ ID NO: 234 |
| Ikzf3_DBD_166_2 | CGTCATATTAAACTGCACAC | SEQ ID NO: 235 |
| Ikzf3_DBD_218_1 | GCGTTCCTTGTGCTCCTCCA | SEQ ID NO: 236 |
| Ikzf4_DBD_167_3 | GTCTGCGGCATGGTCTGCAT | SEQ ID NO: 237 |
| Ikzf4_DBD_168_4 | TCTGCGGCATGGTCTGCATT | SEQ ID NO: 238 |
| Ikzf4_DBD_174_5 | CTTGTGTACCATGAGCACAT | SEQ ID NO: 239 |
| Ikzf4_DBD_207_2 | CCCCCGAGTGCAGCTTGATG | SEQ ID NO: 240 |
| Ikzf4_DBD_229_1 | CGCCGGCGTGACGCACTCAC | SEQ ID NO: 241 |
| Irf1_IRF_37_5 | CAGCGTGCTTCCATGGAATC | SEQ ID NO: 242 |
| Irf1_IRF_40_4 | TGCTTAGCAGCGTGCTTCCA | SEQ ID NO: 243 |
| Irf1_IRF_42_3 | TGGAAGCACGCTGCTAAGCA | SEQ ID NO: 244 |
| Irf1_IRF_44_2 | GCACGCTGCTAAGCACGGCT | SEQ ID NO: 245 |
| Irf1_IRF_99_1 | GAAGGATCAGAGTAGGAACA | SEQ ID NO: 246 |
| Irf2_IRF_21_2 | CTTTAGCCCTGGTATCGTAT | SEQ ID NO: 247 |
| Irf2_IRF_39_4 | GCCGAGCCGCATGCATCCAG | SEQ ID NO: 248 |
| Irf2_IRF_39_5 | CCAGATCCCCTGGATGCATG | SEQ ID NO: 249 |
| Irf2_IRF_40_3 | GTGCCGAGCCGCATGCATCC | SEQ ID NO: 250 |
| Irf2_IRF_99_1 | AAGGACAGAAGCATAAAGAA | SEQ ID NO: 251 |
| Irf3_IRF_20_2 | CTGGACCTGGGGCAGCTGGA | SEQ ID NO: 252 |
| Irf3_IRF_39_5 | GCCGTAGGCCATGCTTCCAC | SEQ ID NO: 253 |
| Irf3_IRF_40_4 | TGCCGTAGGCCATGCTTCCA | SEQ ID NO: 254 |
| Irf3_IRF_41_3 | TCCCGTGGAAGCATGGCCTA | SEQ ID NO: 255 |
| Irf3_IRF_96_1 | GTTAGCTGCTGACAATAGCA | SEQ ID NO: 256 |
| Irf4_IRF_106 | GAACAAGAGCAATGACTTTG | SEQ ID NO: 257 |
| Irf4_IRF_58 | GTGGAAACACGCGGGCAAGC | SEQ ID NO: 258 |
| Irf4_IRF_64 | GCAGGACTACAATCGTGAGG | SEQ ID NO: 259 |
| Irf4_IRF_81 | TTTAAAGGCAAGTTCCGAGA | SEQ ID NO: 260 |
| Irf4_IRF_91 | ACAAGCCAGATCCTCCTACT | SEQ ID NO: 261 |
| Irf5_IRF_23_2 | CCCCACCACCCCGCCGTGTG | SEQ ID NO: 262 |
| Irf5_IRF_35_5 | GTACTGGCAGCTGTTCACCT | SEQ ID NO: 263 |
| Irf5_IRF_38_4 | GTGAACAGCTGCCAGTACCC | SEQ ID NO: 264 |
| Irf5_IRF_39_3 | TGAACAGCTGCCAGTACCCA | SEQ ID NO: 265 |
| Irf5_IRF_96_1 | GCGCAGGTTGGCCTTCCACT | SEQ ID NO: 266 |
| Irf6_IRF_26_5 | TGTGTAGCCAGATGAGACCA | SEQ ID NO: 267 |
| Irf6_IRF_27_4 | CTGTGTAGCCAGATGAGACC | SEQ ID NO: 268 |
| Irf6_IRF_36_3 | CCAGGGGATCTGGAAGCGTT | SEQ ID NO: 269 |
| Irf6_IRF_39_2 | TGGCATGTTTCCAGGGGATC | SEQ ID NO: 270 |
| Irf6_IRF_99_1 | CTTGATGTACGATGGCACCA | SEQ ID NO: 271 |
| Irf7_IRF_25_5 | TCAGCAGCGGCCAGTACGAG | SEQ ID NO: 272 |
| Irf7_IRF_26_4 | GCCACTGCAGCCCCTCGTAC | SEQ ID NO: 273 |
| Irf7_IRF_28_3 | GCCAGTACGAGGGGCTGCAG | SEQ ID NO: 274 |
| Irf7_IRF_40_2 | GCACAGTCTTCCGCGTACCC | SEQ ID NO: 275 |
| Irf7_IRF_98_1 | CTGTGCTGTGGAGTGCACAG | SEQ ID NO: 276 |
| Irf8_IRF_100_3 | CAGAAATGTCCAGCTGGGAC | SEQ ID NO: 277 |
| Irf8_IRF_102_4 | ATGGCTCAGAAATGTCCAGC | SEQ ID NO: 278 |
| Irf8_IRF_102_5 | TGGCTCAGAAATGTCCAGCT | SEQ ID NO: 279 |
| Irf8_IRF_93_2 | TCAGTCACTTCTTCAAAATC | SEQ ID NO: 280 |
| Irf8_IRF_99_1 | AGTGACTGACCGGTCCCAGC | SEQ ID NO: 281 |
| Irf9_IRF_107_3 | CTGCATGCTTCCAGGGAATC | SEQ ID NO: 282 |
| Irf9_IRF_109_4 | CGGATTCCCTGGAAGCATGC | SEQ ID NO: 283 |
| Irf9_IRF_110_5 | TTGCTTGCCTGCATGCTTCC | SEQ ID NO: 284 |
| Irf9_IRF_83_2 | GCGGAGCTGGATCGTGGAGC | SEQ ID NO: 285 |
| Irf9_IRF_84_1 | GAGCTGGATCGTGGAGCAGG | SEQ ID NO: 286 |
| Jun_bZIP_258 | AGGAGCGGATCAAGGCAGAG | SEQ ID NO: 287 |
| Jun_bZIP_268 | GGCACTTGGAGGCGGCAATG | SEQ ID NO: 288 |
| Jun_bZIP_273 | CAGCTTCCTTTTCCGGCACT | SEQ ID NO: 289 |
| Jun_bZIP_280 | GGAAGCTGGAGCGGATCGCT | SEQ ID NO: 290 |
| Jun_bZIP_295 | GAAAGCGCAAAACTCCGAGC | SEQ ID NO: 291 |
| Klf4_DBD_454_5 | GGCACTACCGCAAACACACA | SEQ ID NO: 292 |
| Klf4_DBD_456_4 | ACCGCAAACACACAGGGCAC | SEQ ID NO: 293 |
| Klf4_DBD_460_3 | ACTTCTGGCACTGAAAGGGC | SEQ ID NO: 294 |
| Klf4_DBD_465_1 | TCAGTGCCAGAAGTGTGACA | SEQ ID NO: 295 |
| Klf4_DBD_465_2 | AAAAGGCCCTGTCACACTTC | SEQ ID NO: 296 |
| Lef1_HMG_303 | TATAACATGAAAGCATTCAG | SEQ ID NO: 297 |
| Lef1_HMG_329 | TGCAGCTATCAACCAGATCC | SEQ ID NO: 298 |
| Lef1_HMG_340 | GGCCTGCTCTTCCCGGGAGA | SEQ ID NO: 299 |
| Lef1_HMG_347 | CCGTGCTAGTTCATAGTATT | SEQ ID NO: 300 |
| Lef1_HMG_360 | CTACACATGCAGCTTTATCC | SEQ ID NO: 301 |
| Maff_DBD_102_3 | CGTGAAGCGCGTGTGCCAGA | SEQ ID NO: 302 |
| Maff_DBD_103_4 | GAAGCGCGTGTGCCAGAAGG | SEQ ID NO: 303 |
| Maff_DBD_105_5 | GCTTCTGCAGCTCCTCCTTC | SEQ ID NO: 304 |
| Maff_DBD_97_2 | CACGCGCTTCACGCGGCAGC | SEQ ID NO: 305 |
| Maff_DBD_99_1 | TCTGGCACACGCGCTTCACG | SEQ ID NO: 306 |
| Mafg_bZIP_100_3 | GGAGAATGCCAGCATGAAGC | SEQ ID NO: 307 |
| Mafg_bZIP_101_4 | ATCGAGCTCCAGCTTCATGC | SEQ ID NO: 308 |
| Mafg_bZIP_112_5 | GTTCTGCAGGGCCTCGTACT | SEQ ID NO: 309 |
| Mafg_bZIP_89_2 | CCAGCTTCTCCACCTCCTGC | SEQ ID NO: 310 |
| Mafg_bZIP_93_1 | GGAGGTGGAGAAGCTGGCCT | SEQ ID NO: 311 |
| Myb_DBD_108 | ATAGAGCTTGTCCAGAAATA | SEQ ID NO: 312 |
| Myb_DBD_148 | GAAAACCTCCTGGACAGAAG | SEQ ID NO: 313 |
| Myb_DBD_40 | AGCGTCACTTGGGGAAAACT | SEQ ID NO: 314 |
| Myb_DBD_53 | TGAGAAGCTGAAGAAGCTGG | SEQ ID NO: 315 |
| Myb_DBD_97 | TGATCTTCTTCTTTGGTCCA | SEQ ID NO: 316 |
| Mybl2_DBD_123_2 | GGAAGCAGTGCCGTGAGCGC | SEQ ID NO: 317 |
| Mybl2_DBD_142_5 | GCTGGACCGAGGAGGAAGAC | SEQ ID NO: 318 |
| Mybl2_DBD_146_4 | GGAAGACAGGATAATCTGTG | SEQ ID NO: 319 |
| Mybl2_DBD_151_3 | CTGTGAGGCCCATAAAGTCC | SEQ ID NO: 320 |
| Mybl2_DBD_99_1 | ATTGAGTTGGTCAAGAAGTA | SEQ ID NO: 321 |
| Myc_HLH_370 | CGGAGGAAAACGACAAGAGG | SEQ ID NO: 322 |
| Myc_HLH_379 | ACAACGTCTTGGAACGTCAG | SEQ ID NO: 323 |
| Myc_HLH_394 | TTCAGGGATCTGGTCACGCA | SEQ ID NO: 324 |
| Myc_HLH_399 | TTTCGTTGTTTTCCAATTCA | SEQ ID NO: 325 |
| Myc_HLH_408 | TTTGAGGATCACTACCTTGG | SEQ ID NO: 326 |
| Mycl_HLH_315_3 | ACACTGAGGACGTGACCAAG | SEQ ID NO: 327 |
| Mycl_HLH_318_4 | GAAGTTATGGTTCTTCCTCT | SEQ ID NO: 328 |
| Mycl_HLH_321_5 | GAGGAAGAACCATAACTTCT | SEQ ID NO: 329 |
| Mycl_HLH_366_1 | GAATACTTGCAGGCTTTGGT | SEQ ID NO: 330 |
| Mycl_HLH_366_2 | AGAATACTTGCAGGCTTTGG | SEQ ID NO: 331 |
| Nfat5_DBD2_538_5 | TATAACTTTGCCTGTATCAG | SEQ ID NO: 332 |
| Nfat5_DBD2_540_4 | ACATATATTCCCACTGATAC | SEQ ID NO: 333 |
| Nfat5_DBD2_546_3 | ATATATGTTGTGACCAATGC | SEQ ID NO: 334 |
| Nfat5_DBD2_559_1 | TCAGCCATTTACGTACACTC | SEQ ID NO: 335 |
| Nfat5_DBD2_559_2 | GGATCCGGAGTGTACGTAAA | SEQ ID NO: 336 |
| Nfat5_RHD_344_5 | TAATGAACCAGTGGTGTTGC | SEQ ID NO: 337 |
| Nfat5_RHD_345_4 | ACAAATACCTGCAACACCAC | SEQ ID NO: 338 |
| Nfat5_RHD_347_3 | AGTGGTGTTGCAGGTATTTG | SEQ ID NO: 339 |
| Nfat5_RHD_432_2 | ATTAACTCGAAAAACCAATC | SEQ ID NO: 340 |
| Nfat5_RHD_439_1 | GTTAATATCACAAGGAAAGA | SEQ ID NO: 341 |
| Nfatc1_RHD_448_3 | ATGAGACGGAAGGCAGCCGG | SEQ ID NO: 342 |
| Nfatc1_RHD_448_4 | TGAGACGGAAGGCAGCCGGG | SEQ ID NO: 343 |
| Nfatc1_RHD_451_5 | AGGCAGCCGGGGGGCTGTGA | SEQ ID NO: 344 |
| Nfatc1_RHD_579_2 | CCGGACGCTGTCTCTCCAGG | SEQ ID NO: 345 |
| Nfatc1_RHD_582_1 | CGATAGGGTTCGAGGCCACC | SEQ ID NO: 346 |
| Nfatc2_DBD_423 | CTCATAGTGGGCCCGGTGAT | SEQ ID NO: 347 |
| Nfatc2_DBD_427 | CCGGGCCCACTATGAGACGG | SEQ ID NO: 348 |
| Nfatc2_DBD_437 | GGCGCTGTCAAAGCCCCAAC | SEQ ID NO: 349 |
| Nfatc2_DBD_450 | GCTTGTTCTCCATGTAGCCG | SEQ ID NO: 350 |
| Nfatc2_DBD_460 | CTGGGGCTTCAGATCTTCAT | SEQ ID NO: 351 |
| Nfatc3_DBD2_670_5 | TGTAACTGCTGGGTTATGAT | SEQ ID NO: 352 |
| Nfatc3_DBD2_673_4 | GCACGGCAGATGTAACTGCT | SEQ ID NO: 353 |
| Nfatc3_DBD2_674_3 | TGCACGGCAGATGTAACTGC | SEQ ID NO: 354 |
| Nfatc3_DBD2_676_2 | AGCAGTTACATCTGCCGTGC | SEQ ID NO: 355 |
| Nfatc3_DBD2_686_1 | TTTATCTTTGCAATGGCAAG | SEQ ID NO: 356 |
| Nfatc3_RHD_422_3 | GGTAAAGGCCAGTCTAATGG | SEQ ID NO: 357 |
| Nfatc3_RHD_423_4 | GTTGGTAAAGGCCAGTCTAA | SEQ ID NO: 358 |
| Nfatc3_RHD_427_5 | TGGCCTTTACCAACTCACTT | SEQ ID NO: 359 |
| Nfatc3_RHD_453_2 | ATGAAACTGAAGGTAGCCGA | SEQ ID NO: 360 |
| Nfatc3_RHD_575_1 | GTGCACATCCCACAGCCCAG | SEQ ID NO: 361 |
| Nfatc4_DBD2_612_3 | TTTAGAGTCTGGCAGGAAGT | SEQ ID NO: 362 |
| Nfatc4_DBD2_614_4 | ACACCACTTTAGAGTCTGGC | SEQ ID NO: 363 |
| Nfatc4_DBD2_615_5 | CTTCCTGCCAGACTCTAAAG | SEQ ID NO: 364 |
| Nfatc4_DBD2_671_2 | TTTACGTCTCCAATGGACGG | SEQ ID NO: 365 |
| Nfatc4_DBD2_672_1 | ACTGCGCTTCCTCCGTCCAT | SEQ ID NO: 366 |
| Nfatc4_RHD_434_2 | CGAGCTCACTATGAGACTGA | SEQ ID NO: 367 |
| Nfatc4_RHD_472_5 | TCATTGGCACTGCAGATGAG | SEQ ID NO: 368 |
| Nfatc4_RHD_475_4 | CTGCAGATGAGAGGAGCCTG | SEQ ID NO: 369 |
| Nfatc4_RHD_478_3 | GGTAGAAGGCATGGGGCCGC | SEQ ID NO: 370 |
| Nfatc4_RHD_567_1 | CGGGAAGGTCGTGTCTGTGC | SEQ ID NO: 371 |
| Nfkb1_DBD2_291_5 | GAAAATGGCGGAGTTTGGGA | SEQ ID NO: 372 |
| Nfkb1_DBD2_293_4 | GGCGGAGTTTGGGAAGGATT | SEQ ID NO: 373 |
| Nfkb1_DBD2_294_2 | GCGGAGTTTGGGAAGGATTT | SEQ ID NO: 374 |
| Nfkb1_DBD2_294_3 | CGGAGTTTGGGAAGGATTTG | SEQ ID NO: 375 |
| Nfkb1_DBD2_302_1 | CTGTCTATGAACATCTGTGG | SEQ ID NO: 376 |
| Nfkb1_RHD_144_3 | ACTTCATGTGACTAAGAAAA | SEQ ID NO: 377 |
| Nfkb1_RHD_149_4 | GAAAAAGGTATTTGAAACAC | SEQ ID NO: 378 |
| Nfkb1_RHD_152_5 | TATTTGAAACACTGGAAGCA | SEQ ID NO: 379 |
| Nfkb1_RHD_159_2 | GGATGACAGAGGCGTGTATT | SEQ ID NO: 380 |
| Nfkb1_RHD_98_1 | ATCGTTCAGTTGGTCACAAA | SEQ ID NO: 381 |
| Nfkb2_DBD2_262_5 | CATTGAGGTTCGGTTCTATG | SEQ ID NO: 382 |
| Nfkb2_DBD2_271_4 | GAGAATGGATGGCAAGCCTT | SEQ ID NO: 383 |
| Nfkb2_DBD2_272_2 | GAATGGATGGCAAGCCTTTG | SEQ ID NO: 384 |
| Nfkb2_DBD2_272_3 | AGAATGGATGGCAAGCCTTT | SEQ ID NO: 385 |
| Nfkb2_DBD2_275_1 | TGTGGGAGAGAAGTCCCCAA | SEQ ID NO: 386 |
| Nfkb2_RHD_145_2 | TGTAACCAAGAAGAACATGA | SEQ ID NO: 387 |
| Nfkb2_RHD_183_5 | CAAGGAGCTGAAGAAAGTCA | SEQ ID NO: 388 |
| Nfkb2_RHD_189_4 | TCATGGATCTGAGCATTGTA | SEQ ID NO: 389 |
| Nfkb2_RHD_200_3 | AGGAGCCATCGCTAGCTCGA | SEQ ID NO: 390 |
| Nfkb2_RHD_97_1 | GTGGGTCACTGTGTGTCACC | SEQ ID NO: 391 |
| Nr1d1_DBD_167_5 | AACAGAATATCCAGTACAAA | SEQ ID NO: 392 |
| Nr1d1_DBD_180_4 | GTTGCGATTGATGCGAACGA | SEQ ID NO: 393 |
| Nr1d1_DBD_187_3 | TGAAGCGACACTGCTGGCAG | SEQ ID NO: 394 |
| Nr1d1_DBD_189_2 | ACTTCTTGAAGCGACACTGC | SEQ ID NO: 395 |
| Nr1d1_DBD_195_1 | TTCAAGAAGTGTCTCTCCGT | SEQ ID NO: 396 |
| Nr1d2_DBD_143_2 | TGGAACAGTTCTCATTCTTC | SEQ ID NO: 397 |
| Nr1d2_DBD_148_1 | ATGAGAACTGTTCCATCATG | SEQ ID NO: 398 |
| Nr1d2_DBD_165_4 | TTTAAGAAGTGTCTGTCTGT | SEQ ID NO: 399 |
| Nr1d2_DBD_165_5 | CTTTAAGAAGTGTCTGTCTG | SEQ ID NO: 400 |
| Nr1d2_DBD_166_3 | TTAAGAAGTGTCTGTCTGTG | SEQ ID NO: 401 |
| Nr4a1_DBD_271 | GAGGGCCGCTGTGCAGTCTG | SEQ ID NO: 402 |
| Nr4a1_DBD_280 | AATGCTTCGTGTCAGCACTA | SEQ ID NO: 403 |
| Nr4a1_DBD_286 | TATGGGGTCCGCACCTGTGA | SEQ ID NO: 404 |
| Nr4a1_DBD_305 | AAGCGCCAAGTACATCTGCC | SEQ ID NO: 405 |
| Nr4a1_DBD_312 | GGCAAACAAGGATTGCCCTG | SEQ ID NO: 406 |
| Nr4a2_DBD_314_5 | GACAGTACTGACAACGATTT | SEQ ID NO: 407 |
| Nr4a2_DBD_317_4 | GAAATCGTTGTCAGTACTGT | SEQ ID NO: 408 |
| Nr4a2_DBD_325_3 | TTCAGAAGTGCCTAGCTGTT | SEQ ID NO: 409 |
| Nr4a2_DBD_326_1 | GAAGTGCCTAGCTGTTGGGA | SEQ ID NO: 410 |
| Nr4a2_DBD_326_2 | CTTTAACCATCCCAACAGCT | SEQ ID NO: 411 |
| Nr4a3_DBD_344_5 | TGCAGTACTGACATCGGTTT | SEQ ID NO: 412 |
| Nr4a3_DBD_346_4 | GAAATCTGCAGTACTGACAT | SEQ ID NO: 413 |
| Nr4a3_DBD_354_3 | TTTCAGAAGTGTCTCAGTGT | SEQ ID NO: 414 |
| Nr4a3_DBD_356_2 | GAAGTGTCTCAGTGTCGGGA | SEQ ID NO: 415 |
| Nr4a3_DBD_358_1 | TCTCAGTGTCGGGATGGTTA | SEQ ID NO: 416 |
| Pdcd1_e2.2 | CAGCTTGTCCAACTGGTCGG | SEQ ID NO: 417 |
| Pdcd1_IG_39 | TGTGAGCCAGGCTGGGTAGA | SEQ ID NO: 418 |
| Pdcd1_IG_44 | GCCTGGCTCACAGTGTCAGA | SEQ ID NO: 419 |
| Pdcd1_IG_56 | CGACCAGTTGGACAAGCTGC | SEQ ID NO: 420 |
| Pdcd1_IG_65 | CGGAGGATCTTATGCTGAAC | SEQ ID NO: 421 |
| Pparg_DBD_139_3 | CCCACAGACTCGGCACTCAA | SEQ ID NO: 422 |
| Pparg_DBD_140_4 | GCCATTGAGTGCCGAGTCTG | SEQ ID NO: 423 |
| Pparg_DBD_141_5 | CATTGAGTGCCGAGTCTGTG | SEQ ID NO: 424 |
| Pparg_DBD_169_2 | ATAAATAAGCTTCAATCGGA | SEQ ID NO: 425 |
| Pparg_DBD_220_1 | GGCCGAGAAGGAGAAGCTGT | SEQ ID NO: 426 |
| Prdm1_DBD_580 | AATGTCTGTGCCAAGACGTT | SEQ ID NO: 427 |
| Prdm1_DBD_594 | GTCCACCTGAGAGTGCACAG | SEQ ID NO: 428 |
| Prdm1_DBD_603 | TTGCAGGTCTGGCACTTGAA | SEQ ID NO: 429 |
| Prdm1_DBD_617 | CAAGTAGTGTTTCTGCAGGT | SEQ ID NO: 430 |
| Prdm1_DBD_637 | TTGTGCTGCTAAATCTCTTG | SEQ ID NO: 431 |
| Ptpn11_PTPc_349_3 | CAATGACTCGAGAGTTCTCC | SEQ ID NO: 432 |
| Ptpn11_PTPc_356_4 | AGTCATTGTCATGACCACAA | SEQ ID NO: 433 |
| Ptpn11_PTPc_358_5 | TGTCATGACCACAAAGGAAG | SEQ ID NO: 434 |
| Ptpn11_PTPc_378_2 | GAGTATGCGCTCAAAGAATA | SEQ ID NO: 435 |
| Ptpn11_PTPc_518_1 | GGACAGCCATGTAGATAAAC | SEQ ID NO: 436 |
| Rara_DBD_149_5 | GCTGCAGAAATGTTTCGACG | SEQ ID NO: 437 |
| Rara_DBD_94_4 | GTTTGTCAAGACAAATCATC | SEQ ID NO: 438 |
| Rara_DBD_98_3 | AAATCATCCGGCTACCACTA | SEQ ID NO: 439 |
| Rara_DBD_99_1 | ATCATCCGGCTACCACTATG | SEQ ID NO: 440 |
| Rara_DBD_99_2 | AATCATCCGGCTACCACTAT | SEQ ID NO: 441 |
| Rarb_DBD_117_3 | CAAGCCCTGCTTCGTTTGCC | SEQ ID NO: 442 |
| Rarb_DBD_117_4 | CTTGTCCTGGCAAACGAAGC | SEQ ID NO: 443 |
| Rarb_DBD_117_5 | TTGTCCTGGCAAACGAAGCA | SEQ ID NO: 444 |
| Rarb_DBD_176_1 | CTGCAGAAGTGCTTTGAAGT | SEQ ID NO: 445 |
| Rarb_DBD_176_2 | CCTGCAGAAGTGCTTTGAAG | SEQ ID NO: 446 |
| Rarg_DBD_85_5 | GGCTTATAGACCCGAGGAGG | SEQ ID NO: 447 |
| Rarg_DBD_86_4 | CATGGCTTATAGACCCGAGG | SEQ ID NO: 448 |
| Rarg_DBD_87_3 | AAGCATGGCTTATAGACCCG | SEQ ID NO: 449 |
| Rarg_DBD_92_2 | TTGTCATTGCATACAAAGCA | SEQ ID NO: 450 |
| Rarg_DBD_96_1 | GTATGCAATGACAAGTCTTC | SEQ ID NO: 451 |
| Rel_RHD_111_3 | TATTCGGTGTGTAAAGAAAA | SEQ ID NO: 452 |
| Rel_RHD_135_4 | TGTCCAGCAGCTGCTGTTCA | SEQ ID NO: 453 |
| Rel_RHD_136_5 | ATGTCCAGCAGCTGCTGTTC | SEQ ID NO: 454 |
| Rel_RHD_171_2 | ATTGGGTTAGAAACAATGGG | SEQ ID NO: 455 |
| Rel_RHD_91_1 | GGATACTATGAAGCAGAATT | SEQ ID NO: 456 |
| Rela_DBD2_232_5 | TTTCACGGGACCAGGCTGGG | SEQ ID NO: 457 |
| Rela_DBD2_234_3 | GGACCAGGCTGGGAGGCACG | SEQ ID NO: 458 |
| Rela_DBD2_234_4 | GAGCCTCGTGCCTCCCAGCC | SEQ ID NO: 459 |
| Rela_DBD2_241_2 | ATGCACATCAGCTTGAGAAA | SEQ ID NO: 460 |
| Rela_DBD2_246_1 | AGCTGATGTGCATCGGCAAG | SEQ ID NO: 461 |
| Rela_RHD_19_5 | CCAGGCTTCTGGGCCTTATG | SEQ ID NO: 462 |
| Rela_RHD_22_4 | TGTTCGATGATCTCCACATA | SEQ ID NO: 463 |
| Rela_RHD_28_2 | TCATCGAACAGCCGAAGCAA | SEQ ID NO: 464 |
| Rela_RHD_28_3 | ATCGAACAGCCGAAGCAACG | SEQ ID NO: 465 |
| Rela_RHD_99_1 | CTGCCGGGATGGCTACTATG | SEQ ID NO: 466 |
| Relb_DBD2_316_5 | TGGTGTTCAGCACAGCTTCC | SEQ ID NO: 467 |
| Relb_DBD2_317_4 | GGTGTTCAGCACAGCTTCCT | SEQ ID NO: 468 |
| Relb_DBD2_321_3 | GAAGTCGGCACGGCCTTCCC | SEQ ID NO: 469 |
| Relb_DBD2_326_2 | CACATCAGCTTGAGAGAAGT | SEQ ID NO: 470 |
| Relb_DBD2_329_1 | TCTCTCAAGCTGATGTGCAC | SEQ ID NO: 471 |
| Relb_RHD_151_3 | TCGAGACTGTGGCGGGCTGC | SEQ ID NO: 472 |
| Relb_RHD_152_4 | AGACTGTGGCGGGCTGCGGG | SEQ ID NO: 473 |
| Relb_RHD_153_5 | CTGTGGCGGGCTGCGGGAGG | SEQ ID NO: 474 |
| Relb_RHD_162_2 | GACGGCGTGCCTGGTGTGGA | SEQ ID NO: 475 |
| Relb_RHD_267_1 | TGTCGTAGACAGGCTCAGAG | SEQ ID NO: 476 |
| Rnf141_RING_167_5 | TGTGAGCACAGGGCAGGATG | SEQ ID NO: 477 |
| Rnf141_RING_169_4 | AGAAGCTGTGAGCACAGGGC | SEQ ID NO: 478 |
| Rnf141_RING_171_2 | CTGACAGAAGCTGTGAGCAC | SEQ ID NO: 479 |
| Rnf141_RING_171_3 | TGACAGAAGCTGTGAGCACA | SEQ ID NO: 480 |
| Rnf141_RING_191_1 | TCATCTGTAGGCGACAAATG | SEQ ID NO: 481 |
| Rora_DBD_108_3 | CTGACGAGGACAGGAGTAGG | SEQ ID NO: 482 |
| Rora_DBD_109_4 | CTTCTGACGAGGACAGGAGT | SEQ ID NO: 483 |
| Rora_DBD_111_5 | ACAGTTCTTCTGACGAGGAC | SEQ ID NO: 484 |
| Rora_DBD_86_2 | CACACGTAATGACACCATAA | SEQ ID NO: 485 |
| Rora_DBD_89_1 | TATGGTGTCATTACGTGTGA | SEQ ID NO: 486 |
| Rorb_DBD_100_3 | CAGGCTGTCCCGCTGCTTCT | SEQ ID NO: 487 |
| Rorb_DBD_105_4 | GCGGGACAGCCTGTATGCTG | SEQ ID NO: 488 |
| Rorb_DBD_106_5 | GCTTCTGCACCTCAGCATAC | SEQ ID NO: 489 |
| Rorb_DBD_37_2 | TACGGAGTCATCACGTGTGA | SEQ ID NO: 490 |
| Rorb_DBD_99_1 | GAGGATGTCCAAGAAGCAGC | SEQ ID NO: 491 |
| Rorc_DBD_37_2 | ATCTGTGGGGACAAGTCATC | SEQ ID NO: 492 |
| Rorc_DBD_44_5 | CACAGGTGATAACCCCGTAG | SEQ ID NO: 493 |
| Rorc_DBD_47_3 | TACGGGGTTATCACCTGTGA | SEQ ID NO: 494 |
| Rorc_DBD_47_4 | CTACGGGGTTATCACCTGTG | SEQ ID NO: 495 |
| Rorc_DBD_94_1 | CTCGGGACATGCCCAGAGCC | SEQ ID NO: 496 |
| Runx3_Runt_154_4 | TTCAACGACCTTCGATTCGT | SEQ ID NO: 497 |
| Runx3_Runt_154_5 | GTTCAACGACCTTCGATTCG | SEQ ID NO: 498 |
| Runx3_Runt_155_3 | CACTGCGGCCCACGAATCGA | SEQ ID NO: 499 |
| Runx3_Runt_158_2 | TTCGATTCGTGGGCCGCAGT | SEQ ID NO: 500 |
| Runx3_Runt_79_1 | TGCGCACGAGCTCTCCCGCG | SEQ ID NO: 501 |
| Satb1_DBD_652_3 | GCCACGAACCAAAATTTCCG | SEQ ID NO: 502 |
| Satb1_DBD_653_4 | CAGGGCTTCCACGGAAATTT | SEQ ID NO: 503 |
| Satb1_DBD_655_5 | AAAATTTCCGTGGAAGCCCT | SEQ ID NO: 504 |
| Satb1_DBD_702_2 | CGGTACTACCTTAAGCACCA | SEQ ID NO: 505 |
| Satb1_DBD_703_1 | TCAGCTTGCCATGGTGCTTA | SEQ ID NO: 506 |
| Smad1_DBD_127_2 | CTCCACTCGCTTATAGTGGT | SEQ ID NO: 507 |
| Smad1_DBD_20_5 | CATCGCCCTGTTTCCACCCA | SEQ ID NO: 508 |
| Smad1_DBD_27_4 | AGGGCGATGAAGAAGAGAAA | SEQ ID NO: 509 |
| Smad1_DBD_50_3 | GGCCTTCTCCAGCTCTTCCA | SEQ ID NO: 510 |
| Smad1_DBD_99_1 | TCAGTTCATGGTGGCTCTGG | SEQ ID NO: 511 |
| Smad2_MH1_12 | AGAAGTCTCTTCACCACTGG | SEQ ID NO: 512 |
| Smad2_MH1_20 | CTGGGATGGAAAAAATCAGC | SEQ ID NO: 513 |
| Smad2_MH1_28 | GGGTCTGGAGGAGCAGGTGG | SEQ ID NO: 514 |
| Smad2_MH1_46 | TGAAAAAGCAGTGAAAAGTC | SEQ ID NO: 515 |
| Smad2_MH1_67 | ATTGCAATTCTGAGTGGTGA | SEQ ID NO: 516 |
| Smad3_MH1_11 | CAGGCGCTTCACGATCGGGG | SEQ ID NO: 517 |
| Smad3_MH1_16 | TCGTGAAGCGCCTGCTGGGT | SEQ ID NO: 518 |
| Smad3_MH1_24 | GAAGGGCGAGCAGAACGGGC | SEQ ID NO: 519 |
| Smad3_MH1_36 | CGAGAAGGCGGTCAAGAGCT | SEQ ID NO: 520 |
| Smad3_MH1_46 | GCTCAAGAAGACGGGGCAGT | SEQ ID NO: 521 |
| Smad4_DBD_105_3 | AGTTCATTCTTGTGTAGATC | SEQ ID NO: 522 |
| Smad4_DBD_133_4 | ACAACCCGCTCATAGTGATA | SEQ ID NO: 523 |
| Smad4_DBD_133_5 | GAATCCATATCACTATGAGC | SEQ ID NO: 524 |
| Smad4_DBD_28_2 | TGATGTGTCATAGACAAGGT | SEQ ID NO: 525 |
| Smad4_DBD_99_1 | TCTATGCCCGTCTGTGGAGG | SEQ ID NO: 526 |
| Smad5_DBD_28_5 | AGGTGACGAGGAAGAGAAAT | SEQ ID NO: 527 |
| Smad5_DBD_31_4 | GGAAGAGAAATGGGCAGAAA | SEQ ID NO: 528 |
| Smad5_DBD_44_3 | GAAAAAGCTGAAGAAGAAGA | SEQ ID NO: 529 |
| Smad5_DBD_50_2 | GAAGGGTGCTATGGAGGAGC | SEQ ID NO: 530 |
| Smad5_DBD_99_1 | TCGTGATGGCTCTGCAAATC | SEQ ID NO: 531 |
| Smad6_DBD_214_5 | CCGCGCGCCGACCTCCGCTT | SEQ ID NO: 532 |
| Smad6_DBD_215_3 | CTGGCCGCCCAAGCGGAGGT | SEQ ID NO: 533 |
| Smad6_DBD_215_4 | CGCGCCGACCTCCGCTTGGG | SEQ ID NO: 534 |
| Smad6_DBD_223_2 | GAGCAGCAGCTGCGGTGGCG | SEQ ID NO: 535 |
| Smad6_DBD_270_1 | ACCACTTCAGCCGGCTCTGC | SEQ ID NO: 536 |
| Smad7_DBD_137_3 | GGACTGCAGGCTGGGCCCGG | SEQ ID NO: 537 |
| Smad7_DBD_137_4 | TGGACTGCAGGCTGGGCCCG | SEQ ID NO: 538 |
| Smad7_DBD_149_5 | GTAGGACGAGGGCGGCTGCG | SEQ ID NO: 539 |
| Smad7_DBD_155_2 | CAGCAGGAGGGGGAGCGAGT | SEQ ID NO: 540 |
| Smad7_DBD_96_1 | TCTGAAGGCGCTCACGCACT | SEQ ID NO: 541 |
| Sox2_HMG_59_5 | CCGGGGGCAGCGGCGTAAGA | SEQ ID NO: 542 |
| Sox2_HMG_77_4 | GATCAGCAAGCGCCTGGGCG | SEQ ID NO: 543 |
| Sox2_HMG_79_3 | AAAGTTTCCACTCCGCGCCC | SEQ ID NO: 544 |
| Sox2_HMG_96_2 | CGTTCATCGACGAGGCCAAG | SEQ ID NO: 545 |
| Sox2_HMG_99_1 | GTGCAGAGCGCGCAGCCGCT | SEQ ID NO: 546 |
| Sox3_HMG_189_5 | TGCTGACCGATGCGGAGAAG | SEQ ID NO: 547 |
| Sox3_HMG_200_3 | GTGCACCGCACGCAGTCGCT | SEQ ID NO: 548 |
| Sox3_HMG_200_4 | CGAGGCCAAGCGACTGCGTG | SEQ ID NO: 549 |
| Sox3_HMG_204_2 | ACTGCGTGCGGTGCACATGA | SEQ ID NO: 550 |
| Sox3_HMG_207_1 | GGTGCACATGAAGGAGTACC | SEQ ID NO: 551 |
| Stat1_DBD_398_2 | AGAATCCACCAACGGAAGTC | SEQ ID NO: 552 |
| Stat1_DBD_399_3 | CTCAGCTGCCAGACTTCCGT | SEQ ID NO: 553 |
| Stat1_DBD_413_4 | CTGAAGGAACAGAAAAACGC | SEQ ID NO: 554 |
| Stat1_DBD_414_5 | TGAAGGAACAGAAAAACGCT | SEQ ID NO: 555 |
| Stat1_DBD_570_1 | GGTGCTTCTTAATGAGCTCT | SEQ ID NO: 556 |
| Stat2_DBD_387_3 | AAAACCTTGACCCCGGAGGA | SEQ ID NO: 557 |
| Stat2_DBD_387_4 | CTGGCCCTCCTCCGGGGTCA | SEQ ID NO: 558 |
| Stat2_DBD_389_5 | CTGTCTCTGGCCCTCCTCCG | SEQ ID NO: 559 |
| Stat2_DBD_395_2 | GCCAGAGACAGGGCTTAATT | SEQ ID NO: 560 |
| Stat2_DBD_559_1 | GCTGGACAAAATTCTGGAGC | SEQ ID NO: 561 |
| Stat3_DBD_13 | GCAGCTGGACACACGCTACC | SEQ ID NO: 562 |
| Stat3_DBD_26 | GTACAGCGACAGCTTCCCCA | SEQ ID NO: 563 |
| Stat3_DBD_36 | GACTCTCAATCCAAGGTGCC | SEQ ID NO: 564 |
| Stat3_DBD_56 | GAGATTATGAAACACCAACG | SEQ ID NO: 565 |
| Stat3_DBD_74 | AGAGGACATTGGACTCTTGC | SEQ ID NO: 566 |
| Stat4_DBD__101 | ATTACCACAGCTACATGCAT | SEQ ID NO: 567 |
| Stat4_DBD_139 | CTTCAGTTTCTGAAAGACAA | SEQ ID NO: 568 |
| Stat4_DBD_26 | CTATGATGACAACTTTCCTA | SEQ ID NO: 569 |
| Stat4_DBD_35 | TCCGGCATCTGCTAGCTCAG | SEQ ID NO: 570 |
| Stat4_DBD_66 | AATACAATTGGATGAACAGT | SEQ ID NO: 571 |
| Stat5a_DBD_466_4 | GGACGATAACGACCACAGGG | SEQ ID NO: 572 |
| Stat5a_DBD_466_5 | GACGATAACGACCACAGGGA | SEQ ID NO: 573 |
| Stat5a_DBD_467_3 | CATGGACGATAACGACCACA | SEQ ID NO: 574 |
| Stat5a_DBD_576_2 | GCAGTGGTTCGACGGGGTGA | SEQ ID NO: 575 |
| Stat5a_DBD_577_1 | GTGGTTCGACGGGGTGATGG | SEQ ID NO: 576 |
| Stat5b_DBD_356_3 | CCGTGCGCCTGCTGGTGGGG | SEQ ID NO: 577 |
| Stat5b_DBD_423_4 | AGACCTCTTGATTCGTTTCA | SEQ ID NO: 578 |
| Stat5b_DBD_423_5 | CAGACCTCTTGATTCGTTTC | SEQ ID NO: 579 |
| Stat5b_DBD_573_2 | ACTTTCTGGCAGTGGTTTGA | SEQ ID NO: 580 |
| Stat5b_DBD_576_1 | GCAGTGGTTTGATGGCGTGA | SEQ ID NO: 581 |
| Stat6_DBD_328_3 | CTAAGCCTGGCCCAGGGGCC | SEQ ID NO: 582 |
| Stat6_DBD_329_4 | TAAGCCTGGCCCAGGGGCCC | SEQ ID NO: 583 |
| Stat6_DBD_342_5 | AGGAGAGATCATGAACAACA | SEQ ID NO: 584 |
| Stat6_DBD_371_2 | AGAAAATAAAGCGCTGTGAG | SEQ ID NO: 585 |
| Stat6_DBD_526_1 | TCCGGAGACAGCGTTTGGTG | SEQ ID NO: 586 |
| Tbx21_TBOX_145 | TGAACTTGGACCACAACAGG | SEQ ID NO: 587 |
| Tbx21_TBOX_155 | TAGTGATGATCATCTCTGTC | SEQ ID NO: 588 |
| Tbx21_TBOX_169 | CCACGGTGAAGGACAGGAAT | SEQ ID NO: 589 |
| Tbx21_TBOX_181 | TGGAGCCCACAAGCCATTAC | SEQ ID NO: 590 |
| Tbx21_TBOX_195 | TGGTGGACCAGCACCACTGG | SEQ ID NO: 591 |
| Tcf19_FHA_24_3 | AGCCAGACTTGGACGGGGGG | SEQ ID NO: 592 |
| Tcf19_FHA_25_4 | TGCAGCCAGACTTGGACGGG | SEQ ID NO: 593 |
| Tcf19_FHA_25_5 | TTCCACCCCCCGTCCAAGTC | SEQ ID NO: 594 |
| Tcf19_FHA_28_2 | TCGATAGGTGCAGCCAGACT | SEQ ID NO: 595 |
| Tcf19_FHA_94_1 | CATCACTCAACTCCAGTCTG | SEQ ID NO: 596 |
| Tcf3_HLH_548_3 | CGGAGCGGGAGAAGGAGCGC | SEQ ID NO: 597 |
| Tcf3_HLH_548_4 | GGAGCGGGAGAAGGAGCGCC | SEQ ID NO: 598 |
| Tcf3_HLH_549_5 | GCGGGAGAAGGAGCGCCGGG | SEQ ID NO: 599 |
| Tcf3_HLH_564_2 | GCGGGTCCGCGACATCAATG | SEQ ID NO: 600 |
| Tcf3_HLH_601_1 | GCACCTGCTGCTCCAGGCTG | SEQ ID NO: 601 |
| Tcf7_HMG_189_3 | TTGAGGGGTTTCTTGATGAC | SEQ ID NO: 602 |
| Tcf7_HMG_194_4 | TAAAGCATGAACGCATTGAG | SEQ ID NO: 603 |
| Tcf7_HMG_194_5 | GTAAAGCATGAACGCATTGA | SEQ ID NO: 604 |
| Tcf7_HMG_223_2 | TCAACCAGATCCTGGGTCGC | SEQ ID NO: 605 |
| Tcf7_HMG_256_1 | ATACCCGGGCTGGTCCGCGC | SEQ ID NO: 606 |
| Tcf7I1_HMG_363_3 | ATACAACATGAAGGCATTCA | SEQ ID NO: 607 |
| Tcf7I1_HMG_363_4 | TACAACATGAAGGCATTCAG | SEQ ID NO: 608 |
| Tcf7I1_HMG_363_5 | TATACAACATGAAGGCATTC | SEQ ID NO: 609 |
| Tcf7I1_HMG_383_2 | TAATGGCTGCACTTTCCTTC | SEQ ID NO: 610 |
| Tcf7I1_HMG_426_1 | ATAGTTGTCCCGGGCTGACC | SEQ ID NO: 611 |
| Tox_HMG_261 | ACAGGCTTCTGGGGCTCATT | SEQ ID NO: 612 |
| Tox_HMG_264 | TAGGCAGACACAGGCTTCTG | SEQ ID NO: 613 |
| Tox_HMG_274 | TTTGTTCTTTCGTGATACCC | SEQ ID NO: 614 |
| Tox_HMG_284 | CAAAAGTAGCATTTGGATTC | SEQ ID NO: 615 |
| Tox_HMG_297 | CGAAGATTGTGGCATCAATG | SEQ ID NO: 616 |
| Tox2_HMG_253_3 | ACTGGCTTCTGTGGCTCATT | SEQ ID NO: 617 |
| Tox2_HMG_256_4 | TAGGCCGACACTGGCTTCTG | SEQ ID NO: 618 |
| Tox2_HMG_256_5 | TGAGCCACAGAAGCCAGTGT | SEQ ID NO: 619 |
| Tox2_HMG_316_1 | TGTAGGCCGCCAAGGCTTTC | SEQ ID NO: 620 |
| Tox2_HMG_316_2 | GGAGTACCTGAAAGCCTTGG | SEQ ID NO: 621 |
| Trp53_DBD_141_3 | AGACGTGCCCTGTGCAGTTG | SEQ ID NO: 622 |
| Trp53_DBD_161_4 | GTGCTGTGACTTCTTGTAGA | SEQ ID NO: 623 |
| Trp53_DBD_165_5 | CAAGAAGTCACAGCACATGA | SEQ ID NO: 624 |
| Trp53_DBD_277_2 | CTGTACGGCGGTCTCTCCCA | SEQ ID NO: 625 |
| Trp53_DBD_278_1 | TCTGTACGGCGGTCTCTCCC | SEQ ID NO: 626 |
| Zbtb25_DBD_243_5 | CCACTTATGCCATTACTGTG | SEQ ID NO: 627 |
| Zbtb25_DBD_244_4 | CAAAGCGCTCCCCACAGTAA | SEQ ID NO: 628 |
| Zbtb25_DBD_253_2 | GTGTTGTCTCAGGTTACTTC | SEQ ID NO: 629 |
| Zbtb25_DBD_253_3 | GGTGTTGTCTCAGGTTACTT | SEQ ID NO: 630 |
| Zbtb25_DBD_256_1 | GGGTATGGAGGTGTTGTCTC | SEQ ID NO: 631 |
| Zc3h12a_Rnase_140_3 | TTGCTTCCGTCGATGACCAC | SEQ ID NO: 632 |
| Zc3h12a_Rnase_143_4 | GGTCATCGACGGAAGCAATG | SEQ ID NO: 633 |
| Zc3h12a_Rnase_151_5 | AGAAGACTTCCTTGTTCCCA | SEQ ID NO: 634 |
| Zc3h12a_Rnase_183_2 | GGCTGTTCCTTCCTCCAAGA | SEQ ID NO: 635 |
| Zc3h12a_Rnase_289 | CGAAGGAAGTTGTCCAGGCT | SEQ ID NO: 636 |
| Zc3h12c_RNase_304 | AAGAGGCCATAAAGATATCA | SEQ ID NO: 637 |
| Zc3h12c_RNase_342 | TCCTGGTATTCACACCATCA | SEQ ID NO: 638 |
| Zc3h12c_RNase_345 | CACACCATCACGGCGTGTCC | SEQ ID NO: 639 |
| Zc3h12c_RNase_345 | ACACCATCACGGCGTGTCCA | SEQ ID NO: 640 |
| Zc3h12c_RNase_419 | CTCAGAAAATTATCCAGACT | SEQ ID NO: 641 |
| Zeb1_NAB_915 | CGAGTGTGGAATCTGTAGAA | SEQ ID NO: 642 |
| Zeb1_NAB_928 | ATCATTTGATTGAGCACATG | SEQ ID NO: 643 |
| Zeb1_NAB_932 | GCACATGCGGCTGCACTCTG | SEQ ID NO: 644 |
| Zeb1_NAB_940 | CCACACTTGTCACATTGATA | SEQ ID NO: 645 |
| Zeb1_NAB_955 | GTGATTCATGTGTTGAGAGT | SEQ ID NO: 646 |
| Zeb2_HD_654 | ACAGACATGTGGTCCTTGTA | SEQ ID NO: 647 |
| Zeb2_HD_665 | AGCATACTATGCTATGAACA | SEQ ID NO: 648 |
| Zeb2_HD_672 | TTCAGCAGTTCATCAGAGTT | SEQ ID NO: 649 |
| Zeb2_HD_681 | CTGAGGAAGGCCCACAGCAA | SEQ ID NO: 650 |
| Zeb2_HD_689 | CTCAGGAATTTGTGAAGGAA | SEQ ID NO: 651 |
| Zfp397_DBD_438_4 | TCCACTGTGTATCCTCTGAT | SEQ ID NO: 652 |
| Zfp397_DBD_438_5 | CTCCACTGTGTATCCTCTGA | SEQ ID NO: 653 |
| Zfp397_DBD_439_3 | ACCCATCAGAGGATACACAG | SEQ ID NO: 654 |
| Zfp397_DBD_457_2 | GAGGTTTGAATTCAGACTGA | SEQ ID NO: 655 |
| Zfp397_DBD_463_1 | GGATTCTTTGATGTCTTATG | SEQ ID NO: 656 |
| Zfp609_PTZ_1072_3 | AGGATCTGTCCCAGGCTCCT | SEQ ID NO: 657 |
| Zfp609_PTZ_1075_4 | GACTTGGCAGGATCTGTCCC | SEQ ID NO: 658 |
| Zfp609_PTZ_1079_5 | GGAATGATGACAGACTTGGC | SEQ ID NO: 659 |
| Zfp609_PTZ_1099_2 | AGCTTCCCTTTAAGGCCTTC | SEQ ID NO: 660 |
| Zfp609_PTZ_1226_1 | GCATGTAGGGGATGTAGGAC | SEQ ID NO: 661 |
| Zfp609_SFP1_470_4 | GGGCCCGGTGGCTGAGCCCA | SEQ ID NO: 662 |
| Zfp609_SFP1_470_5 | AATTCCATGGGCTCAGCCAC | SEQ ID NO: 663 |
| Zfp609_SFP1_471_3 | ATTCCATGGGCTCAGCCACC | SEQ ID NO: 664 |
| Zfp609_SFP1_475_2 | CAGCCACCGGGCCCCTCCCT | SEQ ID NO: 665 |
| Zfp609_SFP1_528_1 | GACAGCAAACCGGAAGCTGA | SEQ ID NO: 666 |
| Zfp638_AF4_553_3 | AGGACTTCGGACTTCTTCTA | SEQ ID NO: 667 |
| Zfp638_AF4_557_4 | GAAGAAGTCCGAAGTCCTAT | SEQ ID NO: 668 |
| Zfp638_AF4_558_5 | CTAGCTGACCGATAGGACTT | SEQ ID NO: 669 |
| Zfp638_AF4_602_2 | ATACTGAAACAGTTGATAAA | SEQ ID NO: 670 |
| Zfp638_AF4_669_1 | CTCAAAGACTGATCCTTCTG | SEQ ID NO: 671 |
| Zfp638_Znf_1875_4 | GGACAGAAAAATCCAGCCTT | SEQ ID NO: 672 |
| Zfp638_Znf_1882_3 | GAGTAGAAGAGGGAGCAAAT | SEQ ID NO: 673 |
| Zfp638_Znf_1884_2 | ATTTGCTCCCTCTTCTACTC | SEQ ID NO: 674 |
| Zfp638_Znf_1889_1 | CTACTCAGGTGAAAAAGCAA | SEQ ID NO: 675 |

What is claimed is:

1. A modified immune cell or precursor thereof, comprising a modification in an endogenous gene locus encoding Fli1.

2. A modified immune cell or precursor thereof, wherein the endogenous Fli1 gene or protein is disrupted.

3. The modified immune cell or precursor thereof of claim 1, wherein the modification or disruption is made by a method selected from the group consisting of a CRISPR system, an antibody, an siRNA, a miRNA, an antagonist, a drug, a small molecule inhibitor, a PROTAC target, a TALEN, and a Zinc Finger Nuclease.

4. The modified immune cell or precursor thereof of claim 3, wherein the CRISPR system comprises at least one sgRNA comprising any one of SEQ ID NOs: 152-156 or SEQ ID NOs: 676-713.

5. The modified immune cell or precursor thereof of claim 1, wherein the cell is a human cell.

6. The modified immune cell or precursor thereof of claim 1, wherein the cell is a T cell.

7. The modified immune cell or precursor thereof of claim 6, wherein the T cell is resistant to T cell exhaustion.

8. A pharmaceutical composition comprising an inhibitor of Fli1.

9. The pharmaceutical composition of claim 8, wherein the inhibitor is selected from the group consisting of a CRISPR system, an antibody, an siRNA, a miRNA, an antagonist, a drug, a small molecule inhibitor, a PROTAC target, a TALEN, and a Zinc Finger Nuclease.

10. The composition of claim 9, wherein the CRISPR system comprises at least one sgRNA comprising any one of SEQ ID NOs: 152-156 or SEQ ID NOs: 676-713.

11. A method of treating a disease or disorder in a subject in need thereof, the method comprising administering to the subject the cell of claim 1.

12. The method of claim 11, wherein the disease or disorder is an infection.

13. The method of claim 11, wherein the disease is cancer.

14. A method of screening a T cell, the method comprising:
i) introducing into an activated T cell a Cas enzyme and an sgRNA library,
ii) administering the T cell to an infected mouse,
iii) isolating the T cell from the infected mouse, and
iv) analyzing the T cell.

15. The method of claim 14, wherein the sgRNA library comprises a plurality of sgRNAs that target a plurality of transcription factors.

16. The method of claim 15, wherein the plurality of transcription factors comprise any of the transcription factors listed in Table 1.

17. The method of claim 15, wherein each sgRNA targets the DNA binding domain of each transcription factor.

18. The method of claim 14, wherein the sgRNA library comprises at least one sequence selected from the group consisting of SEQ ID NOs: 1-675.

19. The method of claim 14, wherein the sgRNA library consists of the nucleotide sequences set forth in SEQ ID NOs: 1-675.

20. The method of claim 14, wherein the screening assesses T cell exhaustion.

21. The method of claim 14, wherein analyzing the cell comprises a method selected from the group consisting of sequencing, PCR, MACS, and FACS.

22. The method of claim 14, wherein the sequencing reveals a target of interest.

23. The method of claim 22, wherein a drug is designed against the target of interest.

24. The method of claim 22, wherein when the drug is administered to the T cell, at least one T cell response is increased.

25. The method of claim 14, wherein 1×10⁵ T cells are administered to the infected mouse.

26. The method of claim 14, wherein the method identifies novel transcription factors governing T_{EFF} and T_{EX} cell differentiation.


SEQUENCE LISTING (721 sequences)

SEQ ID NO: 1 - DNA, 20 bp, Artificial Sequence
  sgRNA
aatggcaactggtccccttc

SEQ ID NO: 2 - DNA, 20 bp, Artificial Sequence
  sgRNA
gaagatgggcgggagtcttc

SEQ ID NO: 3 - DNA, 20 bp, Artificial Sequence
  sgRNA
catcggcccggggcaactcg

SEQ ID NO: 4 - DNA, 20 bp, Artificial Sequence
  sgRNA
aaagtccttcatccggctta

SEQ ID NO: 5 - DNA, 20 bp, Artificial Sequence
  sgRNA
cgtgtccgaactctgcatcc

SEQ ID NO: 6 - DNA, 20 bp, Artificial Sequence
  sgRNA
ctgatcaacatgtacctgcc

SEQ ID NO: 7 - DNA, 20 bp, Artificial Sequence
  sgRNA
gtgagcgcaccgcagtgccc

SEQ ID NO: 8 - DNA, 20 bp, Artificial Sequence
  sgRNA
tagttgggtgcgcccgcgta

SEQ ID NO: 9 - DNA, 20 bp, Artificial Sequence
  sgRNA
aacatgcgcggcagcggcac

SEQ ID NO: 10 - DNA, 20 bp, Artificial Sequence
  sgRNA
cgagcagcgggtcaccgtcc

SEQ ID NO: 11 - DNA, 20 bp, Artificial Sequence
  sgRNA
agtcttgtcaagcttaactc

SEQ ID NO: 12 - DNA, 20 bp, Artificial Sequence
  sgRNA
gcttattaataacatcttgc

SEQ ID NO: 13 - DNA, 20 bp, Artificial Sequence
  sgRNA
gcaagatgttattaataagc

SEQ ID NO: 14 - DNA, 20 bp, Artificial Sequence
  sgRNA
tggacaaactctctgttctt

SEQ ID NO: 15 - DNA, 20 bp, Artificial Sequence
  sgRNA
ggctcagcgtcagctacctg

SEQ ID NO: 16 - DNA, 20 bp, Artificial Sequence
  sgRNA
gctcagcgtcagctacctga

SEQ ID NO: 17 - DNA, 20 bp, Artificial Sequence
  sgRNA
caagtgtcgaaacaagaaaa

SEQ ID NO: 18 - DNA, 20 bp, Artificial Sequence
  sgRNA
gagtgtgaatgctgagctga

SEQ ID NO: 19 - DNA, 20 bp, Artificial Sequence
  sgRNA
tgagctgaaggcccagattg

SEQ ID NO: 20 - DNA, 20 bp, Artificial Sequence
  sgRNA
tatacatgctcaacctgcac

SEQ ID NO: 21 - DNA, 20 bp, Artificial Sequence
  sgRNA
ctgaagaagatgagaggaaa

SEQ ID NO: 22 - DNA, 20 bp, Artificial Sequence
  sgRNA
tggatgaagatccagatgag

SEQ ID NO: 23 - DNA, 20 bp, Artificial Sequence
  sgRNA
aaccgctgccgccgctcatc

SEQ ID NO: 24 - DNA, 20 bp, Artificial Sequence
  sgRNA
atgaagatccagatgagcgg

SEQ ID NO: 25 - DNA, 20 bp, Artificial Sequence
  sgRNA
atccagatgagcggcggcag

SEQ ID NO: 26 - DNA, 20 bp, Artificial Sequence
  sgRNA
gagctgcagcctctcgatgc

SEQ ID NO: 27 - DNA, 20 bp, Artificial Sequence
  sgRNA
ctgtgctgaggagacagcag

SEQ ID NO: 28 - DNA, 20 bp, Artificial Sequence
  sgRNA
gcgactgacaagcagactct

SEQ ID NO: 29 - DNA, 20 bp, Artificial Sequence
  sgRNA
aagaagaaagagtatatgct

SEQ ID NO: 30 - DNA, 20 bp, Artificial Sequence
  sgRNA
aggactggaggccaggctca

SEQ ID NO: 31 - DNA, 20 bp, Artificial Sequence
  sgRNA
cctgaagcgacagctggacg

SEQ ID NO: 32 - DNA, 20 bp, Artificial Sequence
  sgRNA
caagctaacctcagaacagt

SEQ ID NO: 33 - DNA, 20 bp, Artificial Sequence
  sgRNA
tggagttcatccacgacatc

SEQ ID NO: 34 - DNA, 20 bp, Artificial Sequence
  sgRNA
tcatccacgacatccggagg

SEQ ID NO: 35 - DNA, 20 bp, Artificial Sequence
  sgRNA
gcttcctctttcgacagcgc

SEQ ID NO: 36 - DNA, 20 bp, Artificial Sequence
  sgRNA
cgagaaggaaaaactgttgt

SEQ ID NO: 37 - DNA, 20 bp, Artificial Sequence
  sgRNA
atgatgtgaggaaagttcag

SEQ ID NO: 38 - DNA, 20 bp, Artificial Sequence
  sgRNA
ctgtctctgtcggctcttct

SEQ ID NO: 39 - DNA, 20 bp, Artificial Sequence
  sgRNA
gagctgcgttctgtttctcc

SEQ ID NO: 40 - DNA, 20 bp, Artificial Sequence
  sgRNA
tgagctgtttgatctctttg

SEQ ID NO: 41 - DNA, 20 bp, Artificial Sequence
  sgRNA
agagatcaaacagctcaccg

SEQ ID NO: 42 - DNA, 20 bp, Artificial Sequence
  sgRNA
gaattcgagtgtgggtcttc

SEQ ID NO: 43 - DNA, 20 bp, Artificial Sequence
  sgRNA
acccacactcgaattcactc

SEQ ID NO: 44 - DNA, 20 bp, Artificial Sequence
  sgRNA
cccacaggtttcacatttgt

SEQ ID NO: 45 - DNA, 20 bp, Artificial Sequence
  sgRNA
ctgaacaaacctggccccac

SEQ ID NO: 46 - DNA, 20 bp, Artificial Sequence
  sgRNA
ggatgagcacgtgggcacgg

SEQ ID NO: 47 - DNA, 20 bp, Artificial Sequence
  sgRNA
aaacttacaaactgccgcac

SEQ ID NO: 48 - DNA, 20 bp, Artificial Sequence
  sgRNA
tttttctcaatcagccggtg

SEQ ID NO: 49 - DNA, 20 bp, Artificial Sequence
  sgRNA
ttgagaaaaagagacgtgac

SEQ ID NO: 50 - DNA, 20 bp, Artificial Sequence
  sgRNA
gggcaatgcactcgttaatc

SEQ ID NO: 51 - DNA, 20 bp, Artificial Sequence
  sgRNA
cgagtgcattgcccagctga

SEQ ID NO: 52 - DNA, 20 bp, Artificial Sequence
  sgRNA
ctgatgctgctgctcagtta

SEQ ID NO: 53 - DNA, 20 bp, Artificial Sequence
  sgRNA
taatctgtgcggtaacttgt

SEQ ID NO: 54 - DNA, 20 bp, Artificial Sequence
  sgRNA
ttcttttctattaatctgtg

SEQ ID NO: 55 - DNA, 20 bp, Artificial Sequence
  sgRNA
gagcaatgcattcattaatt

SEQ ID NO: 56 - DNA, 20 bp, Artificial Sequence
  sgRNA
tttggagaaagcagtagtct

SEQ ID NO: 57 - DNA, 20 bp, Artificial Sequence
  sgRNA
acaagaacagcaacgagtac

SEQ ID NO: 58 - DNA, 20 bp, Artificial Sequence
  sgRNA
caagaacagcaacgagtacc

SEQ ID NO: 59 - DNA, 20 bp, Artificial Sequence
  sgRNA
acagcaacgagtaccgggta

SEQ ID NO: 60 - DNA, 20 bp, Artificial Sequence
  sgRNA
gccgtgaactggacacgctg

SEQ ID NO: 61 - DNA, 20 bp, Artificial Sequence
  sgRNA
cgtgaactggacacgctgcg

SEQ ID NO: 62 - DNA, 20 bp, Artificial Sequence
  sgRNA
gtccaccgtcttcttggcct

SEQ ID NO: 63 - DNA, 20 bp, Artificial Sequence
  sgRNA
ggccaaggccaagaagacgg

SEQ ID NO: 64 - DNA, 20 bp, Artificial Sequence
  sgRNA
cagcttgtccaccgtcttct

SEQ ID NO: 65 - DNA, 20 bp, Artificial Sequence
  sgRNA
gctgcttgaacaagttccgc

SEQ ID NO: 66 - DNA, 20 bp, Artificial Sequence
  sgRNA
ctgcttgaacaagttccgca

SEQ ID NO: 67 - DNA, 20 bp, Artificial Sequence
  sgRNA
aatttcctccgcctctcatc

SEQ ID NO: 68 - DNA, 20 bp, Artificial Sequence
  sgRNA
aggacccagatgagaggcgg

SEQ ID NO: 69 - DNA, 20 bp, Artificial Sequence
  sgRNA
tgagaggcggaggaaatttc

SEQ ID NO: 70 - DNA, 20 bp, Artificial Sequence
  sgRNA
cttctgtctgcagcgggtgg

SEQ ID NO: 71 - DNA, 20 bp, Artificial Sequence
  sgRNA
ttaacaacaactgcttcagc

SEQ ID NO: 72 - DNA, 20 bp, Artificial Sequence
  sgRNA
tttcgctttcaagtcgattt

SEQ ID NO: 73 - DNA, 20 bp, Artificial Sequence
  sgRNA
aagcgaaacttgagcgaagc

SEQ ID NO: 74 - DNA, 20 bp, Artificial Sequence
  sgRNA
ggcattctctcgcactctgc

SEQ ID NO: 75 - DNA, 20 bp, Artificial Sequence
  sgRNA
aaagctgaggtaccagtact

SEQ ID NO: 76 - DNA, 20 bp, Artificial Sequence
  sgRNA
tggtgcatggcaatggacca

SEQ ID NO: 77 - DNA, 20 bp, Artificial Sequence
  sgRNA
cagaagaagcaactcgcaag

SEQ ID NO: 78 - DNA, 20 bp, Artificial Sequence
  sgRNA
agaagaagcaactcgcaagc

SEQ ID NO: 79 - DNA, 20 bp, Artificial Sequence
  sgRNA
caactcgcaagcgggagctg

SEQ ID NO: 80 - DNA, 20 bp, Artificial Sequence
  sgRNA
tgagagtcgagtcgcagtgc

SEQ ID NO: 81 - DNA, 20 bp, Artificial Sequence
  sgRNA
tcagaacaagaagcttatag

SEQ ID NO: 82 - DNA, 20 bp, Artificial Sequence
  sgRNA
tttggtggtcagatttagtg

SEQ ID NO: 83 - DNA, 20 bp, Artificial Sequence
  sgRNA
cagctccaagaagcgtttgg

SEQ ID NO: 84 - DNA, 20 bp, Artificial Sequence
  sgRNA
tctgaccaccaaacgcttct

SEQ ID NO: 85 - DNA, 20 bp, Artificial Sequence
  sgRNA
gatatgattcttggacttct

SEQ ID NO: 86 - DNA, 20 bp, Artificial Sequence
  sgRNA
agtccaagaatcatatccag

SEQ ID NO: 87 - DNA, 20 bp, Artificial Sequence
  sgRNA
acacgctatgacacgtcgct

SEQ ID NO: 88 - DNA, 20 bp, Artificial Sequence
  sgRNA
gacacgctatgacacgtcgc

SEQ ID NO: 89 - DNA, 20 bp, Artificial Sequence
  sgRNA
cacgctatgacacgtcgctg

SEQ ID NO: 90 - DNA, 20 bp, Artificial Sequence
  sgRNA
gtccaaaaacaacatccagt

SEQ ID NO: 91 - DNA, 20 bp, Artificial Sequence
  sgRNA
agtccaaaaacaacatccag

SEQ ID NO: 92 - DNA, 20 bp, Artificial Sequence
  sgRNA
acgcggtatgatacgtccct

SEQ ID NO: 93 - DNA, 20 bp, Artificial Sequence
  sgRNA
acttcttggtgagcagaccg

SEQ ID NO: 94 - DNA, 20 bp, Artificial Sequence
  sgRNA
cttcttggtgagcagaccga

SEQ ID NO: 95 - DNA, 20 bp, Artificial Sequence
  sgRNA
tcctgagccagtctcctgat

SEQ ID NO: 96 - DNA, 20 bp, Artificial Sequence
  sgRNA
agtctaagaacaacgtccag

SEQ ID NO: 97 - DNA, 20 bp, Artificial Sequence
  sgRNA
ggaggcgcaggacggcgtcc

SEQ ID NO: 98 - DNA, 20 bp, Artificial Sequence
  sgRNA
ctgcagataccttggctgtg

SEQ ID NO: 99 - DNA, 20 bp, Artificial Sequence
  sgRNA
tgatatcaccaatgtcttag

SEQ ID NO: 100 - DNA, 20 bp, Artificial Sequence
  sgRNA
gatatcaccaatgtcttaga

SEQ ID NO: 101 - DNA, 20 bp, Artificial Sequence
  sgRNA
atcaattccctctaagacat

SEQ ID NO: 102 - DNA, 20 bp, Artificial Sequence
  sgRNA
gtatgacatcaccaatgtct

SEQ ID NO: 103 - DNA, 20 bp, Artificial Sequence
  sgRNA
ttcgatgccatccaagacat

SEQ ID NO: 104 - DNA, 20 bp, Artificial Sequence
  sgRNA
tgtcttggatggcatcgaac

SEQ ID NO: 105 - DNA, 20 bp, Artificial Sequence
  sgRNA
ttgccacaaaactgggtgtt

SEQ ID NO: 106 - DNA, 20 bp, Artificial Sequence
  sgRNA
caaaactgggtgttcggaag

SEQ ID NO: 107 - DNA, 20 bp, Artificial Sequence
  sgRNA
cagatgtcgttattcacagc

SEQ ID NO: 108 - DNA, 20 bp, Artificial Sequence
  sgRNA
tgtgaataacgacatctgcc

SEQ ID NO: 109 - DNA, 20 bp, Artificial Sequence
  sgRNA
taacgacatctgcctggacg

SEQ ID NO: 110 - DNA, 20 bp, Artificial Sequence
  sgRNA
gctcctcggccacctcgtcc

SEQ ID NO: 111 - DNA, 20 bp, Artificial Sequence
  sgRNA
gggcccgtccatttaaaagc

SEQ ID NO: 112 - DNA, 20 bp, Artificial Sequence
  sgRNA
ccttctgctgggcagggata

SEQ ID NO: 113 - DNA, 20 bp, Artificial Sequence
  sgRNA
atcacaaccttctgctgggc

SEQ ID NO: 114 - DNA, 20 bp, Artificial Sequence
  sgRNA
tcacaaccttctgctgggca

SEQ ID NO: 115 - DNA, 20 bp, Artificial Sequence
  sgRNA
gcgcttcctttcgtcactcc

SEQ ID NO: 116 - DNA, 20 bp, Artificial Sequence
  sgRNA
agtttgccaggagtgacgaa

SEQ ID NO: 117 - DNA, 20 bp, Artificial Sequence
  sgRNA
ggtccagcagcaagtgcaag

SEQ ID NO: 118 - DNA, 20 bp, Artificial Sequence
  sgRNA
gttcctcttgcacttgctgc

SEQ ID NO: 119 - DNA, 20 bp, Artificial Sequence
  sgRNA
agatgaggtgctcatgtttc

SEQ ID NO: 120 - DNA, 20 bp, Artificial Sequence
  sgRNA
tggccacttcttccgccttg

SEQ ID NO: 121 - DNA, 20 bp, Artificial Sequence
  sgRNA
gtacaagttcgtctctttcc

SEQ ID NO: 122 - DNA, 20 bp, Artificial Sequence
  sgRNA
gctcattctgaggctcctgc

SEQ ID NO: 123 - DNA, 20 bp, Artificial Sequence
  sgRNA
cagatcatgtgctcattctg

SEQ ID NO: 124 - DNA, 20 bp, Artificial Sequence
  sgRNA
agaatgagcacatgatctgc

SEQ ID NO: 125 - DNA, 20 bp, Artificial Sequence
  sgRNA
cgtagtagtatcgcagggct

SEQ ID NO: 126 - DNA, 20 bp, Artificial Sequence
  sgRNA
ctttacgtagtagtatcgca

SEQ ID NO: 127 - DNA, 20 bp, Artificial Sequence
  sgRNA
tgcacaggtagacgtgggcg

SEQ ID NO: 128 - DNA, 20 bp, Artificial Sequence
  sgRNA
cccacgtctacctgtgcaac

SEQ ID NO: 129 - DNA, 20 bp, Artificial Sequence
  sgRNA
ggaatttgagccataggggc

SEQ ID NO: 130 - DNA, 20 bp, Artificial Sequence
  sgRNA
tgatcatctcagtttggtgc

SEQ ID NO: 131 - DNA, 20 bp, Artificial Sequence
  sgRNA
tgagatgatcatcaccaaac

SEQ ID NO: 132 - DNA, 20 bp, Artificial Sequence
  sgRNA
ggaactgccacagctggatc

SEQ ID NO: 133 - DNA, 20 bp, Artificial Sequence
  sgRNA
gtagtggccagatccagctg

SEQ ID NO: 134 - DNA, 20 bp, Artificial Sequence
  sgRNA
cgagcaggaactgccacagc

SEQ ID NO: 135 - DNA, 20 bp, Artificial Sequence
  sgRNA
cccgtggaagtcaaacttgt

SEQ ID NO: 136 - DNA, 20 bp, Artificial Sequence
  sgRNA
cctacaagtttgacttccac

SEQ ID NO: 137 - DNA, 20 bp, Artificial Sequence
  sgRNA
caaaaatatcatccacaaga

SEQ ID NO: 138 - DNA, 20 bp, Artificial Sequence
  sgRNA
aatatcatccacaagacggc

SEQ ID NO: 139 - DNA, 20 bp, Artificial Sequence
  sgRNA
aaatatcatccacaagacgg

SEQ ID NO: 140 - DNA, 20 bp, Artificial Sequence
  sgRNA
agcgcttgcccgccgtcttg

SEQ ID NO: 141 - DNA, 20 bp, Artificial Sequence
  sgRNA
tctgcaggtcgcacacaaag

SEQ ID NO: 142 - DNA, 20 bp, Artificial Sequence
  sgRNA
gaagcggaccaatccagttg

SEQ ID NO: 143 - DNA, 20 bp, Artificial Sequence
  sgRNA
ccagctgatgaaagattgac

SEQ ID NO: 144 - DNA, 20 bp, Artificial Sequence
  sgRNA
cctgtcaatctttcatcagc

SEQ ID NO: 145 - DNA, 20 bp, Artificial Sequence
  sgRNA
agcgcttgcccgaagtcttg

SEQ ID NO: 146 - DNA, 20 bp, Artificial Sequence
  sgRNA
tctgcaggtcacatacgaaa

SEQ ID NO: 147 - DNA, 20 bp, Artificial Sequence
  sgRNA
aggatccttcataaaacaaa

SEQ ID NO: 148 - DNA, 20 bp, Artificial Sequence
  sgRNA
ggatccttcataaaacaaaa

SEQ ID NO: 149 - DNA, 20 bp, Artificial Sequence
  sgRNA
ttcataaaacaaaagggaaa

SEQ ID NO: 150 - DNA, 20 bp, Artificial Sequence
  sgRNA
cttccttctgcagcagttcc

SEQ ID NO: 151 - DNA, 20 bp, Artificial Sequence
  sgRNA
gctgccaggcgatgacatgg

SEQ ID NO: 152 - DNA, 20 bp, Artificial Sequence
  sgRNA
cgctgtcggacagtagttcc

SEQ ID NO: 153 - DNA, 20 bp, Artificial Sequence
  sgRNA
acagctggcgttggcgctgt

SEQ ID NO: 154 - DNA, 20 bp, Artificial Sequence
  sgRNA
ccaggtgatacagctggcgt

SEQ ID NO: 155 - DNA, 20 bp, Artificial Sequence
  sgRNA
tgaccaaagtgcatggcaaa

SEQ ID NO: 156 - DNA, 20 bp, Artificial Sequence
  sgRNA
gccatggaagtcaaacttgt

SEQ ID NO: 157 - DNA, 20 bp, Artificial Sequence
  sgRNA
aagagaaacggagaatccga

SEQ ID NO: 158 - DNA, 20 bp, Artificial Sequence
  sgRNA
ccatcttattccgttccctt

SEQ ID NO: 159 - DNA, 20 bp, Artificial Sequence
  sgRNA
ctgcagccaagtgccggaat

SEQ ID NO: 160 - DNA, 20 bp, Artificial Sequence
  sgRNA
ttccttctctttcagcagat

SEQ ID NO: 161 - DNA, 20 bp, Artificial Sequence
  sgRNA
ggaaaaactggagtttattt

SEQ ID NO: 162 - DNA, 20 bp, Artificial Sequence
  sgRNA
cagaagaagaagaaaagcga

SEQ ID NO: 163 - DNA, 20 bp, Artificial Sequence
  sgRNA
agcgaagggttcgcagagag

SEQ ID NO: 164 - DNA, 20 bp, Artificial Sequence
  sgRNA
tcgcagagagcggaacaagc

SEQ ID NO: 165 - DNA, 20 bp, Artificial Sequence
  sgRNA
gatctgtcagctccctccga

SEQ ID NO: 166 - DNA, 20 bp, Artificial Sequence
  sgRNA
agcccggtttgtgggccacc

SEQ ID NO: 167 - DNA, 20 bp, Artificial Sequence
  sgRNA
aggagaagcgtcgaatccgg

SEQ ID NO: 168 - DNA, 20 bp, Artificial Sequence
  sgRNA
agctagctgcagccaagtgt

SEQ ID NO: 169 - DNA, 20 bp, Artificial Sequence
  sgRNA
tctctgtcagctcccggcga

SEQ ID NO: 170 - DNA, 20 bp, Artificial Sequence
  sgRNA
ggagctgacagagaagctgc

SEQ ID NO: 171 - DNA, 20 bp, Artificial Sequence
  sgRNA
cttctcctcttccagctcct

SEQ ID NO: 172 - DNA, 20 bp, Artificial Sequence
  sgRNA
atggccatataagagtaggg

SEQ ID NO: 173 - DNA, 20 bp, Artificial Sequence
  sgRNA
gaggccaccctactcttata

SEQ ID NO: 174 - DNA, 20 bp, Artificial Sequence
  sgRNA
atcatggccatataagagta

SEQ ID NO: 175 - DNA, 20 bp, Artificial Sequence
  sgRNA
ccaagtgtagatgtccttca

SEQ ID NO: 176 - DNA, 20 bp, Artificial Sequence
  sgRNA
ccaatggcaaggtctccttc

SEQ ID NO: 177 - DNA, 20 bp, Artificial Sequence
  sgRNA
tgatgaggtcggcgtacgac

SEQ ID NO: 178 - DNA, 20 bp, Artificial Sequence
  sgRNA
cttctcggctgagctctcga

SEQ ID NO: 179 - DNA, 20 bp, Artificial Sequence
  sgRNA
cgacagggtgagcctcttct

SEQ ID NO: 180 - DNA, 20 bp, Artificial Sequence
  sgRNA
ccctgtcgcagatctacgag

SEQ ID NO: 181 - DNA, 20 bp, Artificial Sequence
  sgRNA
gaagagcgtgccctacttca

SEQ ID NO: 182 - DNA, 20 bp, Artificial Sequence
  sgRNA
ggcataggacaggttccccc

SEQ ID NO: 183 - DNA, 20 bp, Artificial Sequence
  sgRNA
tgatcaggtcggcataggac

SEQ ID NO: 184 - DNA, 20 bp, Artificial Sequence
  sgRNA
gcgggtgatcaggtcggcat

SEQ ID NO: 185 - DNA, 20 bp, Artificial Sequence
  sgRNA
gacaaagtgagccgtttgtc

SEQ ID NO: 186 - DNA, 20 bp, Artificial Sequence
  sgRNA
gcacgggcaagagctcttgg

SEQ ID NO: 187 - DNA, 20 bp, Artificial Sequence
  sgRNA
ctgtggcggcgagatggtac

SEQ ID NO: 188 - DNA, 20 bp, Artificial Sequence
  sgRNA
ataaaatgaatgggcagaac

SEQ ID NO: 189 - DNA, 20 bp, Artificial Sequence
  sgRNA
gcttgggcttgataaggggc

SEQ ID NO: 190 - DNA, 20 bp, Artificial Sequence
  sgRNA
ccttcgcttgggcttgataa

SEQ ID NO: 191 - DNA, 20 bp, Artificial Sequence
  sgRNA
acatcttccggtttcgggtc

SEQ ID NO: 192 - DNA, 20 bp, Artificial Sequence
  sgRNA
caagatgtgagctcacattg

SEQ ID NO: 193 - DNA, 20 bp, Artificial Sequence
  sgRNA
aagatgtgagctcacattgt

SEQ ID NO: 194 - DNA, 20 bp, Artificial Sequence
  sgRNA
agatgtgagctcacattgtg

SEQ ID NO: 195 - DNA, 20 bp, Artificial Sequence
  sgRNA
ttgataaagcttctgttatg

SEQ ID NO: 196 - DNA, 20 bp, Artificial Sequence
  sgRNA
tctcacacgtaaataactga

SEQ ID NO: 197 - DNA, 20 bp, Artificial Sequence
  sgRNA
tcagagaggtaactcgttcc

SEQ ID NO: 198 - DNA, 20 bp, Artificial Sequence
  sgRNA
ccaattatatactttcagag

SEQ ID NO: 199 - DNA, 20 bp, Artificial Sequence
  sgRNA
cctctctgaaagtatataat

SEQ ID NO: 200 - DNA, 20 bp, Artificial Sequence
  sgRNA
ataattggtttgctaatcga

SEQ ID NO: 201 - DNA, 20 bp, Artificial Sequence
  sgRNA
ttggtttgctaatcgacgga

SEQ ID NO: 202 - DNA, 20 bp, Artificial Sequence
  sgRNA
ctgcacgaagaaggcgtacg

SEQ ID NO: 203 - DNA, 20 bp, Artificial Sequence
  sgRNA
cttcgtgcagacctgccgcg

SEQ ID NO: 204 - DNA, 20 bp, Artificial Sequence
  sgRNA
gaagaagcatcccgactcgt

SEQ ID NO: 205 - DNA, 20 bp, Artificial Sequence
  sgRNA
gcgaagttcaccgacgagtc

SEQ ID NO: 206 - DNA, 20 bp, Artificial Sequence
  sgRNA
ggcgaagttcaccgacgagt

SEQ ID NO: 207 - DNA, 20 bp, Artificial Sequence
  sgRNA
gagcggccccacggaggacc

SEQ ID NO: 208 - DNA, 20 bp, Artificial Sequence
  sgRNA
tctccacctggtcctccgtg

SEQ ID NO: 209 - DNA, 20 bp, Artificial Sequence
  sgRNA
atctccacctggtcctccgt

SEQ ID NO: 210 - DNA, 20 bp, Artificial Sequence
  sgRNA
cgatgaggcacagcgtggtg

SEQ ID NO: 211 - DNA, 20 bp, Artificial Sequence
  sgRNA
gcagagtggcggcggtcaga

SEQ ID NO: 212 - DNA, 20 bp, Artificial Sequence
  sgRNA
ctactccaagctcaaggaac

SEQ ID NO: 213 - DNA, 20 bp, Artificial Sequence
  sgRNA
tgtaatcgatgacgtgctgc

SEQ ID NO: 214 - DNA, 20 bp, Artificial Sequence
  sgRNA
gcacgtcatcgattacatct

SEQ ID NO: 215 - DNA, 20 bp, Artificial Sequence
  sgRNA
cttggacctgcagatcgccc

SEQ ID NO: 216 - DNA, 20 bp, Artificial Sequence
  sgRNA
cgatagtgggatgcgagtcc

SEQ ID NO: 217 - DNA, 20 bp, Artificial Sequence
  sgRNA
accactgctactcgcgcctg

SEQ ID NO: 218 - DNA, 20 bp, Artificial Sequence
  sgRNA
ctcccggcaccagttcccgc

SEQ ID NO: 219 - DNA, 20 bp, Artificial Sequence
  sgRNA
ctggtgccgggagtcccgcg

SEQ ID NO: 220 - DNA, 20 bp, Artificial Sequence
  sgRNA
tggctaagctgagtgcctcg

SEQ ID NO: 221 - DNA, 20 bp, Artificial Sequence
  sgRNA
cacgctgcaggatttccacc

SEQ ID NO: 222 - DNA, 20 bp, Artificial Sequence
  sgRNA
atctgtgggatcgtttgcat

SEQ ID NO: 223 - DNA, 20 bp, Artificial Sequence
  sgRNA
tttgtgaaccatgagcacat

SEQ ID NO: 224 - DNA, 20 bp, Artificial Sequence
  sgRNA
ccacactggttgcactggaa

SEQ ID NO: 225 - DNA, 20 bp, Artificial Sequence
  sgRNA
agcttcgctgtttatagctc

SEQ ID NO: 226 - DNA, 20 bp, Artificial Sequence
  sgRNA
taaacagcgaagctctttag

SEQ ID NO: 227 - DNA, 20 bp, Artificial Sequence
  sgRNA
aagctccgcactggttacag

SEQ ID NO: 228 - DNA, 20 bp, Artificial Sequence
  sgRNA
gggtaaaagaagctccgcac

SEQ ID NO: 229 - DNA, 20 bp, Artificial Sequence
  sgRNA
tctcagaaggttgcccttct

SEQ ID NO: 230 - DNA, 20 bp, Artificial Sequence
  sgRNA
gtctcagaaggttgcccttc

SEQ ID NO: 231 - DNA, 20 bp, Artificial Sequence
  sgRNA
agaagaagggacgctctcac

SEQ ID NO: 232 - DNA, 20 bp, Artificial Sequence
  sgRNA
aagatgaactgcgacgtgtg

SEQ ID NO: 233 - DNA, 20 bp, Artificial Sequence
  sgRNA
agatgaactgcgacgtgtgc

SEQ ID NO: 234 - DNA, 20 bp, Artificial Sequence
  sgRNA
caagacgttgaagctaatgc

SEQ ID NO: 235 - DNA, 20 bp, Artificial Sequence
  sgRNA
cgtcatattaaactgcacac

SEQ ID NO: 236 - DNA, 20 bp, Artificial Sequence
  sgRNA
gcgttccttgtgctcctcca

SEQ ID NO: 237 - DNA, 20 bp, Artificial Sequence
  sgRNA
gtctgcggcatggtctgcat

SEQ ID NO: 238 - DNA, 20 bp, Artificial Sequence
  sgRNA
tctgcggcatggtctgcatt

SEQ ID NO: 239 - DNA, 20 bp, Artificial Sequence
  sgRNA
cttgtgtaccatgagcacat

SEQ ID NO: 240 - DNA, 20 bp, Artificial Sequence
  sgRNA
cccccgagtgcagcttgatg

SEQ ID NO: 241 - DNA, 20 bp, Artificial Sequence
  sgRNA
cgccggcgtgacgcactcac

SEQ ID NO: 242 - DNA, 20 bp, Artificial Sequence
  sgRNA
cagcgtgcttccatggaatc

SEQ ID NO: 243 - DNA, 20 bp, Artificial Sequence
  sgRNA
tgcttagcagcgtgcttcca

SEQ ID NO: 244 - DNA, 20 bp, Artificial Sequence
  sgRNA
tggaagcacgctgctaagca

SEQ ID NO: 245 - DNA, 20 bp, Artificial Sequence
  sgRNA
gcacgctgctaagcacggct

SEQ ID NO: 246 - DNA, 20 bp, Artificial Sequence
  sgRNA
gaaggatcagagtaggaaca

SEQ ID NO: 247 - DNA, 20 bp, Artificial Sequence
  sgRNA
ctttagccctggtatcgtat

SEQ ID NO: 248 - DNA, 20 bp, Artificial Sequence
  sgRNA
gccgagccgcatgcatccag

SEQ ID NO: 249 - DNA, 20 bp, Artificial Sequence
  sgRNA
ccagatcccctggatgcatg

SEQ ID NO: 250 - DNA, 20 bp, Artificial Sequence
  sgRNA
gtgccgagccgcatgcatcc

SEQ ID NO: 251 - DNA, 20 bp, Artificial Sequence
  sgRNA
aaggacagaagcataaagaa

SEQ ID NO: 252 - DNA, 20 bp, Artificial Sequence
  sgRNA
ctggacctggggcagctgga

SEQ ID NO: 253 - DNA, 20 bp, Artificial Sequence
  sgRNA
gccgtaggccatgcttccac

SEQ ID NO: 254 - DNA, 20 bp, Artificial Sequence
  sgRNA
tgccgtaggccatgcttcca

SEQ ID NO: 255 - DNA, 20 bp, Artificial Sequence
  sgRNA
tcccgtggaagcatggccta

SEQ ID NO: 256 - DNA, 20 bp, Artificial Sequence
  sgRNA
gttagctgctgacaatagca

SEQ ID NO: 257 - DNA, 20 bp, Artificial Sequence
  sgRNA
gaacaagagcaatgactttg

SEQ ID NO: 258 - DNA, 20 bp, Artificial Sequence
  sgRNA
gtggaaacacgcgggcaagc

SEQ ID NO: 259 - DNA, 20 bp, Artificial Sequence
  sgRNA
gcaggactacaatcgtgagg

SEQ ID NO: 260 - DNA, 20 bp, Artificial Sequence
  sgRNA
tttaaaggcaagttccgaga

SEQ ID NO: 261 - DNA, 20 bp, Artificial Sequence
  sgRNA
acaagccagatcctcctact

SEQ ID NO: 262 - DNA, 20 bp, Artificial Sequence
  sgRNA
ccccaccaccccgccgtgtg

SEQ ID NO: 263 - DNA, 20 bp, Artificial Sequence
  sgRNA
gtactggcagctgttcacct

SEQ ID NO: 264 - DNA, 20 bp, Artificial Sequence
  sgRNA
gtgaacagctgccagtaccc

SEQ ID NO: 265 - DNA, 20 bp, Artificial Sequence
  sgRNA
tgaacagctgccagtaccca

SEQ ID NO: 266 - DNA, 20 bp, Artificial Sequence
  sgRNA
gcgcaggttggccttccact

SEQ ID NO: 267 - DNA, 20 bp, Artificial Sequence
  sgRNA
tgtgtagccagatgagacca

SEQ ID NO: 268 - DNA, 20 bp, Artificial Sequence
  sgRNA
ctgtgtagccagatgagacc

SEQ ID NO: 269 - DNA, 20 bp, Artificial Sequence
  sgRNA
ccaggggatctggaagcgtt

SEQ ID NO: 270 - DNA, 20 bp, Artificial Sequence
  sgRNA
tggcatgtttccaggggatc

SEQ ID NO: 271 - DNA, 20 bp, Artificial Sequence
  sgRNA
cttgatgtacgatggcacca

SEQ ID NO: 272 - DNA, 20 bp, Artificial Sequence
  sgRNA
tcagcagcggccagtacgag

SEQ ID NO: 273 - DNA, 20 bp, Artificial Sequence
  sgRNA
gccactgcagcccctcgtac

SEQ ID NO: 274 - DNA, 20 bp, Artificial Sequence
  sgRNA
gccagtacgaggggctgcag

SEQ ID NO: 275 - DNA, 20 bp, Artificial Sequence
  sgRNA
gcacagtcttccgcgtaccc

SEQ ID NO: 276 - DNA, 20 bp, Artificial Sequence
  sgRNA
ctgtgctgtggagtgcacag

SEQ ID NO: 277 - DNA, 20 bp, Artificial Sequence
  sgRNA
cagaaatgtccagctgggac

SEQ ID NO: 278 - DNA, 20 bp, Artificial Sequence
  sgRNA
atggctcagaaatgtccagc

SEQ ID NO: 279 - DNA, 20 bp, Artificial Sequence
  sgRNA
tggctcagaaatgtccagct

SEQ ID NO: 280 - DNA, 20 bp, Artificial Sequence
  sgRNA
tcagtcacttcttcaaaatc

SEQ ID NO: 281 - DNA, 20 bp, Artificial Sequence
  sgRNA
agtgactgaccggtcccagc

SEQ ID NO: 282 - DNA, 20 bp, Artificial Sequence
  sgRNA
ctgcatgcttccagggaatc

SEQ ID NO: 283 - DNA, 20 bp, Artificial Sequence
  sgRNA
cggattccctggaagcatgc

SEQ ID NO: 284 - DNA, 20 bp, Artificial Sequence
  sgRNA
ttgcttgcctgcatgcttcc

SEQ ID NO: 285 - DNA, 20 bp, Artificial Sequence
  sgRNA
gcggagctggatcgtggagc

SEQ ID NO: 286 - DNA, 20 bp, Artificial Sequence
  sgRNA
gagctggatcgtggagcagg

SEQ ID NO: 287 - DNA, 20 bp, Artificial Sequence
  sgRNA
aggagcggatcaaggcagag

SEQ ID NO: 288 - DNA, 20 bp, Artificial Sequence
  sgRNA
ggcacttggaggcggcaatg

SEQ ID NO: 289 - DNA, 20 bp, Artificial Sequence
  sgRNA
cagcttccttttccggcact

SEQ ID NO: 290 - DNA, 20 bp, Artificial Sequence
  sgRNA
ggaagctggagcggatcgct

SEQ ID NO: 291 - DNA, 20 bp, Artificial Sequence
  sgRNA
gaaagcgcaaaactccgagc

SEQ ID NO: 292 - DNA, 20 bp, Artificial Sequence
  sgRNA
ggcactaccgcaaacacaca

SEQ ID NO: 293 - DNA, 20 bp, Artificial Sequence
  sgRNA
accgcaaacacacagggcac

SEQ ID NO: 294 - DNA, 20 bp, Artificial Sequence
  sgRNA
acttctggcactgaaagggc

SEQ ID NO: 295 - DNA, 20 bp, Artificial Sequence
  sgRNA
tcagtgccagaagtgtgaca

SEQ ID NO: 296 - DNA, 20 bp, Artificial Sequence
  sgRNA
aaaaggccctgtcacacttc

SEQ ID NO: 297 - DNA, 20 bp, Artificial Sequence
  sgRNA
tataacatgaaagcattcag

SEQ ID NO: 298 - DNA, 20 bp, Artificial Sequence
  sgRNA
tgcagctatcaaccagatcc

SEQ ID NO: 299 - DNA, 20 bp, Artificial Sequence
  sgRNA
ggcctgctcttcccgggaga

SEQ ID NO: 300 - DNA, 20 bp, Artificial Sequence
  sgRNA
ccgtgctagttcatagtatt

SEQ ID NO: 301 - DNA, 20 bp, Artificial Sequence
  sgRNA
ctacacatgcagctttatcc

SEQ ID NO: 302 - DNA, 20 bp, Artificial Sequence
  sgRNA
cgtgaagcgcgtgtgccaga

SEQ ID NO: 303 - DNA, 20 bp, Artificial Sequence
  sgRNA
gaagcgcgtgtgccagaagg

SEQ ID NO: 304 - DNA, 20 bp, Artificial Sequence
  sgRNA
gcttctgcagctcctccttc

SEQ ID NO: 305 - DNA, 20 bp, Artificial Sequence
  sgRNA
cacgcgcttcacgcggcagc

SEQ ID NO: 306 - DNA, 20 bp, Artificial Sequence
  sgRNA
tctggcacacgcgcttcacg

SEQ ID NO: 307 - DNA, 20 bp, Artificial Sequence
  sgRNA
ggagaatgccagcatgaagc

SEQ ID NO: 308 - DNA, 20 bp, Artificial Sequence
  sgRNA
atcgagctccagcttcatgc

SEQ ID NO: 309 - DNA, 20 bp, Artificial Sequence
  sgRNA
gttctgcagggcctcgtact

SEQ ID NO: 310 - DNA, 20 bp, Artificial Sequence
  sgRNA
ccagcttctccacctcctgc

SEQ ID NO: 311 - DNA, 20 bp, Artificial Sequence
  sgRNA
ggaggtggagaagctggcct

SEQ ID NO: 312 - DNA, 20 bp, Artificial Sequence
  sgRNA
atagagcttgtccagaaata

SEQ ID NO: 313 - DNA, 20 bp, Artificial Sequence
  sgRNA
gaaaacctcctggacagaag

SEQ ID NO: 314 - DNA, 20 bp, Artificial Sequence
  sgRNA
agcgtcacttggggaaaact

SEQ ID NO: 315 - DNA, 20 bp, Artificial Sequence
  sgRNA
tgagaagctgaagaagctgg

SEQ ID NO: 316 - DNA, 20 bp, Artificial Sequence
  sgRNA
tgatcttcttctttggtcca

SEQ ID NO: 317 - DNA, 20 bp, Artificial Sequence
  sgRNA
ggaagcagtgccgtgagcgc

SEQ ID NO: 318 - DNA, 20 bp, Artificial Sequence
  sgRNA
gctggaccgaggaggaagac

SEQ ID NO: 319 - DNA, 20 bp, Artificial Sequence
  sgRNA
ggaagacaggataatctgtg

SEQ ID NO: 320 - DNA, 20 bp, Artificial Sequence
  sgRNA
ctgtgaggcccataaagtcc

SEQ ID NO: 321 - DNA, 20 bp, Artificial Sequence
  sgRNA
attgagttggtcaagaagta

SEQ ID NO: 322 - DNA, 20 bp, Artificial Sequence
  sgRNA
cggaggaaaacgacaagagg

SEQ ID NO: 323 - DNA, 20 bp, Artificial Sequence
  sgRNA
acaacgtcttggaacgtcag

SEQ ID NO: 324 - DNA, 20 bp, Artificial Sequence
  sgRNA
ttcagggatctggtcacgca

SEQ ID NO: 325 - DNA, 20 bp, Artificial Sequence
  sgRNA
tttcgttgttttccaattca

SEQ ID NO: 326 - DNA, 20 bp, Artificial Sequence
  sgRNA
tttgaggatcactaccttgg

SEQ ID NO: 327 - DNA, 20 bp, Artificial Sequence
  sgRNA
acactgaggacgtgaccaag

SEQ ID NO: 328 - DNA, 20 bp, Artificial Sequence
  sgRNA
gaagttatggttcttcctct

SEQ ID NO: 329 - DNA, 20 bp, Artificial Sequence
  sgRNA
gaggaagaaccataacttct

SEQ ID NO: 330 - DNA, 20 bp, Artificial Sequence
  sgRNA
gaatacttgcaggctttggt

SEQ ID NO: 331 - DNA, 20 bp, Artificial Sequence
  sgRNA
agaatacttgcaggctttgg

SEQ ID NO: 332 - DNA, 20 bp, Artificial Sequence
  sgRNA
tataactttgcctgtatcag

SEQ ID NO: 333 - DNA, 20 bp, Artificial Sequence
  sgRNA
acatatattcccactgatac

SEQ ID NO: 334 - DNA, 20 bp, Artificial Sequence
  sgRNA
atatatgttgtgaccaatgc

SEQ ID NO: 335 - DNA, 20 bp, Artificial Sequence
  sgRNA
tcagccatttacgtacactc

SEQ ID NO: 336 - DNA, 20 bp, Artificial Sequence
  sgRNA
ggatccggagtgtacgtaaa

SEQ ID NO: 337 - DNA, 20 bp, Artificial Sequence
  sgRNA
taatgaaccagtggtgttgc

SEQ ID NO: 338 - DNA, 20 bp, Artificial Sequence
  sgRNA
acaaatacctgcaacaccac

SEQ ID NO: 339 - DNA, 20 bp, Artificial Sequence
  sgRNA
agtggtgttgcaggtatttg

SEQ ID NO: 340 - DNA, 20 bp, Artificial Sequence
  sgRNA
attaactcgaaaaaccaatc

SEQ ID NO: 341 - DNA, 20 bp, Artificial Sequence
  sgRNA
gttaatatcacaaggaaaga

SEQ ID NO: 342 - DNA, 20 bp, Artificial Sequence
  sgRNA
atgagacggaaggcagccgg

SEQ ID NO: 343 - DNA, 20 bp, Artificial Sequence
  sgRNA
tgagacggaaggcagccggg

SEQ ID NO: 344 - DNA, 20 bp, Artificial Sequence
  sgRNA
aggcagccggggggctgtga

SEQ ID NO: 345 - DNA, 20 bp, Artificial Sequence
  sgRNA
ccggacgctgtctctccagg

SEQ ID NO: 346 - DNA, 20 bp, Artificial Sequence
  sgRNA
cgatagggttcgaggccacc

SEQ ID NO: 347 - DNA, 20 bp, Artificial Sequence
  sgRNA
ctcatagtgggcccggtgat

SEQ ID NO: 348 - DNA, 20 bp, Artificial Sequence
  sgRNA
ccgggcccactatgagacgg

SEQ ID NO: 349 - DNA, 20 bp, Artificial Sequence
  sgRNA
ggcgctgtcaaagccccaac

SEQ ID NO: 350 - DNA, 20 bp, Artificial Sequence
  sgRNA
gcttgttctccatgtagccg

SEQ ID NO: 351 - DNA, 20 bp, Artificial Sequence
  sgRNA
ctggggcttcagatcttcat

SEQ ID NO: 352 - DNA, 20 bp, Artificial Sequence
  sgRNA
tgtaactgctgggttatgat

SEQ ID NO: 353 - DNA, 20 bp, Artificial Sequence
  sgRNA
gcacggcagatgtaactgct

SEQ ID NO: 354 - DNA, 20 bp, Artificial Sequence
  sgRNA
tgcacggcagatgtaactgc

SEQ ID NO: 355 - DNA, 20 bp, Artificial Sequence
  sgRNA
agcagttacatctgccgtgc

SEQ ID NO: 356 - DNA, 20 bp, Artificial Sequence
  sgRNA
tttatctttgcaatggcaag

SEQ ID NO: 357 - DNA, 20 bp, Artificial Sequence
  sgRNA
ggtaaaggccagtctaatgg

SEQ ID NO: 358 - DNA, 20 bp, Artificial Sequence
  sgRNA
gttggtaaaggccagtctaa

SEQ ID NO: 359 - DNA, 20 bp, Artificial Sequence
  sgRNA
tggcctttaccaactcactt

SEQ ID NO: 360 - DNA, 20 bp, Artificial Sequence
  sgRNA
atgaaactgaaggtagccga

SEQ ID NO: 361 - DNA, 20 bp, Artificial Sequence
  sgRNA
gtgcacatcccacagcccag

SEQ ID NO: 362 - DNA, 20 bp, Artificial Sequence
  sgRNA
tttagagtctggcaggaagt

SEQ ID NO: 363 - DNA, 20 bp, Artificial Sequence
  sgRNA
acaccactttagagtctggc

SEQ ID NO: 364 - DNA, 20 bp, Artificial Sequence
  sgRNA
cttcctgccagactctaaag

SEQ ID NO: 365 - DNA, 20 bp, Artificial Sequence
  sgRNA
tttacgtctccaatggacgg

SEQ ID NO: 366 - DNA, 20 bp, Artificial Sequence
  sgRNA
actgcgcttcctccgtccat

SEQ ID NO: 367 - DNA, 20 bp, Artificial Sequence
  sgRNA
cgagctcactatgagactga

SEQ ID NO: 368 - DNA, 20 bp, Artificial Sequence
  sgRNA
tcattggcactgcagatgag

SEQ ID NO: 369 - DNA, 20 bp, Artificial Sequence
  sgRNA
ctgcagatgagaggagcctg

SEQ ID NO: 370 - DNA, 20 bp, Artificial Sequence
  sgRNA
ggtagaaggcatggggccgc

SEQ ID NO: 371 - DNA, 20 bp, Artificial Sequence
  sgRNA
cgggaaggtcgtgtctgtgc

SEQ ID NO: 372 - DNA, 20 bp, Artificial Sequence
  sgRNA
gaaaatggcggagtttggga

SEQ ID NO: 373 - DNA, 20 bp, Artificial Sequence
  sgRNA
ggcggagtttgggaaggatt

SEQ ID NO: 374 - DNA, 20 bp, Artificial Sequence
  sgRNA
gcggagtttgggaaggattt

SEQ ID NO: 375 - DNA, 20 bp, Artificial Sequence
  sgRNA
cggagtttgggaaggatttg

SEQ ID NO: 376 - DNA, 20 bp, Artificial Sequence
  sgRNA
ctgtctatgaacatctgtgg

SEQ ID NO: 377 - DNA, 20 bp, Artificial Sequence
  sgRNA
acttcatgtgactaagaaaa

SEQ ID NO: 378 - DNA, 20 bp, Artificial Sequence
  sgRNA
gaaaaaggtatttgaaacac

SEQ ID NO: 379 - DNA, 20 bp, Artificial Sequence
  sgRNA
tatttgaaacactggaagca

SEQ ID NO: 380 - DNA, 20 bp, Artificial Sequence
  sgRNA
ggatgacagaggcgtgtatt

SEQ ID NO: 381 - DNA, 20 bp, Artificial Sequence
  sgRNA
atcgttcagttggtcacaaa

SEQ ID NO: 382 - DNA, 20 bp, Artificial Sequence
  sgRNA
cattgaggttcggttctatg

SEQ ID NO: 383 - DNA, 20 bp, Artificial Sequence
  sgRNA
gagaatggatggcaagcctt

SEQ ID NO: 384 - DNA, 20 bp, Artificial Sequence
  sgRNA
gaatggatggcaagcctttg

SEQ ID NO: 385 - DNA, 20 bp, Artificial Sequence
  sgRNA
agaatggatggcaagccttt

SEQ ID NO: 386 - DNA, 20 bp, Artificial Sequence
  sgRNA
tgtgggagagaagtccccaa

SEQ ID NO: 387 - DNA, 20 bp, Artificial Sequence
  sgRNA
tgtaaccaagaagaacatga

SEQ ID NO: 388 - DNA, 20 bp, Artificial Sequence
  sgRNA
caaggagctgaagaaagtca

SEQ ID NO: 389 - DNA, 20 bp, Artificial Sequence
  sgRNA
tcatggatctgagcattgta

SEQ ID NO: 390 - DNA, 20 bp, Artificial Sequence
  sgRNA
aggagccatcgctagctcga

SEQ ID NO: 391 - DNA, 20 bp, Artificial Sequence
  sgRNA
gtgggtcactgtgtgtcacc

SEQ ID NO: 392 - DNA, 20 bp, Artificial Sequence
  sgRNA
aacagaatatccagtacaaa

SEQ ID NO: 393 - DNA, 20 bp, Artificial Sequence
  sgRNA
gttgcgattgatgcgaacga

SEQ ID NO: 394 - DNA, 20 bp, Artificial Sequence
  sgRNA
tgaagcgacactgctggcag

SEQ ID NO: 395 - DNA, 20 bp, Artificial Sequence
  sgRNA
acttcttgaagcgacactgc

SEQ ID NO: 396 - DNA, 20 bp, Artificial Sequence
  sgRNA
ttcaagaagtgtctctccgt

SEQ ID NO: 397 - DNA, 20 bp, Artificial Sequence
  sgRNA
tggaacagttctcattcttc

SEQ ID NO: 398 - DNA, 20 bp, Artificial Sequence
  sgRNA
atgagaactgttccatcatg

SEQ ID NO: 399 - DNA, 20 bp, Artificial Sequence
  sgRNA
tttaagaagtgtctgtctgt

SEQ ID NO: 400 - DNA, 20 bp, Artificial Sequence
  sgRNA
ctttaagaagtgtctgtctg

SEQ ID NO: 401 - DNA, 20 bp, Artificial Sequence
  sgRNA
ttaagaagtgtctgtctgtg

SEQ ID NO: 402 - DNA, 20 bp, Artificial Sequence
  sgRNA
gagggccgctgtgcagtctg

SEQ ID NO: 403 - DNA, 20 bp, Artificial Sequence
  sgRNA
aatgcttcgtgtcagcacta

SEQ ID NO: 404 - DNA, 20 bp, Artificial Sequence
  sgRNA
tatggggtccgcacctgtga

SEQ ID NO: 405 - DNA, 20 bp, Artificial Sequence
  sgRNA
aagcgccaagtacatctgcc

SEQ ID NO: 406 - DNA, 20 bp, Artificial Sequence
  sgRNA
ggcaaacaaggattgccctg

SEQ ID NO: 407 - DNA, 20 bp, Artificial Sequence
  sgRNA
gacagtactgacaacgattt

SEQ ID NO: 408 - DNA, 20 bp, Artificial Sequence
  sgRNA
gaaatcgttgtcagtactgt

SEQ ID NO: 409 - DNA, 20 bp, Artificial Sequence
  sgRNA
ttcagaagtgcctagctgtt

SEQ ID NO: 410 - DNA, 20 bp, Artificial Sequence
  sgRNA
gaagtgcctagctgttggga

SEQ ID NO: 411 - DNA, 20 bp, Artificial Sequence
  sgRNA
ctttaaccatcccaacagct

SEQ ID NO: 412 - DNA, 20 bp, Artificial Sequence
  sgRNA
tgcagtactgacatcggttt

SEQ ID NO: 413 - DNA, 20 bp, Artificial Sequence
  sgRNA
gaaatctgcagtactgacat

SEQ ID NO: 414 - DNA, 20 bp, Artificial Sequence
  sgRNA
tttcagaagtgtctcagtgt

SEQ ID NO: 415 - DNA, 20 bp, Artificial Sequence
  sgRNA
gaagtgtctcagtgtcggga

SEQ ID NO: 416 - DNA, 20 bp, Artificial Sequence
  sgRNA
tctcagtgtcgggatggtta

SEQ ID NO: 417 - DNA, 20 bp, Artificial Sequence
  sgRNA
cagcttgtccaactggtcgg

SEQ ID NO: 418 - DNA, 20 bp, Artificial Sequence
  sgRNA
tgtgagccaggctgggtaga

SEQ ID NO: 419 - DNA, 20 bp, Artificial Sequence
  sgRNA
gcctggctcacagtgtcaga

SEQ ID NO: 420 - DNA, 20 bp, Artificial Sequence
  sgRNA
cgaccagttggacaagctgc

SEQ ID NO: 421 - DNA, 20 bp, Artificial Sequence
  sgRNA
cggaggatcttatgctgaac

SEQ ID NO: 422 - DNA, 20 bp, Artificial Sequence
  sgRNA
cccacagactcggcactcaa

SEQ ID NO: 423 - DNA, 20 bp, Artificial Sequence
  sgRNA
gccattgagtgccgagtctg

SEQ ID NO: 424 - DNA, 20 bp, Artificial Sequence
  sgRNA
cattgagtgccgagtctgtg

SEQ ID NO: 425 - DNA, 20 bp, Artificial Sequence
  sgRNA
ataaataagcttcaatcgga

SEQ ID NO: 426 - DNA, 20 bp, Artificial Sequence
  sgRNA
ggccgagaaggagaagctgt

SEQ ID NO: 427 - DNA, 20 bp, Artificial Sequence
  sgRNA
aatgtctgtgccaagacgtt

SEQ ID NO: 428 - DNA, 20 bp, Artificial Sequence
  sgRNA
gtccacctgagagtgcacag

SEQ ID NO: 429 - DNA, 20 bp, Artificial Sequence
  sgRNA
ttgcaggtctggcacttgaa

SEQ ID NO: 430 - DNA, 20 bp, Artificial Sequence
  sgRNA
caagtagtgtttctgcaggt

SEQ ID NO: 431 - DNA, 20 bp, Artificial Sequence
  sgRNA
ttgtgctgctaaatctcttg

SEQ ID NO: 432 - DNA, 20 bp, Artificial Sequence
  sgRNA
caatgactcgagagttctcc

SEQ ID NO: 433 - DNA, 20 bp, Artificial Sequence
  sgRNA
agtcattgtcatgaccacaa

SEQ ID NO: 434 - DNA, 20 bp, Artificial Sequence
  sgRNA
tgtcatgaccacaaaggaag

SEQ ID NO: 435 - DNA, 20 bp, Artificial Sequence
  sgRNA
gagtatgcgctcaaagaata

SEQ ID NO: 436 - DNA, 20 bp, Artificial Sequence
  sgRNA
ggacagccatgtagataaac

SEQ ID NO: 437 - DNA, 20 bp, Artificial Sequence
  sgRNA
gctgcagaaatgtttcgacg

SEQ ID NO: 438 - DNA, 20 bp, Artificial Sequence
  sgRNA
gtttgtcaagacaaatcatc

SEQ ID NO: 439 - DNA, 20 bp, Artificial Sequence
  sgRNA
aaatcatccggctaccacta

SEQ ID NO: 440 - DNA, 20 bp, Artificial Sequence
  sgRNA
atcatccggctaccactatg

SEQ ID NO: 441 - DNA, 20 bp, Artificial Sequence
  sgRNA
aatcatccggctaccactat

SEQ ID NO: 442 - DNA, 20 bp, Artificial Sequence
  sgRNA
caagccctgcttcgtttgcc

SEQ ID NO: 443 - DNA, 20 bp, Artificial Sequence
  sgRNA
cttgtcctggcaaacgaagc

SEQ ID NO: 444 - DNA, 20 bp, Artificial Sequence
  sgRNA
ttgtcctggcaaacgaagca

SEQ ID NO: 445 - DNA, 20 bp, Artificial Sequence
  sgRNA
ctgcagaagtgctttgaagt

SEQ ID NO: 446 - DNA, 20 bp, Artificial Sequence
  sgRNA
cctgcagaagtgctttgaag

SEQ ID NO: 447 - DNA, 20 bp, Artificial Sequence
  sgRNA
ggcttatagacccgaggagg

SEQ ID NO: 448 - DNA, 20 bp, Artificial Sequence
  sgRNA
catggcttatagacccgagg

SEQ ID NO: 449 - DNA, 20 bp, Artificial Sequence
  sgRNA
aagcatggcttatagacccg

SEQ ID NO: 450 - DNA, 20 bp, Artificial Sequence
  sgRNA
ttgtcattgcatacaaagca

SEQ ID NO: 451 - DNA, 20 bp, Artificial Sequence
  sgRNA
gtatgcaatgacaagtcttc

SEQ ID NO: 452 - DNA, 20 bp, Artificial Sequence
  sgRNA
tattcggtgtgtaaagaaaa

SEQ ID NO: 453 - DNA, 20 bp, Artificial Sequence
  sgRNA
tgtccagcagctgctgttca

SEQ ID NO: 454 - DNA, 20 bp, Artificial Sequence
  sgRNA
atgtccagcagctgctgttc

SEQ ID NO: 455 - DNA, 20 bp, Artificial Sequence
  sgRNA
attgggttagaaacaatggg

SEQ ID NO: 456 - DNA, 20 bp, Artificial Sequence
  sgRNA
ggatactatgaagcagaatt

SEQ ID NO: 457 - DNA, 20 bp, Artificial Sequence
  sgRNA
tttcacgggaccaggctggg

SEQ ID NO: 458 - DNA, 20 bp, Artificial Sequence
  sgRNA
ggaccaggctgggaggcacg

SEQ ID NO: 459 - DNA, 20 bp, Artificial Sequence
  sgRNA
gagcctcgtgcctcccagcc

SEQ ID NO: 460 - DNA, 20 bp, Artificial Sequence
  sgRNA
atgcacatcagcttgagaaa

SEQ ID NO: 461 - DNA, 20 bp, Artificial Sequence
  sgRNA
agctgatgtgcatcggcaag

SEQ ID NO: 462 - DNA, 20 bp, Artificial Sequence
  sgRNA
ccaggcttctgggccttatg

SEQ ID NO: 463 - DNA, 20 bp, Artificial Sequence
  sgRNA
tgttcgatgatctccacata

SEQ ID NO: 464 - DNA, 20 bp, Artificial Sequence
  sgRNA
tcatcgaacagccgaagcaa

SEQ ID NO: 465 - DNA, 20 bp, Artificial Sequence
  sgRNA
atcgaacagccgaagcaacg

SEQ ID NO: 466 - DNA, 20 bp, Artificial Sequence
  sgRNA
ctgccgggatggctactatg

SEQ ID NO: 467 - DNA, 20 bp, Artificial Sequence
  sgRNA
tggtgttcagcacagcttcc

SEQ ID NO: 468 - DNA, 20 bp, Artificial Sequence
  sgRNA
ggtgttcagcacagcttcct

SEQ ID NO: 469 - DNA, 20 bp, Artificial Sequence
  sgRNA
gaagtcggcacggccttccc

SEQ ID NO: 470 - DNA, 20 bp, Artificial Sequence
  sgRNA
cacatcagcttgagagaagt

SEQ ID NO: 471 - DNA, 20 bp, Artificial Sequence
  sgRNA
tctctcaagctgatgtgcac

SEQ ID NO: 472 - DNA, 20 bp, Artificial Sequence
  sgRNA
tcgagactgtggcgggctgc

SEQ ID NO: 473 - DNA, 20 bp, Artificial Sequence
  sgRNA
agactgtggcgggctgcggg

SEQ ID NO: 474 - DNA, 20 bp, Artificial Sequence
  sgRNA
ctgtggcgggctgcgggagg

SEQ ID NO: 475 - DNA, 20 bp, Artificial Sequence
  sgRNA
gacggcgtgcctggtgtgga

SEQ ID NO: 476 - DNA, 20 bp, Artificial Sequence
  sgRNA
tgtcgtagacaggctcagag

SEQ ID NO: 477 - DNA, 20 bp, Artificial Sequence
  sgRNA
tgtgagcacagggcaggatg

SEQ ID NO: 478 - DNA, 20 bp, Artificial Sequence
  sgRNA
agaagctgtgagcacagggc

SEQ ID NO: 479 - DNA, 20 bp, Artificial Sequence
  sgRNA
ctgacagaagctgtgagcac

SEQ ID NO: 480 - DNA, 20 bp, Artificial Sequence
  sgRNA
tgacagaagctgtgagcaca

SEQ ID NO: 481 - DNA, 20 bp, Artificial Sequence
  sgRNA
tcatctgtaggcgacaaatg

SEQ ID NO: 482 - DNA, 20 bp, Artificial Sequence
  sgRNA
ctgacgaggacaggagtagg

SEQ ID NO: 483 - DNA, 20 bp, Artificial Sequence
  sgRNA
cttctgacgaggacaggagt

SEQ ID NO: 484 - DNA, 20 bp, Artificial Sequence
  sgRNA
acagttcttctgacgaggac

SEQ ID NO: 485 - DNA, 20 bp, Artificial Sequence
  sgRNA
cacacgtaatgacaccataa

SEQ ID NO: 486 - DNA, 20 bp, Artificial Sequence
  sgRNA
tatggtgtcattacgtgtga

SEQ ID NO: 487 - DNA, 20 bp, Artificial Sequence
  sgRNA
caggctgtcccgctgcttct

SEQ ID NO: 488 - DNA, 20 bp, Artificial Sequence
  sgRNA
gcgggacagcctgtatgctg

SEQ ID NO: 489 - DNA, 20 bp, Artificial Sequence
  sgRNA
gcttctgcacctcagcatac

SEQ ID NO: 490 - DNA, 20 bp, Artificial Sequence
  sgRNA
tacggagtcatcacgtgtga

SEQ ID NO: 491 - DNA, 20 bp, Artificial Sequence
  sgRNA
gaggatgtccaagaagcagc

SEQ ID NO: 492 - DNA, 20 bp, Artificial Sequence
  sgRNA
atctgtggggacaagtcatc

SEQ ID NO: 493 - DNA, 20 bp, Artificial Sequence
  sgRNA
cacaggtgataaccccgtag

SEQ ID NO: 494 - DNA, 20 bp, Artificial Sequence
  sgRNA
tacggggttatcacctgtga

SEQ ID NO: 495 - DNA, 20 bp, Artificial Sequence
  sgRNA
ctacggggttatcacctgtg

SEQ ID NO: 496 - DNA, 20 bp, Artificial Sequence
  sgRNA
ctcgggacatgcccagagcc

SEQ ID NO: 497 - DNA, 20 bp, Artificial Sequence
  sgRNA
ttcaacgaccttcgattcgt

SEQ ID NO: 498 - DNA, 20 bp, Artificial Sequence
  sgRNA
gttcaacgaccttcgattcg

SEQ ID NO: 499 - DNA, 20 bp, Artificial Sequence
  sgRNA
cactgcggcccacgaatcga

SEQ ID NO: 500 - DNA, 20 bp, Artificial Sequence
  sgRNA
ttcgattcgtgggccgcagt

SEQ ID NO: 501 - DNA, 20 bp, Artificial Sequence
  sgRNA
tgcgcacgagctctcccgcg

SEQ ID NO: 502 - DNA, 20 bp, Artificial Sequence
  sgRNA
gccacgaaccaaaatttccg

SEQ ID NO: 503 - DNA, 20 bp, Artificial Sequence
  sgRNA
cagggcttccacggaaattt

SEQ ID NO: 504 - DNA, 20 bp, Artificial Sequence
  sgRNA
aaaatttccgtggaagccct

SEQ ID NO: 505 - DNA, 20 bp, Artificial Sequence
  sgRNA
cggtactaccttaagcacca

SEQ ID NO: 506 - DNA, 20 bp, Artificial Sequence
  sgRNA
tcagcttgccatggtgctta

SEQ ID NO: 507 - DNA, 20 bp, Artificial Sequence
  sgRNA
ctccactcgcttatagtggt

SEQ ID NO: 508 - DNA, 20 bp, Artificial Sequence
  sgRNA
catcgccctgtttccaccca

SEQ ID NO: 509 - DNA, 20 bp, Artificial Sequence
  sgRNA
agggcgatgaagaagagaaa

SEQ ID NO: 510 - DNA, 20 bp, Artificial Sequence
  sgRNA
ggccttctccagctcttcca

SEQ ID NO: 511 - DNA, 20 bp, Artificial Sequence
  sgRNA
tcagttcatggtggctctgg

SEQ ID NO: 512 - DNA, 20 bp, Artificial Sequence
  sgRNA
agaagtctcttcaccactgg

SEQ ID NO: 513 - DNA, 20 bp, Artificial Sequence
  sgRNA
ctgggatggaaaaaatcagc

SEQ ID NO: 514 - DNA, 20 bp, Artificial Sequence
  sgRNA
gggtctggaggagcaggtgg

SEQ ID NO: 515 - DNA, 20 bp, Artificial Sequence
  sgRNA
tgaaaaagcagtgaaaagtc

SEQ ID NO: 516 - DNA, 20 bp, Artificial Sequence
  sgRNA
attgcaattctgagtggtga

SEQ ID NO: 517 - DNA, 20 bp, Artificial Sequence
  sgRNA
caggcgcttcacgatcgggg

SEQ ID NO: 518 - DNA, 20 bp, Artificial Sequence
  sgRNA
tcgtgaagcgcctgctgggt

SEQ ID NO: 519 - DNA, 20 bp, Artificial Sequence
  sgRNA
gaagggcgagcagaacgggc

SEQ ID NO: 520 - DNA, 20 bp, Artificial Sequence
  sgRNA
cgagaaggcggtcaagagct

SEQ ID NO: 521 - DNA, 20 bp, Artificial Sequence
  sgRNA
gctcaagaagacggggcagt

SEQ ID NO: 522 - DNA, 20 bp, Artificial Sequence
  sgRNA
agttcattcttgtgtagatc

SEQ ID NO: 523 - DNA, 20 bp, Artificial Sequence
  sgRNA
acaacccgctcatagtgata

SEQ ID NO: 524 - DNA, 20 bp, Artificial Sequence
  sgRNA
gaatccatatcactatgagc

SEQ ID NO: 525 - DNA, 20 bp, Artificial Sequence
  sgRNA
tgatgtgtcatagacaaggt

SEQ ID NO: 526 - DNA, 20 bp, Artificial Sequence
  sgRNA
tctatgcccgtctgtggagg

SEQ ID NO: 527 - DNA, 20 bp, Artificial Sequence
  sgRNA
aggtgacgaggaagagaaat

SEQ ID NO: 528 - DNA, 20 bp, Artificial Sequence
  sgRNA
ggaagagaaatgggcagaaa

SEQ ID NO: 529 - DNA, 20 bp, Artificial Sequence
  sgRNA
gaaaaagctgaagaagaaga

SEQ ID NO: 530 - DNA, 20 bp, Artificial Sequence
  sgRNA
gaagggtgctatggaggagc

SEQ ID NO: 531 - DNA, 20 bp, Artificial Sequence
  sgRNA
tcgtgatggctctgcaaatc

SEQ ID NO: 532 - DNA, 20 bp, Artificial Sequence
  sgRNA
ccgcgcgccgacctccgctt

SEQ ID NO: 533 - DNA, 20 bp, Artificial Sequence
  sgRNA
ctggccgcccaagcggaggt

SEQ ID NO: 534 - DNA, 20 bp, Artificial Sequence
  sgRNA
cgcgccgacctccgcttggg

SEQ ID NO: 535 - DNA, 20 bp, Artificial Sequence
  sgRNA
gagcagcagctgcggtggcg

SEQ ID NO: 536 - DNA, 20 bp, Artificial Sequence
  sgRNA
accacttcagccggctctgc

SEQ ID NO: 537 - DNA, 20 bp, Artificial Sequence
  sgRNA
ggactgcaggctgggcccgg

SEQ ID NO: 538 - DNA, 20 bp, Artificial Sequence
  sgRNA
tggactgcaggctgggcccg

SEQ ID NO: 539 - DNA, 20 bp, Artificial Sequence
  sgRNA
gtaggacgagggcggctgcg

SEQ ID NO: 540 - DNA, 20 bp, Artificial Sequence
  sgRNA
cagcaggagggggagcgagt

SEQ ID NO: 541 - DNA, 20 bp, Artificial Sequence
  sgRNA
tctgaaggcgctcacgcact

SEQ ID NO: 542 - DNA, 20 bp, Artificial Sequence
  sgRNA
ccgggggcagcggcgtaaga

SEQ ID NO: 543 - DNA, 20 bp, Artificial Sequence
  sgRNA
gatcagcaagcgcctgggcg

SEQ ID NO: 544 - DNA, 20 bp, Artificial Sequence
  sgRNA
aaagtttccactccgcgccc

SEQ ID NO: 545 - DNA, 20 bp, Artificial Sequence
  sgRNA
cgttcatcgacgaggccaag

SEQ ID NO: 546 - DNA, 20 bp, Artificial Sequence
  sgRNA
gtgcagagcgcgcagccgct

SEQ ID NO: 547 - DNA, 20 bp, Artificial Sequence
  sgRNA
tgctgaccgatgcggagaag

SEQ ID NO: 548 - DNA, 20 bp, Artificial Sequence
  sgRNA
gtgcaccgcacgcagtcgct

SEQ ID NO: 549 - DNA, 20 bp, Artificial Sequence
  sgRNA
cgaggccaagcgactgcgtg

SEQ ID NO: 550 - DNA, 20 bp, Artificial Sequence
  sgRNA
actgcgtgcggtgcacatga

SEQ ID NO: 551 - DNA, 20 bp, Artificial Sequence
  sgRNA
ggtgcacatgaaggagtacc

SEQ ID NO: 552 - DNA, 20 bp, Artificial Sequence
  sgRNA
agaatccaccaacggaagtc

SEQ ID NO: 553 - DNA, 20 bp, Artificial Sequence
  sgRNA
ctcagctgccagacttccgt

SEQ ID NO: 554 - DNA, 20 bp, Artificial Sequence
  sgRNA
ctgaaggaacagaaaaacgc

SEQ ID NO: 555 - DNA, 20 bp, Artificial Sequence
  sgRNA
tgaaggaacagaaaaacgct

SEQ ID NO: 556 - DNA, 20 bp, Artificial Sequence
  sgRNA
ggtgcttcttaatgagctct

SEQ ID NO: 557 - DNA, 20 bp, Artificial Sequence
  sgRNA
aaaaccttgaccccggagga

SEQ ID NO: 558 - DNA, 20 bp, Artificial Sequence
  sgRNA
ctggccctcctccggggtca

SEQ ID NO: 559 - DNA, 20 bp, Artificial Sequence
  sgRNA
ctgtctctggccctcctccg

SEQ ID NO: 560 - DNA, 20 bp, Artificial Sequence
  sgRNA
gccagagacagggcttaatt

SEQ ID NO: 561 - DNA, 20 bp, Artificial Sequence
  sgRNA
gctggacaaaattctggagc

SEQ ID NO: 562 - DNA, 20 bp, Artificial Sequence
  sgRNA
gcagctggacacacgctacc

SEQ ID NO: 563 - DNA, 20 bp, Artificial Sequence
  sgRNA
gtacagcgacagcttcccca

SEQ ID NO: 564 - DNA, 20 bp, Artificial Sequence
  sgRNA
gactctcaatccaaggtgcc

SEQ ID NO: 565 - DNA, 20 bp, Artificial Sequence
  sgRNA
gagattatgaaacaccaacg

SEQ ID NO: 566 - DNA, 20 bp, Artificial Sequence
  sgRNA
agaggacattggactcttgc

SEQ ID NO: 567 - DNA, 20 bp, Artificial Sequence
  sgRNA
attaccacagctacatgcat

SEQ ID NO: 568 - DNA, 20 bp, Artificial Sequence
  sgRNA
cttcagtttctgaaagacaa

SEQ ID NO: 569 - DNA, 20 bp, Artificial Sequence
  sgRNA
ctatgatgacaactttccta

SEQ ID NO: 570 - DNA, 20 bp, Artificial Sequence
  sgRNA
tccggcatctgctagctcag

SEQ ID NO: 571 - DNA, 20 bp, Artificial Sequence
  sgRNA
aatacaattggatgaacagt

SEQ ID NO: 572 - DNA, 20 bp, Artificial Sequence
  sgRNA
ggacgataacgaccacaggg

SEQ ID NO: 573 - DNA, 20 bp, Artificial Sequence
  sgRNA
gacgataacgaccacaggga

SEQ ID NO: 574 - DNA, 20 bp, Artificial Sequence
  sgRNA
catggacgataacgaccaca

SEQ ID NO: 575 - DNA, 20 bp, Artificial Sequence
  sgRNA
gcagtggttcgacggggtga

SEQ ID NO: 576 - DNA, 20 bp, Artificial Sequence
  sgRNA
gtggttcgacggggtgatgg

SEQ ID NO: 577 - DNA, 20 bp, Artificial Sequence
  sgRNA
ccgtgcgcctgctggtgggg

SEQ ID NO: 578 - DNA, 20 bp, Artificial Sequence
  sgRNA
agacctcttgattcgtttca

SEQ ID NO: 579 - DNA, 20 bp, Artificial Sequence
  sgRNA
cagacctcttgattcgtttc

SEQ ID NO: 580 - DNA, 20 bp, Artificial Sequence
  sgRNA
actttctggcagtggtttga

SEQ ID NO: 581 - DNA, 20 bp, Artificial Sequence
  sgRNA
gcagtggtttgatggcgtga

SEQ ID NO: 582 - DNA, 20 bp, Artificial Sequence
  sgRNA
ctaagcctggcccaggggcc

SEQ ID NO: 583 - DNA, 20 bp, Artificial Sequence
  sgRNA
taagcctggcccaggggccc

SEQ ID NO: 584 - DNA, 20 bp, Artificial Sequence
  sgRNA
aggagagatcatgaacaaca

SEQ ID NO: 585 - DNA, 20 bp, Artificial Sequence
  sgRNA
agaaaataaagcgctgtgag

SEQ ID NO: 586 - DNA, 20 bp, Artificial Sequence
  sgRNA
tccggagacagcgtttggtg

SEQ ID NO: 587 - DNA, 20 bp, Artificial Sequence
  sgRNA
tgaacttggaccacaacagg

SEQ ID NO: 588 - DNA, 20 bp, Artificial Sequence
  sgRNA
tagtgatgatcatctctgtc

SEQ ID NO: 589 - DNA, 20 bp, Artificial Sequence
  sgRNA
ccacggtgaaggacaggaat

SEQ ID NO: 590 - DNA, 20 bp, Artificial Sequence
  sgRNA
tggagcccacaagccattac

SEQ ID NO: 591 - DNA, 20 bp, Artificial Sequence
  sgRNA
tggtggaccagcaccactgg

SEQ ID NO: 592 - DNA, 20 bp, Artificial Sequence
  sgRNA
agccagacttggacgggggg

SEQ ID NO: 593 - DNA, 20 bp, Artificial Sequence
  sgRNA
tgcagccagacttggacggg

SEQ ID NO: 594 - DNA, 20 bp, Artificial Sequence
  sgRNA
ttccaccccccgtccaagtc

SEQ ID NO: 595 - DNA, 20 bp, Artificial Sequence
  sgRNA
tcgataggtgcagccagact

SEQ ID NO: 596 - DNA, 20 bp, Artificial Sequence
  sgRNA
catcactcaactccagtctg

SEQ ID NO: 597 - DNA, 20 bp, Artificial Sequence
  sgRNA
cggagcgggagaaggagcgc

SEQ ID NO: 598 - DNA, 20 bp, Artificial Sequence
  sgRNA
ggagcgggagaaggagcgcc

SEQ ID NO: 599 - DNA, 20 bp, Artificial Sequence
  sgRNA
gcgggagaaggagcgccggg

SEQ ID NO: 600 - DNA, 20 bp, Artificial Sequence
  sgRNA
gcgggtccgcgacatcaatg

SEQ ID NO: 601 - DNA, 20 bp, Artificial Sequence
  sgRNA
gcacctgctgctccaggctg

SEQ ID NO: 602 - DNA, 20 bp, Artificial Sequence
  sgRNA
ttgaggggtttcttgatgac

SEQ ID NO: 603 - DNA, 20 bp, Artificial Sequence
  sgRNA
taaagcatgaacgcattgag

SEQ ID NO: 604 - DNA, 20 bp, Artificial Sequence
  sgRNA
gtaaagcatgaacgcattga

SEQ ID NO: 605 - DNA, 20 bp, Artificial Sequence
  sgRNA
tcaaccagatcctgggtcgc

SEQ ID NO: 606 - DNA, 20 bp, Artificial Sequence
  sgRNA
atacccgggctggtccgcgc

SEQ ID NO: 607 - DNA, 20 bp, Artificial Sequence
  sgRNA
atacaacatgaaggcattca

SEQ ID NO: 608 - DNA, 20 bp, Artificial Sequence
  sgRNA
tacaacatgaaggcattcag

SEQ ID NO: 609 - DNA, 20 bp, Artificial Sequence
  sgRNA
tatacaacatgaaggcattc

SEQ ID NO: 610 - DNA, 20 bp, Artificial Sequence
  sgRNA
taatggctgcactttccttc

SEQ ID NO: 611 - DNA, 20 bp, Artificial Sequence
  sgRNA
atagttgtcccgggctgacc

SEQ ID NO: 612 - DNA, 20 bp, Artificial Sequence
  sgRNA
acaggcttctggggctcatt

SEQ ID NO: 613 - DNA, 20 bp, Artificial Sequence
  sgRNA
taggcagacacaggcttctg

SEQ ID NO: 614 - DNA, 20 bp, Artificial Sequence
  sgRNA
tttgttctttcgtgataccc

SEQ ID NO: 615 - DNA, 20 bp, Artificial Sequence
  sgRNA
caaaagtagcatttggattc

SEQ ID NO: 616 - DNA, 20 bp, Artificial Sequence
  sgRNA
cgaagattgtggcatcaatg

SEQ ID NO: 617 - DNA, 20 bp, Artificial Sequence
  sgRNA
actggcttctgtggctcatt

SEQ ID NO: 618 - DNA, 20 bp, Artificial Sequence
  sgRNA
taggccgacactggcttctg

SEQ ID NO: 619 - DNA, 20 bp, Artificial Sequence
  sgRNA
tgagccacagaagccagtgt

SEQ ID NO: 620 - DNA, 20 bp, Artificial Sequence
  sgRNA
tgtaggccgccaaggctttc

SEQ ID NO: 621 - DNA, 20 bp, Artificial Sequence
  sgRNA
ggagtacctgaaagccttgg

SEQ ID NO: 622 - DNA, 20 bp, Artificial Sequence
  sgRNA
agacgtgccctgtgcagttg

SEQ ID NO: 623 - DNA, 20 bp, Artificial Sequence
  sgRNA
gtgctgtgacttcttgtaga

SEQ ID NO: 624 - DNA, 20 bp, Artificial Sequence
  sgRNA
caagaagtcacagcacatga

SEQ ID NO: 625 - DNA, 20 bp, Artificial Sequence
  sgRNA
ctgtacggcggtctctccca

SEQ ID NO: 626 - DNA, 20 bp, Artificial Sequence
  sgRNA
tctgtacggcggtctctccc

SEQ ID NO: 627 - DNA, 20 bp, Artificial Sequence
  sgRNA
ccacttatgccattactgtg

SEQ ID NO: 628 - DNA, 20 bp, Artificial Sequence
  sgRNA
caaagcgctccccacagtaa

SEQ ID NO: 629 - DNA, 20 bp, Artificial Sequence
  sgRNA
gtgttgtctcaggttacttc

SEQ ID NO: 630 - DNA, 20 bp, Artificial Sequence
  sgRNA
ggtgttgtctcaggttactt

SEQ ID NO: 631 - DNA, 20 bp, Artificial Sequence
  sgRNA
gggtatggaggtgttgtctc

SEQ ID NO: 632 - DNA, 20 bp, Artificial Sequence
  sgRNA
ttgcttccgtcgatgaccac

SEQ ID NO: 633 - DNA, 20 bp, Artificial Sequence
  sgRNA
ggtcatcgacggaagcaatg

SEQ ID NO: 634 - DNA, 20 bp, Artificial Sequence
  sgRNA
agaagacttccttgttccca

SEQ ID NO: 635 - DNA, 20 bp, Artificial Sequence
  sgRNA
ggctgttccttcctccaaga

SEQ ID NO: 636 - DNA, 20 bp, Artificial Sequence
  sgRNA
cgaaggaagttgtccaggct

SEQ ID NO: 637 - DNA, 20 bp, Artificial Sequence
  sgRNA
aagaggccataaagatatca

SEQ ID NO: 638 - DNA, 20 bp, Artificial Sequence
  sgRNA
tcctggtattcacaccatca

SEQ ID NO: 639 - DNA, 20 bp, Artificial Sequence
  sgRNA
cacaccatcacggcgtgtcc

SEQ ID NO: 640 - DNA, 20 bp, Artificial Sequence
  sgRNA
acaccatcacggcgtgtcca

SEQ ID NO: 641 - DNA, 20 bp, Artificial Sequence
  sgRNA
ctcagaaaattatccagact

SEQ ID NO: 642 - DNA, 20 bp, Artificial Sequence
  sgRNA
cgagtgtggaatctgtagaa

SEQ ID NO: 643 - DNA, 20 bp, Artificial Sequence
  sgRNA
atcatttgattgagcacatg

SEQ ID NO: 644 - DNA, 20 bp, Artificial Sequence
  sgRNA
gcacatgcggctgcactctg

SEQ ID NO: 645 - DNA, 20 bp, Artificial Sequence
  sgRNA
ccacacttgtcacattgata

SEQ ID NO: 646 - DNA, 20 bp, Artificial Sequence
  sgRNA
gtgattcatgtgttgagagt

SEQ ID NO: 647 - DNA, 20 bp, Artificial Sequence
  sgRNA
acagacatgtggtccttgta

SEQ ID NO: 648 - DNA, 20 bp, Artificial Sequence
  sgRNA
agcatactatgctatgaaca

SEQ ID NO: 649 - DNA, 20 bp, Artificial Sequence
  sgRNA
ttcagcagttcatcagagtt

SEQ ID NO: 650 - DNA, 20 bp, Artificial Sequence
  sgRNA
ctgaggaaggcccacagcaa

SEQ ID NO: 651 - DNA, 20 bp, Artificial Sequence
  sgRNA
ctcaggaatttgtgaaggaa

SEQ ID NO: 652 - DNA, 20 bp, Artificial Sequence
  sgRNA
tccactgtgtatcctctgat

SEQ ID NO: 653 - DNA, 20 bp, Artificial Sequence
  sgRNA
ctccactgtgtatcctctga

SEQ ID NO: 654 - DNA, 20 bp, Artificial Sequence
  sgRNA
acccatcagaggatacacag

SEQ ID NO: 655 - DNA, 20 bp, Artificial Sequence
  sgRNA
gaggtttgaattcagactga

SEQ ID NO: 656 - DNA, 20 bp, Artificial Sequence
  sgRNA
ggattctttgatgtcttatg

SEQ ID NO: 657 - DNA, 20 bp, Artificial Sequence
  sgRNA
aggatctgtcccaggctcct

SEQ ID NO: 658 - DNA, 20 bp, Artificial Sequence
  sgRNA
gacttggcaggatctgtccc

SEQ ID NO: 659 - DNA, 20 bp, Artificial Sequence
  sgRNA
ggaatgatgacagacttggc

SEQ ID NO: 660 - DNA, 20 bp, Artificial Sequence
  sgRNA
agcttccctttaaggccttc

SEQ ID NO: 661 - DNA, 20 bp, Artificial Sequence
  sgRNA
gcatgtaggggatgtaggac

SEQ ID NO: 662 - DNA, 20 bp, Artificial Sequence
  sgRNA
gggcccggtggctgagccca

SEQ ID NO: 663 - DNA, 20 bp, Artificial Sequence
  sgRNA
aattccatgggctcagccac

SEQ ID NO: 664 - DNA, 20 bp, Artificial Sequence
  sgRNA
attccatgggctcagccacc

SEQ ID NO: 665 - DNA, 20 bp, Artificial Sequence
  sgRNA
cagccaccgggcccctccct

SEQ ID NO: 666 - DNA, 20 bp, Artificial Sequence
  sgRNA
gacagcaaaccggaagctga

SEQ ID NO: 667 - DNA, 20 bp, Artificial Sequence
  sgRNA
aggacttcggacttcttcta

SEQ ID NO: 668 - DNA, 20 bp, Artificial Sequence
  sgRNA
gaagaagtccgaagtcctat

SEQ ID NO: 669 - DNA, 20 bp, Artificial Sequence
  sgRNA
ctagctgaccgataggactt

SEQ ID NO: 670 - DNA, 20 bp, Artificial Sequence
  sgRNA
atactgaaacagttgataaa

SEQ ID NO: 671 - DNA, 20 bp, Artificial Sequence
  sgRNA
ctcaaagactgatccttctg

SEQ ID NO: 672 - DNA, 20 bp, Artificial Sequence
  sgRNA
ggacagaaaaatccagcctt

SEQ ID NO: 673 - DNA, 20 bp, Artificial Sequence
  sgRNA
gagtagaagagggagcaaat

SEQ ID NO: 674 - DNA, 20 bp, Artificial Sequence
  sgRNA
atttgctccctcttctactc

SEQ ID NO: 675 - DNA, 20 bp, Artificial Sequence
  sgRNA
ctactcaggtgaaaaagcaa

SEQ ID NO: 676 - DNA, 20 bp, Artificial Sequence
  sgRNA
gaagcgggcagatccagctg

SEQ ID NO: 677 - DNA, 20 bp, Artificial Sequence
  sgRNA
ccaggaggaattgccacagc

SEQ ID NO: 678 - DNA, 20 bp, Artificial Sequence
  sgRNA
ccagctgtggcaattcctcc

SEQ ID NO: 679 - DNA, 20 bp, Artificial Sequence
  sgRNA
tgtcggagagcagctccagg

SEQ ID NO: 680 - DNA, 20 bp, Artificial Sequence
  sgRNA
cgctgtcggagagcagctcc

SEQ ID NO: 681 - DNA, 20 bp, Artificial Sequence
  sgRNA
acagctggcgttggcgctgt

SEQ ID NO: 682 - DNA, 20 bp, Artificial Sequence
  sgRNA
ccaggtgatacagctggcgt

SEQ ID NO: 683 - DNA, 20 bp, Artificial Sequence
  sgRNA
ccaacgccagctgtatcacc

SEQ ID NO: 684 - DNA, 20 bp, Artificial Sequence
  sgRNA
cccctcccaggtgatacagc

SEQ ID NO: 685 - DNA, 20 bp, Artificial Sequence
  sgRNA
caacgccagctgtatcacct

SEQ ID NO: 686 - DNA, 20 bp, Artificial Sequence
  sgRNA
cgccagctgtatcacctggg

SEQ ID NO: 687 - DNA, 20 bp, Artificial Sequence
  sgRNA
gccagctgtatcacctggga

SEQ ID NO: 688 - DNA, 20 bp, Artificial Sequence
  sgRNA
ccagctgtatcacctgggag

SEQ ID NO: 689 - DNA, 20 bp, Artificial Sequence
  sgRNA
ctccccgttggtcccctccc

SEQ ID NO: 690 - DNA, 20 bp, Artificial Sequence
  sgRNA
atcacctgggaggggaccaa

SEQ ID NO: 691 - DNA, 20 bp, Artificial Sequence
  sgRNA
tcacctgggaggggaccaac

SEQ ID NO: 692 - DNA, 20 bp, Artificial Sequence
  sgRNA
cacctgggaggggaccaacg

SEQ ID NO: 693 - DNA, 20 bp, Artificial Sequence
  sgRNA
cgtcattttgaactccccgt

SEQ ID NO: 694 - DNA, 20 bp, Artificial Sequence
  sgRNA
caacggggagttcaaaatga

SEQ ID NO: 695 - DNA, 20 bp, Artificial Sequence
  sgRNA
caaaatgacggaccccgatg

SEQ ID NO: 696 - DNA, 20 bp, Artificial Sequence
  sgRNA
aatgacggaccccgatgagg

SEQ ID NO: 697 - DNA, 20 bp, Artificial Sequence
  sgRNA
agcgcctggccacctcatcg

SEQ ID NO: 698 - DNA, 20 bp, Artificial Sequence
  sgRNA
cggaccccgatgaggtggcc

SEQ ID NO: 699 - DNA, 20 bp, Artificial Sequence
  sgRNA
cagcgcctggccacctcatc

SEQ ID NO: 700 - DNA, 20 bp, Artificial Sequence
  sgRNA
ccagcgcctggccacctcat

SEQ ID NO: 701 - DNA, 20 bp, Artificial Sequence
  sgRNA
ccgatgaggtggccaggcgc

SEQ ID NO: 702 - DNA, 20 bp, Artificial Sequence
  sgRNA
cgatgaggtggccaggcgct

SEQ ID NO: 703 - DNA, 20 bp, Artificial Sequence
  sgRNA
gatgaggtggccaggcgctg

SEQ ID NO: 704 - DNA, 20 bp, Artificial Sequence
  sgRNA
tttccgctcgccccagcgcc

SEQ ID NO: 705 - DNA, 20 bp, Artificial Sequence
  sgRNA
tggccaggcgctggggcgag

SEQ ID NO: 706 - DNA, 20 bp, Artificial Sequence
  sgRNA
agcttgtcgtaattcatgtt

SEQ ID NO: 707 - DNA, 20 bp, Artificial Sequence
  sgRNA
catagtaataacggagggcc

SEQ ID NO: 708 - DNA, 20 bp, Artificial Sequence
  sgRNA
tttatcatagtaataacgga

SEQ ID NO: 709 - DNA, 20 bp, Artificial Sequence
  sgRNA
ttttatcatagtaataacgg

SEQ ID NO: 710 - DNA, 20 bp, Artificial Sequence
  sgRNA
tgtttttatcatagtaataa

SEQ ID NO: 711 - DNA, 20 bp, Artificial Sequence
  sgRNA
aacattatgaccaaagtgca

SEQ ID NO: 712 - DNA, 20 bp, Artificial Sequence
  sgRNA
atatcttttgccgtgcactt

SEQ ID NO: 713 - DNA, 20 bp, Artificial Sequence
  sgRNA
gcttacaaatttgacttcca

SEQ ID NO: 714 - DNA, 20 bp, Artificial Sequence
  sgRNA
agtggaagccattgctctcg

SEQ ID NO: 715 - DNA, 20 bp, Artificial Sequence
  sgRNA
aatggcaactggtccccttc

SEQ ID NO: 716 - DNA, 20 bp, Artificial Sequence
  sgRNA
gaagatgggcgggagtcttc

SEQ ID NO: 717 - DNA, 20 bp, Artificial Sequence
  sgRNA
cagcttgtccaactggtcgg

SEQ ID NO: 718 - DNA, 20 bp, Artificial Sequence
  sgRNA
ccaaagccaccattgcaaag

SEQ ID NO: 719 - DNA, 20 bp, Artificial Sequence
  sgRNA
gaacatcggctacagccagg

SEQ ID NO: 720 - DNA, 20 bp, Artificial Sequence
  sgRNA
cgctgtcggacagtagttcc

SEQ ID NO: 721 - DNA, 20 bp, Artificial Sequence
  sgRNA
gccatggaagtcaaacttgt